(19) United States
(12) Patent Application Publication
(10) Pub. No.: US 2025/0223590 A1
(43) Pub. Date: Jul. 10, 2025

(54) Nucleic acid regulation of ApoE

(71) Applicant: uniQure biopharma B.V., Amsterdam (NL)

(72) Inventors: Lisa Maria Hentschel, Amsterdam (NL); Amila Zuko, Amsterdam (NL); Ying Pui Liu, Amsterdam (NL); - Anggakusuma, Amsterdam (NL); Javier Gustavo Villamil, Amsterdam (NL)

(73) Assignee: uniQure biopharma B.V., Amsterdam (NL)

(21) Appl. No.: 18/851,078

(22) PCT Filed: Apr. 12, 2023
(86) PCT No.: PCT/EP2023/059507

§ 371 Date: Sep. 26, 2024

(30) Foreign Application Priority Data
Apr. 12, 2022 (EP) 22167926.9
Oct. 7, 2022 (EP) 22200272.7
Mar. 17, 2023 (EP) 23162681.3

(51) Int. Cl. C12N 15/113; C12N 15/86

(52) U.S. Cl. CPC C12N 15/113; C12N 15/86; C12N 2310/14; C12N 2310/531; C12N 2750/14143

(57) ABSTRACT

The present invention relates to a nucleic acid comprising two RNA encoding sequences, wherein the each of the RNA comprises a hairpin and a guide sequence substantially complementary to part of APOE gene, and to associated AAVs, compositions, pharmaceutical compositions and uses in treatments thereof.

### Filed of Invention

The present invention relates to the fields of biotechnology, medicine and gene therapy. The invention relates to a nucleic acid comprising two or more RNA encoding sequences, one which contains a guide sequence substantially complementary to part of an APOE gene, associated compositions, pharmaceutical composition and uses in treatment thereof.

### BACKGROUND OF THE INVENTION

Alzheimer's disease (AD) is a progressive neurodegenerative disorder that is considered to be the leading cause of dementia in adults (Van Cauwenberghe et al. 2016, Genet Med.; 18:421:30). The disease is characterized by the accumulation of extracellular amyloid-β (Δβ) plaques and intracellular neurofibrillary tangles that cause neuronal cell death and glial cell activation, finally leading to a reduction in brain function. The formation of amyloid plaques is due to the cleavage of the amyloid precursor protein (APP) by β- and γ-secretase. The formation of neurofibrillary tangles is due, instead, to the misfolding of tau proteins (Pospich & Raunser 2017, Science; 358:45-46).

One of the genetic factors that increase the risk of developing AD relates to the human apolipoprotein (APOE) gene. The human APOE gene has single nucleotide polymorphisms (SNPs) that create three major allelic variants: 82, 83, and 84 (i.e. APOE isoforms E2, E3, and E4) (Belbin et al. 2007, Hum Mol Genet.; 16:2199-208). The &4 variant is involved in late-onset AD (LOAD) and a carrier of APOEε4 increases the risk of AD onset in an allelic number-dependent manner i.e. having one APOEε4 allele shifts the age of onset an average of 2-5 years earlier, whereas the presence of two APOEε4 alleles shifts onset to 5-10 years earlier. Importantly, 40-65% of patients with AD carry at least one APOEε4 allele. In addition, APOE4 has also been observed to be associated with atherosclerosis, unfavourable outcomes in traumatic brain injury (TBI) and other diseases. In contrast, the APOEε2 and/or APOEε3 alleles exert a protective or a neutral effect against AD development (Yamazaki et al. 2016, CNS Drugs.; 30:773-89).

The APOE protein, encoded by the APOE gene, is a secreted, lipid-transporting protein that is found in peripheral and central body systems, such as in the central nervous system (CNS). In the periphery, APOE is mainly secreted by hepatocytes, while in the CNS, astrocytes are the major source of APOE (Chernick et al. 2019, Neurosci Lett.; 708:134306).

The SNPs in the APOE gene induce differences at the amino acid residues located at positions 130 and 176, also respectively referred to as position 112 and 158 when the signal peptide of the protein is excluded (APOE: 2, Cys112/Cys158; APOE: 3, Cys112/Arg158; APOEε4, Arg112/Arg158). These single amino acid polymorphisms are considered to severely affect the structure and function of APOE, thereby affecting AB metabolism, aggregation, deposition, and tau phosphorylation. (Liu et al. 2013, Nat Rev Neurol.; 9:106-18).

Given the complex pathophysiology of AD and the associated gene regulatory networks, there is currently no promising therapy for treating AD, and therefore there is an urgent need to find such a therapy.

### SUMMARY OF INVENTION

The present invention solves the problem by applying nucleic acid technology, wherein said nucleic acid include two or more RNA encoding sequences, one which contains a guide sequence substantially complementary to part of an APOE gene, and the other supporting the expression of the first, possibly through cluster formation. Therefore, increasing the silencing of an APOE gene.

In one aspect, there is provided, an expression cassette comprising the nucleic acid according to the invention, wherein the expression cassette is a DNA molecule.

In one aspect, there is provided, an adeno-associated virus (AAV) vector comprising the expression cassette according to the invention.

In one aspect, there is provided, a pharmaceutical composition comprising the nucleic acid according to the invention, an expression cassette according to the invention, or an AAV vector according to the invention and at least one pharmaceutically acceptable excipient.

In one aspect, there is provided a nucleic acid according to the invention, or the expression cassette according to the invention, or the AAV vector according to the invention, of the pharmaceutical composition according to the invention for use as a medicament.

In one aspect, there is provided, a kit comprising the nucleic acid according to the invention, or the expression cassette according to the invention, or the AAV vector according to the invention, or the pharmaceutical composition according to the invention, wherein the kit further comprises an immunosuppressive agent.

In one aspect, there is provided an expression cassette comprising a nucleic acid which encodes one or more APOE2 and APOE3 proteins selected from SEQ ID NOs. 249-254 for use in gene therapy.

### DETAILED DESCRIPTION OF THE INVENTION

### Definitions

Unless defined otherwise, technical and scientific terms used herein have the same meaning as commonly understood by one of ordinary skill in the art to which this disclosure belongs. One skilled in the art will recognize many methods and materials similar or equivalent to those described herein, which could be used in the practice of the present invention. Indeed, the present invention is in no way limited to the method.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”. For the purposes of the present invention, the term “obtained” is considered to be a preferred embodiment of the term “obtainable”. If hereinafter e.g. an antibody is defined to be obtainable from a specific source, this is also to be understood to disclose an antibody that is obtained from this source.

As used herein, the term “and/or” indicates that one or more of the stated cases may occur, alone or in combination with at least one of the stated cases, up to with all of the stated cases.

As used herein, with “At least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

The word “about” or “approximately” when used in association with a numerical value (e.g. about 10) preferably means that the value may be the given value (of 10) more or less 0.1% of the value. As used herein, “an effective amount” is meant the amount of an agent required to ameliorate the symptoms of a disease relative to an untreated patient. The effective amount of active agent(s) used to practice the present invention for therapeutic treatment of, for example a cancer, varies depending upon the manner of administration, the age, body weight, and general health of the subject. Ultimately, the attending physician or veterinarian will decide the appropriate amount and dosage regimen. Such amount is referred to as an “effective” amount, which may be determined as genome copies per kilogram (GC/kg). Thus, in connection with the administration of a drug which, in the context of the current disclosure, is “effective against” a disease or condition indicates that administration in a clinically appropriate manner results in a beneficial effect for at least a statistically significant fraction of patients, such as an improvement of symptoms, a cure, a reduction in at least one disease sign or symptom, extension of life, improvement in quality of life, or other effect generally recognized as positive by medical doctors familiar with treating the particular type of disease or condition.

The use of a substance as a medicament as described in this document can also be interpreted as the use of said substance in the manufacture of a medicament. Similarly, whenever a substance is used for treatment or as a medicament, it can also be used for the manufacture of a medicament for treatment. Products for use as a medicament described herein can be used in methods of treatments, wherein such methods of treatment comprise the administration of the product for use.

The terms “homology”, “sequence identity” and the like are used interchangeably herein. Sequence identity is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods.

“Sequence identity” and “sequence similarity” can be determined by alignment of two peptides or two nucleotide sequences using global or local alignment algorithms, depending on the length of the two sequences. Sequences of similar lengths are preferably aligned using global alignment algorithms (e.g. Needleman Wunsch) which align the sequences optimally over the entire length, while sequences of substantially different lengths are preferably aligned using local alignment algorithms (e.g. Smith Waterman). Sequences may then be referred to as “substantially identical” or “essentially similar” when they (when optimally aligned by for example the programs GAP or BESTFIT using default parameters) share at least a certain minimum percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins, the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). When sequences have a substantially different overall length, local alignments, such as those using the Smith Waterman algorithm, are preferred.

5 Alternatively, percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, 10 wordlength=12 to obtain nucleotide sequences homologous to oxidoreductase nucleic acid molecules of the invention. BLAST protein searches can be performed with the BLASTx program, score=50, wordlength =3 to obtain amino acid sequences homologous to protein molecules of the invention. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default 15 parameters of the respective programs (e.g., BLASTx and BLASTn) can be used. See the homepage of the National Center for Biotechnology Information at http://www.ncbi.nlm.nih.gov/.

As used herein, the term “selectively hybridizing”, “hybridizes selectively” and similar terms are 20 intended to describe conditions for hybridization and washing under which nucleotide sequences at least 66%, at least 70%, at least 75%, at least 80%, more preferably at least 85%, even more preferably at least 90%, preferably at least 95%, more preferably at least 98% or more preferably at least 99% homologous to each other typically remain hybridized to each other. That is to say, such hybridizing sequences may share at least 45%, at least 50%, at least 55%, at least 60%, at least 65, at least 70%, at least 75%, at least 25 80%, more preferably at least 85%, even more preferably at least 90%, more preferably at least 95%, more preferably at least 98% or more preferably at least 99% sequence identity.

A preferred, non-limiting example of such hybridization conditions is hybridization in 6× sodium chloride/sodium citrate (SSC) at about 45° C., followed by one or more washes in 1×SSC, 0.1% SDS at about 50° C., preferably at about 55° C., preferably at about 60° C. and even more preferably at about 65° C. Highly stringent conditions include, for example, hybridization at about 68° C. in 5×SSC/5×Denhardt's solution/1.0% SDS and washing in 0.2×SSC/0.1% SDS at room temperature. Alternatively, washing may be performed at 42° C.

The skilled artisan will know which conditions to apply for stringent and highly stringent hybridization conditions. Additional guidance regarding such conditions is readily available in the art, for example, in Sambrook et al., 1989, Molecular Cloning, A Laboratory Manual, Cold Spring Harbor Press, N.Y.; and Ausubel et al. (eds.), Sambrook and Russell (2001) “Molecular Cloning: A Laboratory Manual (3rd edition), Cold Spring Harbor Laboratory, Cold Spring Harbor Laboratory Press, New York 1995, Current Protocols in Molecular Biology, (John Wiley & Sons, N.Y.).

Of course, a polynucleotide which hybridizes only to a poly-A sequence (such as the 3′ terminal poly(A) tract of mRNAs), or to a complementary stretch of T (or U) residues, would not be included in a polynucleotide of the invention used to specifically hybridize to a portion of a nucleic acid of the invention, since such a polynucleotide would hybridize to any nucleic acid molecule containing a poly(A) stretch or the complement thereof (e.g., practically any double-stranded cDNA clone).

A “nucleic acid construct” or “nucleic acid vector” is herein understood to mean a man-made nucleic acid molecule resulting from the use of recombinant DNA technology. The term “nucleic acid construct” therefore does not include naturally occurring nucleic acid molecules although a nucleic acid construct may comprise (parts of) naturally occurring nucleic acid molecules. A “vector” is a nucleic acid construct (typically DNA or RNA) that serves to transfer an exogenous nucleic acid sequence (i.e. DNA or RNA) into a host cell. A vector is preferably maintained in the host by at least one of autonomous replication and integration into the host cell's genome. The terms “expression vector” or “expression construct” refer to nucleotide sequences that are capable of affecting the expression of a gene in host cells or host organisms compatible with such sequences. These expression vectors typically include at least one “expression cassette” that is the functional unit capable of affecting the expression of a sequence encoding a product to be expressed and wherein the coding sequence is operably linked to the appropriate expression control sequences, which at least comprises a suitable transcription regulatory sequence and optionally, 3′ transcription termination signals. Additional factors necessary or helpful in effecting expression may also be present, such as expression enhancer elements. The expression vector will be introduced into a suitable host cell and be able to affect the expression of the coding sequence in an in vitro cell culture of the host cell. A preferred expression vector will be suitable for the expression of viral proteins and/or nucleic acids, particularly recombinant AAV proteins and/or nucleic acids.

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer or biological entity.

The term “reporter” may be used interchangeably with marker, although it is mainly used to refer to visible markers, such as green fluorescent protein (GFP) or luciferase.

The terms “protein” or “polypeptide” are used interchangeably and refer to molecules consisting of a chain of amino acids, without reference to a specific mode of action, size, 3-dimensional structure or origin.

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′-nontranslated sequence (3′-end) comprising a polyadenylation site. “Expression of a gene” refers to the process wherein a DNA region which is operably linked to appropriate regulatory regions, particularly a promoter, is transcribed into an RNA, which is biologically active, i.e. which is capable of being translated into a biologically active protein or peptide.

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain. If homologous to a host cell, a nucleic acid sequence encoding a polypeptide will typically (but not necessarily) be operably linked to another (heterologous) promoter sequence and, if applicable, another (heterologous) secretory signal sequence and/or terminator sequence than in its natural environment. It is understood that the regulatory sequences, signal sequences, terminator sequences, etc. may also be homologous to the host cell. In this context, the use of only “homologous” sequence elements allows the construction of “self-cloned” genetically modified organisms (GMO's) (self-cloning is defined herein as in European Directive 98/81/EC Annex II). When used to indicate the relatedness of two nucleic acid sequences the term “homologous” means that one single-stranded nucleic acid sequence may hybridize to a complementary single-stranded nucleic acid sequence. The degree of hybridization may depend on a number of factors including the amount of identity between the sequences and the hybridization conditions such as temperature and salt concentration as discussed later.

The terms “heterologous” and “exogenous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous and exogenous nucleic acids or proteins are not endogenous to the cell into which they are introduced but have been obtained from another cell or are synthetically or recombinantly produced. Generally, though not necessarily, such nucleic acids encode proteins, i.e. exogenous proteins, that are not normally produced by the cell in which the DNA is transcribed or expressed. Similarly, exogenous RNA encodes for proteins not normally expressed in the cell in which the exogenous RNA is present. Heterologous/exogenous nucleic acids and proteins may also be referred to as foreign nucleic acids or proteins. Any nucleic acid or protein that one of skill in the art would recognize as foreign to the cell in which it is expressed is herein encompassed by the term heterologous or exogenous nucleic acid or protein. The terms heterologous and exogenous also apply to non-natural combinations of nucleic acid or amino acid sequences, i.e. combinations where at least two of the combined sequences are foreign with respect to each other.

As used herein, the term “non-naturally occurring” when used in reference to an organism means that the organism has at least one genetic alternation that is not normally found in a naturally occurring strain of the referenced species, including wild-type strains of the referenced species. Genetic alterations include, for example, modifications introducing expressible nucleic acids encoding proteins or enzymes, other nucleic acid additions, nucleic acid deletions, nucleic acid substitutions, or other functional disruption of the organism's genetic material. Such modifications include, for example, coding regions and functional fragments thereof for heterologous or homologous polypeptides for the referenced species. Additional modifications include, for example, non-coding regulatory regions in which the modifications alter the expression of a gene or operon. Genetic modifications to nucleic acid molecules encoding enzymes, or functional fragments thereof, can confer a biochemical reaction capability or a metabolic pathway capability to the non-naturally occurring organism that is altered from its naturally occurring state.

As used herein, the term “operably linked” refers to a linkage of polynucleotide (or polypeptide) elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame.

An expression control sequence is “operably linked” to a nucleotide sequence when the expression control sequence controls and regulates the transcription and/or the translation of the nucleotide sequence. Thus, an expression control sequence can include promoters, enhancers, internal ribosome entry sites (IRES), transcription terminators, a start codon in front of a protein-encoding gene, splicing signal for introns, and stop codons.

The term “expression control sequence” is intended to include, at a minimum, a sequence whose presence is designed to influence expression, and can also include additional advantageous components. For example, leader sequences and fusion partner sequences are expression control sequences. The term can also include the design of the nucleic acid sequence such that undesirable, potential initiation codons in and out of frame, are removed from the sequence. It can also include the design of the nucleic acid sequence such that undesirable potential splice sites are removed. It includes sequences or polyadenylation sequences (pA) which direct the addition of a polyA tail, i.e., a string of adenine residues at the 3′-end of a mRNA, sequences referred to as polyA sequences. It also can be designed to enhance mRNA stability. Expression control sequences that affect the transcription and translation stability, e.g., promoters, as well as sequences that affect the translation, e.g., Kozak sequences, are known in insect cells. Expression control sequences can be of such nature as to modulate the nucleotide sequence to which it is operably linked such that lower expression levels or higher expression levels are achieved.

As used herein the term “codon optimization” refers to experimental approaches designed to improve the codon composition of a recombinant gene based on various criteria without altering the amino acid sequence. This is possible because most amino acids are encoded by more than one codon. Most codon-optimization approaches avoid the use of rare codons. However, different approaches vary in the extent of other features considered, including mRNA elements that can inhibit expression, nucleotide context of the initiation codon, mRNA secondary structures, sequence repeats, nucleotide composition, internal ribosome entry sites, promoter sequences, and putative splice donor and acceptor sites. In addition, some programs consider protein structural information, intragenic poly(A) sites, stop codons in alternative reading frames, and dinucleotides that are targets for RNase cleavage, mutation, and methylation-dependent gene silencing. The person skilled in the art has within their understanding the requirements needed to design such a codon-optimised nucleic acid.

### DETAILED DESCRIPTION OF THE INVENTION

The present inventors have set out to develop nucleic acids comprising RNA encoding sequences to modify APOE expression in a cell. The human APOE gene is associated with an increase in the risk of developing AD. In particular, the 84 variant is involved in late-onset AD (LOAD) and a carrier of APOEε4 increases the risk of AD onset in an allelic number-dependent manner i.e. having one APOEε4 allele shifts the age of onset an average of 2-5 years earlier, whereas the presence of two APOEε4 alleles shifts onset to 5-10 years earlier. Importantly, 40-65% of patients with AD carry at least one APOEε4 allele. In contrast, the APOEε2 and/or APOEε3 alleles exert a protective or a neutral effect against AD development. Hence, reducing the RNA expression levels is aimed to reduce the neuropathology associated with at least APOE4 expression. Using a gene therapy approach as outlined herein is to thereby significantly benefit affected human patients by reversing, preventing, slowing down the progression of, or completely halting neuropathologies.

In a first aspect of the invention, there is provided a nucleic acid comprising a sequence encoding a first RNA and a sequence encoding a second RNA, wherein the second RNA comprises a guide sequence of at least 19 nucleotides substantially complementary to part of an APOE gene, and wherein the first RNA and second RNA each comprise a hairpin.

### RNA

The term “nucleic acid” as used herein takes its regular meaning in the art. Thus, the term “RNA” or “RNA molecule” or “ribonucleic acid molecule” as used herein refers to a polymer of ribonucleotides (e.g., 2, 3, 4, 5, 10, 15, 20, 25, 30, or more ribonucleotides) and the term “DNA” or “DNA molecule” or “deoxyribonucleic acid molecule” as used herein refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized DNA and RNA can be single-stranded (i.e., ssRNA and ssDNA, respectively) or multi-stranded (e.g., double stranded, i.e., dsRNA and dsDNA, respectively). “mRNA” or “messenger RNA” is single-stranded RNA that specifies the amino acid sequence of one or more polypeptide chains. This information is translated during protein synthesis when ribosomes bind to the mRNA.

As used herein, the term “small interfering RNA” (“siRNA”) (also referred to in the art as “short interfering RNAs”) refers to an RNA (or RNA analogue) comprising between about 10-50 nucleotides (or nucleotide analogues) which is capable of directing or mediating RNA interference. Preferably, an siRNA comprises between about 15-30 nucleotides or nucleotide analogues, more preferably between about 16-25 nucleotides (or nucleotide analogues), even more preferably between about 18-23 nucleotides (or nucleotide analogues), and even more preferably between about 19-22 nucleotides (or nucleotide analogues) (e.g., 19, 20, 21 or 22 nucleotides or nucleotide analogues). The term “short” siRNA refers to a siRNA comprising about 21 nucleotides (or nucleotide analogues), for example, 19, 20, 21 or 22 nucleotides. The term “long” siRNA refers to a siRNA comprising about 24-25 nucleotides, for example, 23, 24, 25 or 26 nucleotides. Short siRNAs may, in some instances, include fewer than 19 nucleotides, e.g., 16, 17 or 18 nucleotides, provided that the shorter siRNA retains the ability to mediate RNAi. Likewise, long siRNAs may, in some instances, include more than 26 nucleotides, provided that the longer siRNA retains the ability to mediate RNAi absent further processing, e.g., enzymatic processing, to a short siRNA.

As used herein, the term “RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be induced, for example, to silence the expression of target genes. Double stranded RNA structures that are suitable for inducing RNAi are well known in the art. For example, a small interfering RNA (siRNA) can induce RNAi. An siRNA comprises two separate RNA strands, one strand comprising a first RNA sequence and the other strand comprising a second RNA sequence, thus a first and a second strand. An siRNA design that is often used involves consecutive base pairs with a 3′ overhang. The first and/or second strand may comprise a 3′-overhang. The 3′-overhang preferably is a dinucleotide overhang on both strands of the siRNA. Such a design is based on observed Dicer processing of larger double stranded RNAs that results in siRNAs having these features. The 3′-overhang may be comprised in the first strand. The 3′-overhang may be in addition to the first strand. The length of the two strands of which an siRNA is composed may be 19, 20, 21, 22, 23, 24, 25, 26 or 27 nucleotides or more.

siRNAs may also serve as Dicer substrates. For example, a Dicer substrate may be a 27-mer consisting of two strands of RNA that have 27 consecutive base pairs. The first strand is positioned at the 3′-end of the 27-mer duplex. At the 3′-ends, like with siRNAs, each or one of the strands may comprise a two nucleotide overhang. The 3′-overhang may be comprised in the first strand. The 3′-overhang may be in addition to the first strand. 5′ from the first strand, additional sequences may be included that are either complementary to the target RNA sequence adjacent or not. The other end of the siRNA dicer substrate is blunt ended. This dicer substrate design may result in a preference in processing by Dicer such that an siRNA can be formed like the siRNA design as described above, having 19 consecutive base pairs and 2 nucleotide overhangs at both 3′-ends. In any case, siRNAs, or the like, are composed of two separate RNA strands (Fire et al. 1998, Nature. 1998 Feb. 19; 391 (6669): 806-1 1) each RNA strand comprising or consisting of the first and second RNA strand in accordance with to the invention. Thus, the nucleic acid of the invention may be said to derive a first and second RNA strand, which is the first RNA, and a third and fourth RNA strand, which is the second RNA. Alternative naming conventions for each first, second, third and fourth RNA strand are within the scope of the invention, which may be complementary, substantially complementary, or unique to each other, or in any other required arrangement as discussed herein.

The loop sequence may also be a stem-loop sequence, whereby the double stranded region of the shRNA is extended. Like the siRNA dicer substrate as described above, a shRNA can be processed by e.g. Dicer to provide for an siRNA having an siRNA design such as described above, having e.g. 19 consecutive base pairs and 2 nucleotide overhangs at both 3′-ends. In case the shRNA is to be processed by Dicer, it is preferred to have the first and second strands at the end of the shRNA, i.e. such that the putative strands of the siRNA are linked via a stem loop sequence: 5′-first strand-apical loop sequence-second strand-optional 2 nt overhang sequence-3′. Or, conversely, 5′-second strand-apical loop sequence-first strand-optional 2 nt overhang sequence-3′. Another shRNA design may be a shRNA structure that is processed by the RNAi machinery to provide for an activated RISC complex that does not require Dicer processing (Liu et al., Nucleic Acids Res. 2013 April 1; 41 (6): 3723-33, incorporated herein by reference), so called AgoshRNAs or Ago2 processed shRNAs, which are based on a structure very similar to the miR-451 scaffold as described below. Such a shRNA structure comprises in its loop sequence part of the first RNA sequence. Such a shRNA structure may also consist of the first strand, followed immediately by the second strand.

In one embodiment, there is provided a nucleic acid, wherein the sequence encoding the first RNA is followed by a spacer and the sequence encoding the second RNA, wherein the spacer is at least 50 nucleotides. Thus, preferably, in a 5′ to 3′ direction, the sequence encoding the first RNA is followed by a first spacer comprising at least 50 nucleotides, which spacer is followed by the sequence encoding the second RNA. It is understood that the 5′ to 3′ direction refers to the coding strand in case of a ds nucleic acid. It was fortuitously identified that the use of the spacer as described herein enables the downstream processing of the sequences encoding the first and second RNAs allowing their combinatorial effect. As above, said nucleic acid may be said to derive a first and second RNA strand, which is the first RNA, and a third and fourth RNA strand, which is the second RNA, wherein the sequences encoding the first and second RNA strands are followed by a spacer and the sequences encoding the third and fourth RNA strands, wherein the spacer is at least 50 nucleotides, such as at least 60, 65, 70, 75, 80, 85, 90, 95, 100, 105, 110, 115, 120, 125 or 130 nucleotides. In some embodiments, the spacer is between 60 to 130 nucleotides. In a preferred embodiment, the spacer is between 90 and 105 nucleotides. In a more preferred embodiment, the spacer is 92 nucleotides. In some embodiments of the invention, the spacer comprises SEQ ID NO. 237. Thus, the first and second RNA may also be known as an RNA cluster, when transcribed from physically adjacent genes. In a further embodiment, in case one or both RNAs are shRNA to be processed by Dicer, the relevant above mentioned shRNA structures are also applicable. In an alternative embodiment, in case one or both RNAs are AgoshRNAs, the relevant above mentioned shRNA structures are also applicable. Thus, one or both RNAs may be processed by the same or different RNAi machinery, such as Dicer and/or Ago2 of the RNAi machinery. In a preferred embodiment, the first RNA is processed by Dicer, therefore, the putative strands of the subsequent siRNA are linked via a stem loop sequence: 5′-first strand-apical loop sequence-second strand-optional 2 nt overhang sequence-3′ or, conversely, 5′ second strand-apical loop sequence-first strand-optional 2 nt overhang sequence-3′. As shown in the examples, the first and second strands of the invention, may be preferably incorporated in a pre-miRNA or a pri-miRNA scaffold derived from a pri-miRNA or pre-miRNA scaffold derived from miR-144 (SEQ ID NO. 235). In a preferred embodiment, the third and fourth strands of the invention are incorporated in a pri-miRNA or pre-miRNA scaffold derived from miR-451 (SEQ ID NO. 190). Thus the first and/or second RNA can be described as a hairpin or a double stranded RNA that is substantially complementary to itself. In a more preferred embodiment, the first RNA comprises SEQ ID NO. 235 (miR-144) or a variant thereof.

In some embodiments of the invention where the first RNA is incorporated in a pre-miRNA or a pri-miRNA scaffold derived from miR-144, the first RNA is mutated to reduce processing and/or expression of the first RNA. In some specific embodiments, SEQ ID NO. 235 or the variant thereof is mutated to reduce processing and/or expression of the first RNA. In some embodiments, the mutation is a single point mutation. In other words, the first RNA comprises a single point mutation to reduce processing and/or expression of the first RNA. In some preferred embodiments, the single point mutation is A-T-position 19 (SEQ ID NO. 236). The skilled person can easily determine whether this is the case by using standard assays and appropriate controls such as described in the examples and as known in the art.

Thus, in some embodiments, the first RNA is processed by Dicer and the second RNA is processed by Ago2, such as an AgoshRNA, or miR-451 mimic RNA and may be said to comprise, in its loop sequence, part of the second RNA sequence. Such a shRNA structure may also consist of the third strand, followed immediately by the fourth strand of the invention.

A double stranded RNA according to the invention may also be incorporated in a pre-miRNA or pri-miRNA scaffold. MicroRNAs, i.e. miRNA, are guide strands that originate from double stranded RNA molecules that are endogenously expressed e.g. in mammalian cells. Two or more miRNAs may also be comprised in a miRNA cluster, wherein the miRNAs in the cluster are transcribed in the same orientation and are not separated by a transcription unit or a miRNA in the opposite orientation. A miRNA is processed from a pre-miRNA precursor molecule, similar to the processing of a shRNA or an extended siRNA as described above, by the RNAi machinery and incorporated in an activated RNA-induced silencing complex (RISC) (Tijsterman M, Plasterk RH. Dicers at RISC; the mechanism of RNAi. Cell. 2004 Apr. 2; 1 17 (1): 1-3). A pre-miRNA is a hairpin RNA molecule that can be part of a larger RNA molecule (pri-miRNA), e.g.

comprised in an intron, which is first processed by Drosha to form a pre-miRNA hairpin molecule. In one embodiment, the hairpin in the second RNA comprises at least 40 nucleotides. The pre-miRNA molecule is a shRNA-like molecule that can subsequently be processed by Dicer or Ago2 to result in an siRNA-like double stranded RNA duplex (see FIGS. 2A and B).

The miRNA, i.e. the guide strand, that is part of the double stranded RNA duplex is subsequently incorporated in RISC. In one embodiment, the second RNA comprises a guide sequence of at least 19 nucleotides substantially complementary to part of an APOE gene, such as 20, 21, 22, 23, 24, 25 or 26 nucleotides. In a further embodiment, the second RNA comprises a guide sequence of at least 22 nucleotides substantially complementary to part of an APOE gene, preferably the guide sequence is specific to part of the APOE4 gene. It was fortuitously identified that the length of guide sequence as described herein provided to be the optimal length specific to part of the APOE4 gene to have the desired effect. Shorter sequences may lead to off target effects. An RNA molecule such as present in nature, i.e. a pri-miRNA, a pre-miRNA or a miRNA duplex, may be used as a scaffold for producing an artificial miRNA that specifically targets a gene of choice. Based on the predicted RNA structure of the RNA molecule as present in nature, e.g. as predicted using e.g. m-fold software using standard settings (Zuker. Nucleic Acids Res. 31 (13), 3406-3415, 2003), the natural miRNA sequence as it is present in the RNA structure (i.e. duplex, pre-miRNA or pri-miRNA), and the sequence present in the structure that is substantially complementary therewith are removed and replaced with a first strand and a second strand according to the invention, that are the first strand and second strand of the first RNA, or the first strand and second strand of the second RNA, which may also be referred to as the third and fourth strands. Thus, using the first and second strand for solely exemplary purpose, the first strand and the second strand are preferably selected such that the predicted secondary RNA structures that are formed, i.e. of the pre-miRNA, pri-miRNA and/or miRNA duplex, resemble the corresponding predicted original secondary structure of the natural RNA sequences. pre-miRNA, pri-miRNA and miRNA duplexes (that consist of two separate RNA strands that are hybridized via complementary base pairing) as found in nature, are often not fully base paired, i.e. not all nucleotides that correspond with the first and second strand as defined above are base paired, and the first and second strand are often not of the same length. How to use miRNA precursor molecules as scaffolds for any selected target RNA sequence and substantially complementary first strand is described e.g. in Liu YP Nucleic Acids Res. 2008 May; 36 (9): 281 1-24, which is incorporated herein by reference.

A pri-miRNA can be processed by the RNAi machinery of the cell. The pri-miRNA comprises flanking sequences at the 5′-end and the 3′-end of a pre-miRNA hairpin and/or shRNA like molecule. Such a pri-miRNA hairpin can be processed by Drosha to produce a pre-miRNA. The length of the flanking sequences can vary but may be around 80 nt in length (Zeng and Cullen, J Biol Chem. 2005 Jul. 29; 280 (30): 27595-603; Cullen, Mol Cell. 2004 Dec. 22; 16 (6): 861-5). The minimal length of the single-stranded flanks can easily be determined as when it becomes too short, the RNA molecule may lose its function because e.g. Drosha processing fails to result in sequence specific inhibition being reduced or even absent. In one embodiment, the pri-miRNA scaffold carrying the first and second strand according to the invention has a 5′-sequence flank and a 3′ sequence flank relative to the predicted pre-miRNA structure of at least 5, at least 10, at least 15, at least 20, at least 30, at least 40, or at least 50 nucleotides. Preferably, the pri-miRNA derived flanking sequences (5′ and 3′) comprised in the miRNA scaffold are derived from the same naturally occurring pri-miRNA sequence. Preferably, pre-miRNA and/or the pri-miRNA derived flanking sequences (5′ and 3′) and/or loop sequences comprised in the miRNA scaffold are derived from the same naturally occurring pri-miRNA sequence. As the (putative) guide strand RNA as comprised in the endogenous miRNA sequence can be replaced by a sequence including (or consisting of) the first strand, and the passenger strand sequence replaced by a sequence including (or consisting of) the second strand, it is understood that flanking sequences and/or loop sequences of the pri-miRNA or pre-miRNA sequences of the endogenous sequence may include minor sequence modifications such that the predicted structure of the scaffold miRNA sequence (e.g. M-fold predicted structure) is the same as the predicted structure of the endogenous miRNA sequence.

The first and second strands and the third and fourth strands, such as to form two double stranded RNAs, i.e. the first and second RNA of the invention, are encoded by an expression cassette. It is understood that when the double stranded RNAs are to be e.g. two siRNAs, consisting of two strands each, that there may be two or more expression cassettes required. When each double stranded RNA is comprised in a single RNA molecule, e.g. encoding a shRNA, pre-miRNA or pri-miRNA, one expression cassette per RNA molecule may suffice. A pol II expression cassette may comprise a promoter sequence a sequence encoding an RNA to be expressed followed by a polyadenylation sequence. In case the double stranded RNAs that are expressed comprises a pri-miRNA scaffold, the encoded RNA sequence may encode for intron sequences and exon sequences and 3′-UTR's and 5′-UTRs. A pol Ill expression cassette in general comprises a promoter sequence, followed by a sequence encoding an RNA (e.g. shRNA sequence, pre-miRNA, or a strand of the double stranded RNAs to be comprised in e.g. an siRNA or 5T extended siRNA). A pol I expression cassette may comprise a pol I promoter, followed by an RNA encoding sequence and a 3′-sequence Expression cassettes for double stranded RNAs are well known in the art, and any type of expression cassette can suffice, e.g. one may use a pol III promoter, a pol II promoter or a pol I promoter (i.a. ter Brake et al., Mol Ther. 2008 March; 16 (3): 557-64, Maczuga et al., BMC Biotechnol. 2012 Jul. 24; 12:42). In one embodiment, the expression cassette is a DNA molecule. Such DNA molecules may be useful in the application of further technologies and applications, providing a vector for the nucleic acids as described herein.

As is clear from the above, the first and second strands, thus, also the third and fourth strands, that are comprised in a double stranded RNA can contain additional nucleotides and/or nucleotide sequences. The double stranded RNA may be comprised in a single RNA sequence or comprised in two separate RNA strands. Whatever design is used, it is designed such that from the first and second RNA sequence an antisense RNA molecule comprising the first strand, thus, also the third strand, in whole or a substantial part thereof, of the invention can be processed by the RNAi machinery such that it is incorporated in the RISC complex to have its action, i.e. to induce RNAi against the RNA target sequence comprised in an RNA encoded by the APOE gene. The sequence comprising or consisting of the first strand, thus, also the third strand, in whole or a substantial part thereof, being capable of sequence specifically targeting RNA encoded by a human APOE gene. Hence, as long as the double stranded RNA is capable of inducing RNAi, such a double stranded RNA is contemplated in the invention. In one embodiment, the double stranded RNA according to the invention is comprised in a pre-miRNA scaffold, a pri-miRNA scaffold, a shRNA, or an siRNA. Preferably the first and second strand or third and fourth or all four strands as encoded by the expressed cassette are to be contained in a single transcript. It is understood that the expressed transcript in subsequent processing, i.e. cleavage, results in the single transcript being processed into multiple separate RNA molecules.

The term complementary is herein defined herein as nucleotides of a nucleic acid sequence that can bind to another nucleic acid sequence through hydrogen bonds, i.e. nucleotides that are capable of base pairing. Ribonucleotides, the building blocks of RNA are composed of monomers (nucleotides) containing a sugar, phosphate and a base that is either a purine (guanine, adenine) or pyrimidine (uracil, cytosine). Complementary RNA strands form double stranded RNA. A double stranded RNA may be formed from two separate complementary RNA strands or the two complementary RNA strands may be comprised in one RNA strand. In complementary RNA strands, the nucleotides cytosine and guanine (C and G) can form a base pair, guanine and uracil (G and U), and uracil and adenine (U and A) can form a base pair as well. The term substantial complementarity means that is not required to have the first and second RNA sequence to be fully complementary, or to have the first RNA sequence and target RNA sequence or sequences of RNA encoded by a human APOE gene to be fully complementary.

### Target Sequence

The second RNA that is to be expressed in accordance with the invention comprises, in whole or a substantial part thereof, a guide strand, also referred to as antisense strand as it is complementary (“anti”) to a sense target RNA sequence, the sense target RNA sequence being comprised in an RNA encoded by a human APOE gene. Thus, the second RNA also comprises a “sense strand”, that may have substantial sequence identity with, or be identical to, the target RNA sequence. Thus the second RNA can be described as a hairpin or a double stranded RNA that is substantially complementary to itself. Said double stranded RNA according to the invention is to induce RNA interference, thereby reducing expression of APOE transcripts, which includes knocking down of APOE derived transcripts. Transcripts that may be targeted may include spliced, including splice variants, and unspliced RNA transcripts such as encoded by SEQ ID NO.1. Hence, an RNA encoded by a human APOE gene is understood to comprise unspliced mRNAs comprising a 5′ untranslated region (UTR), intron and exon sequences, followed by a 3′ UTR and a polyA tail, and also splice variants thereof. Said double stranded RNA according to the invention may also induce transcriptional silencing. It is understood that in accordance with the invention, instead of providing an expression cassette, the third and fourth strands, which together encode the second RNA, may be provided.

In one embodiment the double stranded RNA according to the invention comprises a first RNA sequence and a second RNA sequence, i.e. the third and fourth RNA strands, wherein the first and second RNA sequence are substantially complementary, and wherein the first RNA sequence has a sequence length of at least 19 nucleotides and is substantially complementary to a target RNA sequence of an RNA encoded by a human APOE gene, which first RNA sequence is capable of inducing RNA interference to sequence specifically reduce expression of an RNA transcript comprising the target RNA sequence. In a further embodiment, said induction of RNA interference to reduce expression of an RNA transcript comprising the target RNA sequence means that it is to reduce APOE gene expression. As previously discussed, the APOE gene comprises single nucleotide polymorphisms (SNPs) that induce differences at the amino acid residues located at positions 112 and 158 in APOE isoforms (APOEε2, Cys112/Cys158; APOE: 3, Cys112/Arg158; APOEε4, Arg112/Arg158). These single amino acid polymorphisms are considered to severely affect the structure and function of APOE, thereby affecting AB metabolism, aggregation, deposition, and tau phosphorylation. (Liu et al. 2013, Nat Rev Neurol.; 9:106-18). The small difference between the APOE isoforms means that targeting using the RNA sequence as defined herein may lead to the reduced expression of all APOE, preferably that targeting using the RNA sequence as defined leads to the reduced expression of all APOE. However, in alternative embodiments, individual isoforms may be targeted. Design of the individual RNA sequences is within the expertise of the skilled person in the art. In some embodiments, the target RNA sequence targets part of an APOE gene, preferably the target RNA sequence targets part of the APOE4 gene. Thus, the invention looks to reduce the APOE 84 variant that is involved in late-onset AD (LOAD) since a carrier of APOEε4 is at increased risk of AD onset in an allelic number-dependent. In addition, the invention looks to reduce the development associated with atherosclerosis and the unfavourable outcomes in traumatic brain injury (TBI) associated with the APOEε4 variant (APOE4). Said “reducing” of the APOE4 involves the use of the target RNA sequence as described herein targeting part of the APOE4 gene. The length and target site of the target RNA sequence have been identified by the inventors to have the desired result to ameliorate the diseases associated with APOE4 expression as discussed above and shown in the examples.

Reducing expression of an APOE transcript is herein thus preferably understood as reducing the steady state level of a functional APOE mRNA in a target cell such that less of the mRNA is available in the cell for translation into the APOE protein, thereby reducing the steady state level of the protein in the target cell. Reducing expression of an APOE transcript therefore does not necessarily involve reducing de novo transcription of the APOE gene but rather increased degradation of an APOE mRNA and/or its precursors, e.g. unspliced RNA transcripts.

One can easily determine the extent of reduction of APOE gene expression, also for individual isotypes, by using standard luciferase reporter assays and appropriate controls such as described in the examples and as known in the art (Zhuang et al. 2006 Methods Mol Biol. 2006; 342:181-7). For example, a luciferase reporter comprising a target RNA sequence can be used to show that the double stranded RNA according to the invention is capable of sequence specific knock down. Furthermore, such as shown i.a. in the example section, levels of APOE expression can be determined by detecting APOE RNA (nuclear and/or cytoplasmic), or APOE protein.

As used herein, the term “RNA silencing” refers to a group of sequence-specific regulatory mechanisms (e.g. RNA interference (RNAi), transcriptional gene silencing (TGS), post-transcriptional gene silencing (PTGS), quelling, co-suppression, and translational repression) mediated by RNA molecules which result in the inhibition or “silencing” of the expression of a corresponding protein-coding gene. RNA silencing has been observed in many types of organisms, including plants, animals, and fungi.

It is understood that “substantially complementary” in this context means that it is not required to have all the nucleotides of the guide sequence and the target sequence to be base paired, i.e. to be fully complementary, or all the nucleotides of the guide sequence and the target sequence to be base paired. As long as the second RNA is capable of inducing RNA interference to thereby sequence-specifically target a sequence comprising the target RNA sequence, such substantial complementarity is contemplated in accordance with the invention.

The substantial complementarity between the strand complementary to the target RNA sequence, also referred to as part of an APOE gene, preferably consists of at most two mismatched nucleotides, more preferably having one mismatched nucleotide, most preferably having no mismatches. It is understood that one mismatched nucleotide means that over the entire length of the strand complementary to the target RNA sequence when base paired with the target RNA sequence one nucleotide does not base pair with the target RNA sequence. Having no mismatches means that all nucleotides of the strand complementary to the target RNA base pair with the target RNA sequence, having 2 mismatches means two nucleotides of the strand complementary to the target RNA do not base pair with the target RNA sequence. The strand complementary to the target RNA may also comprise additional nucleotides that do not have complementarity to the target RNA sequence, and may be longer than e.g. 21 nucleotides, in such a scenario, the substantial complementarity is determined over the entire length of the target RNA sequence. This means that the target RNA sequence in this embodiment has either no, one or two mismatches over its entire length when base paired with the strand complementary to the target RNA.

As shown in the example section, double stranded RNAs comprising a strand complementary to the target RNA length of 22 nucleotides were tested. These strands complementary to the target RNA had no mismatches and were fully complementary with the target RNA sequence. Having a few mismatches between the strand complementary to the target RNA and the target RNA sequence may however be allowed according to the invention, as long as the double stranded RNA according to the invention is capable of reducing the expression of transcripts comprising the target RNA sequence, such as a luciferase reporter or e.g. a transcript comprising the target RNA sequence. In this embodiment, substantial complementarity between the strand complementary to the target RNA and the target RNA sequence consists of having no, one or two mismatches over the entire length of either the strand complementary to the target RNA or the target RNA sequence encoded by an RNA of the human APOE, whichever is the shortest.

As said, a mismatch according to the invention means that a nucleotide of the first or third strand does not base pair with the target RNA sequence encoded by an RNA of the human APOE. Nucleotides that do not base pair are A and A, G and G, C and C, U and U, A and C, C and U, or A and G. A mismatch may also result from a deletion of a nucleotide, or an insertion of a nucleotide. When the mismatch is a deletion in the first or third strand sequence, this means that a nucleotide of the target RNA sequence is not base paired with the first or third strand sequence when compared with the entire length of the first or third strand sequence. Nucleotides that can base pair are A-U, G-C and G-U. A G-U base pair is also referred to as a G-U wobble, or wobble base pair. In one embodiment the number of G-U base pairs between the first or third strand sequence and the target RNA sequence is 0, 1 or 2. In one embodiment, there are no mismatches between the first or third strand sequence and the target RNA sequence and a G-U base pair or G-U pairs are allowed. Preferably, there may be no G-U base pairs between the first or third strand sequence and the target RNA sequence, or the first or third strand sequence and the target RNA sequence only have base pairs that are A-U or G-C. Preferably, there are no G-U base pairs and no mismatches between the first or third strand sequence and the target RNA sequence. The first or third strand sequence of the double stranded RNA according to the invention preferably is fully complementary to the target RNA sequence, said complementarity consisting of G-U, G-C and A-U base pairs. The first or third strand sequence of the double stranded RNA according to the invention more preferably is fully complementary to the target RNA sequence, said complementarity consisting of G-C and A-U base pairs. More preferably, it is the third strand, i.e. the first strand of the second RNA of the invention.

Therefore, in one embodiment the first strand of the second RNA and the target RNA sequence have at least 15, 16, 17, 18, or 19 nucleotides that base pair. Preferably the first strand of the second RNA and the target RNA sequence are substantially complementary, said complementarity comprising at least 19 base pairs. In another embodiment, the first strand of the second RNA has at least 8, 9, 10, 11, 12, 13 or 14 consecutive nucleotides that base pair with consecutive nucleotides of the target RNA sequence. In another embodiment, the first strand of the second RNA has at least 19 consecutive nucleotides that base pair with consecutive nucleotides of the target RNA sequence. In another embodiment, the first strand of the second RNA comprises at least 19 consecutive nucleotides that base pair with 19 consecutive nucleotides of the target RNA sequence. In still another embodiment, the first strand of the second RNA has at least 17 nucleotides that base pair with the target RNA sequence and has at least 15 consecutive nucleotides that base pair with consecutive nucleotides of the target RNA sequence. The sequence length of the first strand is preferably at most 21, 22, 23, 24, 25, 26, or 27 nucleotides. In another embodiment, the first strand of the second RNA has at least 20 consecutive nucleotides that base pair with 20 consecutive nucleotides of the target RNA sequence. In another embodiment, the first strand of the second RNA comprises at least 21 consecutive nucleotides that base pair with 21 consecutive nucleotides of the target RNA sequence.

As said, it may be not required to have full complementarity (i.e. full base pairing (no mismatches) and no G-U base pairs) between the first strand of the second RNA and the target RNA sequence as such a first strand of the second RNA can still allow for sufficient suppression of gene expression. Also, not having full complementarity may be contemplated for example to avoid or reduce off-target RNA sequence specific gene suppression while maintaining sequence specific inhibition of transcripts comprising the target RNA sequence. However, it may be preferred to have full complementarity as it may result in more potent inhibition. Without being bound by theory, having full complementarity between the first strand of the second RNA and the target RNA sequence may allow for the activated RISC complex comprising said first strand of the second RNA (or a substantial part thereof) to cleave its target RNA sequence, whereas having mismatches may hamper cleavage and can result in mainly allowing inhibition of translation, of which the latter may result in less potent inhibition.

With regard to the second strand on the second RNA, the second strand is substantially complementary with the first strand. The second strand combined with the first strand forms a double stranded RNA. As said, this is to form a suitable substrate for the RNA interference machinery such that a guide sequence derived from the first strand is comprised in the RISC complex in order to sequence specifically inhibit expression of its target, i.e. RNA encoded by a human APOE gene. The sequence of the second strand has sequence similarity with the target RNA sequence. However, the substantial complementarity if the second strand with the first strand may be selected to have less substantial complementarity as compared with the substantial complementarity between the first strand and the target RNA sequence. Hence, the second strand may comprise 0, 1, 2, 3, 4, or more mismatches, 0, 1, 2, 3, or more GU wobble base pairs, and may comprise insertions of 0, 1, 2, 3, 4, nucleotides and/or deletions of 0, 1, 2, 3, 4, nucleotides. Preferably the first strand and the second strand are substantially complementary, said complementarity comprising 0, 1, 2 or 3 G U base pairs and/or wherein said complementarity comprises at least 17 base pairs. These mismatches, G-U wobble base pairs, insertions and deletions, are with regard to the first strand, i.e. the double stranded region that is formed between the first and second strands. As long as the first and second strands can substantially base pair, and are capable of inducing sequence specific inhibition of an RNA encoded by a human APOE gene, such substantial complementarity is allowed according to the invention. It is also understood that substantially complementarity between the first and the second strands may depend on the double stranded RNA design of choice. It may depend for example on the miRNA scaffold that is chosen for in which the double stranded RNA is to be incorporated.

As is clear from the above, the substantial complementarity between the first strand and second strand of the second RNA, may comprise mismatches, deletions and/or insertions relative to a first and second RNA sequence being fully complementary (i.e. fully base paired). In one embodiment, the first and second strands of the second RNA have at least 11 consecutive base pairs. Hence, at least 11 consecutive nucleotides of the first strand and at least 11 consecutive nucleotides of the second strand are fully complementary. In another embodiment, the first and second strands of the second RNA have at least 15 nucleotides that base pair. Said base pairing between at least 15 nucleotides of the first strand and at least 15 nucleotides of the second strand may consist of G-U, G-C and A-U base pairs, or may consist of G-C and A-U base pairs. In another embodiment, the first and second RNA sequence have at least 15 nucleotides that base pair and have at least 11 consecutive base pairs. In another embodiment, the first RNA sequence and the second RNA sequence are substantially complementary, wherein said complementarity comprises at least 17 base pairs. Said 17 base pairs may preferably be 17 consecutive base pairs, said base pairing consisting of G-U, G-C and A-U base pairs or consisting of G-C and A-U base pairs.

As said, the current invention now provides for an expression cassette encoding the first strand and second strand of the second RNA wherein the first and second strands are substantially complementary, wherein the first strand has a sequence length of at least 19 nucleotides and is substantially complementary to a target RNA sequence comprised in an RNA encoded by a human APOE gene. As shown in the examples, suitable target RNA sequences in accordance with the invention are provided (see e.g. table 1). Hence, in one embodiment, an expression cassette is provided encoding a first strand and a second strand wherein the first and second strands are substantially complementary, wherein the first strand has a sequence length of at least 19 nucleotides, 20 nucleotides, 21 nucleotides, or 22 nucleotides and is substantially complementary to a target RNA sequence selected from the group listed in table 1 comprised in an RNA encoded by the human APOE gene.

TABLE 1
RNA target sequences encoded by the
human APOE gene.

| SEQ ID NO. | RNA TARGET SEQUENCE (5′-3′) |
|---|---|
| 2 | ACUGGCCAAUCACAGGCAGGAA |
| 3 | CUGGCCAAUCACAGGCAGGAAG |
| 4 | UGGCCAAUCACAGGCAGGAAGA |
| 5 | GGCCAAUCACAGGCAGGAAGAU |
| 6 | GCCAAUCACAGGCAGGAAGAUG |
| 7 | CCAAUCACAGGCAGGAAGAUGA |
| 8 | CAAUCACAGGCAGGAAGAUGAA |
| 9 | AAUCACAGGCAGGAAGAUGAAG |
| 10 | AUCACAGGCAGGAAGAUGAAGG |
| 11 | UCACAGGCAGGAAGAUGAAGGU |
| 12 | CACAGGCAGGAAGAUGAAGGUU |
| 13 | CAGGCAGGAAGAUGAAGGUUCU |
| 14 | AGGCAGGAAGAUGAAGGUUCUG |
| 15 | GGCAGGAAGAUGAAGGUUCUGU |
| 16 | GCAGGAAGAUGAAGGUUCUGUG |
| 17 | CAGGAAGAUGAAGGUUCUGUGG |
| 18 | AGGAAGAUGAAGGUUCUGUGGG |
| 19 | GAAGAUGAAGGUUCUGUGGGCU |
| 20 | AAGAUGAAGGUUCUGUGGGCUG |
| 21 | AUGAAGGUUCUGUGGGCUGCGU |
| 22 | UGAAGGUUCUGUGGGCUGCGUU |
| 23 | UGGGCUGCGUUGCUGGUCACAU |
| 24 | GGGCUGCGUUGCUGGUCACAUU |
| 25 | CUGCGUUGCUGGUCACAUUCCU |
| 26 | GCGUUGCUGGUCACAUUCCUGG |
| 27 | UUGCUGGUCACAUUCCUGGCAG |
| 28 | CACAUUCCUGGCAGGAUGCCAG |
| 29 | UUUGGGAUUACCUGCGCUGGGU |
| 30 | GCUGGGUGCAGACACUGUCUGA |
| 31 | CUGGGUGCAGACACUGUCUGAG |
| 32 | GGUGCAGACACUGUCUGAGCAG |
| 33 | GUGCAGACACUGUCUGAGCAGG |
| 34 | UGCAGACACUGUCUGAGCAGGU |
| 35 | GCAGACACUGUCUGAGCAGGUG |
| 36 | AGACACUGUCUGAGCAGGUGCA |
| 37 | GACACUGUCUGAGCAGGUGCAG |
| 38 | ACACUGUCUGAGCAGGUGCAGG |
| 39 | GAGACCAUGAAGGAGUUGAAGG |
| 40 | ACCAUGAAGGAGUUGAAGGCCU |
| 41 | CCAUGAAGGAGUUGAAGGCCUA |
| 42 | AUGAAGGAGUUGAAGGCCUACA |
| 43 | UGAAGGAGUUGAAGGCCUACAA |
| 44 | GAAGGAGUUGAAGGCCUACAAA |
| 45 | AAGGAGUUGAAGGCCUACAAAU |
| 46 | AGGAGUUGAAGGCCUACAAAUC |
| 47 | GGAGUUGAAGGCCUACAAAUCG |
| 48 | GAGUUGAAGGCCUACAAAUCGG |
| 49 | AGUUGAAGGCCUACAAAUCGGA |
| 50 | GUUGAAGGCCUACAAAUCGGAA |
| 51 | UGAAGGCCUACAAAUCGGAACU |
| 52 | GAAGGCCUACAAAUCGGAACUG |
| 53 | AAGGCCUACAAAUCGGAACUGG |
| 54 | AGGCCUACAAAUCGGAACUGGA |
| 55 | GGCCUACAAAUCGGAACUGGAG |
| 56 | GCCUACAAAUCGGAACUGGAGG |
| 57 | CCUACAAAUCGGAACUGGAGGA |
| 58 | CUACAAAUCGGAACUGGAGGAA |
| 59 | ACAAAUCGGAACUGGAGGAACA |
| 60 | CACGGCUGUCCAAGGAGCUGCA |
| 61 | ACGGCUGUCCAAGGAGCUGCAG |
| 62 | UCGCCUCCCACCUGCGCAAGCU |
| 63 | GAUGACCUGCAGAAGCGCCUGG |
| 64 | GACCUGCAGAAGCGCCUGGCAG |
| 65 | ACCUGCAGAAGCGCCUGGCAGU |
| 66 | CUGCAGAAGCGCCUGGCAGUGU |
| 67 | UGCAGAAGCGCCUGGCAGUGUA |
| 68 | ACCGCCUGGACGAGGUGAAGGA |
| 69 | CUGGACGAGGUGAAGGAGCAGG |
| 70 | GACGAGGUGAAGGAGCAGGUGG |
| 71 | CCCGCCUCAAGAGCUGGUUCGA |
| 72 | CCGCCUCAAGAGCUGGUUCGAG |
| 73 | GCUGGUGGAGAAGGUGCAGGCU |
| 74 | UGGAGAAGGUGCAGGCUGCCGU |
| 75 | CCUGUGCCCAGCGACAAUCACU |
| 76 | UGUGCCCAGCGACAAUCACUGA |
| 77 | GUGCCCAGCGACAAUCACUGAA |
| 78 | GCCCAGCGACAAUCACUGAACG |
| 79 | CAGCCUGCAGCGGGAGACCCUG |
| 80 | AGCCUGCAGCGGGAGACCCUGU |
| 81 | GGCGCGGACAUGGAGGACGUGC |
| 82 | GCGCGGACAUGGAGGACGUGCG |
| 83 | CGCGGACAUGGAGGACGUGCGC |
| 84 | GCGGACAUGGAGGACGUGCGCG |
| 85 | CGGACAUGGAGGACGUGCGCGG |
| 86 | GGACAUGGAGGACGUGCGCGGC |
| 87 | GACAUGGAGGACGUGCGCGGCC |
| 88 | ACAUGGAGGACGUGCGCGGCCG |
| 89 | CAUGGAGGACGUGCGCGGCCGC |
| 90 | UGCGCGGCCGCCUGGUGCAGUA |
| 91 | GGGUGGACCCUAGUUUAAUAAA |
| 92 | GGUGGACCCUAGUUUAAUAAAG |
| 93 | GUGGACCCUAGUUUAAUAAAGA |

Table 1. SEQ ID NOs. 2-93 correspond with target RNA sequences of transcripts encoded by the human APOE gene. For these target RNA sequences, it was found that surprisingly highly advantageous suitable third and fourth strands of the second RNA could be made in accordance with the invention to provide for an expression cassette encoding said second RNA, wherein said third and fourth are substantially complementary, wherein the third strand RNA sequence has a sequence length of at least 19 nucleotides and is substantially complementary to one of said target RNA sequences to highly efficiently induce RNAi to reduce APOE gene expression. It is understood that the reduction of APOE gene expression may include a reduction of transcripts that are related to tauopathies.

As shown in the examples, the third and fourth strands of the invention, may be preferably incorporated in a pre-miRNA or a pri-miRNA scaffold derived from miR-101 or a pri-miRNA or pre-miRNA scaffold derived from miR-451. In a preferred embodiment, the third and fourth strands of the invention are incorporated in a pri-miRNA or pre-miRNA scaffold derived from miR-451. These scaffolds were found to be in particular useful as these scaffolds both can induce RNA interference and can be combined in a single transcript. These scaffolds also allow to induce RNA interference that can result in mainly guide strand induced RNA interference. The pri-miR451 scaffold does not result in a passenger strand because the processing is different from the canonical miRNA processing pathway (Cheloufi et al.,2010 Jun. 3; 465 (7298): 584-9 and Yang et al., Proc Natl Acad Sci USA. 2010 Aug. 24; 107 (34): 15163-8). The pri-miR-101 scaffold is produced by the canonical miRNA processing pathway but it was found that many of the miR-101 scaffolds produced mainly guide strands (see e.g. C2, C4, C32, and C33) and very low amounts of passenger strands. Hence, both scaffolds represent excellent candidates to develop a gene therapy product as unwanted potential off-targeting by passenger strands can be largely, if not completely, avoided. As the passenger strand (corresponding to the second sequence) may result in the targeting of transcripts other than APOE RNA, using such scaffolds may allow one to avoid such unwanted targeting. Hence, it is preferred that scaffolds are selected that produce less than 15%, less than 10%, less than 5%, less than 4%, or less than 3% of passenger strands. In some embodiments, the second RNA is derived from a pri-miRNA scaffold is selected from the group consisting of pri-miR-21, pri-miR-22, pri-miR-26a, pri-miR-30a, pri-miR-33, pri-miR-122, pri-miR-375, pri-miR-199, pri-miR-99, pri-miR-194, pri-miR-155, and pri-miR-451. In a preferred embodiment, the second RNA is derived from a scaffold comprising SEQ ID NO. 190 (miR-451) or a variant thereof. Consequently, in one embodiment, the first RNA comprises SEQ ID NO. 235 (miR-144) or a variant thereof and the second RNA comprises SEQ ID NO. 190 (miR-451) or a variant thereof). The experimental data enclosed herein support the surprising finding that improved silencing of APOE is achieved when a miRNA cluster approach is applied. The specific combination of 144/451 scaffolds within the nucleic acid of the invention is particularly helpful within the context of gene therapy and RNA silencing. miR-144 and miR-451 are examples of clustered miRNAs regulated in trans, wherein miR-144 regulates the processing of miR-451 by Ago2. Specifically, miR-144 enhances miR-451 biogenesis in trans by repressing Dicer and, in turn, repressing global canonical miRNA processing (Kretov et al., 2020, Molecular Cell 78, 317-328). While it has been demonstrated that miR451 is the most abundant miRNA in erythrocytes and is clustered with miR144, wherein this cluster plays an intricate role during erythropoiesis, the inventors have further developed this cluster and now shown for the first time that these miRNAs can also be used as scaffolds for targeted RNAs that affect their expression when clustered in a similar way. Thus, where this combination of scaffolds is used, the first RNA of the invention plays a key role in enhancing the biogenesis of the second RNA of the invention, and therefore, the delivery of the guide sequences comprised in said first and second RNAs. Therefore, in one embodiment, reducing and/or silencing the expression of APOE is increased when the second RNA is expressed with the first RNA, when compared to the decreased expression of APOE achieved with the expression of the second RNA alone.

As is shown in the examples, a first strand of the second RNA, or the third strand of the invention, of 22 nucleotides (e.g. for a miR-451) in length, may be selected and incorporated in a miRNA scaffold. Such a miRNA scaffold sequence is subsequently processed by the RNAi machinery as present in the cell. When reference is made to miRNA scaffold it is understood to comprise pri-miRNA structures or pre-miRNA structures. As shown in the examples, such miRNA scaffolds, when processed in a cell, resulting in guide sequences comprising the first strand of the second RNA, or a substantial part thereof, in the range of 21-30 nucleotides in length for the miR-451 scaffolds. Such guide strands are capable of reducing APOE transcript expression by targeting the selected target sequences. As is clear from the above, and as shown in the examples, the first strand of the second RNA as it is encoded by the expression cassette of the invention, is comprised in part or in whole, in a guide strand when it has been processed by the RNAi machinery of the cell. Hence, the guide strand that is to be generated from the RNA encoded by the expression cassette, comprising the first strand of the second RNA and the second strand of the second RNA is to comprise at least 18 nucleotides of the first RNA sequence. Preferably, such a guide strand comprises at least 19 nucleotides, 20 nucleotides, 21 nucleotides, or 22 nucleotides. A guide strand can comprise the first strand of the second RNA sequence also as a whole. In selecting a miRNA scaffold, the first strand of the second RNA sequence can be selected such that it is to replace the original guide strand. As shown in the example section, this does not necessarily mean that a guide strand produced from such an artificial scaffold are identical in length as the first strand of the second RNA selected, nor that the first strand of the second RNA is in its entirety to be found in the guide strand that is produced. A miR-451 scaffold, as shown in the examples, and as shown in FIG. 2a preferably comprises from 5′ to 3′, firstly 5′-CUUGGGAAUGGCAAGG-3′ (SEQ ID NO. 233), followed by a sequence of 22 nucleotides, comprising or consisting of the first RNA sequence, followed a sequence of 17 nucleotides, which can be regarded to be the second RNA sequence, which is complementary over its entire length with nucleotides 2-18 of said sequence of 22 nucleotides, subsequently followed by sequence 5′-MWCUUGCUAUACCCAGA-3′ (wherein M is an A or a C and W is an A or a U) (SEQ ID NO. 234). Preferably the first 5′-C nucleotide of the latter sequence is not to base pair with the first nucleotide of the first strand of the second RNA. Such a scaffold may comprise further flanking sequences as found in the original pri-miR-451 scaffold. Alternatively, the flanking sequences, 5′-CUUGGGAAUGGCAAGG′-3′ and 5′-MWCUUGCUAUACCCAGA-3′ may be replaced by flanking sequences of other pri-mRNA structures. As is clear from the above, the sequence of the scaffold may differ not only with regard to the (putative) guide strand sequence, and sequence complementary thereto, as present in the wild-type scaffold (FIG. 2a), but may also comprise additional mutations in the 5′, loop and 3′ sequence as well, as additional mutations may be required to provide for an RNA structure that is predicted to mimic the secondary structure of the wild-type scaffold. Such a scaffold may be comprised in a larger RNA transcript, e.g. a pol II expressed transcript, comprising e.g. a 5′ UTR and a 3′UTR and a poly A. Flanking structures may also be absent. An expression cassette in accordance with the invention thus expressing an shRNA-like structure having a sequence of 22 nucleotides, comprising or consisting of the first strand of the second RNA, followed a sequence of 17 nucleotides, which can be regarded to be the second strand of the second RNA, which is complementary over its entire length with nucleotides 2-18 of said sequence of 22 nucleotides. The latter shRNA-like structure derived from the miR-451 scaffold can be referred to as a pre-miRNA scaffold from miR-451.

Alternatively, the flanking sequences, may be replaced by flanking sequences of other pre-miRNA structures. Flanking structures may also be absent. An expression cassette in accordance with the invention thus expressing an shRNA like structure, a pre-miRNA structure of miR-101. Such an shRNA-like structure consisting of, starting at the 5′-end, a second RNA sequence of 22 nucleotides in length, subsequently followed by a loop sequence, wherein the last 3′ two nucleotides of the loop sequence are to base pair with the last 3′ nucleotides of the second strand of the second RNA, followed by a first strand of the second RNA of 21 nucleotides in length, wherein the first 20 consecutive nucleotides are complementary to the second strand of the second RNA. The second strand of the second RNA comprises a bulge (non-base paired nucleotide) at position 5, counting from the 3′-end, of the said 22 nucleotides.

In one embodiment, an expression cassette according to the invention is provided, wherein said first strand of the second RNA is substantially complementary to a target RNA sequence comprised in antisense RNA transcripts encoded by the human APOE gene. Preferably said first strand of the second RNA is substantially complementary to SEQ ID NOs. 4, 16, 24, 41, 44, 46, 54, 59, 93. More preferably said first strand of the second RNA has a length of 19, 20, 21, or 22 nucleotides. More preferably said first strand of the second RNA is fully complementary over its entire length with said target sequence. Most preferably said first strand of the second RNA has a length of 19, 20, 21, or 22 nucleotides, wherein said first strand of the second RNA is fully complementary over its entire length with said target sequence. Said first strand of the second RNA can be SEQ ID NOs. 94-185, table 2.

TABLE 2
miAPOE guide strand sequences.

| SEQ | | |
|---|---|---|
| ID | | RNA GUIDE SEQUENCE |
| NO. | ID | (5′-3′) |
| 94 | miAPOE_001 | UUCCUGCCUGUGAUUGGCCAGU |
| 95 | miAPOE_002 | CUUCCUGCCUGUGAUUGGCCAG |
| 96 | miAPOE_003 | UCUUCCUGCCUGUGAUUGGCCA |
| 97 | miAPOE_004 | AUCUUCCUGCCUGUGAUUGGCC |
| 98 | miAPOE_005 | CAUCUUCCUGCCUGUGAUUGGC |
| 99 | miAPOE_006 | UCAUCUUCCUGCCUGUGAUUGG |
| 100 | miAPOE_007 | UUCAUCUUCCUGCCUGUGAUUG |
| 101 | miAPOE_008 | CUUCAUCUUCCUGCCUGUGAUU |
| 102 | miAPOE_009 | CCUUCAUCUUCCUGCCUGUGAU |
| 103 | miAPOE_010 | ACCUUCAUCUUCCUGCCUGUGA |
| 104 | miAPOE_011 | AACCUUCAUCUUCCUGCCUGUG |
| 105 | miAPOE_013 | AGAACCUUCAUCUUCCUGCCUG |
| 106 | miAPOE_014 | CAGAACCUUCAUCUUCCUGCCU |
| 107 | miAPOE_015 | ACAGAACCUUCAUCUUCCUGCC |
| 108 | miAPOE_016 | CACAGAACCUUCAUCUUCCUGC |
| 109 | miAPOE_017 | CCACAGAACCUUCAUCUUCCUG |
| 110 | miAPOE_018 | CCCACAGAACCUUCAUCUUCCU |
| 111 | miAPOE_020 | AGCCCACAGAACCUUCAUCUUC |
| 112 | miAPOE_021 | CAGCCCACAGAACCUUCAUCUU |
| 113 | miAPOE_024 | ACGCAGCCCACAGAACCUUCAU |
| 114 | miAPOE_025 | AACGCAGCCCACAGAACCUUCA |
| 115 | miAPOE_036 | AUGUGACCAGCAACGCAGCCCA |
| 116 | miAPOE_037 | AAUGUGACCAGCAACGCAGCCC |
| 117 | miAPOE_040 | AGGAAUGUGACCAGCAACGCAG |
| 118 | miAPOE_042 | CCAGGAAUGUGACCAGCAACGC |
| 119 | miAPOE_045 | CUGCCAGGAAUGUGACCAGCAA |
| 120 | miAPOE_053 | CUGGCAUCCUGCCAGGAAUGUG |
| 121 | miAPOE_085 | ACCCAGCGCAGGUAAUCCCAAA |
| 122 | miAPOE_100 | UCAGACAGUGUCUGCACCCAGC |
| 123 | miAPOE_101 | CUCAGACAGUGUCUGCACCCAG |
| 124 | miAPOE_104 | CUGCUCAGACAGUGUCUGCACC |
| 125 | miAPOE_105 | CCUGCUCAGACAGUGUCUGCAC |
| 126 | miAPOE_106 | ACCUGCUCAGACAGUGUCUGCA |
| 127 | miAPOE_107 | CACCUGCUCAGACAGUGUCUGC |
| 128 | miAPOE_109 | UGCACCUGCUCAGACAGUGUCU |
| 129 | miAPOE_110 | CUGCACCUGCUCAGACAGUGUC |
| 130 | miAPOE_111 | CCUGCACCUGCUCAGACAGUGU |
| 131 | miAPOE_126 | CCUUCAACUCCUUCAUGGUCUC |
| 132 | miAPOE_129 | AGGCCUUCAACUCCUUCAUGGU |
| 133 | miAPOE_130 | UAGGCCUUCAACUCCUUCAUGG |
| 134 | miAPOE_132 | UGUAGGCCUUCAACUCCUUCAU |
| 135 | miAPOE_133 | UUGUAGGCCUUCAACUCCUUCA |
| 136 | miAPOE_134 | UUUGUAGGCCUUCAACUCCUUC |
| 137 | miAPOE_135 | AUUUGUAGGCCUUCAACUCCUU |
| 138 | miAPOE_136 | GAUUUGUAGGCCUUCAACUCCU |
| 139 | miAPOE_137 | CGAUUUGUAGGCCUUCAACUCC |
| 140 | miAPOE_138 | CCGAUUUGUAGGCCUUCAACUC |
| 141 | miAPOE_139 | UCCGAUUUGUAGGCCUUCAACU |
| 142 | miAPOE_140 | UUCCGAUUUGUAGGCCUUCAAC |
| 143 | miAPOE_142 | AGUUCCGAUUUGUAGGCCUUCA |
| 144 | miAPOE_143 | CAGUUCCGAUUUGUAGGCCUUC |
| 145 | miAPOE_144 | CCAGUUCCGAUUUGUAGGCCUU |
| 146 | miAPOE_145 | UCCAGUUCCGAUUUGUAGGCCU |
| 147 | miAPOE_146 | CUCCAGUUCCGAUUUGUAGGCC |
| 148 | miAPOE_147 | CCUCCAGUUCCGAUUUGUAGGC |
| 149 | miAPOE_148 | UCCUCCAGUUCCGAUUUGUAGG |
| 150 | miAPOE_149 | UUCCUCCAGUUCCGAUUUGUAG |
| 151 | miAPOE_151 | UGUUCCUCCAGUUCCGAUUUGU |
| 152 | miAPOE_175 | UGCAGCUCCUUGGACAGCCGUG |
| 153 | miAPOE_176 | CUGCAGCUCCUUGGACAGCCGU |
| 154 | miAPOE_202 | AGCUUGCGCAGGUGGGAGGCGA |
| 155 | miAPOE_206 | CCAGGCGCUUCUGCAGGUCAUC |
| 156 | miAPOE_209 | CUGCCAGGCGCUUCUGCAGGUC |
| 157 | miAPOE_210 | ACUGCCAGGCGCUUCUGCAGGU |
| 158 | miAPOE_212 | ACACUGCCAGGCGCUUCUGCAG |
| 159 | miAPOE_213 | UACACUGCCAGGCGCUUCUGCA |
| 160 | miAPOE_278 | UCCUUCACCUCGUCCAGGCGGU |
| 161 | miAPOE_283 | CCUGCUCCUUCACCUCGUCCAG |
| 162 | miAPOE_286 | CCACCUGCUCCUUCACCUCGUC |
| 163 | miAPOE_336 | UCGAACCAGCUCUUGAGGCGGG |
| 164 | miAPOE_337 | CUCGAACCAGCUCUUGAGGCGG |
| 165 | miAPOE_345 | AGCCUGCACCUUCUCCACCAGC |
| 166 | miAPOE_350 | ACGGCAGCCUGCACCUUCUCCA |
| 167 | miAPOE_360 | AGUGAUUGUCGCUGGGCACAGG |
| 168 | miAPOE_362 | UCAGUGAUUGUCGCUGGGCACA |
| 169 | miAPOE_363 | UUCAGUGAUUGUCGCUGGGCAC |
| 170 | miAPOE_365 | CGUUCAGUGAUUGUCGCUGGGC |
| 171 | miAPOE_368 | CAGGGUCUCCCGCUGCAGGCUG |
| 172 | miAPOE_369 | ACAGGGUCUCCCGCUGCAGGCU |
| 173 | miAPOE_391 | GGCGCGGACAUGGAGGACGUGC |
| 174 | miAPOE_392 | GCGCGGACAUGGAGGACGUGCG |
| 175 | miAPOE_393 | CGCGGACAUGGAGGACGUGCGC |
| 176 | miAPOE_394 | GCGGACAUGGAGGACGUGCGCG |
| 177 | miAPOE_395 | CGGACAUGGAGGACGUGCGCGG |
| 178 | miAPOE_396 | GGACAUGGAGGACGUGCGCGGC |
| 179 | miAPOE_397 | GGCCGCGCACGUCCUCCAUGUC |
| 180 | miAPOE_398 | CGGCCGCGCACGUCCUCCAUGU |
| 181 | miAPOE_399 | GCGGCCGCGCACGUCCUCCAUG |
| 182 | miAPOE_410 | UACUGCACCAGGCGGCCGCGCA |
| 183 | miAPOE_417 | UUUAUUAAACUAGGGUCCACCC |
| 184 | miAPOE_418 | CUUUAUUAAACUAGGGUCCACC |
| 185 | miAPOE_419 | UCUUUAUUAAACUAGGGUCCAC |

As said, such a first strand of the second RNA is to be combined with a second strand of the second RNA, which may also be referred to as the third and fourth strands of the invention. As described herein, the skilled person is capable of designing and selecting a suitable second strand of the second RNA in order to provide for a first and second strand for the second RNA that can induce RNA interference when expressed in a cell. A suitable second strand of the second RNA is complementary over its entire length with the nucleotides 2-15, 2-16,2-17 or 2-18 to the first strand of the second RNA having a length of 19, 20, 21, or 22 nucleotides.

Said first strand of the second RNA is preferably comprised in a miRNA scaffold, more preferably a miR-451 scaffold, such as shown in the examples. A suitable scaffold comprising a first and second strand for the second RNA in accordance with the invention can be a sequence such as SEQ ID NO. 190.

Such first strand of the second RNA as described above can be comprised in expression cassettes.

Such first strand of the second RNA can be comprised in RNA structures that are encoded by expression cassettes.

Such first and second strands of the second RNA sequences as described above can be comprised in expression cassettes. Such first and second strands of the second RNA can be comprised in RNA structures that are encoded by expression cassettes. Therefore, in some embodiments, the sequence encoding the first RNA and the sequence encoding the second RNA are comprised in an intronic sequence. By incorporating the sequences encoding the first and second RNA sequences the inventors identified the beneficial effect of deriving a smaller construct for expression of the RNAs of the invention, particularly useful for downstream processing steps, such as in the application of viral vector technology. In a further embodiment, the sequence encoding the first RNA and the sequence encoding the second RNA are present in the promoter, wherein preferably the intronic sequence has SEQ ID NO. 231.

Accordingly, targeting these target RNA sequences, utilizing such first and second strands of the second RNA, were found to be in particular useful for reducing the expression of RNA transcripts encoded by the human APOE gene. By targeting human APOE this way, the current inventors were able to highly efficiently reduce human APOE gene expression and thus may reduce the formation of amyloid plaques. Ultimately this may reverse, prevent, slow down the progression of, or completely halt neuropathologies, such as neurodegeneration and/or tauopathies.

### APOE Expression

In a further aspect, there is provided an expression cassette encoding at least one of APOE2 and APOE3. The APOEε2 and/or APOEε3 alleles exert a protective or a neutral effect against AD development (Yamazaki et al. 2016, CNS Drugs.; 30:773-89). Therefore, in some embodiments, the expression cassette comprises a nucleic acid that encodes at least one of APOE2 and APOE3 having an amino acid sequence selected from SEQ ID NOs. 249-254. The expression of at least one of APOE2 and APOE3 is neuroprotective against tauopathies. The term “tauopathies” as used herein describes neurodegenerative disorders characterised by the deposition of abnormal tau protein in the brain. In an embodiment, the expression cassette encoding at least one of APOE2 and APOE3, further comprises a promoter and a poly-A signal. In a further embodiment, the expression cassette comprises a nucleic acid which encodes one or more APOE2 and APOE3 proteins having an amino acid sequence selected from one or more of SEQ ID NOs. 249-254 or a variant thereof, for use in gene therapy.

In some embodiments, the expression cassette comprises one or more nucleic acid sequences that comprise at least partial wild type sequences. With at least partial wild type sequences it is meant a sequence with codon optimization only in specific regions, such as can be found in SEQ ID NOs: 212-217. In a preferred embodiment, the expression cassette comprises one or more nucleic acid sequences that comprise full length wild type coding sequences (e.g., SEQ ID NOs: 195-201). The invention of this application surprisingly has shown that the (at least partial) wild type versions of the nucleic acid sequences showed highest protein expression amongst the different variants. In a more preferred embodiment the expression cassette comprises a nucleic acid selected from one of SEQ ID NOs. 195, 197, 200. In a still a more preferred embodiment, the expression cassette comprises nucleic acids comprising SEQ ID NOs. 197 and 200, because of the known beneficial properties of the encoded proteins. In an alternative embodiment, the expression cassette comprises a nucleic acid selected from one of SEQ ID NOs. 197 and 200. The inventors have surprisingly shown that the wild type versions of the nucleic acid sequences encoding for APOE2 or APOE3 proteins are the ones resulting in the highest protein expression and secretion amongst the different variants. The codon optimization of the full length of the nucleic acid sequences resulted in a lower level of expression of APOE2 or APOE3 proteins.

In a further embodiment, the expression cassette encoding at least one of APOE2 and APOE3 comprises one or more nucleic acid sequences selected from one or more of SEQ ID NO 195-217, or a variant thereof. For example, in still a further embodiment, the expression cassette encoding at least one of APOE2 and APO3 comprises 2, or 3, or 4, or more nucleic acid sequences selected from one or more of SEQ ID NO. 195-217, or a variant thereof. In a further aspect, there is provided the expression cassette as disclosed herein comprising a nucleic acid comprising a sequence encoding a first RNA and a sequence encoding a second RNA as disclosed herein, wherein the second RNA comprises a guide sequence of at least 19 nucleotides substantially complementary to part of an APOE gene, and wherein the first RNA and second RNA each comprise a hairpin, further comprising a second nucleic acid encoding at least one of APOE2 and APOE3. In an embodiment, the first and second nucleic acids are operably linked to a promoter and to a poly-A signal. In a further embodiment, the second nucleic acid encodes for a protein comprising one of SEQ ID NOs 249-254 and/or the second nucleic acid comprises one of SEQ ID NOs. 195-217. Thus in some embodiments, the second nucleic acid encodes a protein having an amino acid sequence set forth in SEQ ID NOs. 249-254. In some embodiments, the second nucleic acid is a gene product encoded by a coding portion (e.g. cDNA) of a naturally occurring gene. In some embodiments, the gene product is a protein, or fragment thereof, encoded by the APOE2 and/or APOE3 isoform of the APOE gene. In a further embodiment, the second nucleic acid does not comprise a sequence that is substantially complementary to the guide sequence as defined herein. In a further embodiment, the second nucleic acid comprises a nucleotide sequence that is codon optimised for expression in human cells. In a further embodiment, the second nucleic acid is codon optimised to differ sufficiently from the endogenous APOE2 and/or APOE3 sequence in cells such that it would not be recognised by shRNAs targeting wild-type APOE, APOE2 and/or APOE3. The person skilled in the art has within their understanding the requirements needed to design such a nucleic acid. In a further embodiment, the second nucleic acid comprises a sequence selected from table 3 (SEQ ID NOs. 195-217).

TABLE 3
Nucleic acid expressing APOE2 or APOE3.

| SEQ | | |
|---|---|---|
| ID | | |
| NO. | NAME | DNA SEQUENCE |
| 195 | APOE2-WT | ATGAAGGTTCTGTGGGCTGCGTTGCTGGTCACATTCCTGGCAGGATGCC |
| | | AGGCCAAGGTGGAGCAAGCGGTGGAGACAGAGCCGGAGCCCGAGCTGC |
| | | GCCAGCAGACCGAGTGGCAGAGCGGCCAGCGCTGGGAACTGGCACTGG |
| | | GTCGCTTTTGGGATTACCTGCGCTGGGTGCAGACACTGTCTGAGCAGGT |
| | | GCAGGAGGAGCTGCTCAGCTCCCAGGTCACCCAGGAACTGAGGGCGCT |
| | | GATGGACGAGACCATGAAGGAGTTGAAGGCCTACAAATCGGAACTGGAG |
| | | GAACAACTGACCCCGGTGGCGGAGGAGACGCGGGCACGGCTGTCCAAG |
| | | GAGCTGCAGGCGGCGCAGGCCCGGCTGGGCGCGGACATGGAGGACGT |
| | | GTGCGGCCGCCTGGTGCAGTACCGCGGCGAGGTGCAGGCCATGCTCGG |
| | | CCAGAGCACCGAGGAGCTGCGGGTGCGCCTCGCCTCCCACCTGCGCAA |
| | | GCTGCGTAAGCGGCTCCTCCGCGATGCCGATGACCTGCAGAAGTGCCTG |
| | | GCAGTGTACCAGGCCGGGGCCCGCGAGGGCGCCGAGCGCGGCCTCAG |
| | | CGCCATCCGCGAGCGCCTGGGGCCCCTGGTGGAACAGGGCCGCGTGC |
| | | GGGCCGCCACTGTGGGCTCCCTGGCCGGCCAGCCGCTACAGGAGCGG |
| | | GCCCAGGCCTGGGGCGAGCGGCTGCGCGCGCGGATGGAGGAGATGGG |
| | | CAGCCGGACCCGCGACCGCCTGGACGAGGTGAAGGAGCAGGTGGCGG |
| | | AGGTGCGCGCCAAGCTGGAGGAGCAGGCCCAGCAGATACGCCTGCAGG |
| | | CCGAGGCCTTCCAGGCCCGCCTCAAGAGCTGGTTCGAGCCCCTGGTGG |
| | | AAGACATGCAGCGCCAGTGGGCCGGGCTGGTGGAGAAGGTGCAGGCTG |
| | | CCGTGGGCACCAGCGCCGCCCCTGTGCCCAGCGACAATCACTGA |
| 196 | APOE3-WT | ATGAAGGTTCTGTGGGCTGCGTTGCTGGTCACATTCCTGGCAGGATGCC |
| | | AGGCCAAGGTGGAGCAAGCGGTGGAGACAGAGCCGGAGCCCGAGCTGC |
| | | GCCAGCAGACCGAGTGGCAGAGCGGCCAGCGCTGGGAACTGGCACTGG |
| | | GTCGCTTTTGGGATTACCTGCGCTGGGTGCAGACACTGTCTGAGCAGGT |
| | | GCAGGAGGAGCTGCTCAGCTCCCAGGTCACCCAGGAACTGAGGGCGCT |
| | | GATGGACGAGACCATGAAGGAGTTGAAGGCCTACAAATCGGAACTGGAG |
| | | GAACAACTGACCCCGGTGGCGGAGGAGACGCGGGCACGGCTGTCCAAG |
| | | GAGCTGCAGGCGGCGCAGGCCCGGCTGGGCGCGGACATGGAGGACGT |
| | | GTGCGGCCGCCTGGTGCAGTACCGCGGCGAGGTGCAGGCCATGCTCGG |
| | | CCAGAGCACCGAGGAGCTGCGGGTGCGCCTCGCCTCCCACCTGCGCAA |
| | | GCTGCGTAAGCGGCTCCTCCGCGATGCCGATGACCTGCAGAAGCGCCT |
| | | GGCAGTGTACCAGGCCGGGGCCCGCGAGGGCGCCGAGCGCGGCCTCA |
| | | GCGCCATCCGCGAGCGCCTGGGGCCCCTGGTGGAACAGGGCCGCGTG |
| | | CGGGCCGCCACTGTGGGCTCCCTGGCCGGCCAGCCGCTACAGGAGCGG |
| | | GCCCAGGCCTGGGGCGAGCGGCTGCGCGCGCGGATGGAGGAGATGGG |
| | | CAGCCGGACCCGCGACCGCCTGGACGAGGTGAAGGAGCAGGTGGCGG |
| | | AGGTGCGCGCCAAGCTGGAGGAGCAGGCCCAGCAGATACGCCTGCAGG |
| | | CCGAGGCCTTCCAGGCCCGCCTCAAGAGCTGGTTCGAGCCCCTGGTGG |
| | | AAGACATGCAGCGCCAGTGGGCCGGGCTGGTGGAGAAGGTGCAGGCTG |
| | | CCGTGGGCACCAGCGCCGCCCCTGTGCCCAGCGACAATCACTGA |
| 197 | APOE2 ch- | ATGAAGGTTCTGTGGGCTGCGTTGCTGGTCACATTCCTGGCAGGATGCC |
| | WT | AGGCCAAGGTGGAGCAAGCGGTGGAGACAGAGCCGGAGCCCGAGCTGC |
| | (Christchurch, | GCCAGCAGACCGAGTGGCAGAGCGGCCAGCGCTGGGAACTGGCACTGG |
| | R136S) | GTCGCTTTTGGGATTACCTGCGCTGGGTGCAGACACTGTCTGAGCAGGT |
| | | GCAGGAGGAGCTGCTCAGCTCCCAGGTCACCCAGGAACTGAGGGCGCT |
| | | GATGGACGAGACCATGAAGGAGTTGAAGGCCTACAAATCGGAACTGGAG |
| | | GAACAACTGACCCCGGTGGCGGAGGAGACGCGGGCACGGCTGTCCAAG |
| | | GAGCTGCAGGCGGCGCAGGCCCGGCTGGGCGCGGACATGGAGGACGT |
| | | GTGCGGCCGCCTGGTGCAGTACCGCGGCGAGGTGCAGGCCATGCTCGG |
| | | CCAGAGCACCGAGGAGCTGCGGGTGAGCCTCGCCTCCCACCTGCGCAA |
| | | GCTGCGTAAGCGGCTCCTCCGCGATGCCGATGACCTGCAGAAGTGCCTG |
| | | GCAGTGTACCAGGCCGGGGCCCGCGAGGGCGCCGAGCGCGGCCTCAG |
| | | CGCCATCCGCGAGCGCCTGGGGCCCCTGGTGGAACAGGGCCGCGTGC |
| | | GGGCCGCCACTGTGGGCTCCCTGGCCGGCCAGCCGCTACAGGAGCGG |
| | | GCCCAGGCCTGGGGCGAGCGGCTGCGCGCGCGGATGGAGGAGATGGG |
| | | CAGCCGGACCCGCGACCGCCTGGACGAGGTGAAGGAGCAGGTGGCGG |
| | | AGGTGCGCGCCAAGCTGGAGGAGCAGGCCCAGCAGATACGCCTGCAGG |
| | | CCGAGGCCTTCCAGGCCCGCCTCAAGAGCTGGTTCGAGCCCCTGGTGG |
| | | AAGACATGCAGCGCCAGTGGGCCGGGCTGGTGGAGAAGGTGCAGGCTG |
| | | CCGTGGGCACCAGCGCCGCCCCTGTGCCCAGCGACAATCACTGA |
| 198 | APOE3 ch- | ATGAAGGTTCTGTGGGCTGCGTTGCTGGTCACATTCCTGGCAGGATGCC |
| | WT | AGGCCAAGGTGGAGCAAGCGGTGGAGACAGAGCCGGAGCCCGAGCTGC |
| | (Christchurch, | GCCAGCAGACCGAGTGGCAGAGCGGCCAGCGCTGGGAACTGGCACTGG |
| | R136S) | GTCGCTTTTGGGATTACCTGCGCTGGGTGCAGACACTGTCTGAGCAGGT |
| | | GCAGGAGGAGCTGCTCAGCTCCCAGGTCACCCAGGAACTGAGGGCGCT |
| | | GATGGACGAGACCATGAAGGAGTTGAAGGCCTACAAATCGGAACTGGAG |
| | | GAACAACTGACCCCGGTGGCGGAGGAGACGCGGGCACGGCTGTCCAAG |
| | | GAGCTGCAGGCGGCGCAGGCCCGGCTGGGCGCGGACATGGAGGACGT |
| | | GTGCGGCCGCCTGGTGCAGTACCGCGGCGAGGTGCAGGCCATGCTCGG |
| | | CCAGAGCACCGAGGAGCTGCGGGTGAGCCTCGCCTCCCACCTGCGCAA |
| | | GCTGCGTAAGCGGCTCCTCCGCGATGCCGATGACCTGCAGAAGCGCCT |
| | | GGCAGTGTACCAGGCCGGGGCCCGCGAGGGCGCCGAGCGCGGCCTCA |
| | | GCGCCATCCGCGAGCGCCTGGGGCCCCTGGTGGAACAGGGCCGCGTG |
| | | CGGGCCGCCACTGTGGGCTCCCTGGCCGGCCAGCCGCTACAGGAGCGG |
| | | GCCCAGGCCTGGGGCGAGCGGCTGCGCGCGCGGATGGAGGAGATGGG |
| | | CAGCCGGACCCGCGACCGCCTGGACGAGGTGAAGGAGCAGGTGGCGG |
| | | AGGTGCGCGCCAAGCTGGAGGAGCAGGCCCAGCAGATACGCCTGCAGG |
| | | CCGAGGCCTTCCAGGCCCGCCTCAAGAGCTGGTTCGAGCCCCTGGTGG |
| | | AAGACATGCAGCGCCAGTGGGCCGGGCTGGTGGAGAAGGTGCAGGCTG |
| | | CCGTGGGCACCAGCGCCGCCCCTGTGCCCAGCGACAATCACTGA |
| 199 | APOE3 ch- | ATGAAGGTTCTGTGGGCTGCGTTGCTGGTCACATTCCTGGCAGGATGCC |
| | HA-WT | AGGCCAAGGTGGAGCAAGCGGTGGAGACAGAGCCGGAGCCCGAGCTGC |
| | (Christchurch, | GCCAGCAGACCGAGTGGCAGAGCGGCCAGCGCTGGGAACTGGCACTGG |
| | R136S) | GTCGCTTTTGGGATTACCTGCGCTGGGTGCAGACACTGTCTGAGCAGGT |
| | | GCAGGAGGAGCTGCTCAGCTCCCAGGTCACCCAGGAACTGAGGGCGCT |
| | | GATGGACGAGACCATGAAGGAGTTGAAGGCCTACAAATCGGAACTGGAG |
| | | GAACAACTGACCCCGGTGGCGGAGGAGACGCGGGCACGGCTGTCCAAG |
| | | GAGCTGCAGGCGGCGCAGGCCCGGCTGGGCGCGGACATGGAGGACGT |
| | | GTGtGGCCGCCTGGTGCAGTACCGCGGCGAGGTGCAGGCCATGCTCGG |
| | | CCAGAGCACCGAGGAGCTGCGGGTGaGCCTCGCCTCCCACCTGCGCAA |
| | | GCTGCGTAAGCGGCTCCTCCGCGATGCCGATGACCTGCAGAAGCGCCT |
| | | GGCAGTGTACCAGGCCGGGGCCCGCGAGGGCGCCGAGCGCGGCCTCA |
| | | GCGCCATCCGCGAGCGCCTGGGGCCCCTGGTGGAACAGGGCCGCGTG |
| | | CGGGCCGCCACTGTGGGCTCCCTGGCCGGCCAGCCGCTACAGGAGCGG |
| | | GCCCAGGCCTGGGGCGAGCGGCTGCGCGCGCGGATGGAGGAGATGGG |
| | | CAGCCGGACCCGCGACCGCCTGGACGAGGTGAAGGAGCAGGTGGCGG |
| | | AGGTGCGCGCCAAGCTGGAGGAGCAGGCCCAGCAGATACGCCTGCAGG |
| | | CCGAGGCCTTCCAGGCCCGCCTCAAGAGCTGGTTCGAGCCCCTGGTGG |
| | | AAGACATGCAGCGCCAGTGGGCCGGGCTGGTGGAGAAGGTGCAGGCTG |
| | | CCGTGGGCACCAGCGCCGCCCCTGTGCCCAGCGACAATCAC |
| 200 | APOE2b- | ATGAAGGTTCTGTGGGCTGCGTTGCTGGTCACATTCCTGGCAGGATGCC |
| | APOE2-Jac- | AGGCCAAGGTGGAGCAAGCGGTGGAGACAGAGCCGGAGCCCGAGCTGC |
| | WT | GCCAGCAGACCGAGTGGCAGAGCGGCCAGCGCTGGGAACTGGCACTGG |
| | (Jacksonville, | GTCGCTTTTGGGATTACCTGCGCTGGGTGCAGACACTGTCTGAGCAGGT |
| | V236E) | GCAGGAGGAGCTGCTCAGCTCCCAGGTCACCCAGGAACTGAGGGCGCT |
| | | GATGGACGAGACCATGAAGGAGTTGAAGGCCTACAAATCGGAACTGGAG |
| | | GAACAACTGACCCCGGTGGCGGAGGAGACGCGGGCACGGCTGTCCAAG |
| | | GAGCTGCAGGCGGCGCAGGCCCGGCTGGGCGCGGACATGGAGGACGT |
| | | GTGCGGCCGCCTGGTGCAGTACCGCGGCGAGGTGCAGGCCATGCTCGG |
| | | CCAGAGCACCGAGGAGCTGCGGGTGCGCCTCGCCTCCCACCTGCGCAA |
| | | GCTGCGTAAGCGGCTCCTCCGCGATGCCGATGACCTGCAGAAGTGCCTG |
| | | GCAGTGTACCAGGCCGGGGCCCGCGAGGGCGCCGAGCGCGGCCTCAG |
| | | CGCCATCCGCGAGCGCCTGGGGCCCCTGGTGGAACAGGGCCGCGTGC |
| | | GGGCCGCCACTGTGGGCTCCCTGGCCGGCCAGCCGCTACAGGAGCGG |
| | | GCCCAGGCCTGGGGCGAGCGGCTGCGCGCGCGGATGGAGGAGATGGG |
| | | CAGCCGGACCCGCGACCGCCTGGACGAGGTGAAGGAGCAGGAGGCGG |
| | | AGGTGCGCGCCAAGCTGGAGGAGCAGGCCCAGCAGATACGCCTGCAGG |
| | | CCGAGGCCTTCCAGGCCCGCCTCAAGAGCTGGTTCGAGCCCCTGGTGG |
| | | AAGACATGCAGCGCCAGTGGGCCGGGCTGGTGGAGAAGGTGCAGGCTG |
| | | CCGTGGGCACCAGCGCCGCCCCTGTGCCCAGCGACAATCACTGA |
| 201 | APOE3b- | ATGAAGGTTCTGTGGGCTGCGTTGCTGGTCACATTCCTGGCAGGATGCC |
| | APOE3-Jac- | AGGCCAAGGTGGAGCAAGCGGTGGAGACAGAGCCGGAGCCCGAGCTGC |
| | WT | GCCAGCAGACCGAGTGGCAGAGCGGCCAGCGCTGGGAACTGGCACTGG |
| | (Jacksonville, | GTCGCTTTTGGGATTACCTGCGCTGGGTGCAGACACTGTCTGAGCAGGT |
| | V236E) | GCAGGAGGAGCTGCTCAGCTCCCAGGTCACCCAGGAACTGAGGGCGCT |
| | | GATGGACGAGACCATGAAGGAGTTGAAGGCCTACAAATCGGAACTGGAG |
| | | GAACAACTGACCCCGGTGGCGGAGGAGACGCGGGCACGGCTGTCCAAG |
| | | GAGCTGCAGGCGGCGCAGGCCCGGCTGGGCGCGGACATGGAGGACGT |
| | | GTGCGGCCGCCTGGTGCAGTACCGCGGCGAGGTGCAGGCCATGCTCGG |
| | | CCAGAGCACCGAGGAGCTGCGGGTGCGCCTCGCCTCCCACCTGCGCAA |
| | | GCTGCGTAAGCGGCTCCTCCGCGATGCCGATGACCTGCAGAAGCGCCT |
| | | GGCAGTGTACCAGGCCGGGGCCCGCGAGGGCGCCGAGCGCGGCCTCA |
| | | GCGCCATCCGCGAGCGCCTGGGGCCCCTGGTGGAACAGGGCCGCGTG |
| | | CGGGCCGCCACTGTGGGCTCCCTGGCCGGCCAGCCGCTACAGGAGCGG |
| | | GCCCAGGCCTGGGGCGAGCGGCTGCGCGCGCGGATGGAGGAGATGGG |
| | | CAGCCGGACCCGCGACCGCCTGGACGAGGTGAAGGAGCAGGAGGCGG |
| | | AGGTGCGCGCCAAGCTGGAGGAGCAGGCCCAGCAGATACGCCTGCAGG |
| | | CCGAGGCCTTCCAGGCCCGCCTCAAGAGCTGGTTCGAGCCCCTGGTGG |
| | | AAGACATGCAGCGCCAGTGGGCCGGGCTGGTGGAGAAGGTGCAGGCTG |
| | | CCGTGGGCACCAGCGCCGCCCCTGTGCCCAGCGACAATCACTGA |
| 202 | APOE2-V1 | ATGAAGGTGCTGTGGGCCGCTCTGCTGGTCACATTTCTGGCCGGCTGTC |
| | | AGGCCAAAGTGGAACAGGCCGTGGAAACCGAGCCTGAGCCTGAACTGA |
| | | GACAGCAGACAGAGTGGCAGTCCGGCCAGAGATGGGAACTTGCCCTGG |
| | | GCAGATTCTGGGACTACCTGAGATGGGTGCAGACCCTGAGCGAACAGGT |
| | | GCAAGAGGAACTGCTGAGCAGCCAAGTGACCCAAGAGCTGAGAGCCCTG |
| | | ATGGACGAGACAATGAAGGAACTGAAGGCCTACAAGAGCGAGCTGGAAG |
| | | AACAGCTGACCCCTGTGGCCGAGGAAACAAGAGCCAGACTGAGCAAAGA |
| | | GCTGCAGGCCGCTCAAGCTAGACTGGGCGCCGATATGGAAGATGTGTGC |
| | | GGCAGACTGGTGCAGTACAGAGGCGAAGTGCAGGCCATGCTGGGCCAG |
| | | TCTACCGAGGAACTGAGAGTGCGGCTGGCCTCTCACCTGAGAAAGCTGA |
| | | GGAAGAGACTGCTGAGGGACGCCGACGACCTGCAGAAATGTCTGGCCG |
| | | TGTATCAGGCAGGCGCCAGAGAAGGTGCAGAGAGAGGACTGTCTGCCAT |
| | | CAGAGAAAGACTGGGCCCTCTGGTCGAGCAGGGCAGAGTTAGAGCTGC |
| | | CACAGTGGGAAGTCTGGCAGGCCAGCCTCTGCAAGAAAGAGCACAGGC |
| | | CTGGGGCGAGAGACTGAGGGCCAGAATGGAAGAGATGGGCAGCAGAAC |
| | | CCGGGACAGACTGGACGAAGTGAAAGAACAGGTGGCCGAAGTGCGCGC |
| | | CAAGCTCGAAGAACAAGCCCAGCAGATTCGGCTGCAGGCTGAAGCCTTT |
| | | CAGGCCCGGCTGAAGAGTTGGTTCGAGCCCCTGGTGGAAGATATGCAGA |
| | | GACAGTGGGCTGGACTGGTGGAAAAGGTGCAGGCTGCCGTGGGAACAT |
| | | CTGCTGCTCCAGTGCCTAGCGACAACCACTGA |
| 203 | APOE3-V1 | ATGAAGGTGCTGTGGGCCGCTCTGCTGGTCACATTTCTGGCCGGCTGTC |
| | | AGGCCAAAGTGGAACAGGCCGTGGAAACCGAGCCTGAGCCTGAACTGA |
| | | GACAGCAGACAGAGTGGCAGTCCGGCCAGAGATGGGAACTTGCCCTGG |
| | | GCAGATTCTGGGACTACCTGAGATGGGTGCAGACCCTGAGCGAACAGGT |
| | | GCAAGAGGAACTGCTGAGCAGCCAAGTGACCCAAGAGCTGAGAGCCCTG |
| | | ATGGACGAGACAATGAAGGAACTGAAGGCCTACAAGAGCGAGCTGGAAG |
| | | AACAGCTGACCCCTGTGGCCGAGGAAACAAGAGCCAGACTGAGCAAAGA |
| | | GCTGCAGGCCGCTCAAGCTAGACTGGGCGCCGATATGGAAGATGTGTGC |
| | | GGCAGACTGGTGCAGTACAGAGGCGAAGTGCAGGCCATGCTGGGCCAG |
| | | TCTACCGAGGAACTGAGAGTGCGGCTGGCCTCTCACCTGAGAAAGCTGA |
| | | GGAAGAGACTGCTGAGGGACGCCGACGACCTGCAGAAAAGACTGGCCG |
| | | TGTATCAGGCAGGCGCCAGAGAAGGTGCAGAGAGAGGACTGTCTGCCAT |
| | | CAGAGAAAGGCTGGGCCCTCTGGTTGAGCAGGGCAGAGTTAGAGCTGC |
| | | CACAGTGGGAAGTCTGGCAGGCCAGCCTCTGCAAGAAAGAGCACAGGC |
| | | CTGGGGCGAGAGACTGAGGGCCAGAATGGAAGAGATGGGCAGCAGAAC |
| | | CCGGGACAGACTGGACGAAGTGAAAGAACAGGTGGCCGAAGTGCGCGC |
| | | CAAGCTCGAAGAACAAGCCCAGCAGATTCGGCTGCAGGCTGAAGCCTTT |
| | | CAGGCCCGGCTGAAGAGTTGGTTCGAGCCCCTGGTGGAAGATATGCAGA |
| | | GACAGTGGGCTGGACTGGTGGAAAAGGTGCAGGCTGCCGTGGGAACAT |
| | | CTGCTGCTCCAGTGCCTAGCGACAACCACTGA |
| 204 | APOE2 ch-V1 | ATGAAGGTGCTGTGGGCCGCTCTGCTGGTCACATTTCTGGCCGGCTGTC |
| | (Christchurch, | AGGCCAAAGTGGAACAGGCCGTGGAAACCGAGCCTGAGCCTGAACTGA |
| | R136S) | GACAGCAGACAGAGTGGCAGTCCGGCCAGAGATGGGAACTTGCCCTGG |
| | | GCAGATTCTGGGACTACCTGAGATGGGTGCAGACCCTGAGCGAACAGGT |
| | | GCAAGAGGAACTGCTGAGCAGCCAAGTGACCCAAGAGCTGAGAGCCCTG |
| | | ATGGACGAGACAATGAAGGAACTGAAGGCCTACAAGAGCGAGCTGGAAG |
| | | AACAGCTGACCCCTGTGGCCGAGGAAACAAGAGCCAGACTGAGCAAAGA |
| | | GCTGCAGGCCGCTCAAGCTAGACTGGGCGCCGATATGGAAGATGTGTGC |
| | | GGCAGACTGGTGCAGTACAGAGGCGAAGTGCAGGCCATGCTGGGCCAG |
| | | TCTACCGAGGAACTGAGAGTGTCTCTGGCCAGCCACCTGAGAAAGCTGC |
| | | GGAAGAGACTGCTGAGAGATGCCGACGACCTGCAGAAATGCCTGGCCGT |
| | | GTATCAGGCTGGCGCTAGAGAAGGCGCTGAGAGAGGACTGTCTGCCATC |
| | | CGGGAAAGACTGGGCCCTCTGGTTGAACAGGGCAGAGTTAGAGCCGCC |
| | | ACAGTGGGATCTCTTGCAGGCCAGCCTCTGCAAGAAAGAGCCCAGGCTT |
| | | GGGGCGAGAGACTGAGGGCCAGAATGGAAGAGATGGGCAGCAGAACCA |
| | | GAGACAGACTGGACGAAGTGAAAGAACAGGTGGCCGAAGTGCGCGCCA |
| | | AGCTCGAAGAACAAGCCCAGCAGATTCGGCTGCAGGCTGAAGCCTTTCA |
| | | GGCCCGGCTGAAGAGTTGGTTCGAGCCCCTGGTGGAAGATATGCAGAGA |
| | | CAGTGGGCTGGACTGGTGGAAAAGGTGCAGGCTGCCGTGGGAACATCT |
| | | GCTGCTCCAGTGCCTAGCGACAACCACTGA |
| 205 | APOE3 ch-V1 | ATGAAGGTGCTGTGGGCCGCTCTGCTGGTCACATTTCTGGCCGGCTGTC |
| | (Christchurch, | AGGCCAAAGTGGAACAGGCCGTGGAAACCGAGCCTGAGCCTGAACTGA |
| | R136S) | GACAGCAGACAGAGTGGCAGTCCGGCCAGAGATGGGAACTTGCCCTGG |
| | | GCAGATTCTGGGACTACCTGAGATGGGTGCAGACCCTGAGCGAACAGGT |
| | | GCAAGAGGAACTGCTGAGCAGCCAAGTGACCCAAGAGCTGAGAGCCCTG |
| | | ATGGACGAGACAATGAAGGAACTGAAGGCCTACAAGAGCGAGCTGGAAG |
| | | AACAGCTGACCCCTGTGGCCGAGGAAACAAGAGCCAGACTGAGCAAAGA |
| | | GCTGCAGGCCGCTCAAGCTAGACTGGGCGCCGATATGGAAGATGTGTGC |
| | | GGCAGACTGGTGCAGTACAGAGGCGAAGTGCAGGCCATGCTGGGCCAG |
| | | TCTACCGAGGAACTGAGAGTGTCTCTGGCCAGCCACCTGAGAAAGCTGC |
| | | GGAAGAGACTGCTGAGAGATGCCGACGACCTGCAGAAAAGACTGGCCGT |
| | | GTATCAGGCAGGCGCCAGAGAAGGTGCAGAGAGAGGACTGTCTGCCATC |
| | | AGAGAAAGGCTGGGCCCTCTGGTTGAGCAGGGCAGAGTTAGAGCTGCCA |
| | | CAGTGGGAAGTCTGGCAGGCCAGCCTCTGCAAGAAAGAGCACAGGCCT |
| | | GGGGCGAGAGACTGAGGGCCAGAATGGAAGAGATGGGCAGCAGAACCC |
| | | GGGACAGACTGGACGAAGTGAAAGAACAGGTGGCCGAAGTGCGCGCCA |
| | | AGCTCGAAGAACAAGCCCAGCAGATTCGGCTGCAGGCTGAAGCCTTTCA |
| | | GGCCCGGCTGAAGAGTTGGTTCGAGCCCCTGGTGGAAGATATGCAGAGA |
| | | CAGTGGGCTGGACTGGTGGAAAAGGTGCAGGCTGCCGTGGGAACATCT |
| | | GCTGCTCCAGTGCCTAGCGACAACCACTGA |
| 206 | APOE2-V2 | ATGAAAGTCCTCTGGGCCGCTCTCCTGGTGACCTTTCTGGCCGGCTGTC |
| | | AGGCCAAAGTGGAACAGGCCGTGGAAACCGAGCCTGAGCCTGAACTGA |
| | | GACAGCAGACAGAGTGGCAGTCCGGCCAGAGATGGGAACTTGCCCTGG |
| | | GCAGATTCTGGGACTACCTGAGATGGGTGCAGACCCTGAGCGAACAGGT |
| | | GCAAGAGGAACTGCTGAGCAGCCAAGTGACCCAAGAGCTGAGAGCCCTG |
| | | ATGGACGAGACAATGAAAGAACTCAAAGCTTATAAGAGCGAGCTCGAAGA |
| | | GCAGCTGACCCCTGTGGCCGAGGAAACAAGAGCCAGACTGAGCAAAGA |
| | | GCTGCAGGCCGCTCAAGCTAGACTGGGCGCCGATATGGAAGATGTGTGC |
| | | GGCAGACTGGTGCAGTACAGAGGCGAAGTGCAGGCCATGCTGGGCCAG |
| | | TCTACCGAGGAACTGAGAGTGCGGCTGGCCAGCCACCTGAGAAAGCTGC |
| | | GGAAGAGACTGCTGAGAGATGCCGACGACCTGCAGAAATGCCTGGCCGT |
| | | GTATCAGGCTGGCGCTAGAGAAGGCGCTGAGAGAGGACTGTCTGCCATC |
| | | CGGGAAAGACTGGGCCCTCTGGTTGAACAGGGCAGAGTTAGAGCCGCC |
| | | ACAGTGGGATCTCTTGCAGGCCAGCCTCTGCAAGAAAGAGCCCAGGCTT |
| | | GGGGCGAGAGACTGAGGGCCAGAATGGAAGAGATGGGCAGCAGAACCA |
| | | GAGACAGACTGGACGAAGTGAAAGAACAGGTGGCCGAAGTGCGCGCCA |
| | | AGCTCGAAGAACAAGCCCAGCAGATTCGGCTGCAGGCTGAAGCCTTTCA |
| | | GGCCCGGCTGAAGAGTTGGTTCGAGCCCCTGGTGGAAGATATGCAGAGA |
| | | CAGTGGGCTGGACTGGTGGAAAAGGTGCAGGCTGCCGTGGGAACATCT |
| | | GCTGCTCCAGTGCCTAGCGACAACCACTGA |
| 207 | APOE3-V2 | ATGAAAGTCCTCTGGGCCGCTCTCCTGGTGACCTTTCTGGCCGGCTGTC |
| | | AGGCCAAAGTGGAACAGGCCGTGGAAACCGAGCCTGAGCCTGAACTGA |
| | | GACAGCAGACAGAGTGGCAGTCCGGCCAGAGATGGGAACTTGCCCTGG |
| | | GCAGATTCTGGGACTACCTGAGATGGGTGCAGACCCTGAGCGAACAGGT |
| | | GCAAGAGGAACTGCTGAGCAGCCAAGTGACCCAAGAGCTGAGAGCCCTG |
| | | ATGGACGAGACAATGAAAGAACTCAAAGCTTATAAGAGCGAGCTCGAAGA |
| | | GCAGCTGACCCCTGTGGCCGAGGAAACAAGAGCCAGACTGAGCAAAGA |
| | | GCTGCAGGCCGCTCAAGCTAGACTGGGCGCCGATATGGAAGATGTGTGC |
| | | GGCAGACTGGTGCAGTACAGAGGCGAAGTGCAGGCCATGCTGGGCCAG |
| | | TCTACCGAGGAACTGAGAGTGCGGCTGGCCAGCCACCTGAGAAAGCTGC |
| | | GGAAGAGACTGCTGAGAGATGCCGACGACCTGCAGAAACGCCTGGCCG |
| | | TGTATCAGGCTGGCGCTAGAGAAGGCGCTGAGAGAGGACTGTCTGCCAT |
| | | CCGGGAAAGACTGGGCCCTCTGGTTGAACAGGGCAGAGTTAGAGCCGC |
| | | CACAGTGGGATCTCTTGCAGGCCAGCCTCTGCAAGAAAGAGCCCAGGCT |
| | | TGGGGCGAGAGACTGAGGGCCAGAATGGAAGAGATGGGCAGCAGAACC |
| | | AGAGACAGACTGGACGAAGTGAAAGAACAGGTGGCCGAAGTGCGCGCC |
| | | AAGCTCGAAGAACAAGCCCAGCAGATTCGGCTGCAGGCTGAAGCCTTTC |
| | | AGGCCCGGCTGAAGAGTTGGTTCGAGCCCCTGGTGGAAGATATGCAGAG |
| | | ACAGTGGGCTGGACTGGTGGAAAAGGTGCAGGCTGCCGTGGGAACATCT |
| | | GCTGCTCCAGTGCCTAGCGACAACCACTGA |
| 208 | APOE2 ch-V2 | ATGAAAGTCCTCTGGGCCGCTCTCCTGGTGACCTTTCTGGCCGGCTGTC |
| | (Christchurch, | AGGCCAAAGTGGAACAGGCCGTGGAAACCGAGCCTGAGCCTGAACTGA |
| | R136S) | GACAGCAGACAGAGTGGCAGTCCGGCCAGAGATGGGAACTTGCCCTGG |
| | | GCAGATTCTGGGACTACCTGAGATGGGTGCAGACCCTGAGCGAACAGGT |
| | | GCAAGAGGAACTGCTGAGCAGCCAAGTGACCCAAGAGCTGAGAGCCCTG |
| | | ATGGACGAGACAATGAAAGAACTCAAAGCTTATAAGAGCGAGCTCGAAGA |
| | | GCAGCTGACCCCTGTGGCCGAGGAAACAAGAGCCAGACTGAGCAAAGA |
| | | GCTGCAGGCCGCTCAAGCTAGACTGGGCGCCGATATGGAAGATGTGTGC |
| | | GGCAGACTGGTGCAGTACAGAGGCGAAGTGCAGGCCATGCTGGGCCAG |
| | | TCTACCGAGGAACTGAGAGTGTCTCTGGCCAGCCACCTGAGAAAGCTGC |
| | | GGAAGAGACTGCTGAGAGATGCCGACGACCTGCAGAAATGCCTGGCCGT |
| | | GTATCAGGCTGGCGCTAGAGAAGGCGCTGAGAGAGGACTGTCTGCCATC |
| | | CGGGAAAGACTGGGCCCTCTGGTTGAACAGGGCAGAGTTAGAGCCGCC |
| | | ACAGTGGGATCTCTTGCAGGCCAGCCTCTGCAAGAAAGAGCCCAGGCTT |
| | | GGGGCGAGAGACTGAGGGCCAGAATGGAAGAGATGGGCAGCAGAACCA |
| | | GAGACAGACTGGACGAAGTGAAAGAACAGGTGGCCGAAGTGCGCGCCA |
| | | AGCTCGAAGAACAAGCCCAGCAGATTCGGCTGCAGGCTGAAGCCTTTCA |
| | | GGCCCGGCTGAAGAGTTGGTTCGAGCCCCTGGTGGAAGATATGCAGAGA |
| | | CAGTGGGCTGGACTGGTGGAAAAGGTGCAGGCTGCCGTGGGAACATCT |
| | | GCTGCTCCAGTGCCTAGCGACAACCACTGA |
| 209 | APOE3 ch-V2 | ATGAAAGTCCTCTGGGCCGCTCTCCTGGTGACCTTTCTGGCCGGCTGTC |
| | (Christchurch, | AGGCCAAAGTGGAACAGGCCGTGGAAACCGAGCCTGAGCCTGAACTGA |
| | R136S) | GACAGCAGACAGAGTGGCAGTCCGGCCAGAGATGGGAACTTGCCCTGG |
| | | GCAGATTCTGGGACTACCTGAGATGGGTGCAGACCCTGAGCGAACAGGT |
| | | GCAAGAGGAACTGCTGAGCAGCCAAGTGACCCAAGAGCTGAGAGCCCTG |
| | | ATGGACGAGACAATGAAAGAACTCAAAGCTTATAAGAGCGAGCTCGAAGA |
| | | GCAGCTGACCCCTGTGGCCGAGGAAACAAGAGCCAGACTGAGCAAAGA |
| | | GCTGCAGGCCGCTCAAGCTAGACTGGGCGCCGATATGGAAGATGTGTGC |
| | | GGCAGACTGGTGCAGTACAGAGGCGAAGTGCAGGCCATGCTGGGCCAG |
| | | TCTACCGAGGAACTGAGAGTGTCTCTGGCCAGCCACCTGAGAAAGCTGC |
| | | GGAAGAGACTGCTGAGAGATGCCGACGACCTGCAGAAACGCCTGGCCG |
| | | TGTATCAGGCTGGCGCTAGAGAAGGCGCTGAGAGAGGACTGTCTGCCAT |
| | | CCGGGAAAGACTGGGCCCTCTGGTTGAACAGGGCAGAGTTAGAGCCGC |
| | | CACAGTGGGATCTCTTGCAGGCCAGCCTCTGCAAGAAAGAGCCCAGGCT |
| | | TGGGGCGAGAGACTGAGGGCCAGAATGGAAGAGATGGGCAGCAGAACC |
| | | AGAGACAGACTGGACGAAGTGAAAGAACAGGTGGCCGAAGTGCGCGCC |
| | | AAGCTCGAAGAACAAGCCCAGCAGATTCGGCTGCAGGCTGAAGCCTTTC |
| | | AGGCCCGGCTGAAGAGTTGGTTCGAGCCCCTGGTGGAAGATATGCAGAG |
| | | ACAGTGGGCTGGACTGGTGGAAAAGGTGCAGGCTGCCGTGGGAACATCT |
| | | GCTGCTCCAGTGCCTAGCGACAACCACTGA |
| 210 | APOE2b- | ATGAAAGTCCTCTGGGCCGCTCTCCTGGTGACCTTTCTGGCCGGCTGTC |
| | APOE2-Jac- | AGGCCAAAGTGGAACAGGCCGTGGAAACCGAGCCTGAGCCTGAACTGA |
| | V2 | GACAGCAGACAGAGTGGCAGTCCGGCCAGAGATGGGAACTTGCCCTGG |
| | (Jacksonville, | GCAGATTCTGGGACTACCTGAGATGGGTGCAGACCCTGAGCGAACAGGT |
| | V236E) | GCAAGAGGAACTGCTGAGCAGCCAAGTGACCCAAGAGCTGAGAGCCCTG |
| | | ATGGACGAGACAATGAAAGAACTCAAAGCTTATAAGAGCGAGCTCGAAGA |
| | | GCAGCTGACCCCTGTGGCCGAGGAAACAAGAGCCAGACTGAGCAAAGA |
| | | GCTGCAGGCCGCTCAAGCTAGACTGGGCGCCGATATGGAAGATGTGTGC |
| | | GGCAGACTGGTGCAGTACAGAGGCGAAGTGCAGGCCATGCTGGGCCAG |
| | | TCTACCGAGGAACTGAGAGTGCGGCTGGCCAGCCACCTGAGAAAGCTGC |
| | | GGAAGAGACTGCTGAGAGATGCCGACGACCTGCAGAAATGCCTGGCCGT |
| | | GTATCAGGCTGGCGCTAGAGAAGGCGCTGAGAGAGGACTGTCTGCCATC |
| | | CGGGAAAGACTGGGCCCTCTGGTTGAACAGGGCAGAGTTAGAGCCGCC |
| | | ACAGTGGGATCTCTTGCAGGCCAGCCTCTGCAAGAAAGAGCCCAGGCTT |
| | | GGGGCGAGAGACTGAGGGCCAGAATGGAAGAGATGGGCAGCAGAACCA |
| | | GAGACAGACTGGACGAAGTGAAAGAACAGGAGGCCGAAGTGCGCGCCA |
| | | AGCTCGAAGAACAAGCCCAGCAGATTCGGCTGCAGGCTGAAGCCTTTCA |
| | | GGCCCGGCTGAAGAGTTGGTTCGAGCCCCTGGTGGAAGATATGCAGAGA |
| | | CAGTGGGCTGGACTGGTGGAAAAGGTGCAGGCTGCCGTGGGAACATCT |
| | | GCTGCTCCAGTGCCTAGCGACAACCACTGA |
| 211 | APOE3b- | ATGAAAGTCCTCTGGGCCGCTCTCCTGGTGACCTTTCTGGCCGGCTGTC |
| | APOE3-Jac- | AGGCCAAAGTGGAACAGGCCGTGGAAACCGAGCCTGAGCCTGAACTGA |
| | V2 | GACAGCAGACAGAGTGGCAGTCCGGCCAGAGATGGGAACTTGCCCTGG |
| | (Jacksonville, | GCAGATTCTGGGACTACCTGAGATGGGTGCAGACCCTGAGCGAACAGGT |
| | V236E) | GCAAGAGGAACTGCTGAGCAGCCAAGTGACCCAAGAGCTGAGAGCCCTG |
| | | ATGGACGAGACAATGAAAGAACTCAAAGCTTATAAGAGCGAGCTCGAAGA |
| | | GCAGCTGACCCCTGTGGCCGAGGAAACAAGAGCCAGACTGAGCAAAGA |
| | | GCTGCAGGCCGCTCAAGCTAGACTGGGCGCCGATATGGAAGATGTGTGC |
| | | GGCAGACTGGTGCAGTACAGAGGCGAAGTGCAGGCCATGCTGGGCCAG |
| | | TCTACCGAGGAACTGAGAGTGCGGCTGGCCAGCCACCTGAGAAAGCTGC |
| | | GGAAGAGACTGCTGAGAGATGCCGACGACCTGCAGAAACGCCTGGCCG |
| | | TGTATCAGGCTGGCGCTAGAGAAGGCGCTGAGAGAGGACTGTCTGCCAT |
| | | CCGGGAAAGACTGGGCCCTCTGGTTGAACAGGGCAGAGTTAGAGCCGC |
| | | CACAGTGGGATCTCTTGCAGGCCAGCCTCTGCAAGAAAGAGCCCAGGCT |
| | | TGGGGCGAGAGACTGAGGGCCAGAATGGAAGAGATGGGCAGCAGAACC |
| | | AGAGACAGACTGGACGAAGTGAAAGAACAGGAGGCCGAAGTGCGCGCC |
| | | AAGCTCGAAGAACAAGCCCAGCAGATTCGGCTGCAGGCTGAAGCCTTTC |
| | | AGGCCCGGCTGAAGAGTTGGTTCGAGCCCCTGGTGGAAGATATGCAGAG |
| | | ACAGTGGGCTGGACTGGTGGAAAAGGTGCAGGCTGCCGTGGGAACATCT |
| | | GCTGCTCCAGTGCCTAGCGACAACCACTGA |
| 212 | APOE2-V3 | ATGAAAGTCCTCTGGGCCGCTCTCCTGGTGACCTTTCTGGCAGGATGCC |
| | | AGGCCAAGGTGGAGCAAGCGGTGGAGACAGAGCCGGAGCCCGAGCTGC |
| | | GCCAGCAGACCGAGTGGCAGAGCGGCCAGCGCTGGGAACTGGCACTGG |
| | | GTCGCTTTTGGGATTACCTGCGCTGGGTGCAGACACTGTCTGAGCAGGT |
| | | GCAGGAGGAGCTGCTCAGCTCCCAGGTCACCCAGGAACTGAGGGCGCT |
| | | GATGGACGAGACCATGAAAGAACTCAAAGCTTATAAGAGCGAGCTCGAA |
| | | GAGCAGCTGACCCCGGTGGCGGAGGAGACGCGGGCACGGCTGTCCAAG |
| | | GAGCTGCAGGCGGCGCAGGCCCGGCTGGGCGCGGACATGGAGGACGT |
| | | GTGCGGCCGCCTGGTGCAGTACCGCGGCGAGGTGCAGGCCATGCTCGG |
| | | CCAGAGCACCGAGGAGCTGCGGGTGCGCCTCGCCTCCCACCTGCGCAA |
| | | GCTGCGTAAGCGGCTCCTCCGCGATGCCGATGACCTGCAGAAGTGCCTG |
| | | GCAGTGTACCAGGCCGGGGCCCGCGAGGGCGCCGAGCGCGGCCTCAG |
| | | CGCCATCCGCGAGCGCCTGGGGCCCCTGGTGGAACAGGGCCGCGTGC |
| | | GGGCCGCCACTGTGGGCTCCCTGGCCGGCCAGCCGCTACAGGAGCGG |
| | | GCCCAGGCCTGGGGCGAGCGGCTGCGCGCGCGGATGGAGGAGATGGG |
| | | CAGCCGGACCCGCGACCGCCTGGACGAGGTGAAGGAGCAGGTGGCGG |
| | | AGGTGCGCGCCAAGCTGGAGGAGCAGGCCCAGCAGATACGCCTGCAGG |
| | | CCGAGGCCTTCCAGGCCCGCCTCAAGAGCTGGTTCGAGCCCCTGGTGG |
| | | AAGACATGCAGCGCCAGTGGGCCGGGCTGGTGGAGAAGGTGCAGGCTG |
| | | CCGTGGGCACCAGCGCCGCCCCTGTGCCCAGCGACAATCACTGA |
| 213 | APOE3-V3 | ATGAAAGTCCTCTGGGCCGCTCTCCTGGTGACCTTTCTGGCAGGATGCC |
| | | AGGCCAAGGTGGAGCAAGCGGTGGAGACAGAGCCGGAGCCCGAGCTGC |
| | | GCCAGCAGACCGAGTGGCAGAGCGGCCAGCGCTGGGAACTGGCACTGG |
| | | GTCGCTTTTGGGATTACCTGCGCTGGGTGCAGACACTGTCTGAGCAGGT |
| | | GCAGGAGGAGCTGCTCAGCTCCCAGGTCACCCAGGAACTGAGGGCGCT |
| | | GATGGACGAGACCATGAAAGAACTCAAAGCTTATAAGAGCGAGCTCGAA |
| | | GAGCAGCTGACCCCGGTGGCGGAGGAGACGCGGGCACGGCTGTCCAAG |
| | | GAGCTGCAGGCGGCGCAGGCCCGGCTGGGCGCGGACATGGAGGACGT |
| | | GTGCGGCCGCCTGGTGCAGTACCGCGGCGAGGTGCAGGCCATGCTCGG |
| | | CCAGAGCACCGAGGAGCTGCGGGTGCGCCTCGCCTCCCACCTGCGCAA |
| | | GCTGCGTAAGCGGCTCCTCCGCGATGCCGATGACCTGCAGAAGCGCCT |
| | | GGCAGTGTACCAGGCCGGGGCCCGCGAGGGCGCCGAGCGCGGCCTCA |
| | | GCGCCATCCGCGAGCGCCTGGGGCCCCTGGTGGAACAGGGCCGCGTG |
| | | CGGGCCGCCACTGTGGGCTCCCTGGCCGGCCAGCCGCTACAGGAGCGG |
| | | GCCCAGGCCTGGGGCGAGCGGCTGCGCGCGCGGATGGAGGAGATGGG |
| | | CAGCCGGACCCGCGACCGCCTGGACGAGGTGAAGGAGCAGGTGGCGG |
| | | AGGTGCGCGCCAAGCTGGAGGAGCAGGCCCAGCAGATACGCCTGCAGG |
| | | CCGAGGCCTTCCAGGCCCGCCTCAAGAGCTGGTTCGAGCCCCTGGTGG |
| | | AAGACATGCAGCGCCAGTGGGCCGGGCTGGTGGAGAAGGTGCAGGCTG |
| | | CCGTGGGCACCAGCGCCGCCCCTGTGCCCAGCGACAATCACTGA |
| 214 | APOE2 ch-V3 | ATGAAAGTCCTCTGGGCCGCTCTCCTGGTGACCTTTCTGGCAGGATGCC |
| | (Christchurch, | AGGCCAAGGTGGAGCAAGCGGTGGAGACAGAGCCGGAGCCCGAGCTGC |
| | R136S) | GCCAGCAGACCGAGTGGCAGAGCGGCCAGCGCTGGGAACTGGCACTGG |
| | | GTCGCTTTTGGGATTACCTGCGCTGGGTGCAGACACTGTCTGAGCAGGT |
| | | GCAGGAGGAGCTGCTCAGCTCCCAGGTCACCCAGGAACTGAGGGCGCT |
| | | GATGGACGAGACCATGAAAGAACTCAAAGCTTATAAGAGCGAGCTCGAA |
| | | GAGCAGCTGACCCCGGTGGCGGAGGAGACGCGGGCACGGCTGTCCAAG |
| | | GAGCTGCAGGCGGCGCAGGCCCGGCTGGGCGCGGACATGGAGGACGT |
| | | GTGCGGCCGCCTGGTGCAGTACCGCGGCGAGGTGCAGGCCATGCTCGG |
| | | CCAGAGCACCGAGGAGCTGCGGGTGAGCCTCGCCTCCCACCTGCGCAA |
| | | GCTGCGTAAGCGGCTCCTCCGCGATGCCGATGACCTGCAGAAGTGCCTG |
| | | GCAGTGTACCAGGCCGGGGCCCGCGAGGGCGCCGAGCGCGGCCTCAG |
| | | CGCCATCCGCGAGCGCCTGGGGCCCCTGGTGGAACAGGGCCGCGTGC |
| | | GGGCCGCCACTGTGGGCTCCCTGGCCGGCCAGCCGCTACAGGAGCGG |
| | | GCCCAGGCCTGGGGCGAGCGGCTGCGCGCGCGGATGGAGGAGATGGG |
| | | CAGCCGGACCCGCGACCGCCTGGACGAGGTGAAGGAGCAGGTGGCGG |
| | | AGGTGCGCGCCAAGCTGGAGGAGCAGGCCCAGCAGATACGCCTGCAGG |
| | | CCGAGGCCTTCCAGGCCCGCCTCAAGAGCTGGTTCGAGCCCCTGGTGG |
| | | AAGACATGCAGCGCCAGTGGGCCGGGCTGGTGGAGAAGGTGCAGGCTG |
| | | CCGTGGGCACCAGCGCCGCCCCTGTGCCCAGCGACAATCACTGA |
| 215 | APOE3 ch-V3 | ATGAAAGTCCTCTGGGCCGCTCTCCTGGTGACCTTTCTGGCAGGATGCC |
| | (Christchurch, | AGGCCAAGGTGGAGCAAGCGGTGGAGACAGAGCCGGAGCCCGAGCTGC |
| | R136S) | GCCAGCAGACCGAGTGGCAGAGCGGCCAGCGCTGGGAACTGGCACTGG |
| | | GTCGCTTTTGGGATTACCTGCGCTGGGTGCAGACACTGTCTGAGCAGGT |
| | | GCAGGAGGAGCTGCTCAGCTCCCAGGTCACCCAGGAACTGAGGGCGCT |
| | | GATGGACGAGACCATGAAAGAACTCAAAGCTTATAAGAGCGAGCTCGAA |
| | | GAGCAGCTGACCCCGGTGGCGGAGGAGACGCGGGCACGGCTGTCCAAG |
| | | GAGCTGCAGGCGGCGCAGGCCCGGCTGGGCGCGGACATGGAGGACGT |
| | | GTGCGGCCGCCTGGTGCAGTACCGCGGCGAGGTGCAGGCCATGCTCGG |
| | | CCAGAGCACCGAGGAGCTGCGGGTGAGCCTCGCCTCCCACCTGCGCAA |
| | | GCTGCGTAAGCGGCTCCTCCGCGATGCCGATGACCTGCAGAAGCGCCT |
| | | GGCAGTGTACCAGGCCGGGGCCCGCGAGGGCGCCGAGCGCGGCCTCA |
| | | GCGCCATCCGCGAGCGCCTGGGGCCCCTGGTGGAACAGGGCCGCGTG |
| | | CGGGCCGCCACTGTGGGCTCCCTGGCCGGCCAGCCGCTACAGGAGCGG |
| | | GCCCAGGCCTGGGGCGAGCGGCTGCGCGCGCGGATGGAGGAGATGGG |
| | | CAGCCGGACCCGCGACCGCCTGGACGAGGTGAAGGAGCAGGTGGCGG |
| | | AGGTGCGCGCCAAGCTGGAGGAGCAGGCCCAGCAGATACGCCTGCAGG |
| | | CCGAGGCCTTCCAGGCCCGCCTCAAGAGCTGGTTCGAGCCCCTGGTGG |
| | | AAGACATGCAGCGCCAGTGGGCCGGGCTGGTGGAGAAGGTGCAGGCTG |
| | | CCGTGGGCACCAGCGCCGCCCCTGTGCCCAGCGACAATCACTGA |
| 216 | APOE2b- | ATGAAAGTCCTCTGGGCCGCTCTCCTGGTGACCTTTCTGGCAGGATGCC |
| | APOE2-Jac- | AGGCCAAGGTGGAGCAAGCGGTGGAGACAGAGCCGGAGCCCGAGCTGC |
| | V3 | GCCAGCAGACCGAGTGGCAGAGCGGCCAGCGCTGGGAACTGGCACTGG |
| | (Jacksonville, | GTCGCTTTTGGGATTACCTGCGCTGGGTGCAGACACTGTCTGAGCAGGT |
| | V236E) | GCAGGAGGAGCTGCTCAGCTCCCAGGTCACCCAGGAACTGAGGGCGCT |
| | | GATGGACGAGACCATGAAAGAACTCAAAGCTTATAAGAGCGAGCTCGAA |
| | | GAGCAGCTGACCCCGGTGGCGGAGGAGACGCGGGCACGGCTGTCCAAG |
| | | GAGCTGCAGGCGGCGCAGGCCCGGCTGGGCGCGGACATGGAGGACGT |
| | | GTGCGGCCGCCTGGTGCAGTACCGCGGCGAGGTGCAGGCCATGCTCGG |
| | | CCAGAGCACCGAGGAGCTGCGGGTGCGCCTCGCCTCCCACCTGCGCAA |
| | | GCTGCGTAAGCGGCTCCTCCGCGATGCCGATGACCTGCAGAAGTGCCTG |
| | | GCAGTGTACCAGGCCGGGGCCCGCGAGGGCGCCGAGCGCGGCCTCAG |
| | | CGCCATCCGCGAGCGCCTGGGGCCCCTGGTGGAACAGGGCCGCGTGC |
| | | GGGCCGCCACTGTGGGCTCCCTGGCCGGCCAGCCGCTACAGGAGCGG |
| | | GCCCAGGCCTGGGGCGAGCGGCTGCGCGCGCGGATGGAGGAGATGGG |
| | | CAGCCGGACCCGCGACCGCCTGGACGAGGTGAAGGAGCAGGAGGCGG |
| | | AGGTGCGCGCCAAGCTGGAGGAGCAGGCCCAGCAGATACGCCTGCAGG |
| | | CCGAGGCCTTCCAGGCCCGCCTCAAGAGCTGGTTCGAGCCCCTGGTGG |
| | | AAGACATGCAGCGCCAGTGGGCCGGGCTGGTGGAGAAGGTGCAGGCTG |
| | | CCGTGGGCACCAGCGCCGCCCCTGTGCCCAGCGACAATCACTGA |
| 217 | APOE3b- | ATGAAAGTCCTCTGGGCCGCTCTCCTGGTGACCTTTCTGGCAGGATGCC |
| | APOE3-Jac- | AGGCCAAGGTGGAGCAAGCGGTGGAGACAGAGCCGGAGCCCGAGCTGC |
| | V3 | GCCAGCAGACCGAGTGGCAGAGCGGCCAGCGCTGGGAACTGGCACTGG |
| | (Jacksonville, | GTCGCTTTTGGGATTACCTGCGCTGGGTGCAGACACTGTCTGAGCAGGT |
| | V236E) | GCAGGAGGAGCTGCTCAGCTCCCAGGTCACCCAGGAACTGAGGGCGCT |
| | | GATGGACGAGACCATGAAAGAACTCAAAGCTTATAAGAGCGAGCTCGAA |
| | | GAGCAGCTGACCCCGGTGGCGGAGGAGACGCGGGCACGGCTGTCCAAG |
| | | GAGCTGCAGGCGGCGCAGGCCCGGCTGGGCGCGGACATGGAGGACGT |
| | | GTGCGGCCGCCTGGTGCAGTACCGCGGCGAGGTGCAGGCCATGCTCGG |
| | | CCAGAGCACCGAGGAGCTGCGGGTGCGCCTCGCCTCCCACCTGCGCAA |
| | | GCTGCGTAAGCGGCTCCTCCGCGATGCCGATGACCTGCAGAAGCGCCT |
| | | GGCAGTGTACCAGGCCGGGGCCCGCGAGGGCGCCGAGCGCGGCCTCA |
| | | GCGCCATCCGCGAGCGCCTGGGGCCCCTGGTGGAACAGGGCCGCGTG |
| | | CGGGCCGCCACTGTGGGCTCCCTGGCCGGCCAGCCGCTACAGGAGCGG |
| | | GCCCAGGCCTGGGGCGAGCGGCTGCGCGCGCGGATGGAGGAGATGGG |
| | | CAGCCGGACCCGCGACCGCCTGGACGAGGTGAAGGAGCAGGAGGCGG |
| | | AGGTGCGCGCCAAGCTGGAGGAGCAGGCCCAGCAGATACGCCTGCAGG |
| | | CCGAGGCCTTCCAGGCCCGCCTCAAGAGCTGGTTCGAGCCCCTGGTGG |
| | | AAGACATGCAGCGCCAGTGGGCCGGGCTGGTGGAGAAGGTGCAGGCTG |
| | | CCGTGGGCACCAGCGCCGCCCCTGTGCCCAGCGACAATCACTGA |

As is known in the art, expression of a gene product requires the presence of expression control and/or regulatory sequences such as one or more promoters and any other nucleic acid sequences, such as introns, necessary for expression of the selected nucleic acid sequence, all operably linked to the selected sequence, and may include an enhancer sequence. Thus, in one embodiment, the expression construct comprising an expression cassette encoding at least one of APOE2 and APOE3 comprises a promoter. In an alternative embodiment, the expression cassette as disclosed herein comprising a nucleic acid comprising a sequence encoding a first RNA and a sequence encoding a second RNA as disclosed herein, wherein the second RNA comprises a guide sequence of at least 19 nucleotides substantially complementary to part of an APOE gene, and wherein the first RNA and second RNA each comprise a hairpin, further comprising a second nucleic acid encoding at least one of APOE2 and APOE3, and comprises one or more promoters, preferably one promoter. Such a design was identified by the current inventors to be advantageous given the capacity of later downstream processing relating to transfection and expression.

### Promoter

The nucleotide sequence comprising an expression cassette or expression cassettes as defined herein above for expression in a mammalian cell, further preferably comprises at least one mammalian cell-compatible expression control sequence, e.g. a promoter, that is/are operably linked to the sequence coding for the gene product of interest, thus forming an expression cassette for expression of the gene product of interest in mammalian target cell to be treated by gene therapy with the gene product of interest. Many such promoters are known in the art (see Sambrook and Russel, 2001, supra). Constitutive promoters that are broadly expressed in many cell types, such as the CMV promoter may be used. However, more preferred will be promoters that are inducible, tissue-specific, cell-type-specific, or cell cycle-specific. Preferably a pol II promoter is used, such as a CAG promoter (SEQ ID NO. 191) (i.a. Miyazaki et al. Gene. 79 (2): 269-77; Niwa, Gene. 108 (2): 193-9), a PGK promoter, a CMV promoter (Such as depicted e.g. in FIG. 2 of WO2016102664, which is herein incorporated by references) or an adapted/synthetic promotor (P1, SEQ ID NO. 192 and P2, SEQ ID NO. 193). As Alzheimer's Disease primarily affects the brain, it may, in particular, be useful to use a neuro specific promoter. Thus, in some embodiment, the promoter is a promoter capable of driving transcription in a brain cell. Examples of suitable neuro specific promoters are Neuron-Specific Enolase (NSE), human synapsin 1, caMK kinase and tubulin (Hioki et al. Gene Ther. 2007 Jun; 14 (11): 872-82). Other suitable promoters that can be contemplated are inducible promoters, i.e. a promoter that initiates transcription only when the host cell is exposed to some particular stimulus.

The expression cassette for expression of at least APOE, as described above, further preferably encodes a polyA signal comprised in the DNA expression cassette operably linked to the 3′ end of the RNA molecule encoded by the transgene, as described above. Preferably, said polyA signal is the simian virus 40 polyadenylations (SV40 polyA, SEQ ID NO. 194), a synthetic polyadenylation signal, the Bovine Growth Hormone polyadenylation signal (BGH polyA), or the Human Growth Hormone polyadenylation signal (HGH polyA).

### Vectors

An isolated nucleic acid as described herein may exist on its own, as part of an expression cassette and/or as part of a vector. Generally, a vector can be a plasmid, cosmid, phagemid, bacterial artificial chromosome (BAC), or a viral vector such as a gene therapy vector. Expression cassettes or vectors according to the invention can be transferred to a cell, using e.g. transfection methods. Any suitable means may suffice to transfer an expression cassette according to the invention. Preferably, viral vectors are used that stably transfer the expression cassette to the cells such that stable expression of the double stranded RNAs that induce sequence specific inhibition of the APOE gene as described above can be achieved. Suitable vectors may be lentiviral vectors, retrotransposon based vector systems, or AAV vectors. It is understood that as e.g. lentiviral vectors carry an RNA genome, the RNA genome will encode for the said expression cassette such that after transduction of a cell, the said DNA sequence and said expression cassette is formed. Preferably a viral vector is used such as AAV. Therefore, in some embodiment, an expression cassette as disclosed herein is flanked by Inverted Terminal Repeats. Preferably the AAV vector that is used is an AAV vector of serotype 5. AAV of serotype 5 (also referred to as AAV5) may be particularly useful for transducing human neurons and human astrocytes such as shown in the examples. Therefore, in some embodiment, there is an AAV comprising an expression cassette as disclosed herein. Thus, AAV5 can efficiently transduce different human cell types of the CNS including FBN, dopaminergic neurons, motor neurons and astrocytes and is therefore a suitable vector candidate to deliver therapeutic genes to the CNS to treat neurogenerative diseases, including but not limited to the treatment of Alzheimer's Disease via targeting e.g. APOE as described herein. The production of AAV vectors comprising any expression cassette of interest is well described in; WO2007/046703, WO2007/148971, WO2009/014445, WO2009/104964, WO2011/122950, WO2013/036118, which are incorporated herein in its entirety.

AAV sequences that may be used in the present invention for the production of AAV vectors, e.g. produced in insect or mammalian cell lines, can be derived from the genome of any AAV serotype. Generally, the AAV serotypes have genomic sequences of significant homology at the amino acid and the nucleic acid levels, provide an identical set of genetic functions, produce virions which are essentially physically and functionally equivalent, and replicate and assemble by practically identical mechanisms. For the genomic sequence of the various AAV serotypes and an overview of the genomic similarities see e.g. GenBank Accession number U89790; GenBank Accession number J01901; GenBank Accession number AF043303; GenBank Accession number AF085716; Chlorini et al. (1997, J. Vir. 71:6823-33); Srivastava et al. (1983, J. Vir. 45:555-64); Chlorini et al. (1999, J. Vir. 73:1309-1319); Rutledge et al. (1998, J. Vir. 72:309-319); and Wu et al. (2000, J. Vir. 74:8635-47). AAV serotypes 1, 2, 3, 4 and 5 are preferred sources of AAV nucleotide sequences for use in the context of the present invention. Preferably the AAV ITR sequences for use in the context of the present invention are derived from AAV1, AAV2, and/or AAV5. Likewise, the Rep52, Rep40, Rep78 and/or Rep68 coding sequences are preferably derived from AAV1, AAV2 and AAV5. The sequences coding for the VP1, VP2, and VP3 capsid proteins for use in the context of the present invention may however be taken from any of the known 42 serotypes, more preferably from AAV1, AAV2, AAV3, AAV4, AAV5, AAV6, AAV7, AAV8 or AAV9 or newly developed AAV-like particles obtained by e.g. capsid shuffling techniques and AAV capsid libraries. AAV capsids may consist of VP1, VP2 and VP3, but may also consist of VP1 and VP3. In some embodiment, the AAV vector according to the inventions comprises AAV5 or AAV9 capsid protein. In some embodiment, the AAV vector according to the inventions comprises AAV5 capsid protein. In some embodiment, the AAV vector according to the inventions comprises AAV9 capsid protein.

In another embodiment, a host cell is provided comprising the said nucleic acid or said expression cassette according to the invention. For example, the said expression cassette or nucleic acid may be comprised in a plasmid contained in bacteria. Said expression cassette or nucleic acid may also be comprised in a production cell that produces e.g. a viral vector. Said expression cassette may also be provided in a baculovirus vector.

Various modifications of the nucleotide sequences as defined above, including e.g. the wildtype AAV sequences, for proper expression in the host cell is achieved by application of well-known genetic engineering techniques such as described e.g. in Sambrook and Russell (2001) “Molecular Cloning: A Laboratory Manual (3rd edition), Cold Spring Harbor Laboratory, Cold Spring Harbor Laboratory Press, New York. Various further modifications of coding regions are known to the skilled artisan which could increase the yield of the encoded proteins. These modifications are within the scope of the present invention.

In one embodiment, any mammalian cell may be infected by an AAV vector of the invention, for example, but not limited to, a muscle cell, a liver cell, a nerve cell, a glial cell and an epithelial cell. In a preferred embodiment, the cell to be infected is a human cell.

It is to be understood that the capsid amino acid sequences and the nucleotide sequences encoding them can be engineered, for example, the sequence may be a hybrid form or may be codon optimized, such as for example by codon usage of AcmNPv or Spodoptera frugiperda. The capsid proteins may be engineered, for example, via DNA shuffling, error prone PCR, bioinformatics rational design or site saturated mutagenesis. Resulting capsids are based on the existing serotypes but contain various amino acid or nucleotide changes that improve the features of such capsids. The resulting capsids can be a combination of various parts of existing serotypes, “shuffled capsids” or contain completely novel changes, i.e. additions, deletions or substitutions of one or more amino acids or nucleotides, organized in groups or spread over the whole length of gene or protein. See for example Schaffer and Maheshri; Proceedings of the 26th Annual International Conference of the IEEE EMBS San Francisco, CA, USA; Sep. 1-5, 2004, pages 3520-3523; Asuri et al., 2012, Molecular Therapy 20 (2): 329-3389; Lisowski et al., 2014, Nature 506 (7488): 382-386, herein incorporated by reference.

In some embodiments, the ITRs and capsid proteins (or parts thereof) in the AAV vector of the invention may be from different AAV serotypes. By way of example, and not limitation, the ITRs may be derived from AAV2, whilst the capsid proteins may be derived from a different serotype, for example AAV5 or AAV9.

### Compositions

In another aspect, the invention pertains to a pharmaceutical composition comprising an AAV vector according to the invention, i.e. an AAV vector comprising the nucleic acid according to the invention, or the expression cassette according to the inventions. In one embodiment, the invention provides a composition comprising an AAV vector according to the invention and suitable excipients, such as buffers and stabilizers, antioxidants etc. In one particular embodiment, these compositions are used to transduce cells in vitro or ex vivo, in which case the excipients will need to be compatible with cell culture. In other preferred embodiments, the compositions are used for the treatment of (human) subjects. For that purpose, the invention provides a pharmaceutical composition comprising an AAV vector according to the invention and at least one pharmaceutically acceptable carrier. In the case of AAV gene delivery vehicles, a pharmaceutical composition typically comprise physiological buffers, such as e.g. PBS, comprising further stabilizing agents such as e.g. sucrose. Such compositions are compatible with and suitable and intended for use in subsequent intravenous, intrathecal, intraparenchymal, intravitreal, subretinal administration or for use in organ-targeted vascular delivery such as intraporal or intracoronary delivery or isolated limb perfusion. Therefore, in some embodiments, the invention provides the nucleic acid according to the invention, or the expression cassette according to the invention, or the AAV vector according to the invention, or the pharmaceutical composition according to the invention for a use as disclosed herein, wherein the nucleic acid, the expression cassette, the AAV vector or the pharmaceutical composition is administered to the central nervous system, preferably by intracerebral injection, intraparenchymal injection, intrathecal injection, intra cisterna magna injection, intracerebroventricular injection or a combination thereof, more preferably by convection enhanced delivery.

### Uses

Another aspect of the invention relates to the use of an AAV vector according to the invention, or a composition comprising the AAV vector. In one embodiment, there is provided an expression cassette according to the invention, an AAV vector according to the invention, or a pharmaceutical composition according to the invention for use as a medicament. In a further embodiment, there is provided a nucleic acid according to the invention, an expression cassette according to the invention, an AAV vector according to the invention, or a pharmaceutical composition according to the invention for use in the treatment of in the treatment and/or prevention of tauopathies in a subject. In a further embodiment, there is provided a nucleic acid according to the invention, an expression cassette according to the invention, an AAV vector according to the invention, or a pharmaceutical composition according to the invention for use in the treatment of and/or prevention of Alzheimer's disease in a subject. In a further embodiment, there is provided a nucleic acid according to the invention, an expression cassette according to the invention, an AAV vector according to the invention, or a pharmaceutical composition according to the invention for use in the treatment as a medicament wherein the subject is a carrier of an APOE4 allele, wherein preferably the subject is homozygous for an APOE4 allele.

### Host Cells and Methods for Producing an AAV Vector and Kits Thereof

In a further aspect, the invention relates to a method for producing a nucleic acid according to the invention, an expression cassette according to the invention, an AAV vector according to the invention, or a pharmaceutical composition according to the invention.

The methods for producing the nucleic acid of the invention comprise any methods for producing nucleic acids, including but not limited to de novo synthesis all of which would be apparent to the skilled person.

The method for producing an AAV vector preferably comprising the steps of: a) culturing a host cell as herein defined above under conditions such that the AAV vector is produced; and, b) optionally, one or more of recovery, purification and formulation of the AAV vector.

The host cell, preferably is a host cell that is suitable for the production of AAV vectors. Accordingly, the host cell is a host cell that is amenable to in vitro culture, preferably at large scale. Host cell that are suitable for the production of AAV vectors are well-known in the art and will typically be a mammalian or an insect cell line. Mammalian cell lines for producing AAV vectors are selected from among any mammalian species, including, without limitation, cells such as A549, WEHI, 3T3, 10T1/2, BHK, MDCK, COS 1, COS 7, BSC 1, BSC 40, BMT 10, VERO, WI38, HeLa, a HEK 293 cell (which express functional adenoviral E1), Saos, C2C12, L cells, HT1080, HepG2 and primary fibroblast, hepatocyte and myoblast cells derived from mammals including human, monkey, mouse, rat, rabbit, and hamster. The selection of the mammalian species providing the cells is not a limitation of this invention; nor is the type of mammalian cell, i.e., fibroblast, hepatocyte, tumour cell. Mammalian cell lines for producing AAV vectors in particular include a broad range of HEK293 cell lines, of which the HEK293T cell line is preferred.

Insect cell lines for producing AAV vectors can be any cell line that is suitable for the production of heterologous proteins. Preferably the insect cell allows for replication of baculoviral vectors and can be maintained in culture, more preferably in suspended culture. In a preferred embodiment, the insect cell allows for replication of recombinant parvoviral vectors, including rAAV vectors. For example, the cell line used can be from Spodoptera frugiperda, Drosophila, or mosquito, e.g., Aedes albopictus derived cell lines. Preferred insect cells or cell lines are cells from the insect species which are susceptible to baculovirus infection, including e.g. S2 (CRL-1963, ATCC), Se301, SelZD2109, SeUCR1, Sf9, Sf900+, Sf21, BTI-TN-5B1-4, MG-1, Tn368, HzAm1, Ha2302, Hz2E5, High Five (Invitrogen, CA, USA) and expresSF+® (U.S. Pat. No. 6,103,526; Protein Sciences Corp., CT, USA).

Thus, in one embodiment, the expression cassette or construct is an insect cell-compatible vector or a mammalian cell-compatible vector. A “mammalian cell compatible vector” is understood to be a nucleic acid molecule capable of productive transformation or transfection of a mammalian cell or cell line. Mammalian cell-compatible vectors are well-known in the art. An “insect cell-compatible vector” is understood to be a nucleic acid molecule capable of productive transformation or transfection of an insect or insect cell. Exemplary insect cell compatible vectors include plasmids, linear nucleic acid molecules, and recombinant viruses, such as baculoviruses. Any vector can be employed as long as it is insect cell-compatible. The mammalian or insect cell-compatible vector may integrate into the cell's genome but the presence of the vector in the cell need not be permanent and transient episomal vectors are also included. The vectors can be introduced by any means known, for example by chemical treatment of the cells, electroporation, or infection.

The AAV in the supernatant can be recovered and/or purified using suitable techniques which are known to those of skill in the art. For example, monolith columns (e.g., in ion exchange, affinity or IMAC mode), chromatography (e.g., capture chromatography, fixed method chromatography, and expanded bed chromatography), centrifugation, filtration and precipitation, can be used for purification and concentration. These methods may be used alone or in combination. In one embodiment, capture chromatography methods, including column-based or membrane-based systems, are utilized in combination with filtration and precipitation. Suitable precipitation methods, e.g., utilizing polyethylene glycol (PEG) 8000 and NH₃SO₄, can be readily selected by one of skill in the art. Thereafter, the precipitate can be treated with benzonase and purified using suitable techniques. In addition, recovery may preferably comprises the step of affinity-purification of the (virions comprising the) recombinant parvoviral (rAAV) vector using an anti-AAV antibody, preferably an immobilised antibody. The anti-AAV antibody preferably is a monoclonal antibody. A particularly suitable antibody is a single chain camelid antibody or a fragment thereof as e.g. obtainable from camels or llamas (see e.g. Muyldermans, 2001, Biotechnol. 74:277-302). The antibody for affinity-purification of rAAV preferably is an antibody that specifically binds an epitope on an AAV capsid protein, whereby preferably the epitope is an epitope that is present on capsid protein of more than one AAV serotype. E.g. the antibody may be raised or selected on the basis of specific binding to AAV2 capsid but at the same time also it may also specifically bind to AAV1, AAV3 and AAV5 capsids.

In general, suitable methods for producing an AAV vector according to the invention in mammalian or insect host cells, and means, therefore (such as expression constructs for expression of AAV rep proteins), are described, for mammalian cells in: Clark et al. (1995, Hum. Gene Ther. 6, 1329-134), Gao et al. (1998, Hum. Gene Ther. 9, 2353-2362), Inoue and Russell (1998, J. Virol. 72, 7024-7031), Grimm et al. (1998, Hum. Gene Ther. 9, 2745-2760), Xiao et al. (1998, J. Virol. 72, 2224-2232) and Judd et al. (Mol Ther Nucleic Acids. 2012; 1: e54), and for insect cells in: Urabe et al. (2002, Hum. Gene Ther. 13:1935-1943), WO2007/046703, WO2007/148971, WO2009/014445, WO2009/104964, WO2011/122950, WO2013/036118, WO2015/137802, WO2019/016349 and in co-pending applications EP21177449.2, PCT/EP2021/058794 and PCT/EP2021/058798, all of which are incorporated herein in their entirety.

In a further aspect, there is provided a kit comprising a nucleic acid according to the invention, an expression cassette according to the invention, an AAV vector according to the invention, or a pharmaceutical composition according to the invention, wherein the kit further comprises an immunosuppressive agent.

In certain embodiments of the invention, the immunosuppressive compound may reduce and/or prevent an immune response induced by administration of the nucleic acid, the AAV vector, or the pharmaceutical composition of the invention.

In another aspect, the invention relates to a cell comprising the nucleic acid of the invention or the AAV of the invention, or a host cell.

In some embodiments, the cell of the invention is a prokaryote cell. In some specific embodiments, the cell of the invention is a bacterial cell. In some embodiments, the cell of the invention is a eukaryote cell. In some embodiments, the cell of the invention is a mammalian cell. In some embodiments, the cell of the invention is an insect cell.

The nucleic acid of the invention or the AAV vector of the invention may be delivered into the cell of the invention by any suitable methods, included but not limited to, transfection, transformation transduction, nucleofection, electroporation, microinjection. For example, the expression cassette or nucleic acid of the invention may be comprised in a plasmid contained in bacteria. The expression cassette or nucleic acid of the invention may also be comprised in a production cell that produces e.g. a viral vector. Said expression cassette may also be provided in a baculovirus vector.

Further detail on host cells comprising an AAV vector according to the invention has been provide elsewhere in this application. Any mammalian cell may be infected by the AAV vector of the invention, including but not limited to a muscle cell, a liver cell, a nerve cell, a glial cell or an epithelial cell. In some preferred embodiments of the invention, the cell to be infected is a human cell.

In another aspect, the present invention relates to a method of treating or preventing a disorder, wherein the method comprises administering the nucleic acid of the invention or the AAV of the invention to a subject, thereby treating or preventing the disorder.

In another aspect, the invention relates to the nucleic acid of the invention or the AAV of the invention for use in the manufacture of a medicament for the treatment of a disorder as detailed herein.

The present invention has been described above with reference to a number of exemplary embodiments as shown in the drawings. Modifications and alternative implementations of some parts or elements are possible, and are included in the scope of protection as defined in the appended claims.

### Examples

### Materials and Methods

Design of miRNA Guide Strands Targeting APOE

The miAPOE, miRNA guide strands, were designed targeting coding or non-coding RNA sequences of one of the human APOE transcript (FIG. 1, SEQ ID NO. 1). Regions conserved between humans and non-human primates were identified and 22 nt target sequences selected (SEQ ID NOs. 2-93, table 1) to generate different miRNA guide strands (SEQ ID NOs. 94-185, table 2). Scrambled miRNA guide strands were designed to generate negative controls (SEQ ID NOs. 186-189). In addition, a scramble miRNA guide that was previously designed for a different research program was used for in vivo testing (SEQ ID NO. 294).

### DNA Constructs

The miAPOE and the scrambled control guide sequences were embedded in the human pri-miR-451 scaffold (FIG. 2a, SEQ ID NO. 190), flanked by 178 or 206 nts of 5′ and 139 or 205 nts 3′ flanking sequences. The pri-miAPOE cassettes were expressed from the CMV immediate-early enhancer fused to chicken β-actin promoter (CAG promoter, SEQ ID NO. 191) or an adapted/synthetic promotor (P1, SEQ ID NO. 192 and P2, SEQ ID NO. 193) and terminated by the simian virus 40 polyadenylation signal (SV40 polyA, SEQ ID NO. 194).

The APOE transgenes were expressed from the CAG promotor (SEQ ID NO. 191) or the adapted/synthetic promotors (P1, SEQ ID NO. 192 and P2, SEQ ID NO. 193) and terminated by SV40 polyA (SEQ ID NO. 194). APOE sequences were codon optimized for Homo Sapiens using an online codon optimization tool from ThermoFisher) and the Nhel, Notl and Spel sites were left intact during codon optimization. miAPOE (SEQ ID NOs. 108, 116, 146) or scrambled negative control (SEQ ID NO. 186) and APOE transgene (SEQ ID NOs. 195, 197, 200, 196, 198, 201, 206, 208, 210, 207, 209, 211, 214, 216, 216) were combined to facilitate simultaneous knock-down of APOE4 and overexpression of a protective APOE variant (combined approach). Plasmid DNA constructs containing an intronic element of the P2 promotor, harboring a pri-miAPOE cassette, and an APOE transgene cassette were synthesized with added 5′ (Blpl) and 3′ (EcoRV) sequences and subcloned by Genewiz (Azenta Life Sciences). The expression of the combined approach constructs was driven by a P4 promoter (P3, SEQ ID NO. 232 plus the intronic element, SEQ ID NO. 231) and terminated by a SV40 polyA signal (SEQ ID NO. 194). Each of the construct identity was confirmed by sequencing.

An APOE4 luciferase reporter was generated containing complementary APOE target regions (1166 bp, SEQ ID NO. 218) fused to the renilla luciferase (RL) gene (FIG. 3). Said target regions sequence was synthesized with added 5′ (Xhol) and 3′ (Pmel) sequences and cloned into the 3′UTR of the renilla luciferase (RL) gene of the psiCHECK-2 vector (Promega, Madison, WI) by GeneWiz (Azenta Life Sciences).

### AAV5 Vectors

Recombinant AAV5 particles were produced by infecting serum-free SF+insect cells (Protein Sciences Corporation, Meriden, Connecticut, USA) with two Baculoviruses, one encoding Rep/Cap combination, with the second carrying a transgene construct. Following standard protein purification procedures on a fast protein liquid chromatography system (AKTA Avant 150, GE 30 Healthcare) using AVB sepharose (GE Healthcare) the titer of the purified AAV was determined using QPCR.

Recombinant AAV5 and AAV9 were also produced by PEI transfection of HEK293T cells with two plasmids encoding for Rep-Cap and the transgene (Sirion Biotech). Following two step purification with primary capture with POROS™ CaptureSelect™ AAV-X resin (Thermo Fisher Scientific) and iodixanol gradient the titer of the purified AAV was determined using QPCR.

### Transfection Experiments

Human hepatocellular carcinoma (Huh7), human embryonic kidney 293T (HEK293T) or U118 astrocytoma cells were maintained in Dulbecco's modified Eagle's medium (DMEM) supplemented with 10% fetal bovine serum without antibiotics. Cells were seeded in 24-well plates at a density of 1E+05 cells per well the day before transfection or transduction experiments.

Transfections were performed using Lipofectamine@ 2000 or Lipofectamine@ 3000 according to the manufacturers' protocol.

### Luciferase Assays

HEK293T cells were co-transfected with the miAPOE or the combined constructs together with the luciferase reporters containing both the RL gene fused to APOE target sequences and the Firefly luciferase (FL) gene. pBluescript was added to transfer equal amounts of DNA. Transfected cells were harvested 48 hours post-transfection in 100 μl 1× passive lysis buffer (Promega, Thermo Fisher Scientific) by gentle rocking for 15 minutes at room temperature. The cell lysates were centrifuged for 5 minutes at 4,000 rpm and 10 μl of the supernatant was used to measure FL and RL activities with the Dual-Luciferase Reporter Assay System (Promega, Thermo Fisher Scientific). Relative luciferase activity was calculated as the ratio between RL and FL activities.

### Transduction Experiments

For transduction HEK293T cells were seeded in 24-wells plates at a density of 1E+05 cells per well 1 day prior to transduction. The next day cells were incubated with AAV vectors at a multiplicity of infection (MOI) of 1E+04, 1E+05E and 1E+06 gc/cell. Cells and culture supernatants were harvested 2 days post-transduction.

For transduction of U-118 MG cells (ATCC® HTB-15™), cells were seeded in 12- or 24-wells plates at a density of 2.63E+4 cells/cm² 1 day prior to transduction. The next day cells were incubated with AAV vectors at a multiplicity of infection (MOI) of 1.55E+06 gc/cell. The medium of the cells was replaced 2 days post-transduction and the cells harvested 3 days post-transduction for isolation of DNA and RNA.

### Pulverization of Mouse Tissues Using a Cryogenic Pulverization System

Frozen tissue samples were pulverized using an automated cryogenic sample pulverization system. Snap frozen tissues were crushed by exerting one or several punches of varying impacts with the CryoPREP system type CP02 (Covaris). Before and after each impact the tissueTUBE (Covaris) with tissue was dipped into liquid nitrogen, the procedure was repeated until the sample was pulverized. Powder was stored at −80° C. in cryovials (Covaris or Corning) until further use (e.g. DNA and RNA extraction, lysate production for APOE protein measurement).

Vector DNA and mRNA Isolation and Quantification

DNA and RNA extraction for in vitro experiments was performed using either the All prep 96 DNA/RNA isolation kit (Qiagen, 80311), the All Prep DNA/RNA Mini Kit (Qiagen, 80204) or MagMAX™ mirVana™ Total RNA Isolation Kit (Thermo Fisher Scientific) according to the manufacturer's instructions. Cells were harvested in the lysis buffer according to the manufacturer's protocol.

DNA and RNA extraction for in vivo experiments was performed using All prep 96 DNA/RNA isolation kit (Qiagen, 80311) or All Prep DNA/RNA Mini kit (Qiagen, 80204). Tissue lysates were produced by adding pulverized tissue to Lysing Matrix D tubes (MP Biomedicals) with the supplied RLT lysis buffer and β mercaptoethanol (Sigma), and homogenizing in the TissueLyser II (QIAGEN) at a frequency of 30 Hz for 60 seconds according to the manufacturer's protocol.

DNA and RNA concentrations and purity ratios were quantified in duplicate with spectrophotometer (either the NanoPhotometer® N120 (IMPLEN), or the Synergy HT in the Take3 Microvolume Plate (BioTek) or the NanoDrop One or 2000 (Thermo Scientific)). Samples were stored at −80° C. until further use. After extraction, DNA concentrations were normalized to ensure equal input for the quantitative polymerase chain reaction (qPCR). Vector genome copies were quantified by using TaqMan qPCR assay (SEQ ID NOs. 219-222, and SEQ ID NO. 277; table 4) using a linearized plasmid standard line (5E+0 to 5E+7) and ACTB TaqMan [Human ACTB Primer/Probe Mix Hs01060665_g1 (Thermo Fisher Scientific) and (SEQ ID NOs. 225-227; table 4) or SybrGreen qPCR (SEQ ID NOs. 223-224; Table 4) assay as the loading control.

After extraction, RNA concentrations were normalized prior to DNase treatment to ensure equal input for cDNA synthesis and subsequently qPCR. DNase treatment of isolated RNA was performed by using TURBO DNAse™ provided in the RNA isolation kit (Thermo Fisher Scientific) or DNase provided with Maxima First Strand cDNA Synthesis Kit (K1672), for cDNA synthesis Maxima First Strand cDNA Synthesis Kit (Thermo Fisher Scientific) was used according to manufacturer's instructions. WT and codon optimized variants V2 and V3 APOE mRNA expression was quantified performed using single or duplex qPCR was performed using TaqMan qPCR assay (SEQ ID NOs. 228-230 for WT APOE, and SEQ ID NOs. 287-291 for the codon optimized variants; table 4) and β-actin ACTB TaqMan [Human ACTB Primer/Probe Mix Hs01060665_g1 (Thermo Fisher Scientific), Mouse ACTB Primer/Probe Mix mm01205647_g1 (Thermo Fisher Scientific)] or SybrGreen qPCR assay (SEQ ID NOs. 223-224; table 4) as the reference gene for normalization. Either absolute or relative gene expression levels were calculated using either ddCT (Livak's Method) or using a linearized plasmid standard line (1E+8 to 1E+3 or 1E+2).

qPCR was performed using a QuantStudio 5 Real-Time PCR System or 7500 Fast Real-Time PCR System (ThermoFisher). The lower limit of quantification (LLOQ) is determined by calculating the amount of copies at the mean Ct value of the lowest point of the standard line and corrected according to the input of (c) DNA (ng) used.

TABLE 4
Primes and probes.

| SEQ | | |
|---|---|---|
| ID | | |
| NO. | ID | SEQUENCE |
| 219 | prPromotor FW | 5′-AGTAACGCCAATAGGGACTTTC-3′ |
| 220 | prPromotor RV | 5′-GGCGTACTTGGCATATGATACA-3′ |
| 221 | pBbPromotor | Cy5 5′-TTACGGTAAACTGCCCACTTGGC |
| | Cy5 | A-3′ MGBNFQ |
| 222 | pBbPromotor | FAM 5′-CTA GCG TGT AGA CTC T-3′ |
| | FAM | MGBNFQ |
| 223 | prhACTB FW | 5′-AGGTGCACAGTAGGTCTGAACAGA-3′ |
| 224 | prhACTB RV | 5′-TGCAAAGAACACGGCTAAGTGT-3′ |
| 225 | prmsACTB FW | 5′-GACACTGGCACSGCCAACTTT-3′ |
| 226 | prmsACTB RV | 5′-GTGTCTACACCGCGGGAATG-3′ |
| 227 | pbmsACTB FAM | FAM 5′-CTA GCG TGT AGA CTC T-3′ |
| | | MGBNFQ |
| 228 | prAPOE FW | 5′- GGAACTGAGGGCGCTGATG -3′ |
| 229 | prAPOE RV | 5′- GCCTGCAGCTCCTTGGAC -3′ |
| 230 | pbAPOE | Cy5 5′- TCG GAA CTG GAG GAA CAA |
| | | CTG ACC -3′ MGBNFQ |
| 277 | pBbPromotor | 56FAM/TTACGGTAA/ZEN/ACTGCCCACTT |
| | 56FAM | GGCA/3IABKFQ |
| 287 | prAPOE coV2 FW | GACCCAAGAGCTGAGAGCC |
| 288 | prAPOE coV2 RV | CTGCAGCTCTTTGCTCAGTCTG |
| 289 | prAPOE coV3 FW | GGAACTGAGGGCGCTGATG |
| 290 | prAPOE coV3 RV | GCCTGCAGCTCCTTGGAC |
| 291 | pbAPOE co | FAM 5′-AAG AAC TCA AAG CTT ATA |
| | | AGA GC-3′ MGBEQ |

miRNA RT-@PCR and Quantification

DNase treatment and cDNA synthesis of isolated RNA from in vivo experiments was performed using the Maxima First Strand cDNA Synthesis Kit (Thermo Fisher Scientific) and the E. coli Poly(A) Polymerase kit (New England Biolabs) according to manufacturer's instructions. For Reverse Transcription, the RT-primer indicated in table 5 was deployed.

miRNA expression was quantified employing a SybrGreen qPCR assay specific for each miRNA (SEQ ID NOs. 278-286 and SEQ ID NOs. 292-293 in table 5). For the in vivo hAPOE4-Tr mouse study, relative miRNA expression was calculated using a standard line obtained respectively from each RNA oligo (22NT for miAPOE-016,-037, and -145, and 23NT for miSCR1-C90). Based on in vitro small RNA sequencing data, the assay was optimized for the in vivo WT mouse study, and standard lines were produced from RNA oligo with the size of the most abundant isoform (24NT for miAPOE-016, 25NT for miAPOE-037, 24NT for miAPOE-145 and 23NT for miAPOE_SCR).

The lower limit of quantification (LLOQ) is the lowest amount of analyte in a sample, which can be reliably quantified with an acceptable level of precision. This is calculated using the lower point (reliably quantifiable) of the standard line and obtaining the corresponding copies per ng of RNA.

The lower limit of detection (LLOD) is the lowest amount of analyte in a sample, which can be reliably detected but not necessarily quantified. This is calculated using the lowest Ct value detectable by the machine and obtaining the corresponding copies per ng of RNA.

TABLE 5
List of primers used for the miRNA RT-qPCR and
quantification.

| SEQ | | |
|---|---|---|
| ID | | |
| NO. | ID | SEQUENCE (5′→3′) |
| 278 | RT-primer | CAGGTCCAGTTTTTTTTTTTTTTTVN |
| 279 | prmiAPOE16FW22 | AGCACAGAACCTTCATCTTC |
| 280 | prmiAPOE16RV22 | GTCCAGTTTTTTTTTTTTTTTGCAG |
| 281 | prmiAPOE37FW222324 | GTGACCAGCAACGCA |
| 282 | prmiAPOE37RV23 | AGGTCCAGTTTTTTTTTTTTTTTAGG |
| 283 | prmiAPOE145FW222324 | CAGTCCAGTTCCGATTTGTAG |
| 284 | prmiAPOE145RV23 | GGTCCAGTTTTTTTTTTTTTTTGAG |
| 285 | miSCR1-C9O_23nt-FW | AGGCGACTTGTAACGTCA |
| 286 | miSCR1-C9O_23nt-RV | CAGTTTTTTTTTTTTTTTACGCCTCT |
| 292 | prmiAPOESCRFW222324 | CGCGTTCTCGCTGTTC |
| 293 | prmiAPOESCRRV22 | GGTCCAGTTTTTTTTTTTTTTTAGTC |

RNA Isolation for Small RNA Sequencing and polyA-Enriched mRNA Sequencing

For in vivo, pulverized tissues were lysed in Lysing Matrix D tubes (MP Biomedicals) with RLT lysis buffer and β mercaptoethanol (Sigma) using the TissueLyser II (QIAGEN) at a frequency of 30 Hz for 60 seconds. RNA was extracted using the miRNeasy Tissue/Cells Advanced Mini Kit (QIAGEN) according to the manufacturer's protocol. For in vitro, transduced cells were harvested and lysed in TRIzol™ Reagent (Invitrogen). RNA was extracted using the Direct-zol™ RNA MiniPrep Kit (Zymo Research R2052) according to the manufacturer's protocol. RNA quantification was performed with NanoDrop™ One (Thermo Scientific) by measuring nucleic acid concentrations and purity ratios in duplicate. The quality of the RNA samples was analyzed using the Agilent High Sensitivity RNA ScreenTape (Agilent) prior to shipment. Samples were stored at −80° C. and shipped on dry ice to GenomeScan for small RNA sequencing (Illumina NovaSeq6000 sequencing, Paired-End, 150 bp. Per sample ˜ 3 Gb, 10 million Paired-End reads) and polyA-enriched mRNA sequencing (Illumina NovaSeq6000 sequencing, Paired-End, 150 bp. Per sample ˜ 9 Gb, 30 million Paired-End reads).

### RNAseg Data Analysis

Raw RNA seq data was provided by GenomeScan. For data analysis, CLC Genomics Workbench version 21.0.5 was used. For the small RNA sequencing analysis of both in vivo and in vitro derived samples (dosed hAPOE4-Tr mouse caudal cortex or transduced U-118 MG cells resp.), the acquired data was aligned and annotated to miAPOE or miSCR reference sequence (SEQ ID nos. 295-297; table 7). The length and matching of mature miRNA isoforms were identified and their abundancy were calculated as the percentage of total reads mapping to the reference miAPOE or miSCR sequence. During the analysis, a threshold of 2% of mature form was taken for convenience. The ranking of endogenously expressed miRNA levels together with miAPOE or miSCR transcript levels was performed by correcting the reads in counts per million (CPM) using the CLC Genomics Workbench Toolbox for small RNA sequencing analysis. miR-29a-3p and miR-16-5p were used as internal control, which are miRNAs expressed in the brain.

For the mRNA sequencing analysis of the in vitro derived samples (transduced U-118 MG cells), the acquired data was aligned and annotated to the host species (Homo Sapiens for the transduced U-118 MG experiment). The CLC Genomics Workbench Toolbox for differential expression for RNA-Seq was used in which datasets obtained from samples transduced with AAV5 combined approach or single constructs were compared to the datasets obtained from samples transduced with AAV5-miSCR (control).

TABLE 7
miAPOE and miSCR reference sequences

| SEQ | | |
|---|---|---|
| ID | | |
| NO. | ID | SEQUENCE |
| 295 | miAPOE_ | CACAGAACCTTCATCTTCCTGCGAAGATGAAGGTTCTGT |
| | 016 | |
| 296 | miAPOE_ | AATGTGACCAGCAACGCAGCCCTGCGTTGCTGGTCACAT |
| | 037 | |
| 297 | miAPOE_ | TCCAGTTCCGATTTGTAGGCCTCTACAAATCGGAACTGG |
| | 145 | |
| 298 | miC9o_ | GCGACTTGTAACGTCAGAGGCGTCTGACGTTACAAGTCG |
| | SCR | |
| 299 | miAPOE_ | ATCGCGTTCTCGCTGTTCGACTGAACAGCGAGAACGCG |
| | SCR | |

### APOE Protein Measurements

A human Apolipoprotein E (APOE) ELISA kit (ab108813, Abcam) was used to quantify the APOE concentration in cell culture supernatants. This kit recognizes all three human APOE isoforms (APOE2, APOE3 and APOE4). Several dilutions of cell culture supernatant samples were tested to measure accurately within the range of the provided standard. For HEK293T cells that are transfected with APOE expression constructs the cell culture supernatant were diluted 100 times in the supplied ELISA assay buffer. After sample preparation the supplied protocol of the kit was used to measure APOE and to quantify the concentration using the four-parameter logistic curve-fit in PRISM.

A human Apolipoprotein E (APOE) MSD R-PLEX assay (K1512IR-2, Meso Scale Discovery) was used to quantify the APOE concentration in tissue lysates. Tissues were lysed in MSD Tris Lysis Buffer (R60TX-2, Meso Scale Discovery) and corrected for the total protein concentration using Pierce™ 660 nm Protein Assay Reagent (22660, ThermoFisher). Lysates of 2 mg/ml total protein were diluted 50× in MSD Diluent 100 (R50AA-2, Meso Scale Discovery). After sample preparation the supplied protocol of the kit was used to measure APOE and to quantify the concentration using MSD Discovery Workbench (software version 4.0).

Phosphorylated Tau (pTau181) and Total Tau Protein Measurements

A human pTau181 MSD S-PLEX assay (K151AGMS, Meso Scale Discovery) was used to quantify the pTau181 protein concentration and a muti-spot phospho (Thr231)/total tau assay (K15121D, Meso Scale Discovery) was used to measure total tau in mouse hippocampal tissue lysates. Tissues were lysed in MSD Tris Lysis Buffer (R60TX-2, Meso Scale Discovery) and normalized for the total protein concentration using Pierce™ 660 nm Protein Assay Reagent (22660, ThermoFisher). Hippocampal lysates were diluted 10000× in dPBS for the pTau181 assay or 200× in dPBS for the total tau assay. Then the samples were further diluted 2× in blocking buffer supplied with the respective kits. After sample preparation, the supplied protocols of the two kits were used to measure pTau181 and total tau protein concentration using MSD Methodological Minds software and further processed using MSD Discovery Workbench (software version 4.0).

SDS-PAGE and Western blot

Samples were prepared in 1× Laemmli sample buffer (1610747; Bio-Rad) and heated to 95° C. for 5 minutes. Proteins were separated on a Mini-PROTEAN TGX Stain-Free Protein Gel 4-20% (4568093; Bio-Rad) and transferred to PVDF-membrane using Trans-blot Turbo Mini PVDF Transfer packs (1704156; Bio-Rad) and the Trans-Blot Turbo™ Transfer System (1704150; Bio-Rad).

The PVDF-membrane was blocked with SuperBlock T20 (PBS) Blocking Buffer (37516; Bio-Rad) and stained with the primary antibody in blocking buffer. After washing with 0.5% Tween-20 in phosphate buffered saline (PBS), the membrane was incubated with the secondary antibody in Blocking Buffer. Following extensive washing with PBS-0.5% Tween20, the proteins were visualized using SuperSignal™ West Pico PLUS Chemiluminescent Substrate (34580; Thermo Scientific) and the ChemiDoc Touch Gel Imaging System (1708370; Bio-Rad).

The following primary and secondary antibodies were used against the respective protein: APOE (Sigma Aldrich; HPA065539 & HPA068768), Polyclonal Goat Anti-Rabbit Immunoglobulins-HRP (DAKO, P0448) (DAKO), Polyclonal Rabbit Anti-Mouse Immunoglobulins-HRP (DAKO, P0260) Known concentrations of recombinant human APOE protein (Sigma Aldrich; SRP4760, SRP4696, A3234) or Apolipoprotein E from human plasma (Sigma Aldrich; SRP6303) were used as a reference.

### Animal Studies

To study the potency of miAPOE, a transgenic model harbouring the human APOE4 gene; B6.129P2-APOE^{tm3(APOE*4)Mae} N8, was used. At eight-weeks of age, male B6.129P2-Apoe^{tm3(APOE*4)Mae} N8 mice were treated with a single bilateral intrastriatal (IS) administration of empty AAV5, AAV5-miSCR (SEQ ID NO. 186), or AAV5-miAPOE_016 (SEQ ID NO. 108), (mid and high dose), or AAV5-miQURE-miAPOE_037 (SEQ ID NO. 116), (mid and high dose) or AAV5-miAPOE_145 (mid and high dose) or AAV9-miAPOE_145 (mid dose) (SEQ ID NO. 146). Each group contains of 8 animals. Body weight (individual) is determined pre-treatment and weekly thereafter. The mice were subjected to blood sample collection for plasma preparation before the treatment, 3, 6 and 8 (sacrifice) weeks after treatment. Plasma total cholesterol, triglycerides, low-density lipoprotein (LDL), high-density lipoprotein (HDL) (all per individual mouse) were determined for all timepoints in the plasma samples collected. Mice were sacrificed 8 weeks post-treatment. Multiple organs and CSF are collected for analysis of AAV transduction by qPCR, APOE mRNA expression, hAPOE4 expression levels and miAPOE transgene expression.

To study the expression of APOE3ch in C57BI6 mice, 8 mice per group were injected with either a single bilateral intrastriatal dose of vehicle, AAV5 or AAV9 vectors or intracerebroventricularly with a single bilateral dose of AAV5 or AAV9 vectors. All AAV vectors harbor the APOE3ch expression cassette with a double HA tag. Blood samples were taken at predose, at 2 weeks and at 4 weeks (sacrifice) post-administration. At week 4 post-treatment, animals were sacrificed. After perfusion a terminal blood and CSF sample were taken for each animal. Several brain regions, spinal cord and the liver were collected and snap frozen for 5 animals per group. For 3 animals per group, the brains were fixated for immunohistochemistry and FISH purposes.

The efficacy of AAV5 vectors encoding different APOE variants to modify the phenotype of a tauopathy model P301S mice (PS19, JAX strain: 008169) was assessed by a single bilateral intrastriatal injection of AAV vectors. Twelve male mice per group will be treated at 8 weeks of age. Body weights will be measured prior to treatment and thereafter once every week. Blood samples will be taken prior treatment and at week 2, 4, 12 and 20 weeks after treatment. A panel of markers will be analyzed from plasma for the following parameters: LDL, HDL, cholesterol and triglyceride, on the terminal plasma samples. The mice will be subjected to behavioral tests to assess motor and cognitive function. At sacrifice at 28 weeks post-treatment, terminal blood and CSF samples will be collected. Several different brain areas, distinct spinal cord segments and the livers of the animals will be collected and snap frozen for 8 mice of each group. The brains of the remaining 4 animals will be perfused and fixated for IHC and FISH purposes.

The in vivo expression levels of transgenes by combined approach constructs as compared to the single constructs was assessed by a single bilateral intrastriatal administration of AAV vectors in WT mice. Six mice per group were treated at eight weeks of age. Animals were sacrificed one month post-treatment. Multiple brain regions and the liver were collected and snap frozen for 5-6 animals per group.

The in vivo expression levels and impact on tauopathy of AAV encoding transgenes derived from combined approach constructs as compared to the single constructs are assessed using a single bilateral intrastriatal administration of AAV vectors in the tauopathy model P301S mice (PS19, JAX strain: 008169) also harboring hAPOE4 (B6.129P2-APOEtm3 (APOE*4) Mae N8). Six mice per group are treated at eight weeks of age. Animals are sacrificed several months post-treatment. Multiple brain regions, liver, and biofluids are collected and snap frozen for all animals per group.

In Silico Assessment of Potential Off-Target Transcripts of miAPOE Guides

In silico off-target prediction for miAPOE miRNA guide sequences was performed using BLASTN against the human reference transcriptome (via ENSEMBL, https://www.ensembl.org/Homo_sapiens/Tools/Blast, see table 8 for specifications, Bedell et al., 2003) to identify transcripts with partial complementary with the 22 nucleotide miAPOE guide sequence (SEQ ID NOs. 108, 116, and 146).

TABLE 8
BLASTN specifications for in silico off target prediction

| | Short |
|---|---|
| Search Sensitivity: | sequences |
| Search type | BLASTN |
| Maximum number of hits to report | 100 |
| Maximum E-Value for reported alignments | 1000 |
| Word size for seeding alignments | 7 |
| Match/Mismatch scores | 1, −3 |
| Filter low complexity regions | No |
| Filter query sequences using Repeat masker | Yes |

The “siRNA Seed Potential of Off-Target Reduction”-tool (siSPOTR, Boudreau et al., 2013) was used to predict the binding of the miAPOE miRNA guide seed sequence (nucleotide 2-8) to off-target transcripts.

### Results

### Example 1: In Vitro Testing of miAPOE Constructs on APOE4 Luc Reporter System

To test the knockdown efficiency of designed miAPOE constructs, HEK293T cells were co-transfected with miAPOE (SEQ ID NOs. 94-185) expression constructs and APOE 4 luciferase reporter bearing the complementary APOE target regions (SEQ ID NOs. 2-93).

First, since an exogenous expression of miR-144 (SEQ ID NO. 235) would lead to side-effects (e.g. miR-144 targets engagement) (Huang et al. 2021; H. Li et al. 2016; Lin et al. 2020), a novel strategy to introduce modifications within the scaffold in order to abrogate miR-144 expression was designed. A mutant version of miR-144 (SEQ ID NO. 236) hairpin was engineered, wherein adenosine at position 5 from Drosha cleavage site was substituted by a thymidine (T), creating a bulge in the vicinity of the cleavage site, which disrupted miR-144 expression (S. Li et al. 2020, 2021).

Any mismatch, bulge or GU wobble introduced within positions 4-8 from the Drosha cleavage site impairs the enzymatic activity of Drosha. Double and triple mismatches, bulges or wobbles within the 4-8 stretch further decrease the activity of Drosha. Therefore, any of the following SNP and combinations thereof within the 4-8 nucleotide stretch of miR-144 may alter (pre-) miR-144 expression. Those mutations include U>G at position 4, and/or A>U or G at position 5 and/or U>A at position 6 and/or C>G or U at position 7 and/or A>U or G at position 8.

Next, a set of scaffolds expressing a synthetic miRNA6 (=miHTT) from miR-451 was generated, with miRNA6 being expressed as a single hairpin (SEQ ID NO. 238) or associated with either miR-144 wild-type (miR-144WT helper) (SEQ ID NO. 239) or −144 mutant (miR-144A>T helper) (SEQ ID NO. 240). miRNA6 expression was assessed across the different scaffolds by small RNA sequencing (FIG. 3A). The introduced bulge abrogated miR-144 5p and 3p expression without affecting its helper function onto miR-451, as shown by the expression values of miRNA6 remaining unaffected when associated with either miR-144WT helper or miR-144A>T (FIG. 3A).

Following that, the expression values of an array of various synthetic miRNA sequences (SEQ ID NOs. 241-248) expressed from miR-451 Scaffold (without and with miR-144A>T helper) (SEQ ID NO. 190) were also analyzed, confirming an unexpected and tremendous improvement of miR-451 expressed synthetic miRNAs in the presence of miR-144A>T (SEQ ID NO. 236) (FIG. 3B). Therefore, the use of mutant helper miR-144 enables to take advantage of the miR-144-miR-451 activity without expression of unwanted microRNAs, and allows for higher synthetic miRNA expression levels. miR-144A >T mutant-miR-451 cluster was therefore selected for use.

Initial screening was performed on HEK293T cells co-transfected with 250 ng of miAPOE (SEQ ID NOS. 94-185) or miSCR constructs (SEQ ID NOs. 186-189) and 50 ng reporter construct. Two days post-transfection, cells were harvested to measure luciferase expression. Luciferase expression in the presence of miSCR was set at 100%.

Most of the transfected miAPOE constructs showed a reduction of relative luciferase activity compared to a miSCR construct (SEQ ID NO. 186). miAPOE with SEQ ID NOs. 96, 105, 106, 108, 113, 115, 116, 131, 132, 133, 134, 135, 136, 137, 139, 146, 149 resulted in a knockdown of the reporter activity of ≥70% (FIG. 4).

To accurately determine the potency of the miAPOE constructs further, the potent miAPOE constructs with more >60% knockdown efficiency were tested in a titration experiment using 2, 10 or 50 ng of the miAPOE (SEQ ID NOs. 96, 99, 100, 105,106, 108, 109, 113, 114, 115, 116, 117, 131, 132, 133,134, 135, 136, 137, 139, 143, 146, 149, 151, 185) or miSCR construct (SEQ ID NO. 186) and 10 ng reporter construct (n=2). Luciferase expression in the presence of miSCR was set at 100%. All constructs showed dose-dependent reporter knockdown (FIG. 5).

### Example 2: In Vitro Testing of miAPOE-Knockdown of Endogenous APOE (mRNA and Protein)

The ability of miAPOE candidates to silence endogenously expressed APOE mRNA and to reduce APOE protein levels was tested in Huh7 cells.

Huh7 cells were co-transfected using 250 ng of miAPOE (SEQ ID NOs. 94-185) or miSCR constructs (SEQ ID NOs. 186-189). APOE mRNA expression was determined using RT-QPCR and calculated by using a ddCT method using B-actin gene expression as the reference gene. APOE mRNA was reduced for most of the transfected miAPOE constructs compared to the average of 4 SCR constructs, pBluescript and non-transfected set to 100%. miAPOE SEQ ID NOs. 96, 98, 99, 108, 109, 113, 115, 116, 136, 138, 144, 146, 149, 151, 160, 163, 171 and 185 reduced endogenous APOE mRNA expression by >50% or more (FIG. 6).

Nine miAPOE constructs, which showed a reduction of the reporter activity of >60% in the previous experiment, were selected to test targeting in a lower doses. Huh7 cells were co-transfected as described above using 50 ng or 250 ng of miAPOE (SEQ ID NOs. 96, 108, 116, 133, 136, 138, 146, 151, 185) or miSCR constructs (SEQ ID NOs. 186-187) and 10 ng pFI reporter construct. APOE mRNA expression of the average miSCR samples was set at 100% expression. All constructs showed >60% lowering of APOE mRNA after transfection with 250 ng of the plasmid, and >50% lowering after transfection of 50 ng of the plasmid (FIG. 7). In addition, the APOE protein levels in the culture supernatants were quantified in 250 ng transfected samples using Western blots. APOE protein levels in the supernatants of transfected cells were quantified relative to un-transfected cells, showing a decrease of 70-90% for all tested miAPOE (FIG. 8).

### Example 3: In Vitro Testing—of Endogenous APOE mRNA Expression in Huh7 Cells Upon Transduction with AAV-miAPOE

Recombinant AAV (5 or 9) vectors were generated harboring the expression cassettes of miAPOE_016 (SEQ ID NO. 108), miAPOE_037 (SEQ ID NO. 116), miAPOE_145 (SEQ ID NO. 146) and miSCR_144 (SEQ ID NO. 186). The ability of obtained AAVs to transduce and deliver the packaged expression cassette were tested by transducing Huh7 cells at a Multiplicity of Infection (MOI) of 5E+06, 3.6+06, 1E+06, 5E+05 or 5E+04 gc/cell. Vector DNA copies and residual APOE mRNA were evaluated 48 h post-transduction by qPCR and RT-QPCR using TaqMan qPCR duplex. Vector DNA levels were quantified using a standard line ranging from 1E+07- to 5E+01 copies. A dose-dependent increase in detected vector genome DNA copies was observed. The transduction efficiency of the Huh7 cells was comparable for all AAV5 batches with the same MOI. The AAV9 vectors show lower vector genome copies in the Huh7 cells upon transduction. Similar to the AAV5 vectors, there is a dose-dependent increase in detected vector copies upon transduction (FIG. 9A).

Endogenous APOE mRNA expression was calculated by using ddCT method using B-actin as a reference gene. APOE mRNA expression in the presence of AAV5-miSCR was set at 100% expression. The results are depicted in FIG. 9B.

All AAV batches resulted in a dose-dependent lowering of APOE mRNA expression which confirms the functionality of the AAV delivered expression cassette and the potency of the miAPOEs. AAV5-miAPOE_037 (AAV5) and AAV5-miAPOE_016 also showed a clear dose-dependent lowering: miAPOE_037 showed the greatest potency in lowering the APOE mRNA expression with MOI 5E+06 (˜55% lowering) and MOI 1E+06 (˜35% lowering), little lowering was observed with MOI 5E+05 (˜10% lowering) and none with 5E+04 gc/cell. Similarly, miAPOE_016 showed the highest lowering for MOI 5E+06 (˜35% lowering), MOI 1E+06 (˜25% lowering) and 5E+05 (˜15% lowering) for MOI 5E+05. For AAV5-miAPOE_145 a lowering of the APOE mRNA expression of ˜15% for MOI 5E+06 and ˜25% for MOI 1E+06 was observed, a similar lowering range was seen with AAV9-miAPOE_145.

### Example 4: Testing of AAV-miAPOEs Targeting APOE in hAPOE4TR Mice

To examine the ability of selected miAPOE candidates to silence human APOE4 expression in vivo, a hAPOE4 targeted replacement (TR) mouse model was used. Male mice were treated with a single bilateral intrastriatal (IS) administration of empty AAV5, AAV5-miSCR (SEQ ID NO. 294, previously incorrectly labeled as SEQ ID NO. 186), or AAV5-miAPOE_016 (SEQ ID NO. 108), (mid and high dose), or AAV5-miQURE-miAPOE_037 (SEQ ID NO. 116), (mid and high dose) or AAV5-miAPOE_145 (mid and high dose) or AAV9-miAPOE_145 (mid dose) (SEQ ID NO. 146). Mid dose was set at a total of 6E10 gc total per mouse, and high dose was set at 3E11 gc total per mouse. Each group contained 8 animals. At week 8 post-treatment, animals were sacrificed and the levels of vector DNA, APOE mRNA, miAPOE or miSCR, and hAPOE4 protein in the brain were determined.

Vector DNA levels were quantified by qPCR to analyze AAV transduction. Mice treated with AAV5-miSCR, AAV5-miAPOE_016, AAV5-miAPOE_037, AAV5-miAPOE_145, and AAV9-miAPOE_145 all showed a significant increase of vector DNA levels in the striatum and cortex reaching up to 2.05E+08 gc/μg DNA and 2.54E+07 gc/μg DNA, respectively (FIG. 16A-B). The absence of a dose-dependent increase in vDNA indicates that both doses reach transduction saturation.

Silencing of human APOE mRNA by selected miAPOE candidates (miAPOE_016, miAPOE_037, and miAPOE_145) was assessed via RT-qPCR. In the striatum, AAV5-miSCR showed similar mRNA levels compared to the empty AAV5 group at 3.82E+07 copies/μg RNA. A significant reduction of hAPOE4 mRNA levels of up to >80-90% was observed in mice injected with AAV-miAPOE candidates compared to AAV5-miSCR (FIG. 17A-B). In the frontal cortex, a variation was found between the hAPOE4 mRNA levels in mice injected with empty AAV5 or AAV5-miSCR. A reduction of >50-60% of hAPOE4 mRNA was observed in mice injected with AAV-miAPOE candidates compared to AAV5-miSCR (FIG. 17C-D).

Additionally, miRNA (miAPOE or miSCR) levels were quantified by a SYBR green RT-qPCR assay using specific primers for the 22 or 23 nucleotide long mature miAPOEs and miSCR. miRNA levels in the striatum of AAV-miAPOE injected mice reached up to 2.45E+11 copies/μg total RNA (FIG. 18). 1.45E+08 miRNA copies/μg total RNA was found for mice injected with AAV5-miSCR. Striatal tissue of mice injected with empty AAV5 was also taken along in all RT-qPCR experiments using specific primers against either the miAPOE or miSCR. In all experiments, the amount of miRNA copies/μg total RNA in the striatum of mice injected with empty AAV5 was below the LLOQ (not shown).

To examine whether the silencing of hAPOE4 mRNA correlates with reduced hAPOE4 protein levels, hAPOE-MSD was performed on frontal cortical tissue. hAPOE4 protein levels of mice injected with AAV5-miSCR was equal to control levels (empty AAV5) at 41 ng/mg, again confirming that AAV5-miSCR does not impact hAPOE4 protein levels (FIG. 19). A significant protein reduction of up to >50-60% was observed in mice injected with AAV-miAPOE candidates compared to AAV5-miSCR.

In order to examine the processing and abundancy of the expressed miAPOEs compared to the expression levels of endogenous miRNAs, RNA was isolated from the caudal cortex of two mice from the high dose groups (AAV5-miAPOE_016, AAV5-miAPOE_037, and AAV5-miAPOE_145) and analyzed by small RNA sequencing. Data analysis revealed expected miAPOE transcripts with read counts ranging from 4.57-0.32% when compared to total miRNA reads (FIG. 20B). The most abundant isomIR produced by miAPOE_016, miAPOE_037 and miAPOE_145 are 23, 25 and 24 nucleotides long, respectively (FIG. 20A). The total miSCR and miAPOE reads of all samples were compared to expression levels of endogenous miRNAs (FIG. 20C). Considering that miAPOEs are not among the most abundant endogenously expressed miRNAs, the risk for oversaturation of the RNAi pathway by the miAPOEs is very low. The two internal controls, miR-29a-3p and miR-16-5p demonstrated consistent abundancy ranking 10.5+1 (SD) and 37.37+2.44 (SD) respectively amongst all samples (marked in FIG. 20C). Taken together, these results demonstrate in vivo high levels of vDNA and miRNA, and the significant reduction in hAPOE4 mRNA and protein levels of the AAV-miAPOE candidates. Thus, these results demonstrate that upon miAPOE expression delivered via AAV, high levels of miRNAs are being expressed, leading to a significant reduction in hAPOE4 mRNA and protein levels.

### Example 5: In Vitro Testing of Overexpression of APOE Variants

APOE expression constructs were transfected into HEK293T cells and APOE protein expression was measured in the supernatant two days post-transfection. The following APOE constructs were tested: APOE2ch WT (R136S mutation, SEQ ID NO. 197), APOE2ch v1 and APOE2ch v2 (codon optimized for Homo Sapiens using an online codon optimization tool from ThermoFisher) variants, SEQ ID NOs. 204, 208), APOE2 WT (SEQ ID NO. 195) and APOE2 v1 and v2 (2 codon optimized variants, SEQ ID NOs. 202, 206), APOE3 WT (SEQ ID NO. 196) and APOE3 v1 and v2 (codon optimized variants, SEQ ID NOs. 203, 207) and APOE3ch WT (R136S mutation, SEQ ID NO. 198), APOE3ch v1 and APOE3ch v2 (codon optimized variants, SEQ ID NOs. 205, 209).

APOE protein expression was detected in all cells transfected with the different APOE constructs (FIG. 10A). Recombinant APOE3 protein served as a positive control. The WT version for all constructs of APOE2ch, APOE3ch, APOE2 and APOE3 showed highest protein expression amongst the different variants. In addition, expression of the APOE constructs was also confirmed in astrocytoma cells (FIG. 10B).

### Example 6: In Vitro Testing-APOE Variant Expression in HEK293T Cells Upon Transduction with AAV5-APOE Variants

To investigate the ability of AAV5-APOE variants, to transduce and deliver the packaged expression cassette, HEK293T cells were transduced (n=1) at a Multiplicity of Infection (MOI) of 1E+04, 1E+05, 1E+06 gc/cell. Vector DNA level in the cells was quantified by qPCR. APOE variant protein expression was analyzed in culture supernatant of cells transduced with 1E+05, 1E+06 gc/cell via Western blot as described previously. The results are depicted in FIG. 11A. All five tested AAV5-APOE batches resulted in dose-dependent vector DNA levels in the cell lysates. Untransduced cells showed a background level of ˜E3 gc/μg DNA. APOE variant protein expression was confirmed in culture supernatant of cells transduced with 1E+06 gc/cell via Western blot (FIG. 11B).

### Example 7: WT Mouse Study to Investigate the Expression of AAV5/AAV9 APOE3ch-HA

To study the expression of APOE3ch in C57BI6 mice, 8 mice per group were injected with either a single bilateral intrastriatal dose of AAV5 or AAV9 vectors or intracerebroventricularly with a single bilateral dose of AAV5 or AAV9 vectors. All AAV vectors harbor the APOE3ch expression cassette with a double HA tag. At week 4 post-treatment, animals were sacrificed and the vector DNA levels and mRNA levels in the striatum of the brain were determined. On average the AAV5 and AAV9 group that received the vectors intrastriatally (IS) expressed 6e7 and 2e7 genomic copies per μg DNA. The intracerebroventricularly (ICV) delivered AAV groups showed a slightly lower vector genome copies with 4e6 copies for AAV5 and 3e6 copies for AAV9, respectively (12A). mRNA copy numbers were determined in the striatum of the brain for all animals. The IS AAV5 and AAV9 groups expressed on average 6e8 and 8e8 copies of mRNA in the striatum of the brain, whereas the ICV groups showed a slightly lower mRNA expression level; 1.1e8 and 1.5e8 copies for the AAV5 and AAV9 group (FIG. 12B).

### Example 8: Effect of APOE Overexpression in Different Brain Areas in P301S Tauopathy Model

The efficacy of AAV5 vectors encoding different APOE variants to modify the phenotype of a tauopathy model P301S mice was assessed by a single bilateral intrastriatal injection of AAV5 vectors. Two months old male mice were treated with a single bilateral intrastriatal administration of vehicle (0.001% Pluronic F-68), and empty AAV5, AAV5-APOE2ch WT, AAV5-APOE2ch V2, AAV5-APOE2b V2, AAV5-APOE3ch V2 and AAV5-APOE3b V2 (SEQ ID NOs. 197, 208, 210, 209, and 211) at a dose of 3E11gc/mouse. An untreated wild-type (WT) littermate control group was included as well. The effect of the AAV5-APOE vectors will be evaluated by behavioral tests. At seven months post-infusion, the molecular analysis of different brain areas, CSF, plasma and liver was performed for 8-11 animals per group. For 1-4 animals per group, the brains were fixed for histological purposes.

Vector DNA and APOE mRNA levels in the striatum were quantified by RT-qPCR to analyze AAV transduction and APOE transcript expression. Mice treated with AAV5-APOE2ch WT, AAV5-APOE2ch V2, AAV5-APOE2b V2, AAV5-APOE3ch V2 and AAV5-APOE3b V2 all showed a significant increase of vector DNA levels of up to 4.67E+07 gc/μg DNA and APOE mRNA levels of up to 4.48E+09 copies/μg RNA, compared to the WT, vehicle and empty AAV5 groups (FIG. 21A-B).

To examine whether the elevated mRNA copy levels in the APOE variant treated groups are in line with increased APOE protein expression, hAPOE-MSD was performed on hippocampal and frontal cortical tissues (FIG. 21C-D). No hAPOE protein levels were detected in the WT, vehicle and empty AAV5 groups. Elevated hAPOE protein levels were detected in the AAV5-APOE2ch WT, AAV5-APOE2ch V2, AAV5-APOE2b V2, AAV5-APOE3ch V2 and AAV5-APOE3b V2 groups both in the hippocampus (up to 511.09 ng/mg) and in the frontal cortex (up to 2086.41 ng/mg). AAV5-APOE2ch WT administration resulted into the highest yield of APOE protein compared to the other groups, a pattern previously seen in vitro (FIG. 15).

Soluble total and phosphorylated Tau were examined to understand whether expression of the APOE variants impact the levels of pathogenic phosphorylated Tau. Separate MSD assays were performed to quantify total Tau and pTau181 on hippocampal tissue. Besides the WT group, total Tau was detected in all P301S mouse groups at comparable levels (FIG. 22A). Significant decrease was detected in pTau181 levels of the AAV5-APOE2ch WT, AAV5-APOE2ch V2, AAV5-APOE2b V2, AAV5-APOE3ch V2 and AAV5-APOE3b V2 groups when compared to the vehicle group (up to 55%, FIG. 22B-C). The quantitative relationship between pTau181 and total Tau levels were examined (FIG. 22D-E), which revealed up to 70% reduction in the pTau181/Total Tau ratio for AAV5-APOE2ch V2, AAV5-APOE2b V2, and AAV5-APOE3b V2 groups when compared to the vehicle group.

Based on these results, we expect that immunohistochemistry will reveal a change in phosphorylated Tau levels, neurofibrillary tangle levels, and in the numbers of astrocytes and microglia in the hippocampi of mice injected with AAV5 vectors encoding different APOE variants compared to vehicle and/or empty AAV5 injected groups. As a co-finding of these immunohistochemistry studies, we expect to detect a change in the size of the ventricular cavities in the brains of the vehicle and/or empty AAV5 injected groups as compared to the WT group and in mice injected with AAV5 vectors encoding different APOE variants compared to vehicle and/or empty AAV5 injected groups.

Taken together, these results demonstrate that administration with AAV5-APOE variants lead to high and comparable vDNA and mRNA levels, resulting in elevated APOE protein levels and reduction of pTau181 levels.

### Example 9: In Vitro Testing of Combined Constructs-Reporter Lowering

To test the ability to silence APOE4 and to overexpress APOE2 or APOE3 variants simultaneously HEK293T cells are co-transfected with combined expression constructs and APOE luciferase reporters. miAPOE or APOE variants expression plasmids are used as controls.

In a first screening HEK293T cells were co-transfected using 86.54 fmol of the combined constructs (SEQ ID NOs. 255-276), APOE variants (SEQ ID NOs. 197, 208, 210, 211) or miAPOE (SEQ ID NOs. 108, 116, 146, 186). and 10 fmol of reporter. Expression of all combined constructs (SEQ ID NOs. 255-276; Table 6) resulted in a knockdown of the luciferase reporter (FIG. 13). To further assess the potency of the different constructs, a titration experiment was carried out using (0.69, 3.46 and 17.31 fmol (corresponding to 2, 10 or 50 ng, see Example 1) of DNA constructs. A dose-dependent lowering was visible for all constructs tested (FIG. 14).

TABLE 6
Combined constructs.

| SEQ ID | | |
|---|---|---|
| NO. | ID | SEQUENCE |
| 255 | 1 | GGAGTCGCTGCGCGCTGCCTTCGCCCCGTGCCCCGCTCCGCCGCCGCCTCG |
| | | CGCCGCCCGCCCCGGCTCTGACTGACCGCGTTACTCCCACAGGTGAGCGGG |
| | | CGGGACGGCCCTTCTCCTCCGGGCTGTAATTAGCTGAGCAAGAGGTAAGGGT |
| | | TTAAGGGATGGTTGGTTGGTGGGGTATGGGGCCCTGGCTGGGATTTCATCAT |
| | | ATACTGTAAGTTTGCGATGAGACACTACAGTATAGATGATGTACTAGTCCGGG |
| | | CACCCCCAGCTCTGGAGCCTGACAAGGAGGACAGGAGAGATGCTGCAAGCCC |
| | | AAGAAGCTCTCTGCTCAGCCTGTCACAACCTACTGACTGCCAGGGCACTTGGG |
| | | AATGGCAAGGCACAGAACCTTCATCTTCCTGCGAAGATGAAGGTTCTGTCTCT |
| | | TGCTATACCCAGAAAACGTGCCAGGAAGAGAACTCAGGACCCTGAAGCAGAC |
| | | TACTGGAAGGGAGACTCCAGCTCAAACAAGGCAGGGGTGGGGGCGTGGGAT |
| | | TGGGGGTAGGGGAGGGAATAGATACATTTTCTCTTTCCTGTTGTAAAGATAAT |
| | | GTTTAATTACCTGGAGCACCTGCCTGAAATCACTTTTTTTCAGGTTGGGCTAGC |
| | | GCCACCATGAAGGTTCTGTGGGCTGCGTTGCTGGTCACATTCCTGGCAGGAT |
| | | GCCAGGCCAAGGTGGAGCAAGCGGTGGAGACAGAGCCGGAGCCCGAGCTGC |
| | | GCCAGCAGACCGAGTGGCAGAGCGGCCAGCGCTGGGAACTGGCACTGGGTC |
| | | GCTTTTGGGATTACCTGCGCTGGGTGCAGACACTGTCTGAGCAGGTGCAGGA |
| | | GGAGCTGCTCAGCTCCCAGGTCACCCAGGAACTGAGGGCGCTGATGGACGA |
| | | GACCATGAAGGAGTTGAAGGCCTACAAATCGGAACTGGAGGAACAACTGACC |
| | | CCGGTGGCGGAGGAGACGCGGGCACGGCTGTCCAAGGAGCTGCAGGCGGC |
| | | GCAGGCCCGGCTGGGCGCGGACATGGAGGACGTGTGCGGCCGCCTGGTGC |
| | | AGTACCGCGGCGAGGTGCAGGCCATGCTCGGCCAGAGCACCGAGGAGCTGC |
| | | GGGTGCGCCTCGCCTCCCACCTGCGCAAGCTGCGTAAGCGGCTCCTCCGCG |
| | | ATGCCGATGACCTGCAGAAGTGCCTGGCAGTGTACCAGGCCGGGGCCCGCG |
| | | AGGGCGCCGAGCGCGGCCTCAGCGCCATCCGCGAGCGCCTGGGGCCCCTG |
| | | GTGGAACAGGGCCGCGTGCGGGCCGCCACTGTGGGCTCCCTGGCCGGCCAG |
| | | CCGCTACAGGAGCGGGCCCAGGCCTGGGGCGAGCGGCTGCGCGCGCGGAT |
| | | GGAGGAGATGGGCAGCCGGACCCGCGACCGCCTGGACGAGGTGAAGGAGCA |
| | | GGTGGCGGAGGTGCGCGCCAAGCTGGAGGAGCAGGCCCAGCAGATACGCCT |
| | | GCAGGCCGAGGCCTTCCAGGCCCGCCTCAAGAGCTGGTTCGAGCCCCTGGT |
| | | GGAAGACATGCAGCGCCAGTGGGCCGGGCTGGTGGAGAAGGTGCAGGCTGC |
| | | CGTGGGCACCAGCGCCGCCCCTGTGCCCAGCGACAATCACTGA |
| 256 | 2 | GGAGTCGCTGCGCGCTGCCTTCGCCCCGTGCCCCGCTCCGCCGCCGCCTCG |
| | | CGCCGCCCGCCCCGGCTCTGACTGACCGCGTTACTCCCACAGGTGAGCGGG |
| | | CGGGACGGCCCTTCTCCTCCGGGCTGTAATTAGCTGAGCAAGAGGTAAGGGT |
| | | TTAAGGGATGGTTGGTTGGTGGGGTATGGGGCCCTGGCTGGGATTTCATCAT |
| | | ATACTGTAAGTTTGCGATGAGACACTACAGTATAGATGATGTACTAGTCCGGG |
| | | CACCCCCAGCTCTGGAGCCTGACAAGGAGGACAGGAGAGATGCTGCAAGCCC |
| | | AAGAAGCTCTCTGCTCAGCCTGTCACAACCTACTGACTGCCAGGGCACTTGGG |
| | | AATGGCAAGGCACAGAACCTTCATCTTCCTGCGAAGATGAAGGTTCTGTCTCT |
| | | TGCTATACCCAGAAAACGTGCCAGGAAGAGAACTCAGGACCCTGAAGCAGAC |
| | | TACTGGAAGGGAGACTCCAGCTCAAACAAGGCAGGGGTGGGGGCGTGGGAT |
| | | TGGGGGTAGGGGAGGGAATAGATACATTTTCTCTTTCCTGTTGTAAAGATAAT |
| | | GTTTAATTACCTGGAGCACCTGCCTGAAATCACTTTTTTTCAGGTTGGGCTAGC |
| | | GCCACCATGAAGGTTCTGTGGGCTGCGTTGCTGGTCACATTCCTGGCAGGAT |
| | | GCCAGGCCAAGGTGGAGCAAGCGGTGGAGACAGAGCCGGAGCCCGAGCTGC |
| | | GCCAGCAGACCGAGTGGCAGAGCGGCCAGCGCTGGGAACTGGCACTGGGTC |
| | | GCTTTTGGGATTACCTGCGCTGGGTGCAGACACTGTCTGAGCAGGTGCAGGA |
| | | GGAGCTGCTCAGCTCCCAGGTCACCCAGGAACTGAGGGCGCTGATGGACGA |
| | | GACCATGAAGGAGTTGAAGGCCTACAAATCGGAACTGGAGGAACAACTGACC |
| | | CCGGTGGCGGAGGAGACGCGGGCACGGCTGTCCAAGGAGCTGCAGGCGGC |
| | | GCAGGCCCGGCTGGGCGCGGACATGGAGGACGTGTGCGGCCGCCTGGTGC |
| | | AGTACCGCGGCGAGGTGCAGGCCATGCTCGGCCAGAGCACCGAGGAGCTGC |
| | | GGGTGAGCCTCGCCTCCCACCTGCGCAAGCTGCGTAAGCGGCTCCTCCGCG |
| | | ATGCCGATGACCTGCAGAAGTGCCTGGCAGTGTACCAGGCCGGGGCCCGCG |
| | | AGGGCGCCGAGCGCGGCCTCAGCGCCATCCGCGAGCGCCTGGGGCCCCTG |
| | | GTGGAACAGGGCCGCGTGCGGGCCGCCACTGTGGGCTCCCTGGCCGGCCAG |
| | | CCGCTACAGGAGCGGGCCCAGGCCTGGGGCGAGCGGCTGCGCGCGCGGAT |
| | | GGAGGAGATGGGCAGCCGGACCCGCGACCGCCTGGACGAGGTGAAGGAGCA |
| | | GGTGGCGGAGGTGCGCGCCAAGCTGGAGGAGCAGGCCCAGCAGATACGCCT |
| | | GCAGGCCGAGGCCTTCCAGGCCCGCCTCAAGAGCTGGTTCGAGCCCCTGGT |
| | | GGAAGACATGCAGCGCCAGTGGGCCGGGCTGGTGGAGAAGGTGCAGGCTGC |
| | | CGTGGGCACCAGCGCCGCCCCTGTGCCCAGCGACAATCACTGA |
| 257 | 3 | GGAGTCGCTGCGCGCTGCCTTCGCCCCGTGCCCCGCTCCGCCGCCGCCTCG |
| | | CGCCGCCCGCCCCGGCTCTGACTGACCGCGTTACTCCCACAGGTGAGCGGG |
| | | CGGGACGGCCCTTCTCCTCCGGGCTGTAATTAGCTGAGCAAGAGGTAAGGGT |
| | | TTAAGGGATGGTTGGTTGGTGGGGTATGGGGCCCTGGCTGGGATTTCATCAT |
| | | ATACTGTAAGTTTGCGATGAGACACTACAGTATAGATGATGTACTAGTCCGGG |
| | | CACCCCCAGCTCTGGAGCCTGACAAGGAGGACAGGAGAGATGCTGCAAGCCC |
| | | AAGAAGCTCTCTGCTCAGCCTGTCACAACCTACTGACTGCCAGGGCACTTGGG |
| | | AATGGCAAGGCACAGAACCTTCATCTTCCTGCGAAGATGAAGGTTCTGTCTCT |
| | | TGCTATACCCAGAAAACGTGCCAGGAAGAGAACTCAGGACCCTGAAGCAGAC |
| | | TACTGGAAGGGAGACTCCAGCTCAAACAAGGCAGGGGTGGGGGCGTGGGAT |
| | | TGGGGGTAGGGGAGGGAATAGATACATTTTCTCTTTCCTGTTGTAAAGATAAT |
| | | GTTTAATTACCTGGAGCACCTGCCTGAAATCACTTTTTTTCAGGTTGGGCTAGC |
| | | GCCACCATGAAGGTTCTGTGGGCTGCGTTGCTGGTCACATTCCTGGCAGGAT |
| | | GCCAGGCCAAGGTGGAGCAAGCGGTGGAGACAGAGCCGGAGCCCGAGCTGC |
| | | GCCAGCAGACCGAGTGGCAGAGCGGCCAGCGCTGGGAACTGGCACTGGGTC |
| | | GCTTTTGGGATTACCTGCGCTGGGTGCAGACACTGTCTGAGCAGGTGCAGGA |
| | | GGAGCTGCTCAGCTCCCAGGTCACCCAGGAACTGAGGGCGCTGATGGACGA |
| | | GACCATGAAGGAGTTGAAGGCCTACAAATCGGAACTGGAGGAACAACTGACC |
| | | CCGGTGGCGGAGGAGACGCGGGCACGGCTGTCCAAGGAGCTGCAGGCGGC |
| | | GCAGGCCCGGCTGGGCGCGGACATGGAGGACGTGTGCGGCCGCCTGGTGC |
| | | AGTACCGCGGCGAGGTGCAGGCCATGCTCGGCCAGAGCACCGAGGAGCTGC |
| | | GGGTGCGCCTCGCCTCCCACCTGCGCAAGCTGCGTAAGCGGCTCCTCCGCG |
| | | ATGCCGATGACCTGCAGAAGTGCCTGGCAGTGTACCAGGCCGGGGCCCGCG |
| | | AGGGCGCCGAGCGCGGCCTCAGCGCCATCCGCGAGCGCCTGGGGCCCCTG |
| | | GTGGAACAGGGCCGCGTGCGGGCCGCCACTGTGGGCTCCCTGGCCGGCCAG |
| | | CCGCTACAGGAGCGGGCCCAGGCCTGGGGCGAGCGGCTGCGCGCGCGGAT |
| | | GGAGGAGATGGGCAGCCGGACCCGCGACCGCCTGGACGAGGTGAAGGAGCA |
| | | GGAGGCGGAGGTGCGCGCCAAGCTGGAGGAGCAGGCCCAGCAGATACGCCT |
| | | GCAGGCCGAGGCCTTCCAGGCCCGCCTCAAGAGCTGGTTCGAGCCCCTGGT |
| | | GGAAGACATGCAGCGCCAGTGGGCCGGGCTGGTGGAGAAGGTGCAGGCTGC |
| | | CGTGGGCACCAGCGCCGCCCCTGTGCCCAGCGACAATCACTGA |
| 258 | 4 | GGAGTCGCTGCGCGCTGCCTTCGCCCCGTGCCCCGCTCCGCCGCCGCCTCG |
| | | CGCCGCCCGCCCCGGCTCTGACTGACCGCGTTACTCCCACAGGTGAGCGGG |
| | | CGGGACGGCCCTTCTCCTCCGGGCTGTAATTAGCTGAGCAAGAGGTAAGGGT |
| | | TTAAGGGATGGTTGGTTGGTGGGGTATGGGGCCCTGGCTGGGATTTCATCAT |
| | | ATACTGTAAGTTTGCGATGAGACACTACAGTATAGATGATGTACTAGTCCGGG |
| | | CACCCCCAGCTCTGGAGCCTGACAAGGAGGACAGGAGAGATGCTGCAAGCCC |
| | | AAGAAGCTCTCTGCTCAGCCTGTCACAACCTACTGACTGCCAGGGCACTTGGG |
| | | AATGGCAAGGCACAGAACCTTCATCTTCCTGCGAAGATGAAGGTTCTGTCTCT |
| | | TGCTATACCCAGAAAACGTGCCAGGAAGAGAACTCAGGACCCTGAAGCAGAC |
| | | TACTGGAAGGGAGACTCCAGCTCAAACAAGGCAGGGGTGGGGGCGTGGGAT |
| | | TGGGGGTAGGGGAGGGAATAGATACATTTTCTCTTTCCTGTTGTAAAGATAAT |
| | | GTTTAATTACCTGGAGCACCTGCCTGAAATCACTTTTTTTCAGGTTGGGCTAGC |
| | | GCCACCATGAAGGTTCTGTGGGCTGCGTTGCTGGTCACATTCCTGGCAGGAT |
| | | GCCAGGCCAAGGTGGAGCAAGCGGTGGAGACAGAGCCGGAGCCCGAGCTGC |
| | | GCCAGCAGACCGAGTGGCAGAGCGGCCAGCGCTGGGAACTGGCACTGGGTC |
| | | GCTTTTGGGATTACCTGCGCTGGGTGCAGACACTGTCTGAGCAGGTGCAGGA |
| | | GGAGCTGCTCAGCTCCCAGGTCACCCAGGAACTGAGGGCGCTGATGGACGA |
| | | GACCATGAAGGAGTTGAAGGCCTACAAATCGGAACTGGAGGAACAACTGACC |
| | | CCGGTGGCGGAGGAGACGCGGGCACGGCTGTCCAAGGAGCTGCAGGCGGC |
| | | GCAGGCCCGGCTGGGCGCGGACATGGAGGACGTGTGCGGCCGCCTGGTGC |
| | | AGTACCGCGGCGAGGTGCAGGCCATGCTCGGCCAGAGCACCGAGGAGCTGC |
| | | GGGTGCGCCTCGCCTCCCACCTGCGCAAGCTGCGTAAGCGGCTCCTCCGCG |
| | | ATGCCGATGACCTGCAGAAGCGCCTGGCAGTGTACCAGGCCGGGGCCCGCG |
| | | AGGGCGCCGAGCGCGGCCTCAGCGCCATCCGCGAGCGCCTGGGGCCCCTG |
| | | GTGGAACAGGGCCGCGTGCGGGCCGCCACTGTGGGCTCCCTGGCCGGCCAG |
| | | CCGCTACAGGAGCGGGCCCAGGCCTGGGGCGAGCGGCTGCGCGCGCGGAT |
| | | GGAGGAGATGGGCAGCCGGACCCGCGACCGCCTGGACGAGGTGAAGGAGCA |
| | | GGTGGCGGAGGTGCGCGCCAAGCTGGAGGAGCAGGCCCAGCAGATACGCCT |
| | | GCAGGCCGAGGCCTTCCAGGCCCGCCTCAAGAGCTGGTTCGAGCCCCTGGT |
| | | GGAAGACATGCAGCGCCAGTGGGCCGGGCTGGTGGAGAAGGTGCAGGCTGC |
| | | CGTGGGCACCAGCGCCGCCCCTGTGCCCAGCGACAATCACTGA |
| 259 | 5 | GGAGTCGCTGCGCGCTGCCTTCGCCCCGTGCCCCGCTCCGCCGCCGCCTCG |
| | | CGCCGCCCGCCCCGGCTCTGACTGACCGCGTTACTCCCACAGGTGAGCGGG |
| | | CGGGACGGCCCTTCTCCTCCGGGCTGTAATTAGCTGAGCAAGAGGTAAGGGT |
| | | TTAAGGGATGGTTGGTTGGTGGGGTATGGGGCCCTGGCTGGGATTTCATCAT |
| | | ATACTGTAAGTTTGCGATGAGACACTACAGTATAGATGATGTACTAGTCCGGG |
| | | CACCCCCAGCTCTGGAGCCTGACAAGGAGGACAGGAGAGATGCTGCAAGCCC |
| | | AAGAAGCTCTCTGCTCAGCCTGTCACAACCTACTGACTGCCAGGGCACTTGGG |
| | | AATGGCAAGGCACAGAACCTTCATCTTCCTGCGAAGATGAAGGTTCTGTCTCT |
| | | TGCTATACCCAGAAAACGTGCCAGGAAGAGAACTCAGGACCCTGAAGCAGAC |
| | | TACTGGAAGGGAGACTCCAGCTCAAACAAGGCAGGGGTGGGGGCGTGGGAT |
| | | TGGGGGTAGGGGAGGGAATAGATACATTTTCTCTTTCCTGTTGTAAAGATAAT |
| | | GTTTAATTACCTGGAGCACCTGCCTGAAATCACTTTTTTTCAGGTTGGGCTAGC |
| | | GCCACCATGAAGGTTCTGTGGGCTGCGTTGCTGGTCACATTCCTGGCAGGAT |
| | | GCCAGGCCAAGGTGGAGCAAGCGGTGGAGACAGAGCCGGAGCCCGAGCTGC |
| | | GCCAGCAGACCGAGTGGCAGAGCGGCCAGCGCTGGGAACTGGCACTGGGTC |
| | | GCTTTTGGGATTACCTGCGCTGGGTGCAGACACTGTCTGAGCAGGTGCAGGA |
| | | GGAGCTGCTCAGCTCCCAGGTCACCCAGGAACTGAGGGCGCTGATGGACGA |
| | | GACCATGAAGGAGTTGAAGGCCTACAAATCGGAACTGGAGGAACAACTGACC |
| | | CCGGTGGCGGAGGAGACGCGGGCACGGCTGTCCAAGGAGCTGCAGGCGGC |
| | | GCAGGCCCGGCTGGGCGCGGACATGGAGGACGTGTGCGGCCGCCTGGTGC |
| | | AGTACCGCGGCGAGGTGCAGGCCATGCTCGGCCAGAGCACCGAGGAGCTGC |
| | | GGGTGAGCCTCGCCTCCCACCTGCGCAAGCTGCGTAAGCGGCTCCTCCGCG |
| | | ATGCCGATGACCTGCAGAAGCGCCTGGCAGTGTACCAGGCCGGGGCCCGCG |
| | | AGGGCGCCGAGCGCGGCCTCAGCGCCATCCGCGAGCGCCTGGGGCCCCTG |
| | | GTGGAACAGGGCCGCGTGCGGGCCGCCACTGTGGGCTCCCTGGCCGGCCAG |
| | | CCGCTACAGGAGCGGGCCCAGGCCTGGGGCGAGCGGCTGCGCGCGCGGAT |
| | | GGAGGAGATGGGCAGCCGGACCCGCGACCGCCTGGACGAGGTGAAGGAGCA |
| | | GGTGGCGGAGGTGCGCGCCAAGCTGGAGGAGCAGGCCCAGCAGATACGCCT |
| | | GCAGGCCGAGGCCTTCCAGGCCCGCCTCAAGAGCTGGTTCGAGCCCCTGGT |
| | | GGAAGACATGCAGCGCCAGTGGGCCGGGCTGGTGGAGAAGGTGCAGGCTGC |
| | | CGTGGGCACCAGCGCCGCCCCTGTGCCCAGCGACAATCACTGA |
| 260 | 6 | GGAGTCGCTGCGCGCTGCCTTCGCCCCGTGCCCCGCTCCGCCGCCGCCTCG |
| | | CGCCGCCCGCCCCGGCTCTGACTGACCGCGTTACTCCCACAGGTGAGCGGG |
| | | CGGGACGGCCCTTCTCCTCCGGGCTGTAATTAGCTGAGCAAGAGGTAAGGGT |
| | | TTAAGGGATGGTTGGTTGGTGGGGTATGGGGCCCTGGCTGGGATTTCATCAT |
| | | ATACTGTAAGTTTGCGATGAGACACTACAGTATAGATGATGTACTAGTCCGGG |
| | | CACCCCCAGCTCTGGAGCCTGACAAGGAGGACAGGAGAGATGCTGCAAGCCC |
| | | AAGAAGCTCTCTGCTCAGCCTGTCACAACCTACTGACTGCCAGGGCACTTGGG |
| | | AATGGCAAGGCACAGAACCTTCATCTTCCTGCGAAGATGAAGGTTCTGTCTCT |
| | | TGCTATACCCAGAAAACGTGCCAGGAAGAGAACTCAGGACCCTGAAGCAGAC |
| | | TACTGGAAGGGAGACTCCAGCTCAAACAAGGCAGGGGTGGGGGCGTGGGAT |
| | | TGGGGGTAGGGGAGGGAATAGATACATTTTCTCTTTCCTGTTGTAAAGATAAT |
| | | GTTTAATTACCTGGAGCACCTGCCTGAAATCACTTTTTTTCAGGTTGGGCTAGC |
| | | GCCACCATGAAGGTTCTGTGGGCTGCGTTGCTGGTCACATTCCTGGCAGGAT |
| | | GCCAGGCCAAGGTGGAGCAAGCGGTGGAGACAGAGCCGGAGCCCGAGCTGC |
| | | GCCAGCAGACCGAGTGGCAGAGCGGCCAGCGCTGGGAACTGGCACTGGGTC |
| | | GCTTTTGGGATTACCTGCGCTGGGTGCAGACACTGTCTGAGCAGGTGCAGGA |
| | | GGAGCTGCTCAGCTCCCAGGTCACCCAGGAACTGAGGGCGCTGATGGACGA |
| | | GACCATGAAGGAGTTGAAGGCCTACAAATCGGAACTGGAGGAACAACTGACC |
| | | CCGGTGGCGGAGGAGACGCGGGCACGGCTGTCCAAGGAGCTGCAGGCGGC |
| | | GCAGGCCCGGCTGGGCGCGGACATGGAGGACGTGTGCGGCCGCCTGGTGC |
| | | AGTACCGCGGCGAGGTGCAGGCCATGCTCGGCCAGAGCACCGAGGAGCTGC |
| | | GGGTGCGCCTCGCCTCCCACCTGCGCAAGCTGCGTAAGCGGCTCCTCCGCG |
| | | ATGCCGATGACCTGCAGAAGCGCCTGGCAGTGTACCAGGCCGGGGCCCGCG |
| | | AGGGCGCCGAGCGCGGCCTCAGCGCCATCCGCGAGCGCCTGGGGCCCCTG |
| | | GTGGAACAGGGCCGCGTGCGGGCCGCCACTGTGGGCTCCCTGGCCGGCCAG |
| | | CCGCTACAGGAGCGGGCCCAGGCCTGGGGCGAGCGGCTGCGCGCGCGGAT |
| | | GGAGGAGATGGGCAGCCGGACCCGCGACCGCCTGGACGAGGTGAAGGAGCA |
| | | GGAGGCGGAGGTGCGCGCCAAGCTGGAGGAGCAGGCCCAGCAGATACGCCT |
| | | GCAGGCCGAGGCCTTCCAGGCCCGCCTCAAGAGCTGGTTCGAGCCCCTGGT |
| | | GGAAGACATGCAGCGCCAGTGGGCCGGGCTGGTGGAGAAGGTGCAGGCTGC |
| | | CGTGGGCACCAGCGCCGCCCCTGTGCCCAGCGACAATCACTGA |
| 261 | 7 | GGAGTCGCTGCGCGCTGCCTTCGCCCCGTGCCCCGCTCCGCCGCCGCCTCG |
| | | CGCCGCCCGCCCCGGCTCTGACTGACCGCGTTACTCCCACAGGTGAGCGGG |
| | | CGGGACGGCCCTTCTCCTCCGGGCTGTAATTAGCTGAGCAAGAGGTAAGGGT |
| | | TTAAGGGATGGTTGGTTGGTGGGGTATGGGGCCCTGGCTGGGATTTCATCAT |
| | | ATACTGTAAGTTTGCGATGAGACACTACAGTATAGATGATGTACTAGTCCGGG |
| | | CACCCCCAGCTCTGGAGCCTGACAAGGAGGACAGGAGAGATGCTGCAAGCCC |
| | | AAGAAGCTCTCTGCTCAGCCTGTCACAACCTACTGACTGCCAGGGCACTTGGG |
| | | AATGGCAAGGAATGTGACCAGCAACGCAGCCCTGCGTTGCTGGTCACATCTCT |
| | | TGCTATACCCAGAAAACGTGCCAGGAAGAGAACTCAGGACCCTGAAGCAGAC |
| | | TACTGGAAGGGAGACTCCAGCTCAAACAAGGCAGGGGTGGGGGCGTGGGAT |
| | | TGGGGGTAGGGGAGGGAATAGATACATTTTCTCTTTCCTGTTGTAAAGATAAT |
| | | GTTTAATTACCTGGAGCACCTGCCTGAAATCACTTTTTTTCAGGTTGGGCTAGC |
| | | GCCACCATGAAAGTCCTCTGGGCCGCTCTCCTGGTGACCTTTCTGGCCGGCT |
| | | GTCAGGCCAAAGTGGAACAGGCCGTGGAAACCGAGCCTGAGCCTGAACTGAG |
| | | ACAGCAGACAGAGTGGCAGTCCGGCCAGAGATGGGAACTTGCCCTGGGCAG |
| | | ATTCTGGGACTACCTGAGATGGGTGCAGACCCTGAGCGAACAGGTGCAAGAG |
| | | GAACTGCTGAGCAGCCAAGTGACCCAAGAGCTGAGAGCCCTGATGGACGAGA |
| | | CAATGAAAGAACTCAAAGCTTATAAGAGCGAGCTCGAAGAGCAGCTGACCCCT |
| | | GTGGCCGAGGAAACAAGAGCCAGACTGAGCAAAGAGCTGCAGGCCGCTCAA |
| | | GCTAGACTGGGCGCCGATATGGAAGATGTGTGCGGCAGACTGGTGCAGTACA |
| | | GAGGCGAAGTGCAGGCCATGCTGGGCCAGTCTACCGAGGAACTGAGAGTGC |
| | | GGCTGGCCAGCCACCTGAGAAAGCTGCGGAAGAGACTGCTGAGAGATGCCG |
| | | ACGACCTGCAGAAATGCCTGGCCGTGTATCAGGCTGGCGCTAGAGAAGGCGC |
| | | TGAGAGAGGACTGTCTGCCATCCGGGAAAGACTGGGCCCTCTGGTTGAACAG |
| | | GGCAGAGTTAGAGCCGCCACAGTGGGATCTCTTGCAGGCCAGCCTCTGCAAG |
| | | AAAGAGCCCAGGCTTGGGGCGAGAGACTGAGGGCCAGAATGGAAGAGATGG |
| | | GCAGCAGAACCAGAGACAGACTGGACGAAGTGAAAGAACAGGTGGCCGAAGT |
| | | GCGCGCCAAGCTCGAAGAACAAGCCCAGCAGATTCGGCTGCAGGCTGAAGCC |
| | | TTTCAGGCCCGGCTGAAGAGTTGGTTCGAGCCCCTGGTGGAAGATATGCAGA |
| | | GACAGTGGGCTGGACTGGTGGAAAAGGTGCAGGCTGCCGTGGGAACATCTG |
| | | CTGCTCCAGTGCCTAGCGACAACCACTGA |
| 262 | 8 | GGAGTCGCTGCGCGCTGCCTTCGCCCCGTGCCCCGCTCCGCCGCCGCCTCG |
| | | CGCCGCCCGCCCCGGCTCTGACTGACCGCGTTACTCCCACAGGTGAGCGGG |
| | | CGGGACGGCCCTTCTCCTCCGGGCTGTAATTAGCTGAGCAAGAGGTAAGGGT |
| | | TTAAGGGATGGTTGGTTGGTGGGGTATGGGGCCCTGGCTGGGATTTCATCAT |
| | | ATACTGTAAGTTTGCGATGAGACACTACAGTATAGATGATGTACTAGTCCGGG |
| | | CACCCCCAGCTCTGGAGCCTGACAAGGAGGACAGGAGAGATGCTGCAAGCCC |
| | | AAGAAGCTCTCTGCTCAGCCTGTCACAACCTACTGACTGCCAGGGCACTTGGG |
| | | AATGGCAAGGAATGTGACCAGCAACGCAGCCCTGCGTTGCTGGTCACATCTCT |
| | | TGCTATACCCAGAAAACGTGCCAGGAAGAGAACTCAGGACCCTGAAGCAGAC |
| | | TACTGGAAGGGAGACTCCAGCTCAAACAAGGCAGGGGTGGGGGCGTGGGAT |
| | | TGGGGGTAGGGGAGGGAATAGATACATTTTCTCTTTCCTGTTGTAAAGATAAT |
| | | GTTTAATTACCTGGAGCACCTGCCTGAAATCACTTTTTTTCAGGTTGGGCTAGC |
| | | GCCACCATGAAAGTCCTCTGGGCCGCTCTCCTGGTGACCTTTCTGGCCGGCT |
| | | GTCAGGCCAAAGTGGAACAGGCCGTGGAAACCGAGCCTGAGCCTGAACTGAG |
| | | ACAGCAGACAGAGTGGCAGTCCGGCCAGAGATGGGAACTTGCCCTGGGCAG |
| | | ATTCTGGGACTACCTGAGATGGGTGCAGACCCTGAGCGAACAGGTGCAAGAG |
| | | GAACTGCTGAGCAGCCAAGTGACCCAAGAGCTGAGAGCCCTGATGGACGAGA |
| | | CAATGAAAGAACTCAAAGCTTATAAGAGCGAGCTCGAAGAGCAGCTGACCCCT |
| | | GTGGCCGAGGAAACAAGAGCCAGACTGAGCAAAGAGCTGCAGGCCGCTCAA |
| | | GCTAGACTGGGCGCCGATATGGAAGATGTGTGCGGCAGACTGGTGCAGTACA |
| | | GAGGCGAAGTGCAGGCCATGCTGGGCCAGTCTACCGAGGAACTGAGAGTGTC |
| | | TCTGGCCAGCCACCTGAGAAAGCTGCGGAAGAGACTGCTGAGAGATGCCGAC |
| | | GACCTGCAGAAATGCCTGGCCGTGTATCAGGCTGGCGCTAGAGAAGGCGCTG |
| | | AGAGAGGACTGTCTGCCATCCGGGAAAGACTGGGCCCTCTGGTTGAACAGGG |
| | | CAGAGTTAGAGCCGCCACAGTGGGATCTCTTGCAGGCCAGCCTCTGCAAGAA |
| | | AGAGCCCAGGCTTGGGGCGAGAGACTGAGGGCCAGAATGGAAGAGATGGGC |
| | | AGCAGAACCAGAGACAGACTGGACGAAGTGAAAGAACAGGTGGCCGAAGTGC |
| | | GCGCCAAGCTCGAAGAACAAGCCCAGCAGATTCGGCTGCAGGCTGAAGCCTT |
| | | TCAGGCCCGGCTGAAGAGTTGGTTCGAGCCCCTGGTGGAAGATATGCAGAGA |
| | | CAGTGGGCTGGACTGGTGGAAAAGGTGCAGGCTGCCGTGGGAACATCTGCTG |
| | | CTCCAGTGCCTAGCGACAACCACTGA |
| 263 | 9 | GGAGTCGCTGCGCGCTGCCTTCGCCCCGTGCCCCGCTCCGCCGCCGCCTCG |
| | | CGCCGCCCGCCCCGGCTCTGACTGACCGCGTTACTCCCACAGGTGAGCGGG |
| | | CGGGACGGCCCTTCTCCTCCGGGCTGTAATTAGCTGAGCAAGAGGTAAGGGT |
| | | TTAAGGGATGGTTGGTTGGTGGGGTATGGGGCCCTGGCTGGGATTTCATCAT |
| | | ATACTGTAAGTTTGCGATGAGACACTACAGTATAGATGATGTACTAGTCCGGG |
| | | CACCCCCAGCTCTGGAGCCTGACAAGGAGGACAGGAGAGATGCTGCAAGCCC |
| | | AAGAAGCTCTCTGCTCAGCCTGTCACAACCTACTGACTGCCAGGGCACTTGGG |
| | | AATGGCAAGGAATGTGACCAGCAACGCAGCCCTGCGTTGCTGGTCACATCTCT |
| | | TGCTATACCCAGAAAACGTGCCAGGAAGAGAACTCAGGACCCTGAAGCAGAC |
| | | TACTGGAAGGGAGACTCCAGCTCAAACAAGGCAGGGGTGGGGGCGTGGGAT |
| | | TGGGGGTAGGGGAGGGAATAGATACATTTTCTCTTTCCTGTTGTAAAGATAAT |
| | | GTTTAATTACCTGGAGCACCTGCCTGAAATCACTTTTTTTCAGGTTGGGCTAGC |
| | | GCCACCATGAAAGTCCTCTGGGCCGCTCTCCTGGTGACCTTTCTGGCAGGAT |
| | | GCCAGGCCAAGGTGGAGCAAGCGGTGGAGACAGAGCCGGAGCCCGAGCTGC |
| | | GCCAGCAGACCGAGTGGCAGAGCGGCCAGCGCTGGGAACTGGCACTGGGTC |
| | | GCTTTTGGGATTACCTGCGCTGGGTGCAGACACTGTCTGAGCAGGTGCAGGA |
| | | GGAGCTGCTCAGCTCCCAGGTCACCCAGGAACTGAGGGCGCTGATGGACGA |
| | | GACCATGAAAGAACTCAAAGCTTATAAGAGCGAGCTCGAAGAGCAGCTGACCC |
| | | CGGTGGCGGAGGAGACGCGGGCACGGCTGTCCAAGGAGCTGCAGGCGGCG |
| | | CAGGCCCGGCTGGGCGCGGACATGGAGGACGTGTGCGGCCGCCTGGTGCA |
| | | GTACCGCGGCGAGGTGCAGGCCATGCTCGGCCAGAGCACCGAGGAGCTGCG |
| | | GGTGAGCCTCGCCTCCCACCTGCGCAAGCTGCGTAAGCGGCTCCTCCGCGAT |
| | | GCCGATGACCTGCAGAAGTGCCTGGCAGTGTACCAGGCCGGGGCCCGCGAG |
| | | GGCGCCGAGCGCGGCCTCAGCGCCATCCGCGAGCGCCTGGGGCCCCTGGT |
| | | GGAACAGGGCCGCGTGCGGGCCGCCACTGTGGGCTCCCTGGCCGGCCAGC |
| | | CGCTACAGGAGCGGGCCCAGGCCTGGGGCGAGCGGCTGCGCGCGCGGATG |
| | | GAGGAGATGGGCAGCCGGACCCGCGACCGCCTGGACGAGGTGAAGGAGCAG |
| | | GTGGCGGAGGTGCGCGCCAAGCTGGAGGAGCAGGCCCAGCAGATACGCCTG |
| | | CAGGCCGAGGCCTTCCAGGCCCGCCTCAAGAGCTGGTTCGAGCCCCTGGTG |
| | | GAAGACATGCAGCGCCAGTGGGCCGGGCTGGTGGAGAAGGTGCAGGCTGCC |
| | | GTGGGCACCAGCGCCGCCCCTGTGCCCAGCGACAATCACTGA |
| 264 | 10 | GGAGTCGCTGCGCGCTGCCTTCGCCCCGTGCCCCGCTCCGCCGCCGCCTCG |
| | | CGCCGCCCGCCCCGGCTCTGACTGACCGCGTTACTCCCACAGGTGAGCGGG |
| | | CGGGACGGCCCTTCTCCTCCGGGCTGTAATTAGCTGAGCAAGAGGTAAGGGT |
| | | TTAAGGGATGGTTGGTTGGTGGGGTATGGGGCCCTGGCTGGGATTTCATCAT |
| | | ATACTGTAAGTTTGCGATGAGACACTACAGTATAGATGATGTACTAGTCCGGG |
| | | CACCCCCAGCTCTGGAGCCTGACAAGGAGGACAGGAGAGATGCTGCAAGCCC |
| | | AAGAAGCTCTCTGCTCAGCCTGTCACAACCTACTGACTGCCAGGGCACTTGGG |
| | | AATGGCAAGGAATGTGACCAGCAACGCAGCCCTGCGTTGCTGGTCACATCTCT |
| | | TGCTATACCCAGAAAACGTGCCAGGAAGAGAACTCAGGACCCTGAAGCAGAC |
| | | TACTGGAAGGGAGACTCCAGCTCAAACAAGGCAGGGGTGGGGGCGTGGGAT |
| | | TGGGGGTAGGGGAGGGAATAGATACATTTTCTCTTTCCTGTTGTAAAGATAAT |
| | | GTTTAATTACCTGGAGCACCTGCCTGAAATCACTTTTTTTCAGGTTGGGCTAGC |
| | | GCCACCATGAAAGTCCTCTGGGCCGCTCTCCTGGTGACCTTTCTGGCCGGCT |
| | | GTCAGGCCAAAGTGGAACAGGCCGTGGAAACCGAGCCTGAGCCTGAACTGAG |
| | | ACAGCAGACAGAGTGGCAGTCCGGCCAGAGATGGGAACTTGCCCTGGGCAG |
| | | ATTCTGGGACTACCTGAGATGGGTGCAGACCCTGAGCGAACAGGTGCAAGAG |
| | | GAACTGCTGAGCAGCCAAGTGACCCAAGAGCTGAGAGCCCTGATGGACGAGA |
| | | CAATGAAAGAACTCAAAGCTTATAAGAGCGAGCTCGAAGAGCAGCTGACCCCT |
| | | GTGGCCGAGGAAACAAGAGCCAGACTGAGCAAAGAGCTGCAGGCCGCTCAA |
| | | GCTAGACTGGGCGCCGATATGGAAGATGTGTGCGGCAGACTGGTGCAGTACA |
| | | GAGGCGAAGTGCAGGCCATGCTGGGCCAGTCTACCGAGGAACTGAGAGTGC |
| | | GGCTGGCCAGCCACCTGAGAAAGCTGCGGAAGAGACTGCTGAGAGATGCCG |
| | | ACGACCTGCAGAAATGCCTGGCCGTGTATCAGGCTGGCGCTAGAGAAGGCGC |
| | | TGAGAGAGGACTGTCTGCCATCCGGGAAAGACTGGGCCCTCTGGTTGAACAG |
| | | GGCAGAGTTAGAGCCGCCACAGTGGGATCTCTTGCAGGCCAGCCTCTGCAAG |
| | | AAAGAGCCCAGGCTTGGGGCGAGAGACTGAGGGCCAGAATGGAAGAGATGG |
| | | GCAGCAGAACCAGAGACAGACTGGACGAAGTGAAAGAACAGGAGGCCGAAGT |
| | | GCGCGCCAAGCTCGAAGAACAAGCCCAGCAGATTCGGCTGCAGGCTGAAGCC |
| | | TTTCAGGCCCGGCTGAAGAGTTGGTTCGAGCCCCTGGTGGAAGATATGCAGA |
| | | GACAGTGGGCTGGACTGGTGGAAAAGGTGCAGGCTGCCGTGGGAACATCTG |
| | | CTGCTCCAGTGCCTAGCGACAACCACTGA |
| 265 | 11 | GGAGTCGCTGCGCGCTGCCTTCGCCCCGTGCCCCGCTCCGCCGCCGCCTCG |
| | | CGCCGCCCGCCCCGGCTCTGACTGACCGCGTTACTCCCACAGGTGAGCGGG |
| | | CGGGACGGCCCTTCTCCTCCGGGCTGTAATTAGCTGAGCAAGAGGTAAGGGT |
| | | TTAAGGGATGGTTGGTTGGTGGGGTATGGGGCCCTGGCTGGGATTTCATCAT |
| | | ATACTGTAAGTTTGCGATGAGACACTACAGTATAGATGATGTACTAGTCCGGG |
| | | CACCCCCAGCTCTGGAGCCTGACAAGGAGGACAGGAGAGATGCTGCAAGCCC |
| | | AAGAAGCTCTCTGCTCAGCCTGTCACAACCTACTGACTGCCAGGGCACTTGGG |
| | | AATGGCAAGGAATGTGACCAGCAACGCAGCCCTGCGTTGCTGGTCACATCTCT |
| | | TGCTATACCCAGAAAACGTGCCAGGAAGAGAACTCAGGACCCTGAAGCAGAC |
| | | TACTGGAAGGGAGACTCCAGCTCAAACAAGGCAGGGGTGGGGGCGTGGGAT |
| | | TGGGGGTAGGGGAGGGAATAGATACATTTTCTCTTTCCTGTTGTAAAGATAAT |
| | | GTTTAATTACCTGGAGCACCTGCCTGAAATCACTTTTTTTCAGGTTGGGCTAGC |
| | | GCCACCATGAAAGTCCTCTGGGCCGCTCTCCTGGTGACCTTTCTGGCAGGAT |
| | | GCCAGGCCAAGGTGGAGCAAGCGGTGGAGACAGAGCCGGAGCCCGAGCTGC |
| | | GCCAGCAGACCGAGTGGCAGAGCGGCCAGCGCTGGGAACTGGCACTGGGTC |
| | | GCTTTTGGGATTACCTGCGCTGGGTGCAGACACTGTCTGAGCAGGTGCAGGA |
| | | GGAGCTGCTCAGCTCCCAGGTCACCCAGGAACTGAGGGCGCTGATGGACGA |
| | | GACCATGAAAGAACTCAAAGCTTATAAGAGCGAGCTCGAAGAGCAGCTGACCC |
| | | CGGTGGCGGAGGAGACGCGGGCACGGCTGTCCAAGGAGCTGCAGGCGGCG |
| | | CAGGCCCGGCTGGGCGCGGACATGGAGGACGTGTGCGGCCGCCTGGTGCA |
| | | GTACCGCGGCGAGGTGCAGGCCATGCTCGGCCAGAGCACCGAGGAGCTGCG |
| | | GGTGCGCCTCGCCTCCCACCTGCGCAAGCTGCGTAAGCGGCTCCTCCGCGAT |
| | | GCCGATGACCTGCAGAAGTGCCTGGCAGTGTACCAGGCCGGGGCCCGCGAG |
| | | GGCGCCGAGCGCGGCCTCAGCGCCATCCGCGAGCGCCTGGGGCCCCTGGT |
| | | GGAACAGGGCCGCGTGCGGGCCGCCACTGTGGGCTCCCTGGCCGGCCAGC |
| | | CGCTACAGGAGCGGGCCCAGGCCTGGGGCGAGCGGCTGCGCGCGCGGATG |
| | | GAGGAGATGGGCAGCCGGACCCGCGACCGCCTGGACGAGGTGAAGGAGCAG |
| | | GAGGCGGAGGTGCGCGCCAAGCTGGAGGAGCAGGCCCAGCAGATACGCCTG |
| | | CAGGCCGAGGCCTTCCAGGCCCGCCTCAAGAGCTGGTTCGAGCCCCTGGTG |
| | | GAAGACATGCAGCGCCAGTGGGCCGGGCTGGTGGAGAAGGTGCAGGCTGCC |
| | | GTGGGCACCAGCGCCGCCCCTGTGCCCAGCGACAATCACTGA |
| 266 | 12 | GGAGTCGCTGCGCGCTGCCTTCGCCCCGTGCCCCGCTCCGCCGCCGCCTCG |
| | | CGCCGCCCGCCCCGGCTCTGACTGACCGCGTTACTCCCACAGGTGAGCGGG |
| | | CGGGACGGCCCTTCTCCTCCGGGCTGTAATTAGCTGAGCAAGAGGTAAGGGT |
| | | TTAAGGGATGGTTGGTTGGTGGGGTATGGGGCCCTGGCTGGGATTTCATCAT |
| | | ATACTGTAAGTTTGCGATGAGACACTACAGTATAGATGATGTACTAGTCCGGG |
| | | CACCCCCAGCTCTGGAGCCTGACAAGGAGGACAGGAGAGATGCTGCAAGCCC |
| | | AAGAAGCTCTCTGCTCAGCCTGTCACAACCTACTGACTGCCAGGGCACTTGGG |
| | | AATGGCAAGGAATGTGACCAGCAACGCAGCCCTGCGTTGCTGGTCACATCTCT |
| | | TGCTATACCCAGAAAACGTGCCAGGAAGAGAACTCAGGACCCTGAAGCAGAC |
| | | TACTGGAAGGGAGACTCCAGCTCAAACAAGGCAGGGGTGGGGGCGTGGGAT |
| | | TGGGGGTAGGGGAGGGAATAGATACATTTTCTCTTTCCTGTTGTAAAGATAAT |
| | | GTTTAATTACCTGGAGCACCTGCCTGAAATCACTTTTTTTCAGGTTGGGCTAGC |
| | | GCCACCATGAAAGTCCTCTGGGCCGCTCTCCTGGTGACCTTTCTGGCCGGCT |
| | | GTCAGGCCAAAGTGGAACAGGCCGTGGAAACCGAGCCTGAGCCTGAACTGAG |
| | | ACAGCAGACAGAGTGGCAGTCCGGCCAGAGATGGGAACTTGCCCTGGGCAG |
| | | ATTCTGGGACTACCTGAGATGGGTGCAGACCCTGAGCGAACAGGTGCAAGAG |
| | | GAACTGCTGAGCAGCCAAGTGACCCAAGAGCTGAGAGCCCTGATGGACGAGA |
| | | CAATGAAAGAACTCAAAGCTTATAAGAGCGAGCTCGAAGAGCAGCTGACCCCT |
| | | GTGGCCGAGGAAACAAGAGCCAGACTGAGCAAAGAGCTGCAGGCCGCTCAA |
| | | GCTAGACTGGGCGCCGATATGGAAGATGTGTGCGGCAGACTGGTGCAGTACA |
| | | GAGGCGAAGTGCAGGCCATGCTGGGCCAGTCTACCGAGGAACTGAGAGTGC |
| | | GGCTGGCCAGCCACCTGAGAAAGCTGCGGAAGAGACTGCTGAGAGATGCCG |
| | | ACGACCTGCAGAAACGCCTGGCCGTGTATCAGGCTGGCGCTAGAGAAGGCGC |
| | | TGAGAGAGGACTGTCTGCCATCCGGGAAAGACTGGGCCCTCTGGTTGAACAG |
| | | GGCAGAGTTAGAGCCGCCACAGTGGGATCTCTTGCAGGCCAGCCTCTGCAAG |
| | | AAAGAGCCCAGGCTTGGGGCGAGAGACTGAGGGCCAGAATGGAAGAGATGG |
| | | GCAGCAGAACCAGAGACAGACTGGACGAAGTGAAAGAACAGGTGGCCGAAGT |
| | | GCGCGCCAAGCTCGAAGAACAAGCCCAGCAGATTCGGCTGCAGGCTGAAGCC |
| | | TTTCAGGCCCGGCTGAAGAGTTGGTTCGAGCCCCTGGTGGAAGATATGCAGA |
| | | GACAGTGGGCTGGACTGGTGGAAAAGGTGCAGGCTGCCGTGGGAACATCTG |
| | | CTGCTCCAGTGCCTAGCGACAACCACTGA |
| 267 | 13 | GGAGTCGCTGCGCGCTGCCTTCGCCCCGTGCCCCGCTCCGCCGCCGCCTCG |
| | | CGCCGCCCGCCCCGGCTCTGACTGACCGCGTTACTCCCACAGGTGAGCGGG |
| | | CGGGACGGCCCTTCTCCTCCGGGCTGTAATTAGCTGAGCAAGAGGTAAGGGT |
| | | TTAAGGGATGGTTGGTTGGTGGGGTATGGGGCCCTGGCTGGGATTTCATCAT |
| | | ATACTGTAAGTTTGCGATGAGACACTACAGTATAGATGATGTACTAGTCCGGG |
| | | CACCCCCAGCTCTGGAGCCTGACAAGGAGGACAGGAGAGATGCTGCAAGCCC |
| | | AAGAAGCTCTCTGCTCAGCCTGTCACAACCTACTGACTGCCAGGGCACTTGGG |
| | | AATGGCAAGGAATGTGACCAGCAACGCAGCCCTGCGTTGCTGGTCACATCTCT |
| | | TGCTATACCCAGAAAACGTGCCAGGAAGAGAACTCAGGACCCTGAAGCAGAC |
| | | TACTGGAAGGGAGACTCCAGCTCAAACAAGGCAGGGGTGGGGGCGTGGGAT |
| | | TGGGGGTAGGGGAGGGAATAGATACATTTTCTCTTTCCTGTTGTAAAGATAAT |
| | | GTTTAATTACCTGGAGCACCTGCCTGAAATCACTTTTTTTCAGGTTGGGCTAGC |
| | | GCCACCATGAAAGTCCTCTGGGCCGCTCTCCTGGTGACCTTTCTGGCCGGCT |
| | | GTCAGGCCAAAGTGGAACAGGCCGTGGAAACCGAGCCTGAGCCTGAACTGAG |
| | | ACAGCAGACAGAGTGGCAGTCCGGCCAGAGATGGGAACTTGCCCTGGGCAG |
| | | ATTCTGGGACTACCTGAGATGGGTGCAGACCCTGAGCGAACAGGTGCAAGAG |
| | | GAACTGCTGAGCAGCCAAGTGACCCAAGAGCTGAGAGCCCTGATGGACGAGA |
| | | CAATGAAAGAACTCAAAGCTTATAAGAGCGAGCTCGAAGAGCAGCTGACCCCT |
| | | GTGGCCGAGGAAACAAGAGCCAGACTGAGCAAAGAGCTGCAGGCCGCTCAA |
| | | GCTAGACTGGGCGCCGATATGGAAGATGTGTGCGGCAGACTGGTGCAGTACA |
| | | GAGGCGAAGTGCAGGCCATGCTGGGCCAGTCTACCGAGGAACTGAGAGTGTC |
| | | TCTGGCCAGCCACCTGAGAAAGCTGCGGAAGAGACTGCTGAGAGATGCCGAC |
| | | GACCTGCAGAAACGCCTGGCCGTGTATCAGGCTGGCGCTAGAGAAGGCGCTG |
| | | AGAGAGGACTGTCTGCCATCCGGGAAAGACTGGGCCCTCTGGTTGAACAGGG |
| | | CAGAGTTAGAGCCGCCACAGTGGGATCTCTTGCAGGCCAGCCTCTGCAAGAA |
| | | AGAGCCCAGGCTTGGGGCGAGAGACTGAGGGCCAGAATGGAAGAGATGGGC |
| | | AGCAGAACCAGAGACAGACTGGACGAAGTGAAAGAACAGGTGGCCGAAGTGC |
| | | GCGCCAAGCTCGAAGAACAAGCCCAGCAGATTCGGCTGCAGGCTGAAGCCTT |
| | | TCAGGCCCGGCTGAAGAGTTGGTTCGAGCCCCTGGTGGAAGATATGCAGAGA |
| | | CAGTGGGCTGGACTGGTGGAAAAGGTGCAGGCTGCCGTGGGAACATCTGCTG |
| | | CTCCAGTGCCTAGCGACAACCACTGA |
| 268 | 14 | GGAGTCGCTGCGCGCTGCCTTCGCCCCGTGCCCCGCTCCGCCGCCGCCTCG |
| | | CGCCGCCCGCCCCGGCTCTGACTGACCGCGTTACTCCCACAGGTGAGCGGG |
| | | CGGGACGGCCCTTCTCCTCCGGGCTGTAATTAGCTGAGCAAGAGGTAAGGGT |
| | | TTAAGGGATGGTTGGTTGGTGGGGTATGGGGCCCTGGCTGGGATTTCATCAT |
| | | ATACTGTAAGTTTGCGATGAGACACTACAGTATAGATGATGTACTAGTCCGGG |
| | | CACCCCCAGCTCTGGAGCCTGACAAGGAGGACAGGAGAGATGCTGCAAGCCC |
| | | AAGAAGCTCTCTGCTCAGCCTGTCACAACCTACTGACTGCCAGGGCACTTGGG |
| | | AATGGCAAGGAATGTGACCAGCAACGCAGCCCTGCGTTGCTGGTCACATCTCT |
| | | TGCTATACCCAGAAAACGTGCCAGGAAGAGAACTCAGGACCCTGAAGCAGAC |
| | | TACTGGAAGGGAGACTCCAGCTCAAACAAGGCAGGGGTGGGGGCGTGGGAT |
| | | TGGGGGTAGGGGAGGGAATAGATACATTTTCTCTTTCCTGTTGTAAAGATAAT |
| | | GTTTAATTACCTGGAGCACCTGCCTGAAATCACTTTTTTTCAGGTTGGGCTAGC |
| | | GCCACCATGAAAGTCCTCTGGGCCGCTCTCCTGGTGACCTTTCTGGCCGGCT |
| | | GTCAGGCCAAAGTGGAACAGGCCGTGGAAACCGAGCCTGAGCCTGAACTGAG |
| | | ACAGCAGACAGAGTGGCAGTCCGGCCAGAGATGGGAACTTGCCCTGGGCAG |
| | | ATTCTGGGACTACCTGAGATGGGTGCAGACCCTGAGCGAACAGGTGCAAGAG |
| | | GAACTGCTGAGCAGCCAAGTGACCCAAGAGCTGAGAGCCCTGATGGACGAGA |
| | | CAATGAAAGAACTCAAAGCTTATAAGAGCGAGCTCGAAGAGCAGCTGACCCCT |
| | | GTGGCCGAGGAAACAAGAGCCAGACTGAGCAAAGAGCTGCAGGCCGCTCAA |
| | | GCTAGACTGGGCGCCGATATGGAAGATGTGTGCGGCAGACTGGTGCAGTACA |
| | | GAGGCGAAGTGCAGGCCATGCTGGGCCAGTCTACCGAGGAACTGAGAGTGC |
| | | GGCTGGCCAGCCACCTGAGAAAGCTGCGGAAGAGACTGCTGAGAGATGCCG |
| | | ACGACCTGCAGAAACGCCTGGCCGTGTATCAGGCTGGCGCTAGAGAAGGCGC |
| | | TGAGAGAGGACTGTCTGCCATCCGGGAAAGACTGGGCCCTCTGGTTGAACAG |
| | | GGCAGAGTTAGAGCCGCCACAGTGGGATCTCTTGCAGGCCAGCCTCTGCAAG |
| | | AAAGAGCCCAGGCTTGGGGCGAGAGACTGAGGGCCAGAATGGAAGAGATGG |
| | | GCAGCAGAACCAGAGACAGACTGGACGAAGTGAAAGAACAGGAGGCCGAAGT |
| | | GCGCGCCAAGCTCGAAGAACAAGCCCAGCAGATTCGGCTGCAGGCTGAAGCC |
| | | TTTCAGGCCCGGCTGAAGAGTTGGTTCGAGCCCCTGGTGGAAGATATGCAGA |
| | | GACAGTGGGCTGGACTGGTGGAAAAGGTGCAGGCTGCCGTGGGAACATCTG |
| | | CTGCTCCAGTGCCTAGCGACAACCACTGA |
| 269 | 15 | GGAGTCGCTGCGCGCTGCCTTCGCCCCGTGCCCCGCTCCGCCGCCGCCTCG |
| | | CGCCGCCCGCCCCGGCTCTGACTGACCGCGTTACTCCCACAGGTGAGCGGG |
| | | CGGGACGGCCCTTCTCCTCCGGGCTGTAATTAGCTGAGCAAGAGGTAAGGGT |
| | | TTAAGGGATGGTTGGTTGGTGGGGTATGGGGCCCTGGCTGGGATTTCATCAT |
| | | ATACTGTAAGTTTGCGATGAGACACTACAGTATAGATGATGTACTAGTCCGGG |
| | | CACCCCCAGCTCTGGAGCCTGACAAGGAGGACAGGAGAGATGCTGCAAGCCC |
| | | AAGAAGCTCTCTGCTCAGCCTGTCACAACCTACTGACTGCCAGGGCACTTGGG |
| | | AATGGCAAGGTCCAGTTCCGATTTGTAGGCCTCTACAAATCGGAACTGGCTCT |
| | | TGCTATACCCAGAAAACGTGCCAGGAAGAGAACTCAGGACCCTGAAGCAGAC |
| | | TACTGGAAGGGAGACTCCAGCTCAAACAAGGCAGGGGTGGGGGCGTGGGAT |
| | | TGGGGGTAGGGGAGGGAATAGATACATTTTCTCTTTCCTGTTGTAAAGATAAT |
| | | GTTTAATTACCTGGAGCACCTGCCTGAAATCACTTTTTTTCAGGTTGGGCTAGC |
| | | GCCACCATGAAAGTCCTCTGGGCCGCTCTCCTGGTGACCTTTCTGGCCGGCT |
| | | GTCAGGCCAAAGTGGAACAGGCCGTGGAAACCGAGCCTGAGCCTGAACTGAG |
| | | ACAGCAGACAGAGTGGCAGTCCGGCCAGAGATGGGAACTTGCCCTGGGCAG |
| | | ATTCTGGGACTACCTGAGATGGGTGCAGACCCTGAGCGAACAGGTGCAAGAG |
| | | GAACTGCTGAGCAGCCAAGTGACCCAAGAGCTGAGAGCCCTGATGGACGAGA |
| | | CAATGAAAGAACTCAAAGCTTATAAGAGCGAGCTCGAAGAGCAGCTGACCCCT |
| | | GTGGCCGAGGAAACAAGAGCCAGACTGAGCAAAGAGCTGCAGGCCGCTCAA |
| | | GCTAGACTGGGCGCCGATATGGAAGATGTGTGCGGCAGACTGGTGCAGTACA |
| | | GAGGCGAAGTGCAGGCCATGCTGGGCCAGTCTACCGAGGAACTGAGAGTGTC |
| | | TCTGGCCAGCCACCTGAGAAAGCTGCGGAAGAGACTGCTGAGAGATGCCGAC |
| | | GACCTGCAGAAATGCCTGGCCGTGTATCAGGCTGGCGCTAGAGAAGGCGCTG |
| | | AGAGAGGACTGTCTGCCATCCGGGAAAGACTGGGCCCTCTGGTTGAACAGGG |
| | | CAGAGTTAGAGCCGCCACAGTGGGATCTCTTGCAGGCCAGCCTCTGCAAGAA |
| | | AGAGCCCAGGCTTGGGGCGAGAGACTGAGGGCCAGAATGGAAGAGATGGGC |
| | | AGCAGAACCAGAGACAGACTGGACGAAGTGAAAGAACAGGTGGCCGAAGTGC |
| | | GCGCCAAGCTCGAAGAACAAGCCCAGCAGATTCGGCTGCAGGCTGAAGCCTT |
| | | TCAGGCCCGGCTGAAGAGTTGGTTCGAGCCCCTGGTGGAAGATATGCAGAGA |
| | | CAGTGGGCTGGACTGGTGGAAAAGGTGCAGGCTGCCGTGGGAACATCTGCTG |
| | | CTCCAGTGCCTAGCGACAACCACTGA |
| 270 | 16 | GGAGTCGCTGCGCGCTGCCTTCGCCCCGTGCCCCGCTCCGCCGCCGCCTCG |
| | | CGCCGCCCGCCCCGGCTCTGACTGACCGCGTTACTCCCACAGGTGAGCGGG |
| | | CGGGACGGCCCTTCTCCTCCGGGCTGTAATTAGCTGAGCAAGAGGTAAGGGT |
| | | TTAAGGGATGGTTGGTTGGTGGGGTATGGGGCCCTGGCTGGGATTTCATCAT |
| | | ATACTGTAAGTTTGCGATGAGACACTACAGTATAGATGATGTACTAGTCCGGG |
| | | CACCCCCAGCTCTGGAGCCTGACAAGGAGGACAGGAGAGATGCTGCAAGCCC |
| | | AAGAAGCTCTCTGCTCAGCCTGTCACAACCTACTGACTGCCAGGGCACTTGGG |
| | | AATGGCAAGGTCCAGTTCCGATTTGTAGGCCTCTACAAATCGGAACTGGCTCT |
| | | TGCTATACCCAGAAAACGTGCCAGGAAGAGAACTCAGGACCCTGAAGCAGAC |
| | | TACTGGAAGGGAGACTCCAGCTCAAACAAGGCAGGGGTGGGGGCGTGGGAT |
| | | TGGGGGTAGGGGAGGGAATAGATACATTTTCTCTTTCCTGTTGTAAAGATAAT |
| | | GTTTAATTACCTGGAGCACCTGCCTGAAATCACTTTTTTTCAGGTTGGGCTAGC |
| | | GCCACCATGAAAGTCCTCTGGGCCGCTCTCCTGGTGACCTTTCTGGCAGGAT |
| | | GCCAGGCCAAGGTGGAGCAAGCGGTGGAGACAGAGCCGGAGCCCGAGCTGC |
| | | GCCAGCAGACCGAGTGGCAGAGCGGCCAGCGCTGGGAACTGGCACTGGGTC |
| | | GCTTTTGGGATTACCTGCGCTGGGTGCAGACACTGTCTGAGCAGGTGCAGGA |
| | | GGAGCTGCTCAGCTCCCAGGTCACCCAGGAACTGAGGGCGCTGATGGACGA |
| | | GACCATGAAAGAACTCAAAGCTTATAAGAGCGAGCTCGAAGAGCAGCTGACCC |
| | | CGGTGGCGGAGGAGACGCGGGCACGGCTGTCCAAGGAGCTGCAGGCGGCG |
| | | CAGGCCCGGCTGGGCGCGGACATGGAGGACGTGTGCGGCCGCCTGGTGCA |
| | | GTACCGCGGCGAGGTGCAGGCCATGCTCGGCCAGAGCACCGAGGAGCTGCG |
| | | GGTGAGCCTCGCCTCCCACCTGCGCAAGCTGCGTAAGCGGCTCCTCCGCGAT |
| | | GCCGATGACCTGCAGAAGTGCCTGGCAGTGTACCAGGCCGGGGCCCGCGAG |
| | | GGCGCCGAGCGCGGCCTCAGCGCCATCCGCGAGCGCCTGGGGCCCCTGGT |
| | | GGAACAGGGCCGCGTGCGGGCCGCCACTGTGGGCTCCCTGGCCGGCCAGC |
| | | CGCTACAGGAGCGGGCCCAGGCCTGGGGCGAGCGGCTGCGCGCGCGGATG |
| | | GAGGAGATGGGCAGCCGGACCCGCGACCGCCTGGACGAGGTGAAGGAGCAG |
| | | GTGGCGGAGGTGCGCGCCAAGCTGGAGGAGCAGGCCCAGCAGATACGCCTG |
| | | CAGGCCGAGGCCTTCCAGGCCCGCCTCAAGAGCTGGTTCGAGCCCCTGGTG |
| | | GAAGACATGCAGCGCCAGTGGGCCGGGCTGGTGGAGAAGGTGCAGGCTGCC |
| | | GTGGGCACCAGCGCCGCCCCTGTGCCCAGCGACAATCACTGA |
| 271 | 17 | GGAGTCGCTGCGCGCTGCCTTCGCCCCGTGCCCCGCTCCGCCGCCGCCTCG |
| | | CGCCGCCCGCCCCGGCTCTGACTGACCGCGTTACTCCCACAGGTGAGCGGG |
| | | CGGGACGGCCCTTCTCCTCCGGGCTGTAATTAGCTGAGCAAGAGGTAAGGGT |
| | | TTAAGGGATGGTTGGTTGGTGGGGTATGGGGCCCTGGCTGGGATTTCATCAT |
| | | ATACTGTAAGTTTGCGATGAGACACTACAGTATAGATGATGTACTAGTCCGGG |
| | | CACCCCCAGCTCTGGAGCCTGACAAGGAGGACAGGAGAGATGCTGCAAGCCC |
| | | AAGAAGCTCTCTGCTCAGCCTGTCACAACCTACTGACTGCCAGGGCACTTGGG |
| | | AATGGCAAGGTCCAGTTCCGATTTGTAGGCCTCTACAAATCGGAACTGGCTCT |
| | | TGCTATACCCAGAAAACGTGCCAGGAAGAGAACTCAGGACCCTGAAGCAGAC |
| | | TACTGGAAGGGAGACTCCAGCTCAAACAAGGCAGGGGTGGGGGCGTGGGAT |
| | | TGGGGGTAGGGGAGGGAATAGATACATTTTCTCTTTCCTGTTGTAAAGATAAT |
| | | GTTTAATTACCTGGAGCACCTGCCTGAAATCACTTTTTTTCAGGTTGGGCTAGC |
| | | GCCACCATGAAAGTCCTCTGGGCCGCTCTCCTGGTGACCTTTCTGGCCGGCT |
| | | GTCAGGCCAAAGTGGAACAGGCCGTGGAAACCGAGCCTGAGCCTGAACTGAG |
| | | ACAGCAGACAGAGTGGCAGTCCGGCCAGAGATGGGAACTTGCCCTGGGCAG |
| | | ATTCTGGGACTACCTGAGATGGGTGCAGACCCTGAGCGAACAGGTGCAAGAG |
| | | GAACTGCTGAGCAGCCAAGTGACCCAAGAGCTGAGAGCCCTGATGGACGAGA |
| | | CAATGAAAGAACTCAAAGCTTATAAGAGCGAGCTCGAAGAGCAGCTGACCCCT |
| | | GTGGCCGAGGAAACAAGAGCCAGACTGAGCAAAGAGCTGCAGGCCGCTCAA |
| | | GCTAGACTGGGCGCCGATATGGAAGATGTGTGCGGCAGACTGGTGCAGTACA |
| | | GAGGCGAAGTGCAGGCCATGCTGGGCCAGTCTACCGAGGAACTGAGAGTGC |
| | | GGCTGGCCAGCCACCTGAGAAAGCTGCGGAAGAGACTGCTGAGAGATGCCG |
| | | ACGACCTGCAGAAATGCCTGGCCGTGTATCAGGCTGGCGCTAGAGAAGGCGC |
| | | TGAGAGAGGACTGTCTGCCATCCGGGAAAGACTGGGCCCTCTGGTTGAACAG |
| | | GGCAGAGTTAGAGCCGCCACAGTGGGATCTCTTGCAGGCCAGCCTCTGCAAG |
| | | AAAGAGCCCAGGCTTGGGGCGAGAGACTGAGGGCCAGAATGGAAGAGATGG |
| | | GCAGCAGAACCAGAGACAGACTGGACGAAGTGAAAGAACAGGAGGCCGAAGT |
| | | GCGCGCCAAGCTCGAAGAACAAGCCCAGCAGATTCGGCTGCAGGCTGAAGCC |
| | | TTTCAGGCCCGGCTGAAGAGTTGGTTCGAGCCCCTGGTGGAAGATATGCAGA |
| | | GACAGTGGGCTGGACTGGTGGAAAAGGTGCAGGCTGCCGTGGGAACATCTG |
| | | CTGCTCCAGTGCCTAGCGACAACCACTGA |
| 272 | 18 | GGAGTCGCTGCGCGCTGCCTTCGCCCCGTGCCCCGCTCCGCCGCCGCCTCG |
| | | CGCCGCCCGCCCCGGCTCTGACTGACCGCGTTACTCCCACAGGTGAGCGGG |
| | | CGGGACGGCCCTTCTCCTCCGGGCTGTAATTAGCTGAGCAAGAGGTAAGGGT |
| | | TTAAGGGATGGTTGGTTGGTGGGGTATGGGGCCCTGGCTGGGATTTCATCAT |
| | | ATACTGTAAGTTTGCGATGAGACACTACAGTATAGATGATGTACTAGTCCGGG |
| | | CACCCCCAGCTCTGGAGCCTGACAAGGAGGACAGGAGAGATGCTGCAAGCCC |
| | | AAGAAGCTCTCTGCTCAGCCTGTCACAACCTACTGACTGCCAGGGCACTTGGG |
| | | AATGGCAAGGTCCAGTTCCGATTTGTAGGCCTCTACAAATCGGAACTGGCTCT |
| | | TGCTATACCCAGAAAACGTGCCAGGAAGAGAACTCAGGACCCTGAAGCAGAC |
| | | TACTGGAAGGGAGACTCCAGCTCAAACAAGGCAGGGGTGGGGGCGTGGGAT |
| | | TGGGGGTAGGGGAGGGAATAGATACATTTTCTCTTTCCTGTTGTAAAGATAAT |
| | | GTTTAATTACCTGGAGCACCTGCCTGAAATCACTTTTTTTCAGGTTGGGCTAGC |
| | | GCCACCATGAAAGTCCTCTGGGCCGCTCTCCTGGTGACCTTTCTGGCAGGAT |
| | | GCCAGGCCAAGGTGGAGCAAGCGGTGGAGACAGAGCCGGAGCCCGAGCTGC |
| | | GCCAGCAGACCGAGTGGCAGAGCGGCCAGCGCTGGGAACTGGCACTGGGTC |
| | | GCTTTTGGGATTACCTGCGCTGGGTGCAGACACTGTCTGAGCAGGTGCAGGA |
| | | GGAGCTGCTCAGCTCCCAGGTCACCCAGGAACTGAGGGCGCTGATGGACGA |
| | | GACCATGAAAGAACTCAAAGCTTATAAGAGCGAGCTCGAAGAGCAGCTGACCC |
| | | CGGTGGCGGAGGAGACGCGGGCACGGCTGTCCAAGGAGCTGCAGGCGGCG |
| | | CAGGCCCGGCTGGGCGCGGACATGGAGGACGTGTGCGGCCGCCTGGTGCA |
| | | GTACCGCGGCGAGGTGCAGGCCATGCTCGGCCAGAGCACCGAGGAGCTGCG |
| | | GGTGCGCCTCGCCTCCCACCTGCGCAAGCTGCGTAAGCGGCTCCTCCGCGAT |
| | | GCCGATGACCTGCAGAAGTGCCTGGCAGTGTACCAGGCCGGGGCCCGCGAG |
| | | GGCGCCGAGCGCGGCCTCAGCGCCATCCGCGAGCGCCTGGGGCCCCTGGT |
| | | GGAACAGGGCCGCGTGCGGGCCGCCACTGTGGGCTCCCTGGCCGGCCAGC |
| | | CGCTACAGGAGCGGGCCCAGGCCTGGGGCGAGCGGCTGCGCGCGCGGATG |
| | | GAGGAGATGGGCAGCCGGACCCGCGACCGCCTGGACGAGGTGAAGGAGCAG |
| | | GAGGCGGAGGTGCGCGCCAAGCTGGAGGAGCAGGCCCAGCAGATACGCCTG |
| | | CAGGCCGAGGCCTTCCAGGCCCGCCTCAAGAGCTGGTTCGAGCCCCTGGTG |
| | | GAAGACATGCAGCGCCAGTGGGCCGGGCTGGTGGAGAAGGTGCAGGCTGCC |
| | | GTGGGCACCAGCGCCGCCCCTGTGCCCAGCGACAATCACTGA |
| 273 | 19 | GGAGTCGCTGCGCGCTGCCTTCGCCCCGTGCCCCGCTCCGCCGCCGCCTCG |
| | | CGCCGCCCGCCCCGGCTCTGACTGACCGCGTTACTCCCACAGGTGAGCGGG |
| | | CGGGACGGCCCTTCTCCTCCGGGCTGTAATTAGCTGAGCAAGAGGTAAGGGT |
| | | TTAAGGGATGGTTGGTTGGTGGGGTATGGGGCCCTGGCTGGGATTTCATCAT |
| | | ATACTGTAAGTTTGCGATGAGACACTACAGTATAGATGATGTACTAGTCCGGG |
| | | CACCCCCAGCTCTGGAGCCTGACAAGGAGGACAGGAGAGATGCTGCAAGCCC |
| | | AAGAAGCTCTCTGCTCAGCCTGTCACAACCTACTGACTGCCAGGGCACTTGGG |
| | | AATGGCAAGGTCCAGTTCCGATTTGTAGGCCTCTACAAATCGGAACTGGCTCT |
| | | TGCTATACCCAGAAAACGTGCCAGGAAGAGAACTCAGGACCCTGAAGCAGAC |
| | | TACTGGAAGGGAGACTCCAGCTCAAACAAGGCAGGGGTGGGGGCGTGGGAT |
| | | TGGGGGTAGGGGAGGGAATAGATACATTTTCTCTTTCCTGTTGTAAAGATAAT |
| | | GTTTAATTACCTGGAGCACCTGCCTGAAATCACTTTTTTTCAGGTTGGGCTAGC |
| | | GCCACCATGAAAGTCCTCTGGGCCGCTCTCCTGGTGACCTTTCTGGCCGGCT |
| | | GTCAGGCCAAAGTGGAACAGGCCGTGGAAACCGAGCCTGAGCCTGAACTGAG |
| | | ACAGCAGACAGAGTGGCAGTCCGGCCAGAGATGGGAACTTGCCCTGGGCAG |
| | | ATTCTGGGACTACCTGAGATGGGTGCAGACCCTGAGCGAACAGGTGCAAGAG |
| | | GAACTGCTGAGCAGCCAAGTGACCCAAGAGCTGAGAGCCCTGATGGACGAGA |
| | | CAATGAAAGAACTCAAAGCTTATAAGAGCGAGCTCGAAGAGCAGCTGACCCCT |
| | | GTGGCCGAGGAAACAAGAGCCAGACTGAGCAAAGAGCTGCAGGCCGCTCAA |
| | | GCTAGACTGGGCGCCGATATGGAAGATGTGTGCGGCAGACTGGTGCAGTACA |
| | | GAGGCGAAGTGCAGGCCATGCTGGGCCAGTCTACCGAGGAACTGAGAGTGC |
| | | GGCTGGCCAGCCACCTGAGAAAGCTGCGGAAGAGACTGCTGAGAGATGCCG |
| | | ACGACCTGCAGAAACGCCTGGCCGTGTATCAGGCTGGCGCTAGAGAAGGCGC |
| | | TGAGAGAGGACTGTCTGCCATCCGGGAAAGACTGGGCCCTCTGGTTGAACAG |
| | | GGCAGAGTTAGAGCCGCCACAGTGGGATCTCTTGCAGGCCAGCCTCTGCAAG |
| | | AAAGAGCCCAGGCTTGGGGCGAGAGACTGAGGGCCAGAATGGAAGAGATGG |
| | | GCAGCAGAACCAGAGACAGACTGGACGAAGTGAAAGAACAGGTGGCCGAAGT |
| | | GCGCGCCAAGCTCGAAGAACAAGCCCAGCAGATTCGGCTGCAGGCTGAAGCC |
| | | TTTCAGGCCCGGCTGAAGAGTTGGTTCGAGCCCCTGGTGGAAGATATGCAGA |
| | | GACAGTGGGCTGGACTGGTGGAAAAGGTGCAGGCTGCCGTGGGAACATCTG |
| | | CTGCTCCAGTGCCTAGCGACAACCACTGA |
| 274 | 20 | GGAGTCGCTGCGCGCTGCCTTCGCCCCGTGCCCCGCTCCGCCGCCGCCTCG |
| | | CGCCGCCCGCCCCGGCTCTGACTGACCGCGTTACTCCCACAGGTGAGCGGG |
| | | CGGGACGGCCCTTCTCCTCCGGGCTGTAATTAGCTGAGCAAGAGGTAAGGGT |
| | | TTAAGGGATGGTTGGTTGGTGGGGTATGGGGCCCTGGCTGGGATTTCATCAT |
| | | ATACTGTAAGTTTGCGATGAGACACTACAGTATAGATGATGTACTAGTCCGGG |
| | | CACCCCCAGCTCTGGAGCCTGACAAGGAGGACAGGAGAGATGCTGCAAGCCC |
| | | AAGAAGCTCTCTGCTCAGCCTGTCACAACCTACTGACTGCCAGGGCACTTGGG |
| | | AATGGCAAGGTCCAGTTCCGATTTGTAGGCCTCTACAAATCGGAACTGGCTCT |
| | | TGCTATACCCAGAAAACGTGCCAGGAAGAGAACTCAGGACCCTGAAGCAGAC |
| | | TACTGGAAGGGAGACTCCAGCTCAAACAAGGCAGGGGTGGGGGCGTGGGAT |
| | | TGGGGGTAGGGGAGGGAATAGATACATTTTCTCTTTCCTGTTGTAAAGATAAT |
| | | GTTTAATTACCTGGAGCACCTGCCTGAAATCACTTTTTTTCAGGTTGGGCTAGC |
| | | GCCACCATGAAAGTCCTCTGGGCCGCTCTCCTGGTGACCTTTCTGGCCGGCT |
| | | GTCAGGCCAAAGTGGAACAGGCCGTGGAAACCGAGCCTGAGCCTGAACTGAG |
| | | ACAGCAGACAGAGTGGCAGTCCGGCCAGAGATGGGAACTTGCCCTGGGCAG |
| | | ATTCTGGGACTACCTGAGATGGGTGCAGACCCTGAGCGAACAGGTGCAAGAG |
| | | GAACTGCTGAGCAGCCAAGTGACCCAAGAGCTGAGAGCCCTGATGGACGAGA |
| | | CAATGAAAGAACTCAAAGCTTATAAGAGCGAGCTCGAAGAGCAGCTGACCCCT |
| | | GTGGCCGAGGAAACAAGAGCCAGACTGAGCAAAGAGCTGCAGGCCGCTCAA |
| | | GCTAGACTGGGCGCCGATATGGAAGATGTGTGCGGCAGACTGGTGCAGTACA |
| | | GAGGCGAAGTGCAGGCCATGCTGGGCCAGTCTACCGAGGAACTGAGAGTGTC |
| | | TCTGGCCAGCCACCTGAGAAAGCTGCGGAAGAGACTGCTGAGAGATGCCGAC |
| | | GACCTGCAGAAACGCCTGGCCGTGTATCAGGCTGGCGCTAGAGAAGGCGCTG |
| | | AGAGAGGACTGTCTGCCATCCGGGAAAGACTGGGCCCTCTGGTTGAACAGGG |
| | | CAGAGTTAGAGCCGCCACAGTGGGATCTCTTGCAGGCCAGCCTCTGCAAGAA |
| | | AGAGCCCAGGCTTGGGGCGAGAGACTGAGGGCCAGAATGGAAGAGATGGGC |
| | | AGCAGAACCAGAGACAGACTGGACGAAGTGAAAGAACAGGTGGCCGAAGTGC |
| | | GCGCCAAGCTCGAAGAACAAGCCCAGCAGATTCGGCTGCAGGCTGAAGCCTT |
| | | TCAGGCCCGGCTGAAGAGTTGGTTCGAGCCCCTGGTGGAAGATATGCAGAGA |
| | | CAGTGGGCTGGACTGGTGGAAAAGGTGCAGGCTGCCGTGGGAACATCTGCTG |
| | | CTCCAGTGCCTAGCGACAACCACTGA |
| 275 | 21 | GGAGTCGCTGCGCGCTGCCTTCGCCCCGTGCCCCGCTCCGCCGCCGCCTCG |
| | | CGCCGCCCGCCCCGGCTCTGACTGACCGCGTTACTCCCACAGGTGAGCGGG |
| | | CGGGACGGCCCTTCTCCTCCGGGCTGTAATTAGCTGAGCAAGAGGTAAGGGT |
| | | TTAAGGGATGGTTGGTTGGTGGGGTATGGGGCCCTGGCTGGGATTTCATCAT |
| | | ATACTGTAAGTTTGCGATGAGACACTACAGTATAGATGATGTACTAGTCCGGG |
| | | CACCCCCAGCTCTGGAGCCTGACAAGGAGGACAGGAGAGATGCTGCAAGCCC |
| | | AAGAAGCTCTCTGCTCAGCCTGTCACAACCTACTGACTGCCAGGGCACTTGGG |
| | | AATGGCAAGGTCCAGTTCCGATTTGTAGGCCTCTACAAATCGGAACTGGCTCT |
| | | TGCTATACCCAGAAAACGTGCCAGGAAGAGAACTCAGGACCCTGAAGCAGAC |
| | | TACTGGAAGGGAGACTCCAGCTCAAACAAGGCAGGGGTGGGGGCGTGGGAT |
| | | TGGGGGTAGGGGAGGGAATAGATACATTTTCTCTTTCCTGTTGTAAAGATAAT |
| | | GTTTAATTACCTGGAGCACCTGCCTGAAATCACTTTTTTTCAGGTTGGGCTAGC |
| | | GCCACCATGAAAGTCCTCTGGGCCGCTCTCCTGGTGACCTTTCTGGCCGGCT |
| | | GTCAGGCCAAAGTGGAACAGGCCGTGGAAACCGAGCCTGAGCCTGAACTGAG |
| | | ACAGCAGACAGAGTGGCAGTCCGGCCAGAGATGGGAACTTGCCCTGGGCAG |
| | | ATTCTGGGACTACCTGAGATGGGTGCAGACCCTGAGCGAACAGGTGCAAGAG |
| | | GAACTGCTGAGCAGCCAAGTGACCCAAGAGCTGAGAGCCCTGATGGACGAGA |
| | | CAATGAAAGAACTCAAAGCTTATAAGAGCGAGCTCGAAGAGCAGCTGACCCCT |
| | | GTGGCCGAGGAAACAAGAGCCAGACTGAGCAAAGAGCTGCAGGCCGCTCAA |
| | | GCTAGACTGGGCGCCGATATGGAAGATGTGTGCGGCAGACTGGTGCAGTACA |
| | | GAGGCGAAGTGCAGGCCATGCTGGGCCAGTCTACCGAGGAACTGAGAGTGC |
| | | GGCTGGCCAGCCACCTGAGAAAGCTGCGGAAGAGACTGCTGAGAGATGCCG |
| | | ACGACCTGCAGAAACGCCTGGCCGTGTATCAGGCTGGCGCTAGAGAAGGCGC |
| | | TGAGAGAGGACTGTCTGCCATCCGGGAAAGACTGGGCCCTCTGGTTGAACAG |
| | | GGCAGAGTTAGAGCCGCCACAGTGGGATCTCTTGCAGGCCAGCCTCTGCAAG |
| | | AAAGAGCCCAGGCTTGGGGCGAGAGACTGAGGGCCAGAATGGAAGAGATGG |
| | | GCAGCAGAACCAGAGACAGACTGGACGAAGTGAAAGAACAGGAGGCCGAAGT |
| | | GCGCGCCAAGCTCGAAGAACAAGCCCAGCAGATTCGGCTGCAGGCTGAAGCC |
| | | TTTCAGGCCCGGCTGAAGAGTTGGTTCGAGCCCCTGGTGGAAGATATGCAGA |
| | | GACAGTGGGCTGGACTGGTGGAAAAGGTGCAGGCTGCCGTGGGAACATCTG |
| | | CTGCTCCAGTGCCTAGCGACAACCACTGA |
| 276 | 22 | GGAGTCGCTGCGCGCTGCCTTCGCCCCGTGCCCCGCTCCGCCGCCGCCTCG |
| | | CGCCGCCCGCCCCGGCTCTGACTGACCGCGTTACTCCCACAGGTGAGCGGG |
| | | CGGGACGGCCCTTCTCCTCCGGGCTGTAATTAGCTGAGCAAGAGGTAAGGGT |
| | | TTAAGGGATGGTTGGTTGGTGGGGTATGGGGCCCTGGCTGGGATTTCATCAT |
| | | ATACTGTAAGTTTGCGATGAGACACTACAGTATAGATGATGTACTAGTCCGGG |
| | | CACCCCCAGCTCTGGAGCCTGACAAGGAGGACAGGAGAGATGCTGCAAGCCC |
| | | AAGAAGCTCTCTGCTCAGCCTGTCACAACCTACTGACTGCCAGGGCACTTGGG |
| | | AATGGCAAGGATCGCGTTCTCGCTGTTCGACTGAACAGCGAGAACGCGACTC |
| | | TTGCTATACCCAGAAAACGTGCCAGGAAGAGAACTCAGGACCCTGAAGCAGA |
| | | CTACTGGAAGGGAGACTCCAGCTCAAACAAGGCAGGGGTGGGGGCGTGGGA |
| | | TTGGGGGTAGGGGAGGGAATAGATACATTTTCTCTTTCCTGTTGTAAAGATAAT |
| | | GTTTAATTACCTGGAGCACCTGCCTGAAATCACTTTTTTTCAGGTTGGGCTAGC |
| | | GCCACCATGAAGGTTCTGTGGGCTGCGTTGCTGGTCACATTCCTGGCAGGAT |
| | | GCCAGGCCAAGGTGGAGCAAGCGGTGGAGACAGAGCCGGAGCCCGAGCTGC |
| | | GCCAGCAGACCGAGTGGCAGAGCGGCCAGCGCTGGGAACTGGCACTGGGTC |
| | | GCTTTTGGGATTACCTGCGCTGGGTGCAGACACTGTCTGAGCAGGTGCAGGA |
| | | GGAGCTGCTCAGCTCCCAGGTCACCCAGGAACTGAGGGCGCTGATGGACGA |
| | | GACCATGAAGGAGTTGAAGGCCTACAAATCGGAACTGGAGGAACAACTGACC |
| | | CCGGTGGCGGAGGAGACGCGGGCACGGCTGTCCAAGGAGCTGCAGGCGGC |
| | | GCAGGCCCGGCTGGGCGCGGACATGGAGGACGTGTGCGGCCGCCTGGTGC |
| | | AGTACCGCGGCGAGGTGCAGGCCATGCTCGGCCAGAGCACCGAGGAGCTGC |
| | | GGGTGAGCCTCGCCTCCCACCTGCGCAAGCTGCGTAAGCGGCTCCTCCGCG |
| | | ATGCCGATGACCTGCAGAAGCGCCTGGCAGTGTACCAGGCCGGGGCCCGCG |
| | | AGGGCGCCGAGCGCGGCCTCAGCGCCATCCGCGAGCGCCTGGGGCCCCTG |
| | | GTGGAACAGGGCCGCGTGCGGGCCGCCACTGTGGGCTCCCTGGCCGGCCAG |
| | | CCGCTACAGGAGCGGGCCCAGGCCTGGGGCGAGCGGCTGCGCGCGCGGAT |
| | | GGAGGAGATGGGCAGCCGGACCCGCGACCGCCTGGACGAGGTGAAGGAGCA |
| | | GGTGGCGGAGGTGCGCGCCAAGCTGGAGGAGCAGGCCCAGCAGATACGCCT |
| | | GCAGGCCGAGGCCTTCCAGGCCCGCCTCAAGAGCTGGTTCGAGCCCCTGGT |
| | | GGAAGACATGCAGCGCCAGTGGGCCGGGCTGGTGGAGAAGGTGCAGGCTGC |
| | | CGTGGGCACCAGCGCCGCCCCTGTGCCCAGCGACAATCACTGA |

### Example 10: In Vitro Testing of the Combined Constructs-APOE Expression

In addition, APOE protein expression was quantified by ELISA in the supernatant of cells transfected with the combined constructs. Protein quantification was only assessed on the supernatant of cells that were transfected with the highest amount of DNA construct (e.g. 86.54 fmol). APOE protein expression was confirmed for all transfected cells (FIG. 15).

### Example 11: In Vivo Testing of the Combined Constructs Delivered by AAV in P301S/hAPOE4-TR Mice

The efficacy of AAV5 vectors encoding the combined expression constructs as well as the single constructs to modify the phenotype of a tauopathy model P301S mice that also harbor the hAPOE4 gene (P301S/hAPOE4-TR mice) will be assessed by a single bilateral intrastriatal injection of AAV vectors. The effect of the AAV vectors will be evaluated by behavioral tests and the molecular analysis of different brain areas, CSF, plasma and liver.

We anticipate that in the groups administered with AAV vectors, vDNA levels and APOE variant mRNA/miRNA expression will be detected within the brains. Indeed, heightened levels of miRNA are expected in the brains of the groups injected with constructs containing a miAPOE, whereas those administered with constructs containing and APOE variant transgene are predicted to demonstrate elevated APOE variant mRNA.

Furthermore, a decrease or modified levels of hAPOE4, tau and phosphorylated tau proteins is expected to be observed in the brains of AAV vector-injected groups in contrast to the vehicle group.

### Example 12: In Vivo Testing of the Combined Approach Constructs Delivered by AAV in WT Mice

The expression levels of transgenes of the combined approach constructs and single constructs mediated by AAV5-delivery (4.3E10 gc/mouse) were assessed by a single bilateral intrastriatal injection in WT mice. Male mice were treated with AAV5 combined approach constructs with IDs 2, 3, 16 and 18 (SEQ ID NOS. 256, 257, 270, and 272, resp.) and single constructs, including AAV5-APOE2ch V2 expressing variant (SEQ ID NO. 208), AAV5-miSCR (SEQ ID NO. 186), AAV5-miAPOE_016 (SEQ ID No. 108) and AAV5-miAPOE_037 (SEQ ID NO. 116). Empty AAV5 was included as control. Animals were sacrificed one month post-treatment. Multiple brain regions and the liver were collected and snap frozen for 5-6 animals per group.

Vector DNA levels were quantified by qPCR to analyze AAV transduction. Besides the empty AAV5 treated group, all groups showed comparable and significant increase of vector DNA levels in the striatum reaching up to 3.74E+07 gc/μg DNA (FIG. 23A). miRNA levels were quantified by a SYBR green RT-qPCR assay using specific primers for the 23, 24 or 25 nucleotide long mature miSCR and miAPOEs (FIG. 23B). The selected primers and standard lines were optimized based on the previous small RNA sequencing results (FIG. 20A). miRNA levels in the striatum of groups injected with combined approach vectors reached up to 1.48E+09 copies/μg total RNA (FIG. 23B). The miRNA levels of the AAV5-miSCR group was similar to the combined approach constructs groups. miAPOE copy levels in the AAV5-miAPOE_016 group reached up to 7.06E+09 copies/μg total RNA, while the miRNA levels for the AAV5-miAPOE_037 and empty AAV5 groups did not result in levels above the LLOQ. Tissues of the AAV5-APOE2ch V2 group were not subjected to this assay since no miRNA is expected.

hAPOE mRNA expression in the striatum of all groups was assessed via RT-qPCR (FIG. 23C). Elevated expression of hAPOE mRNA of up to 1.22E+10 copies/μg RNA was detected in the groups expressing the combined approach constructs, and 7.08E+08 and copies/μg RNA was detected in the AAV5-APOE2ch V2 group. No mRNA detection was expected in the WT, the AAV5-miSCR, AAV5-miAPOE_016, and AAV5-miAPOE_037 groups, although the assay did detect mRNA copies slightly above the LLOQ.

To examine whether the elevated mRNA copy levels in the groups injected with the combined approach vectors and AAV5-APOE2ch V2 are in line with increased APOE protein expression, hAPOE-MSD was performed on rostral cortical tissues (FIG. 23D). No hAPOE protein levels were detected in the empty AAV5, AAV5-miSCR, AAV5-miAPOE_016 and AAV5-miAPOE_037 groups. Up to 478.83 ng/mg hAPOE protein levels were detected in the groups injected with the combined approach vectors, while the hAPOE protein levels in the AAV5-APOE2ch V2 group reached 129.48 ng/mg protein levels.

Taken together, these in vivo results demonstrate high levels of vDNA between all groups, and demonstrate similar or superior transcript and protein levels for the combined approach constructs compared to the constructs expressing a single transcript.

### Example 13: In Vitro Transduction of the AAV5-Combined Approach Constructs and AAV5-miAPOEs to Examine miAPOE Processing and Off-Target Effects

In order to examine the processing and abundancy of miAPOEs expressed by combined approach constructs and single miAPOE constructs in human derived glioblastoma cells, U-118 MG cells were transduced with AAV5-combined approach constructs IDs 2 and 16 (SEQ ID NOs. 256 and 270), AAV5-miSCR (SEQ ID NO. 186), AAV5-miAPOE_016 (SEQ ID NO. 108), AAV5-miAPOE_037 (SEQ ID NO. 116) and AAV5-miAPOE_145 (SEQ ID NO. 146). miAPOE_016 is encoded within combined approach construct ID 2, and miAPOE_145 is encoded within combined approach construct ID 16. Three days post-transduction, RNA was isolated from these cell cultures and analyzed by small RNA sequencing and polyA-enriched mRNA sequencing.

Data analysis revealed for the combined approach constructs, miAPOE_016 and miAPOE_145 expected miAPOE transcripts with read counts ranging from 2.68-0.05% when compared to total miRNA reads (FIG. 24B). Read counts for miAPOE_037 were absent.

The most abundant isomIR produced by combined approach constructs, miAPOE_016 and miAPOE_145 are all 24 nucleotides long (FIG. 24A). The total miAPOE reads of all constructs, except miAPOE_037, were compared to expression levels of endogenous miRNAs (FIG. 24C). Considering that miAPOEs are not among the most abundant endogenously expressed miRNAs, the risk for oversaturation of the RNAi pathway by the miAPOEs is very low. The two internal controls, miR-29a-3p and miR-16-5p demonstrated consistent abundancy ranking 6.33+1.55 (SD) and 19.75+6.13 (SD) respectively amongst all samples (marked in FIG. 24C).

Differential expression analysis was performed to reveal potential off-target effects of the combined approach, miAPOE_016 and miAPOE_145 constructs. miSCR was used as control and analysis was performed to determine to what extent the other constructs differentiate in transcript levels. This revealed very few differentially expressed transcripts for all samples with FDR p-values >0.05 (FIG. 24D). As expected, the combined approach constructs had significantly increased APOE transcripts. While combined approach construct ID 16 had no other differentially expressed transcripts, ID 2 had two genes of which the transcript levels deviated from control. miAPOE_016 and miAPOE_145 both had one and the same differentially expressed gene.

Taken together, these in vitro results revealed normal processing of the miAPOEs expressed by the combined approach and single constructs. Minimal to no off-target effects were observed within the transcriptomes of the samples expressing these miAPOEs.

### Example 14: In Silico Assessment of Potential Off-Target Transcripts of miAPOE miRNA Guide

In silico analysis of possible off-target gene transcripts was performed for miAPOE miRNA guide sequence

(BLASTN) and guide seed sequence (siSPOTR). BLASTN for short sequences was performed against the human reference transcriptome (via ENSEMBL, to identify transcripts with partial complementarity of at least 7 consecutive nucleotides within the 22 nucleotide miAPOE guide sequence (SEQ ID NOs. 108, 116, and 146). No gene transcripts fully complementary to the 22 nucleotide miAPOE miRNA guide sequences were found other than APOE. Gene transcripts with partial complementarity were ranked by alignment score which is calculated from the sum of the rewards for matched nucleotides and penalties for mismatches and gaps (match/mismatch: 1/-3). The top 15 hits are shown below (table 9).

The siSPOTR tool (Boudreau et al., 2013) was used to predict the binding of the miAPOE miRNA guide seed sequence (nucleotides 2-8 of 5′-3′ guide sequence) to potential off-target gene transcripts, which were predominantly found in the 3′ UTR of target transcripts. The potential off-targeting score (POTS) is calculated using seed site type frequency (8mer, 7mer-M8, 7mer-1A and 6 mer) (table 10). A list of probable off-target genes were rank-ordered by individual transcript Probability of Off-Target Score (tPOTS). tPOTS was calculated based on the number and type of seed matches found in individual transcripts. The top 15 hits of potential off-target genes are listed in Table 11.

None of the top 15 hits for potential off-targets for miAPOE guides seed sequences were overlapping with the top 15 hits of the in silico off-target prediction of miAPOE guide sequence by BLASTN for short sequences. The in silico predicted transcripts were found not significantly differentially expressed in the RNA-seq data from AAV5-combined approach constructs IDs 2 and 16 (SEQ ID NOs. 256 and 270), AAV5-miAPOE_016 (SEQ ID NO. 108) and AAV5-miAPOE_145 (SEQ ID NO. 146) (FIG. 24D).

TABLE 9
Top 15 hits in-silico off-target prediction of miAPOE guide sequence by BLASTN for short sequences.
Alignment length: Number of nucleotides of individual gene transcripts complementary with the
22 nucleotide miAPOE guide sequence with 0, 1 or 2 mismatches or gaps; Score: alignment score
calculated from the sum of the rewards for matched nucleotides and penalties for mismatches or gaps;
% ID: Percentage identity complementary with the miAPOE guide sequence relative to the aligned
sequence length.

| | | Alignment | | |
|---|---|---|---|---|
| | Gene hit | Length | Score | % ID |
| | miAPOE_016-SEQ ID NO. 108 | | | |
| | APOE | 22 | 44 | 100 |
| 1 | POLR3A | 16 | 32.1 | 100 |
| 2 | TTN | 15 | 30.2 | 100 |
| 3 | CCDC82 | 15 | 30.2 | 100 |
| 4 | ACBD6 | 15 | 30.2 | 100 |
| 5 | CCNDBP1 | 15 | 30.2 | 100 |
| 6 | PCDH10 | 15 | 30.2 | 100 |
| 7 | CALCOCO1 | 15 | 30.2 | 100 |
| 8 | ENSG00000267140 | 15 | 30.2 | 100 |
| 9 | YWHABP2 | 14 | 28.2 | 100 |
| 10 | SERPINH1P1 | 14 | 28.2 | 100 |
| 11 | HMGB1P45 | 14 | 28.2 | 100 |
| 12 | RCC2P1 | 14 | 28.2 | 100 |
| 13 | ABCA17P | 14 | 28.2 | 100 |
| 14 | GPR161 | 22 | 28.2 | 90.91 |
| 15 | DR1 | 19 | 28.2 | 94.74 |
| | miAPOE_037-SEQ ID NO. 116 | | | |
| | APOE | 22 | 44 | 100 |
| 1 | STIM2 | 16 | 32.1 | 100 |
| 2 | TCP11L1 | 19 | 30.2 | 94.74 |
| 3 | MEGF8 | 14 | 28.2 | 100 |
| 4 | PHTF2 | 14 | 28.2 | 100 |
| 5 | FDFT1 | 14 | 28.2 | 100 |
| 6 | DSCAML1 | 14 | 28.2 | 100 |
| 7 | SESN2 | 14 | 28.2 | 100 |
| 8 | GPT2 | 14 | 28.2 | 100 |
| 9 | SHMT2 | 14 | 28.2 | 100 |
| 10 | TRIM80P | 13 | 26.2 | 100 |
| 11 | PAICSP1 | 13 | 26.2 | 100 |
| 12 | ENSG00000232528 | 13 | 26.2 | 100 |
| 13 | FN1 | 13 | 26.2 | 100 |
| 14 | SYT13 | 13 | 26.2 | 100 |
| 15 | ZSCAN31 | 13 | 26.2 | 100 |
| | miAPOE_145-SEQ ID NO. 146 | | | |
| | APOE | 22 | 44 | 100 |
| 1 | TICRR | 14 | 28.2 | 100 |
| 2 | ENSG00000235707 | 13 | 26.2 | 100 |
| 3 | GDPD1 | 13 | 26.2 | 100 |
| 4 | SH3TC1 | 21 | 26.2 | 90.48 |
| 5 | RASIP1 | 13 | 26.2 | 100 |
| 6 | STEAP4 | 13 | 26.2 | 100 |
| 7 | SVEP1 | 13 | 26.2 | 100 |
| 8 | ATP7B | 13 | 26.2 | 100 |
| 9 | GDPD1 | 12 | 24.2 | 100 |
| 10 | ENSG00000197550 | 12 | 24.2 | 100 |
| 11 | ZNF355P | 12 | 24.2 | 100 |
| 12 | SLC25A5P8 | 12 | 24.2 | 100 |
| 13 | SUPT4H1P2 | 12 | 24.2 | 100 |
| 14 | MIB2 | 12 | 24.2 | 100 |
| 15 | ACADM | 12 | 24.2 | 100 |

TABLE 10
In-silico off-target prediction of miAPOE guides seed sequence by SiSPOTR: Potential
off -targets by type of seed matches. Seed match types: 8mer site (perfect match
to nucleotides 2-8 followed by an A), 7mer-m8 site (perfect match to nucleotides
2-8), 7mer-1A (perfect match to nucleotides 2-7 followed by an A), 6 mer site (perfect
match to nucleotides 2-7); potential off-targeting score (POTS) is calculated using
seed site type frequency (8mer, 7mer-M8, 7mer-1A and 6 mer).

| miAPOE/SEQ ID | Total number predicted | Target genes with seed match in 3′ UTR with | | | | |
|---|---|---|---|---|---|---|
| NO. | target genes | 8mer | 7mer-M8 | 7mer-1A | 6mer | POTS |
| miAPOE_016/108 | 8277 | 580 | 1916 | 2723 | 6414 | 649.5 |
| miAPOE_037/116 | 4936 | 304 | 1067 | 1112 | 3605 | 297.3 |
| miAPOE_145/146 | 4750 | 492 | 1057 | 1492 | 2802 | 314.6 |

TABLE 11
Top 15 hits in-silico off-target prediction of miAPOE guide seed sequence by siSPOTR
tPOTS: Probability of Off-Target Score; Number of seed match types: 8mer site (perfect
match to nucleotides 2-8 followed by an A), 7mer-m8 site (perfect match to nucleotides
2-8), 7mer-1A (perfect match to nucleotides 2-7 followed by an A), 6 mer site (perfect
match to nucleotides 2-7); potential off-targeting score (POTS) is calculated using
seed site type frequency (8mer, 7mer-M8, 7mer-1A and 6 mer).

| miAPOE/SEQ ID NO. | Gene Symbol | TPOTS | 8mer | 7mer-M8 | 7mer-1A | 6mer |
|---|---|---|---|---|---|---|
| miAPOE_016/ 108 | AAK1 | 0.765 | 1 | 2 | 7 | 10 |
| | SLC5A3 | 0.661 | 2 | 1 | 4 | 11 |
| | ZNF704 | 0.615 | 3 | 0 | 2 | 5 |
| | FBXW2 | 0.57 | 1 | 3 | 3 | 5 |
| | IKZF2 | 0.566 | 1 | 2 | 4 | 6 |
| | TPPP | 0.561 | 0 | 8 | 0 | 1 |
| | RFX4 | 0.518 | 2 | 0 | 3 | 3 |
| | KSR2 | 0.507 | 1 | 4 | 1 | 2 |
| | ZNF621 | 0.5 | 1 | 2 | 3 | 5 |
| | DPH3 | 0.5 | 1 | 3 | 2 | 0 |
| | FOXN3 | 0.495 | 1 | 1 | 4 | 5 |
| | ZC3H6 | 0.494 | 1 | 1 | 4 | 4 |
| | MCCC2 | 0.481 | 3 | 0 | 0 | 1 |
| | STK10 | 0.456 | 2 | 1 | 1 | 1 |
| | NDFIP1 | 0.45 | 2 | 0 | 2 | 0 |
| miAPOE_037/ 116 | ONECUT2 | 0.593 | 2 | 2 | 2 | 3 |
| | UHMK1 | 0.393 | 2 | 1 | 0 | 3 |
| | RLIM | 0.368 | 1 | 2 | 1 | 3 |
| | DNM3 | 0.368 | 1 | 2 | 1 | 3 |
| | INO80D | 0.367 | 1 | 2 | 1 | 2 |
| | CREB1 | 0.367 | 1 | 2 | 1 | 2 |
| | OLFML2A | 0.365 | 1 | 2 | 1 | 0 |
| | MFAP3L | 0.362 | 1 | 1 | 2 | 2 |
| | ENAH | 0.36 | 1 | 1 | 2 | 0 |
| | QKI | 0.354 | 0 | 5 | 0 | 4 |
| | OSMR | 0.345 | 0 | 4 | 1 | 0 |
| | SH3TC2 | 0.343 | 0 | 3 | 2 | 3 |
| | CDK6 | 0.322 | 2 | 0 | 0 | 2 |
| | C6orf168 | 0.322 | 2 | 0 | 0 | 2 |
| | PTAR1 | 0.321 | 2 | 0 | 0 | 1 |
| miAPOE_145/ 146 | N6AMT1 | 0.522 | 2 | 1 | 2 | 2 |
| | ZNF704 | 0.433 | 1 | 2 | 2 | 3 |
| | AP1G1 | 0.43 | 1 | 2 | 2 | 0 |
| | SOX11 | 0.425 | 1 | 1 | 3 | 0 |
| | QKI | 0.423 | 1 | 0 | 4 | 3 |
| | SH3TC2 | 0.388 | 2 | 0 | 1 | 3 |
| | HIC2 | 0.386 | 2 | 0 | 1 | 1 |
| | C18orf1 | 0.386 | 2 | 0 | 1 | 1 |
| | TNFRSF10B | 0.385 | 2 | 0 | 1 | 0 |
| | PLXDC1 | 0.385 | 2 | 0 | 1 | 0 |
| | NSL1 | 0.368 | 1 | 2 | 1 | 3 |
| | GFRA1 | 0.367 | 1 | 2 | 1 | 2 |
| | PRKX | 0.366 | 1 | 2 | 1 | 1 |
| | C22orf46 | 0.365 | 1 | 2 | 1 | 0 |
| | SGK269 | 0.36 | 1 | 1 | 2 | 0 |

### REFERENCES

Knouff C, Hinsdale M E, et al. Apo E structure determines VLDL clearance and atherosclerosis risk in mice. J Clin Invest. 1999 June; 103 (11): 1579-86. doi: 10.1172/JCI6172. PMID: 10359567; PMCID: PMC408371.
Sullivan PM, Mezdour H, et al. Targeted replacement of the mouse apolipoprotein E gene with the common human APOE3 allele enhances diet-induced hypercholesterolemia and atherosclerosis. J Biol Chem. 1997 Jul. 18; 272 (29): 17972-80. doi: 10.1074/jbc.272.29.17972. PMID: 9218423.
Arboleda-Velasquez J F, Lopera F, et al. Resistance to autosomal dominant Alzheimer's disease in an APOE3 Christchurch homozygote: a case report. Nat Med. 2019 November; 25 (11): 1680-1683. doi: 10.1038/s41591-019-0611-3. Epub 2019 Nov. 4.PMID: 31686034.
van den Maagdenberg A M, Weng W, et al. Characterization of five new mutants in the carboxyl-terminal domain of human apolipoprotein E: no cosegregation with severe hyperlipidemia. Am J Hum Genet. 1993 May; 52 (5): 937-46.PMID: 8488843.
Liu C C, Murray M E, et al. APOE3-Jacksonville (V236E) variant reduces self-aggregation and risk of dementia. Sci Transl Med. 2021 Sep. 29; 13 (613): eabc9375. doi: 10.1126/scitranslmed.abc9375. Epub 2021 Sep. 29.PMID: 34586832.
BLAST. Joseph Bedell, Ian Korf and Mark Yandell [OReilly & Associates, 2003], Altschul S F, Gish W, Miller W, Myers E W, Lipman D J. Basic local alignment search tool. J Mol Biol. 1990; 215 (3): 403-410. doi: 10.1016/S0022-2836 (05) 80360-2
Boudreau R L, Spengler R M, Hylock R H, Kusenda B J, Davis H A, Eichmann D A, Davidson BL. siSPOTR: a tool for designing highly specific and potent siRNAs for human and mouse. Nucleic Acids Res. 2013 Jan. 7; 41 (1): e9. doi: 10.1093/nar/gks797. Epub 2012 Aug. 31. PMID: 22941647; PMCID: PMC3592398.

### DESCRIPTION OF THE FIGURES

FIG. 1. A schematic of part of the APOE4 cDNA sequence that is part of NCBI Reference Sequence: NM_000041. The sequence depicted is referred to as SEQ ID NO. 1, herein, and corresponds to nucleotides 1-1166 thereof, and represents DNA sequence of (part of) a spliced APOE transcript (transcript 2).

FIG. 2. Schematic of scaffold RNA structure indicating the first RNA sequence as it is designed (a) and (b).

FIG. 3. Validation of the miR-144 A>T mutant scaffold by small mRNA sequencing. A) Comparison of miRNA6 (=miHTT), miR-144 5p, miR-144 3p and miR-16 expression levels across miR-451, miR-144 wild type (WT) and miR-144 A>T mutant scaffolds. B) Comparison of expression values of a panel of different miRNA sequences inserted within the miR-451 and miR-144 A>T mutant scaffolds.

FIG. 4. Knockdown of APOE4 Luc reporter by miAPOE in vitro upon co-transfection with 250 ng DNA constructs (SEQ ID NOs. 94-185) in HEK293T cells.

FIG. 5. Knockdown of APOE4 Luc reporter by 25 miAPOE constructs (SEQ ID NOs. 96, 99, 100, 105, 106, 108, 109, 113, 114, 115, 116, 117, 131, 132, 133,134, 135, 136, 137, 139, 143, 146, 149, 151, 185) in vitro upon co-transfection with 2, 10 or 50 ng of DNA constructs. Relative renilla expression (RL/FL) of the miSCR441 (SEQ ID NO. 186) was set at 100%.

FIG. 6. Endogenous APOE mRNA expression and lowering in vitro upon transfection with 250 ng miAPOE constructs (SEQ ID NOs. 94-185) in Huh7 cells.

FIG. 7. Endogenous APOE mRNA expression and lowering in vitro upon transfection with 50 or 250 ng miAPOE constructs (SEQ ID NOs. 96, 108, 116, 133, 136, 138, 146, 151, 185) or miSCR constructs (SEQ ID NOs. 186-187) (n=2).

FIG. 8. (A) Western blot and (B) relative quantification thereof showing (lowering of) secreted endogenous APOE protein in the supernatant of Huh7 cells transfected with miAPOE constructs (SEQ ID NO 96, 108, 116, 136, 146, 185) or miSCR construct (SEQ ID NO.186).

FIG. 9. Vector DNA copies and knockdown of APOE mRNA expression in Huh7 cells upon transduction with AAV5 or AAV9 containing a miAPOE expression cassette (SEQ ID NOs. 108, 116, 146). Huh7 cells were transduced at a Multiplicity of Infection (MOI) of 5E+06, 3.6+06, 1E+06, 5E+05 or 5E+04 gc/cell. (A) Vector DNA copy numbers were determined at 48 h post-transduction and (B) APOE mRNA expression. APOE mRNA expression in the presence of AAV5-miSCR (SEQ ID No. 186) was set at 100%.

FIG. 10. Western blot of APOE protein in culture supernatant of HEK293T cells and astrocytoma cells 48 h post transfection with 250 ng APOE variant expression constructs in vitro.

FIG. 11. Vector DNA copies and APOE protein expression in culture supernatant of HEK293T cells transduced with AAV5 containing an APOE variant expression cassette. (SEQ ID NO. 197, 208, 210, 209, 211). HEK293T cells were transduced at a Multiplicity of Infection (MOI) of 1E4, 1E5 or 1E6. (A) Vector DNA copies in transduced HEK293T cells. (B) APOE protein detection by Western blot in the supernatant of the AAV transduced HEK293T cells.

FIG. 12. Detection of vector DNA (A) and mRNA copies (B) in the striatum of brain of C57BI6 mice injected with AAV-APOE3ch.-HA.

FIG. 13. Silencing of APOE4 Luc reporter by combined approach and miAPOE constructs in vitro upon co-transfection with the reporter (10 fmol) and the combined approach (SEQ ID NOs. 255-276 labeled as 1-22) or miAPOE constructs (SEQ ID NOs. 108, 116, 146).

FIG. 14. Silencing of APOE4 Luc reporter by combined approach constructs in vitro upon co-transfection of the reporter (2.18 fmol) with 0.69. 3.46 or fmol 17.31 of combined approach DNA constructs (SEQ ID NOs. 255-276 labeled as 1-22) or miAPOE constructs (SEQ ID NOs. 108, 116, 146). Relative Renilla expression (RL/FL) of the combined approach containing miSCR441 was set at 100%.

FIG. 15. APOE transgene expression in the supernatant of transfected Hek293T cells with the combined approach constructs (SEQ ID NOs. 255-276 labeled as 1-22) or APOE transgene constructs (SEQ ID NOs. 197, 208, 210, 211).

FIG. 16. Expression of Vector DNA in striatum (A) and frontal cortex (B) of mice injected with high (gray bars) or medium (white bars) dose of Empty AAV5, AAV5-miSCR, AAV5-miAPOE_016, AAV5-miAPOE_037, AAV5-miAPOE_145, or AAV9-miAPOE_145.

FIG. 17. Knockdown of APOE4 mRNA in striatum (A-B) and frontal cortex (C-D) of mice injected with high (gray bars) or medium (white bars) dose of AAV5-miAPOE_016, AAV5-miAPOE_037, AAV5-miAPOE_145, or AAV9-miAPOE_145 compared to Empty AAV5 and AAV5-miSCR. (B, D) Relative AAV5-miSCR copy levels were set at 100%.

FIG. 18. miRNA copy levels in striatum of mice injected with high (gray bars) or medium (white bars) dose of AAV5-miSCR, AAV5-miAPOE_016, AAV5-miAPOE_037, AAV5-miAPOE_145, or AAV9-miAPOE_145.

FIG. 19. Reduction of hAPOE4 protein in frontal cortex of mice injected with high (gray bars) or medium (white bars) dose of AAV5-miAPOE_016, AAV5-miAPOE_037, AAV5-miAPOE_145, or AAV9-miAPOE_145 compared to Empty AAV5 and AAV5-miSCR.

FIG. 20. (A) Abundancy of read lengths as compared to the total miAPOE reads found in the caudal cortex of hAPOE4-Tr mice injected with AAV5-miAPOE_016, AAV5-miAPOE_037 and AAV5-miAPOE_145. The most abundant read lengths for miAPOE_016, miAPOE_037 and miAPOE_145 were 23, 25 and 24 nucleotides respectively. (B) Read counts of miAPOE transcripts (C) Ranked abundancy of endogenous miRNAs and miAPOEs detected in the caudal cortex of hAPOE4-Tr mice injected with AAV5-miSCR, AAV5-miAPOE_016, AAV5-miAPOE_037 and AAV5-miAPOE_145. The internal controls miR-29a-3p and miR-16-5p are marked with a single or double asterisks, respectively.

FIG. 21. Expression of Vector DNA (A) and APOE mRNA (B) in striatum of WT or P301S mice injected with vehicle, empty AAV5, AAV5-APOE2ch WT, AAV5-APOE2ch V2, AAV5-APOE2b V2, AAV5-APOE3ch V2 and AAV5-APOE3b V2. The dashed lines indicate the LLOQs. (C-D) hAPOE protein levels detected in hippocampus (C) and frontal cortex (D) of WT or P301S mice injected with vehicle, empty AAV5, AAV5-APOE2ch WT, AAV5-APOE2ch V2, AAV5-APOE2b V2, AAV5-APOE3ch V2 and AAV5-APOE3b V2.

FIG. 22. (A) Total Tau protein levels in hippocampus of WT or P301S mice injected with vehicle, empty AAV5, AAV5-APOE2ch WT, AAV5-APOE2ch V2, AAV5-APOE2b V2, AAV5-APOE3ch V2 and AAV5-APOE3b V2. (B-C) pTau181 protein levels in hippocampus of WT or P301S mice injected with vehicle, empty AAV5, AAV5-APOE2ch WT, AAV5-APOE2ch V2, AAV5-APOE2b V2, AAV5-APOE3ch V2 and AAV5-APOE3b V2. The measured (B) and normalized values (C) demonstrate significant decrease in pTau181 levels in all groups injected with an AAV5-APOE variant. ** P<0.01, *** P<0.001 by one-way ANOVA. The dashed line marks 50%. (D-E) pTau181/Total Tau ratio in hippocampus of WT or P301S mice injected with vehicle, empty AAV5, AAV5-APOE2ch WT, AAV5-APOE2ch V2, AAV5-APOE2b V2, AAV5-APOE3ch V2 and AAV5-APOE3b V2. The measured (D) and normalized values (E) reveal significant decrease in pTau181/Total Tau ratio in groups injected with AAV5-APOE2ch V2, AAV5-APOE2b V, and AAV5-APOE3b V2. ** P<0.01, **** P<0.0001 by one-way ANOVA. The dashed line marks 50%.

FIG. 23. Expression of vector DNA (A) and miAPOE copy levels (B) in striatum of WT mice injected with combined approach and single constructs encapsidated in AAV5. APOE mRNA copy levels in striatum (C) and APOE protein levels in the cortex (D) of WT mice injected with combined approach and single constructs encapsidated in AAV5. The dashed lines indicate the LLOQ.

FIG. 24. (A) Abundancy of read lengths as compared to the total miAPOE reads found in the U-118 MG cell cultures transduced with AAV5-combined approach constructs ID 2 and 16, AAV5-miAPOE_016 and AAV5-miAPOE_145. The most abundant read length for all constructs is 24 nucleotides. (B) Read counts of miAPOE transcripts (C) Ranked abundancy of endogenous miRNAs and miAPOEs detected in the U-118 MG cell cultures transduced with AAV5-combined approach constructs ID 2 and 16, AAV5-miSCR, AAV5-miAPOE_016 and AAV5-miAPOE_145. The internal controls miR-29a-3p and miR-16-5p are marked with a single or double asterisks, respectively. (D) Differential expression analysis of the combined approach, miAPOE_016 and miAPOE_145 constructs.

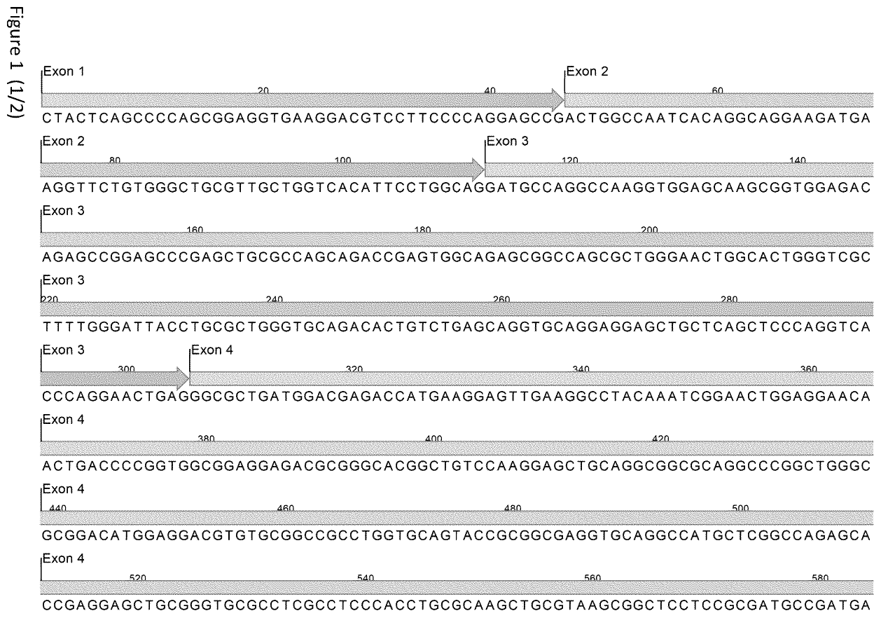

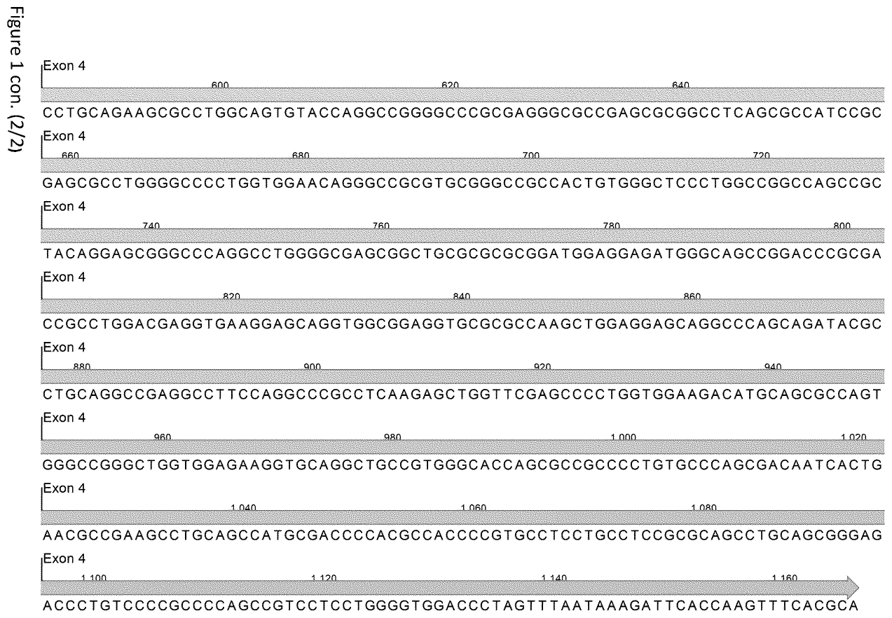

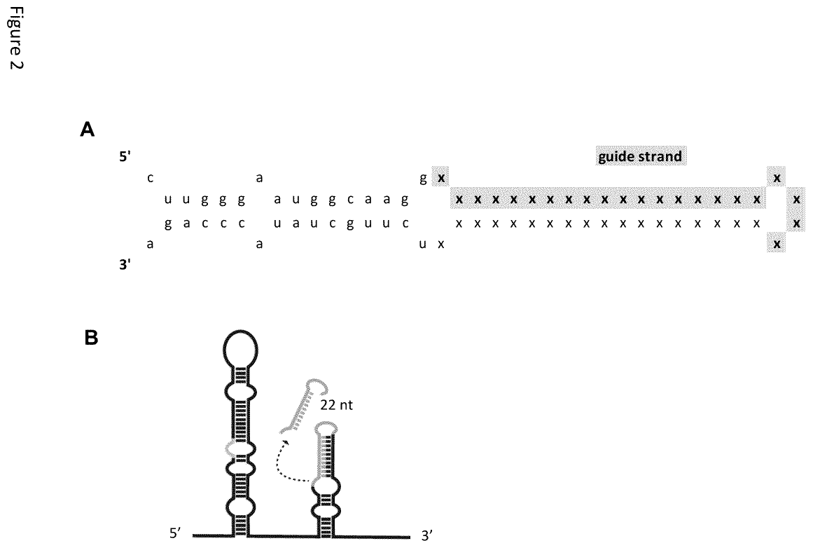

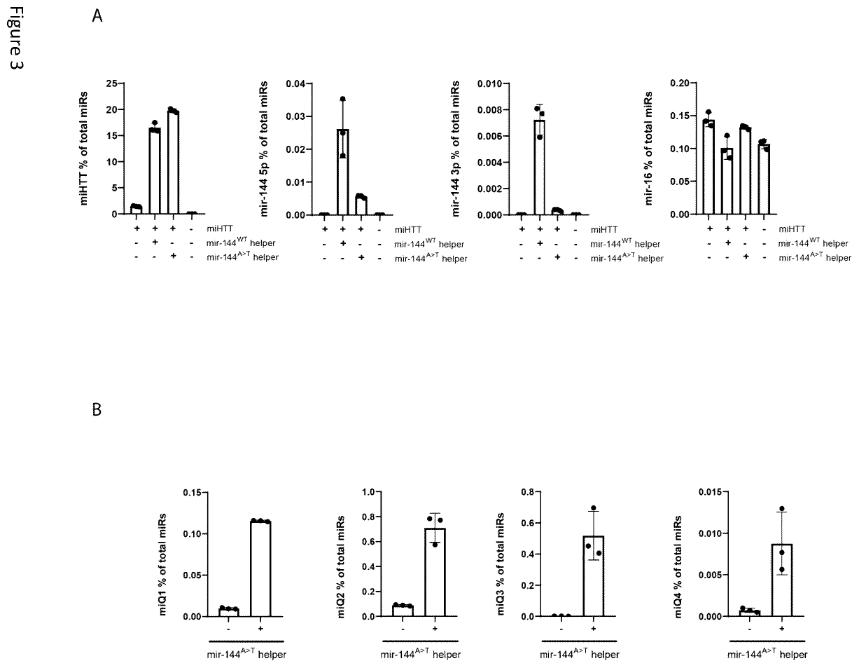

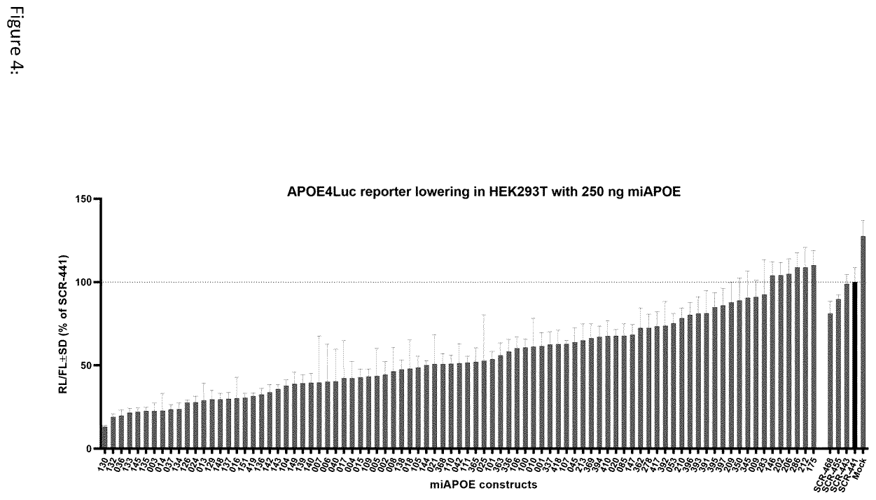

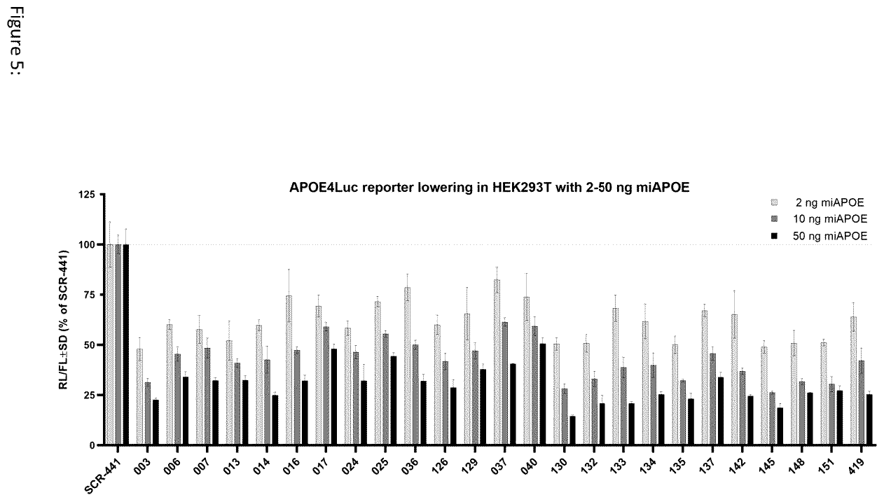

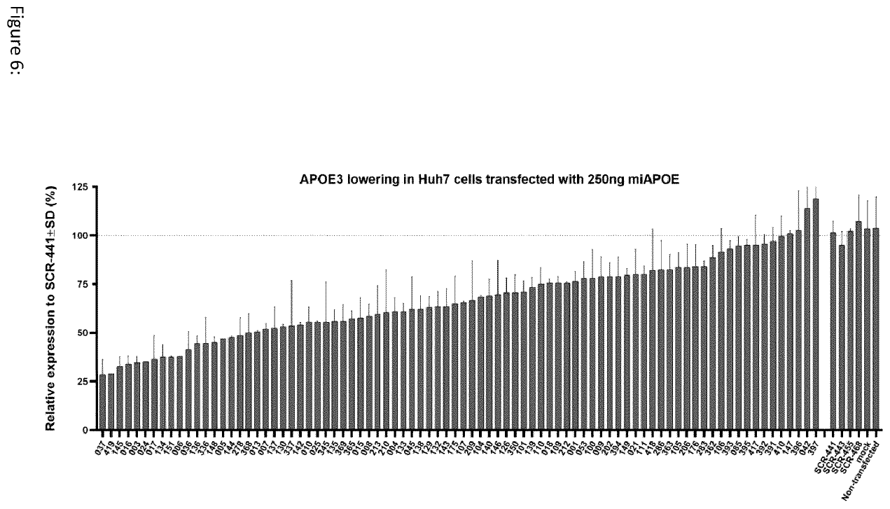

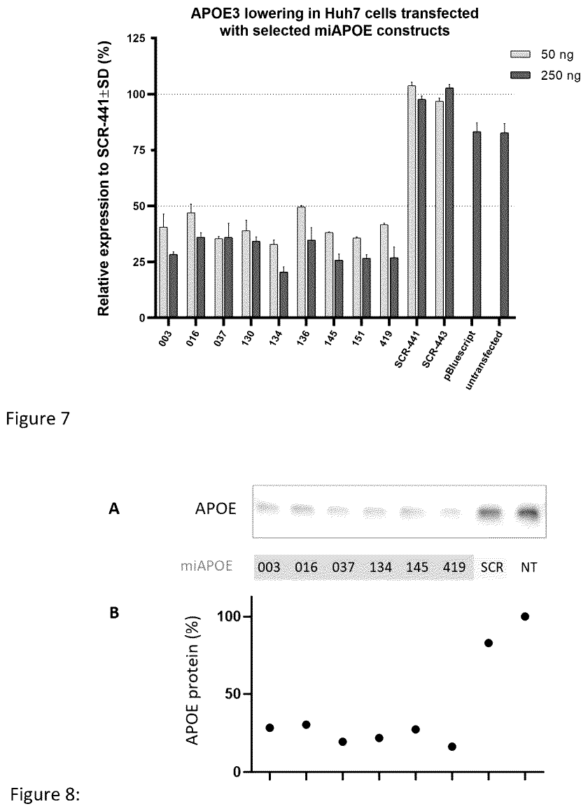

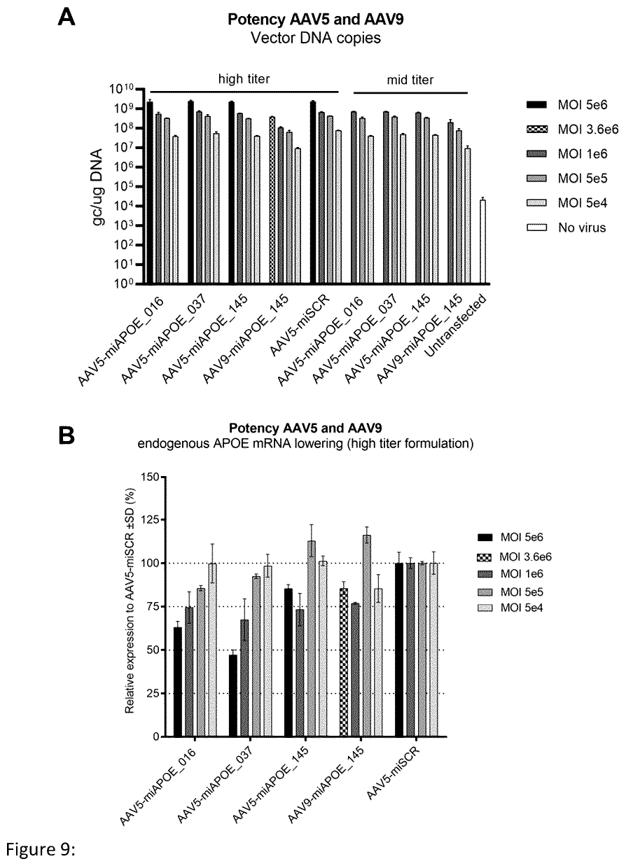

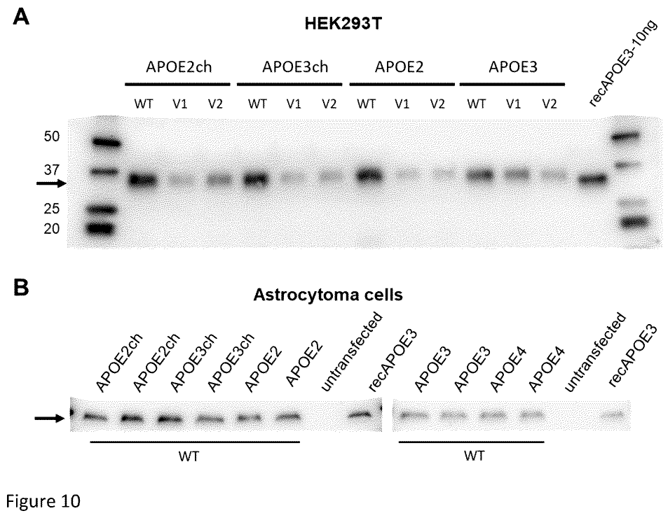

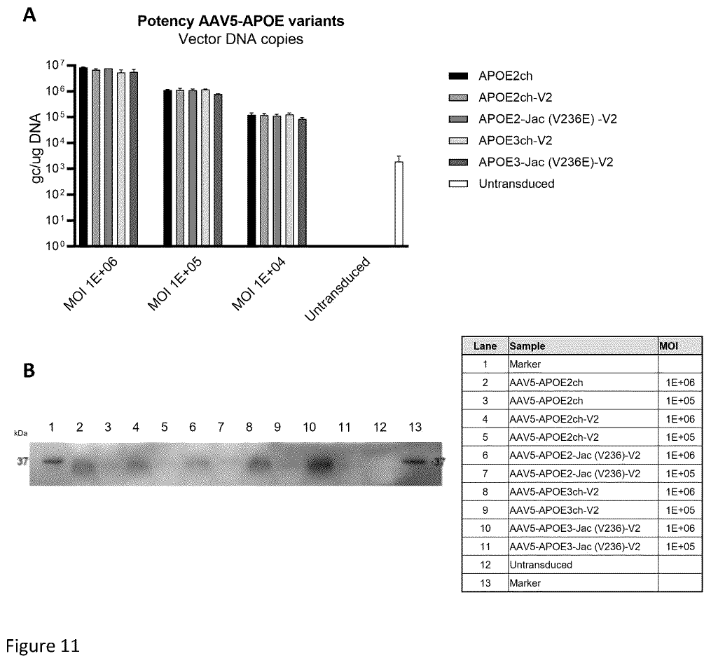

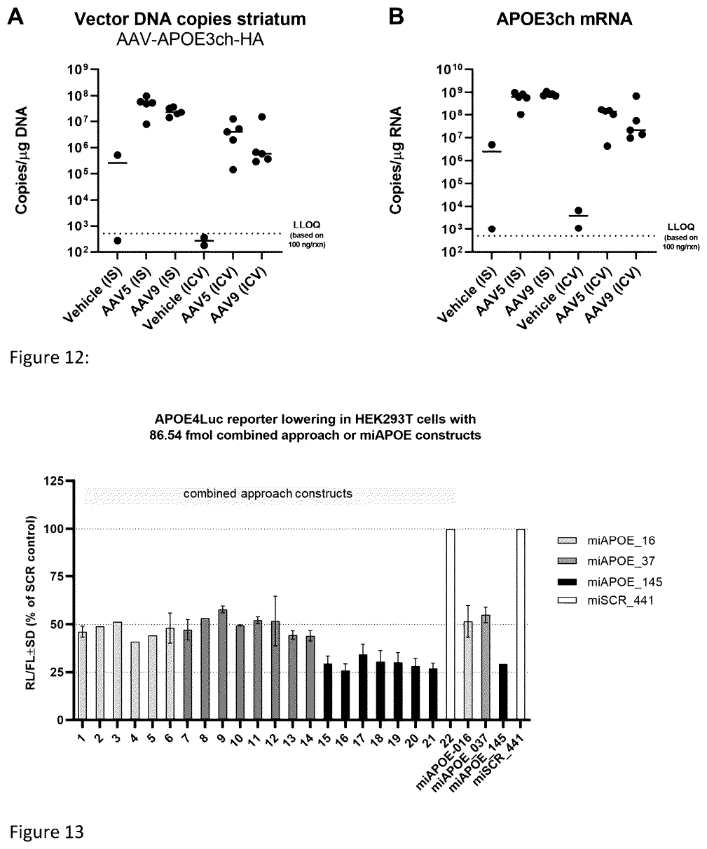

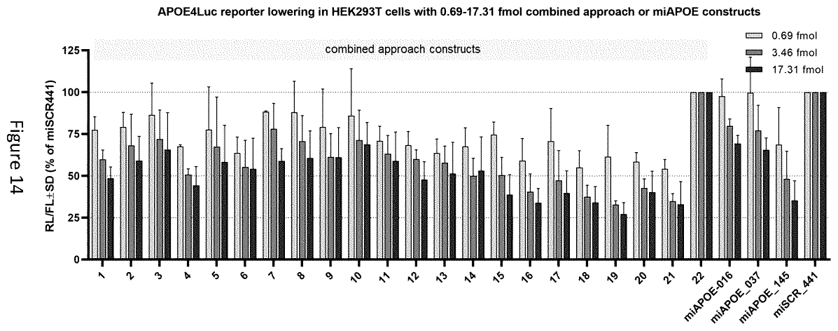

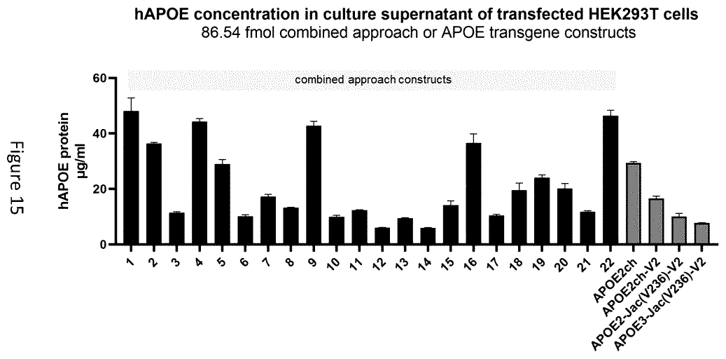

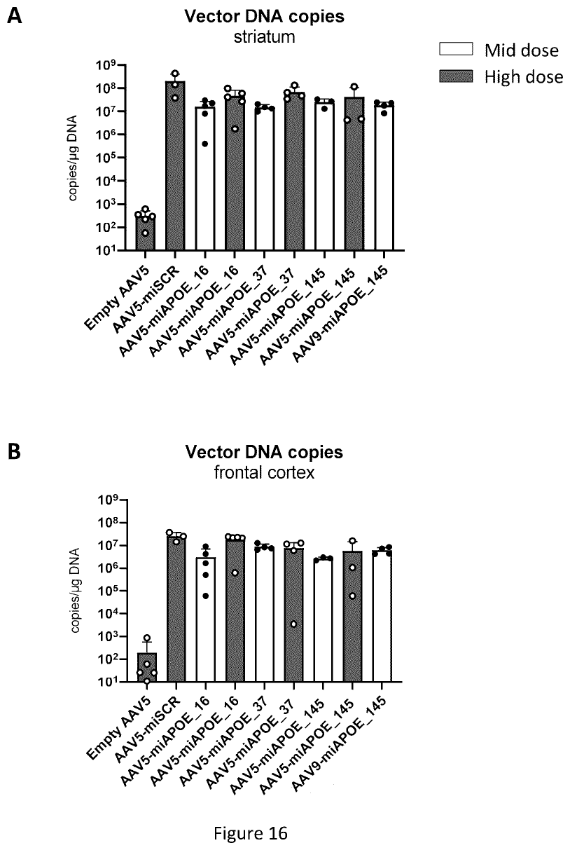

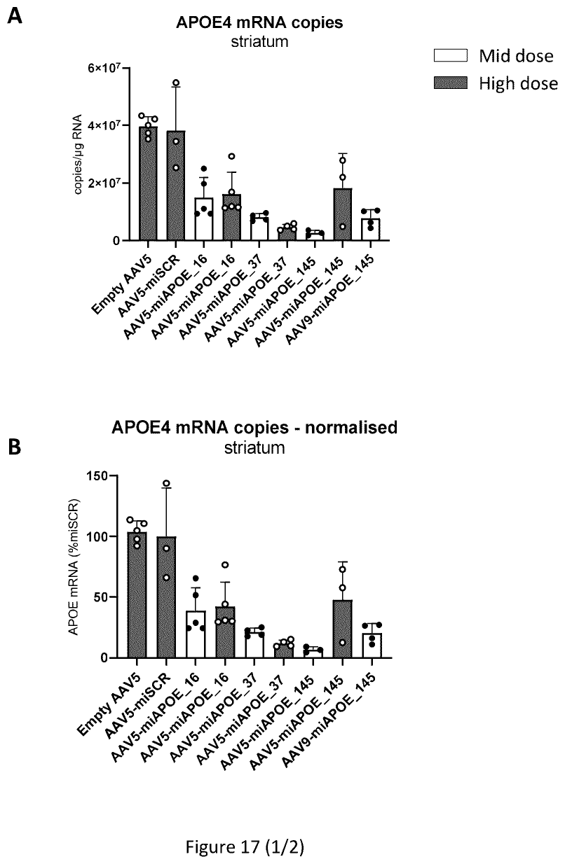

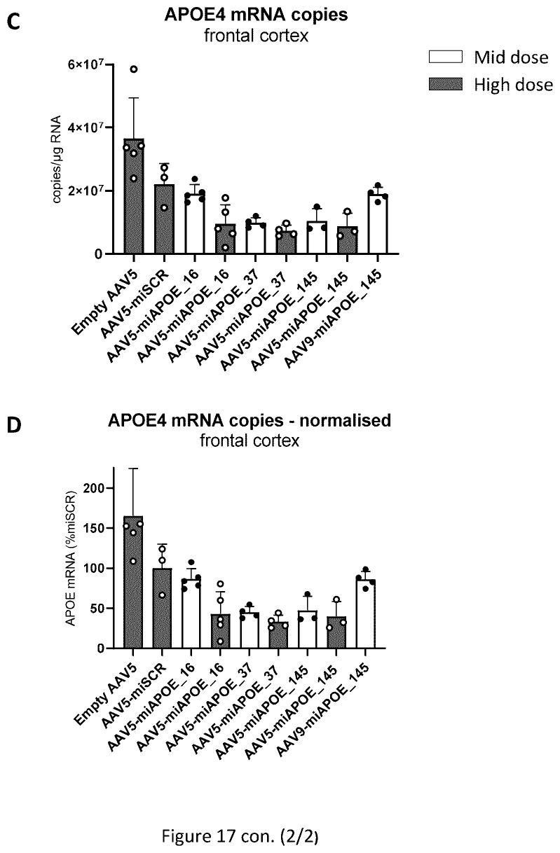

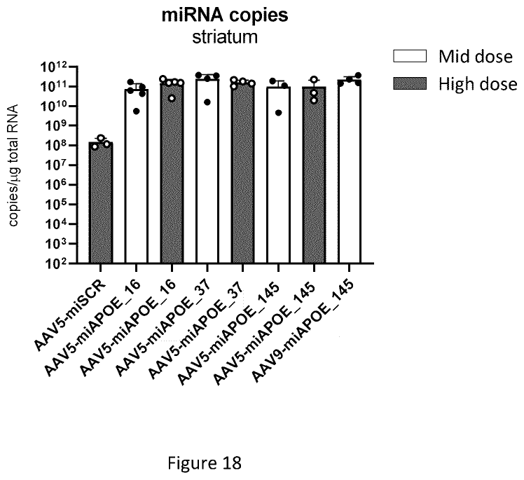

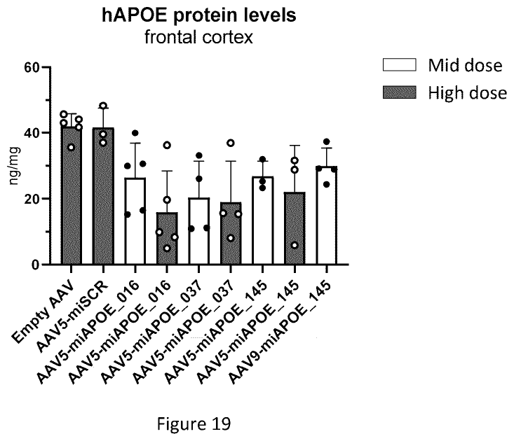

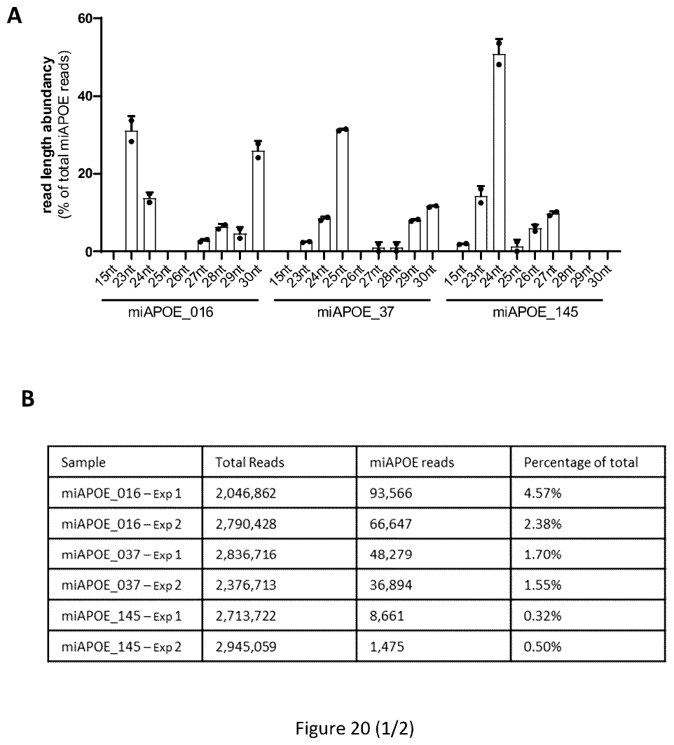

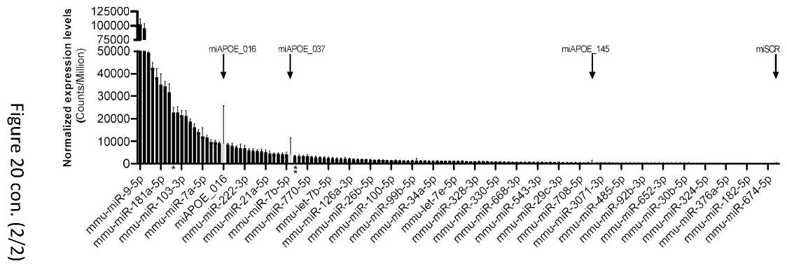

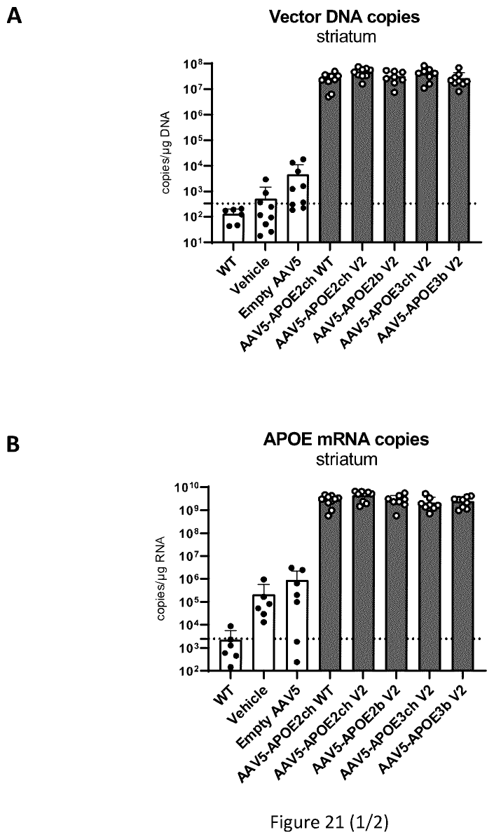

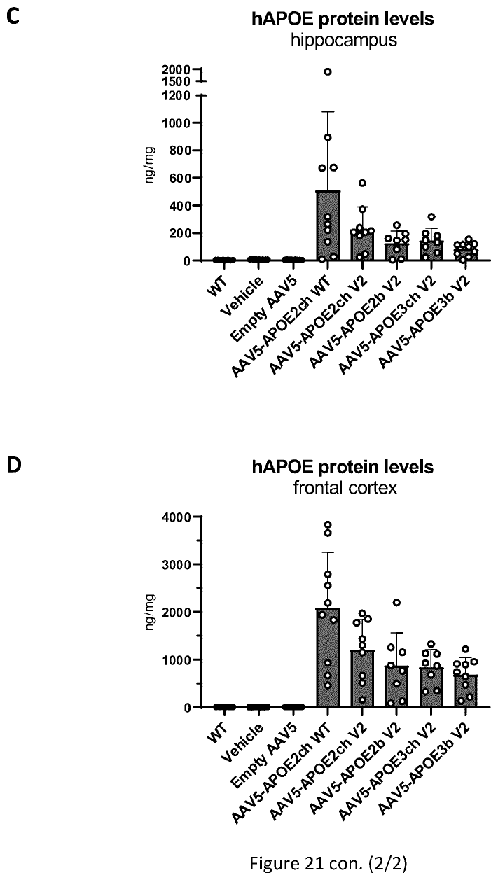

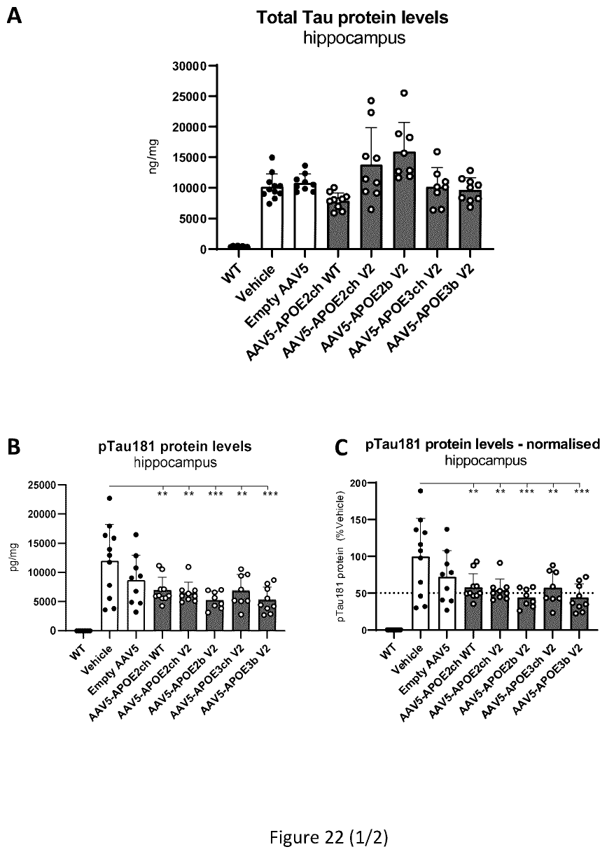

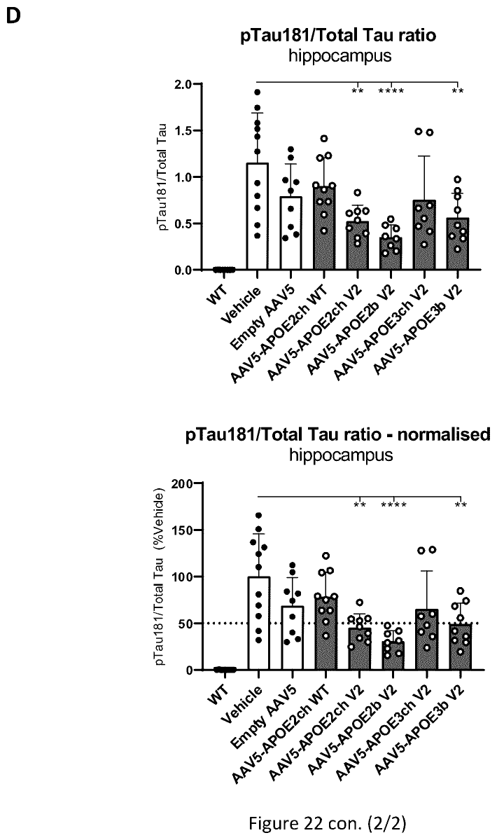

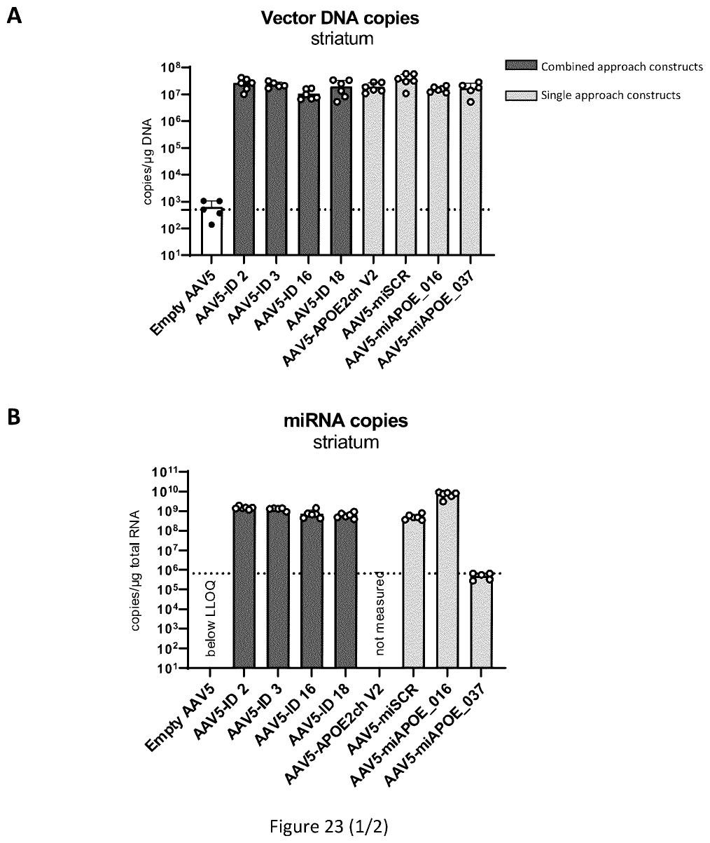

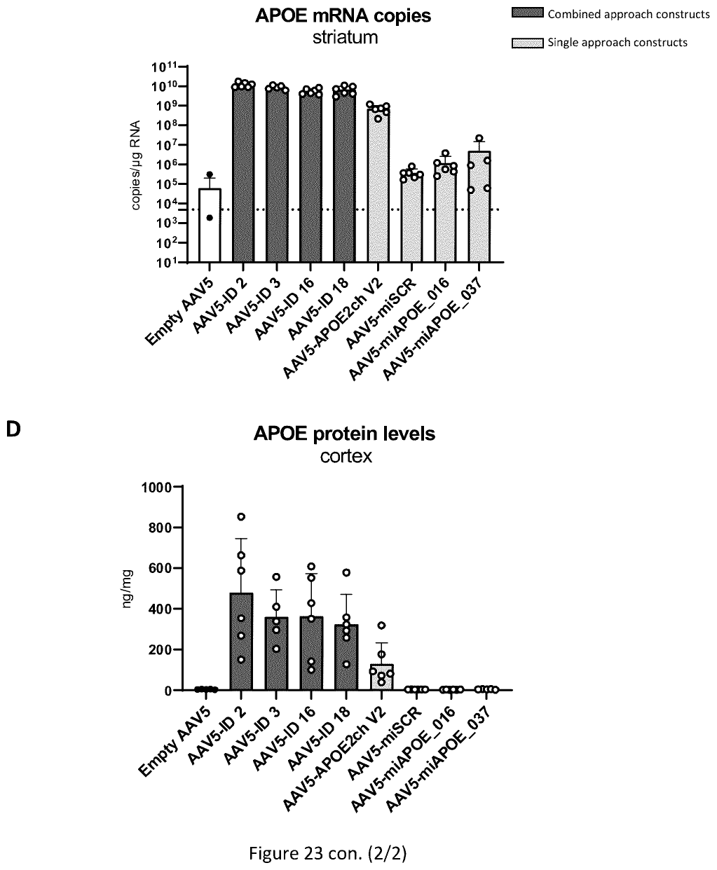

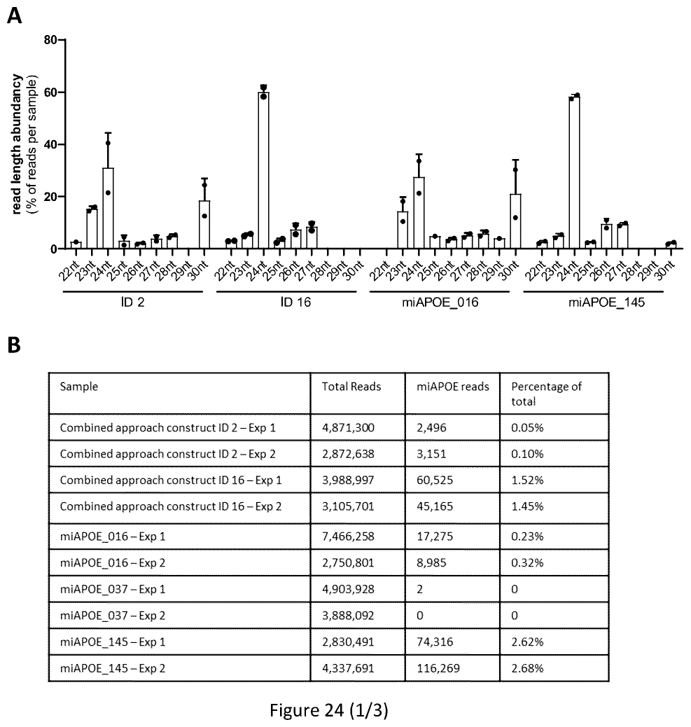

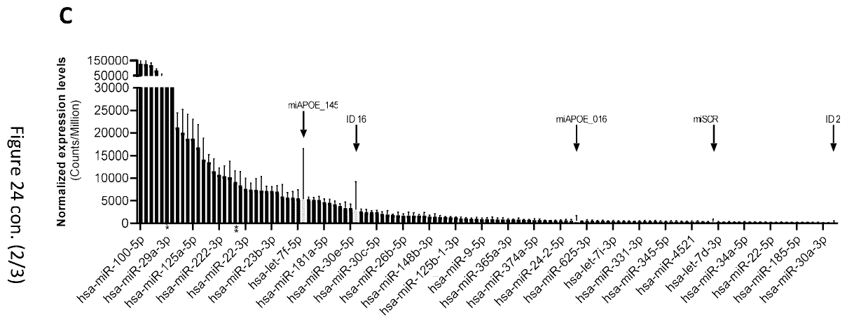

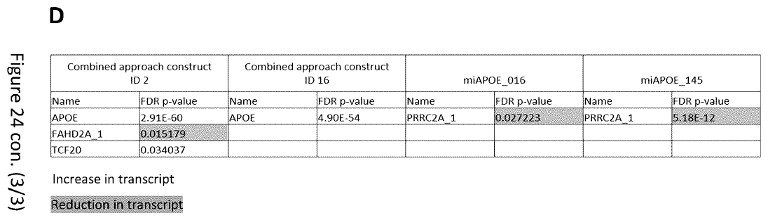

1. A nucleic acid comprising a sequence encoding a first RNA and a sequence encoding a second RNA, wherein the second RNA comprises a guide sequence of at least 19 nucleotides substantially complementary to part of an ApoE gene, and wherein the first RNA and second RNA each comprise a hairpin.

2. The nucleic acid according to claim 1, wherein, in a 5′ to 3′ direction, the sequence encoding the first RNA is followed by a spacer, which spacer is followed by the sequence encoding the second RNA, and wherein the spacer is at least 50 nucleotides.

3. The nucleic acid according to claim 1, wherein the hairpin in the second RNA comprises at least 40 nucleotides.

4. The nucleic acid according to claim 1, wherein the first RNA comprises SEQ ID NO. 235 or a variant thereof and the second RNA comprises SEQ ID NO. 190 or a variant thereof.

5. The nucleic acid according to claim 1, wherein the guide sequence is at least 22 nucleotides.

6. The nucleic acid according to claim 1, wherein the guide sequence is specific to part of the ApoE4 gene.

7. The nucleic acid according to claim 1, wherein the guide sequence is substantially complementary to a sequence selected from SEQ ID NOs. 16, 24, 54.

8. The nucleic acid according to claim 1, wherein the first RNA is mutated to reduce processing and/or expression of the first RNA.

9. An expression cassette comprising the nucleic acid according to claim 1, wherein the expression cassette is a DNA molecule.

10. The expression cassette according to claim 9 further comprising a second nucleic acid encoding at least one of APOE2 and APOE3, wherein the first and second nucleic acids are operably linked to a promoter and optionally to a poly-A signal, wherein preferably, the second nucleic acid encodes for a protein comprising one of SEQ ID NOs 249-254 and/or the second nucleic acid comprises one of SEQ ID NOs. 195-217.

11. The expression cassette according to claim 10, wherein the second nucleic acid does not comprise a sequence that is substantially complementary to the guide sequence as defined in any one of claims 1 to 7, wherein preferably, the nucleotide sequence is codon optimised for expression in human cells.

12. The expression cassette according to claim 10, wherein the second nucleic acid comprises a sequence selected from SEQ ID NOs. 195-217.

13. The expression cassette according to claim 10, wherein the sequence encoding the first RNA and the sequence encoding the second RNA are comprised in an intronic sequence.

14. The expression cassette according to claim 13, wherein intronic sequence is present in the promoter, wherein preferably the intronic sequence comprises SEQ ID NO. 231.

15. The expression cassette according to claim 9, wherein the promoter is a promoter capable of driving transcription in a brain cell, wherein preferably, the promoter comprises one of SEQ ID NOs. 191,192,193 or 232.

16. The expression cassette according to claim 9, wherein the expression cassette is flanked by Inverted Terminal Repeats (ITRs).

17.-24. (canceled)

25. An expression cassette comprising a nucleic acid which encodes one or more APOE2 and APOE3 proteins selected from SEQ ID NOs. 249-254.

26. The expression cassette according to claim 25, wherein said one or more proteins are encoded by nucleic acid sequences that comprise at least partial wild type sequences.

27. The expression cassette according to claim 25, wherein in said one or more proteins are encoded by nucleic acid sequences that comprise full length wild type coding sequences.

28. The expression cassette for use according to claim 25, wherein the expression cassette comprises one or more nucleic acid sequences selected from SEQ ID NOs. 195-201, optionally operably linked to a promoter and a poly-A signal.

29. The expression cassette according to claim 25, wherein the expression cassette is flanked by ITRs.

30. An adeno-associated virus (AAV) vector comprising the expression cassette according to claim 25.

31. The AAV vector according to claim 30, comprising an AAV5 or AAV9 capsid protein.

32. A pharmaceutical composition comprising the AAV vector according to claim 30 and at least one pharmaceutically acceptable excipient.

33. A method of treating and/or preventing Alzheimer's disease in a human, the method comprising the administration of the AAV vector according to claim 30.

34. The method according to claim 33, wherein the human is a carrier of an ApoE4 allele, wherein preferably the human is homozygous for an ApoE4 allele.

35. The method according to claim 33, wherein the AAV vector is administered to the central nervous system, preferably by intracerebral injection, intraparenchymal injection, intrathecal injection, or a combination thereof, more preferably by convection enhanced delivery.

36. (canceled)


SEQUENCE LISTING (299 sequences)

SEQ ID NO: 1 - DNA, 1166 bp, synthetic construct
  misc_feature 1..1166: note = apolipoprotein E (APOE)
  source 1..1166: mol_type = other DNA
ctactcagccccagcggaggtgaaggacgtccttccccaggagccgactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgca

SEQ ID NO: 2 - RNA, 22 nt, synthetic construct
  misc_feature 1..22: note = RNA TARGET SEQUENCE
  source 1..22: mol_type = other RNA
actggccaatcacaggcaggaa

SEQ ID NO: 3 - RNA, 22 nt, synthetic construct
  misc_feature 1..22: note = RNA TARGET SEQUENCE
  source 1..22: mol_type = other RNA
ctggccaatcacaggcaggaag

SEQ ID NO: 4 - RNA, 22 nt, synthetic construct
  misc_feature 1..22: note = RNA TARGET SEQUENCE
  source 1..22: mol_type = other RNA
tggccaatcacaggcaggaaga

SEQ ID NO: 5 - RNA, 22 nt, synthetic construct
  misc_feature 1..22: note = RNA TARGET SEQUENCE
  source 1..22: mol_type = other RNA
ggccaatcacaggcaggaagat

SEQ ID NO: 6 - RNA, 22 nt, synthetic construct
  misc_feature 1..22: note = RNA TARGET SEQUENCE
  source 1..22: mol_type = other RNA
gccaatcacaggcaggaagatg

SEQ ID NO: 7 - RNA, 22 nt, synthetic construct
  misc_feature 1..22: note = RNA TARGET SEQUENCE
  source 1..22: mol_type = other RNA
ccaatcacaggcaggaagatga

SEQ ID NO: 8 - RNA, 22 nt, synthetic construct
  misc_feature 1..22: note = RNA TARGET SEQUENCE
  source 1..22: mol_type = other RNA
caatcacaggcaggaagatgaa

SEQ ID NO: 9 - RNA, 22 nt, synthetic construct
  misc_feature 1..22: note = RNA TARGET SEQUENCE
  source 1..22: mol_type = other RNA
aatcacaggcaggaagatgaag

SEQ ID NO: 10 - RNA, 22 nt, synthetic construct
  misc_feature 1..22: note = RNA TARGET SEQUENCE
  source 1..22: mol_type = other RNA
atcacaggcaggaagatgaagg

SEQ ID NO: 11 - RNA, 22 nt, synthetic construct
  misc_feature 1..22: note = RNA TARGET SEQUENCE
  source 1..22: mol_type = other RNA
tcacaggcaggaagatgaaggt

SEQ ID NO: 12 - RNA, 22 nt, synthetic construct
  misc_feature 1..22: note = RNA TARGET SEQUENCE
  source 1..22: mol_type = other RNA
cacaggcaggaagatgaaggtt

SEQ ID NO: 13 - RNA, 22 nt, synthetic construct
  misc_feature 1..22: note = RNA TARGET SEQUENCE
  source 1..22: mol_type = other RNA
caggcaggaagatgaaggttct

SEQ ID NO: 14 - RNA, 22 nt, synthetic construct
  misc_feature 1..22: note = RNA TARGET SEQUENCE
  source 1..22: mol_type = other RNA
aggcaggaagatgaaggttctg

SEQ ID NO: 15 - RNA, 22 nt, synthetic construct
  misc_feature 1..22: note = RNA TARGET SEQUENCE
  source 1..22: mol_type = other RNA
ggcaggaagatgaaggttctgt

SEQ ID NO: 16 - RNA, 22 nt, synthetic construct
  misc_feature 1..22: note = RNA TARGET SEQUENCE
  source 1..22: mol_type = other RNA
gcaggaagatgaaggttctgtg

SEQ ID NO: 17 - RNA, 22 nt, synthetic construct
  misc_feature 1..22: note = RNA TARGET SEQUENCE
  source 1..22: mol_type = other RNA
caggaagatgaaggttctgtgg

SEQ ID NO: 18 - RNA, 22 nt, synthetic construct
  misc_feature 1..22: note = RNA TARGET SEQUENCE
  source 1..22: mol_type = other RNA
aggaagatgaaggttctgtggg

SEQ ID NO: 19 - RNA, 22 nt, synthetic construct
  misc_feature 1..22: note = RNA TARGET SEQUENCE
  source 1..22: mol_type = other RNA
gaagatgaaggttctgtgggct

SEQ ID NO: 20 - RNA, 22 nt, synthetic construct
  misc_feature 1..22: note = RNA TARGET SEQUENCE
  source 1..22: mol_type = other RNA
aagatgaaggttctgtgggctg

SEQ ID NO: 21 - RNA, 22 nt, synthetic construct
  misc_feature 1..22: note = RNA TARGET SEQUENCE
  source 1..22: mol_type = other RNA
atgaaggttctgtgggctgcgt

SEQ ID NO: 22 - RNA, 22 nt, synthetic construct
  misc_feature 1..22: note = RNA TARGET SEQUENCE
  source 1..22: mol_type = other RNA
tgaaggttctgtgggctgcgtt

SEQ ID NO: 23 - RNA, 22 nt, synthetic construct
  misc_feature 1..22: note = RNA TARGET SEQUENCE
  source 1..22: mol_type = other RNA
tgggctgcgttgctggtcacat

SEQ ID NO: 24 - RNA, 22 nt, synthetic construct
  misc_feature 1..22: note = RNA TARGET SEQUENCE
  source 1..22: mol_type = other RNA
gggctgcgttgctggtcacatt

SEQ ID NO: 25 - RNA, 22 nt, synthetic construct
  misc_feature 1..22: note = RNA TARGET SEQUENCE
  source 1..22: mol_type = other RNA
ctgcgttgctggtcacattcct

SEQ ID NO: 26 - RNA, 22 nt, synthetic construct
  misc_feature 1..22: note = RNA TARGET SEQUENCE
  source 1..22: mol_type = other RNA
gcgttgctggtcacattcctgg

SEQ ID NO: 27 - RNA, 22 nt, synthetic construct
  misc_feature 1..22: note = RNA TARGET SEQUENCE
  source 1..22: mol_type = other RNA
ttgctggtcacattcctggcag

SEQ ID NO: 28 - RNA, 22 nt, synthetic construct
  misc_feature 1..22: note = RNA TARGET SEQUENCE
  source 1..22: mol_type = other RNA
cacattcctggcaggatgccag

SEQ ID NO: 29 - RNA, 22 nt, synthetic construct
  misc_feature 1..22: note = RNA TARGET SEQUENCE
  source 1..22: mol_type = other RNA
tttgggattacctgcgctgggt

SEQ ID NO: 30 - RNA, 22 nt, synthetic construct
  misc_feature 1..22: note = RNA TARGET SEQUENCE
  source 1..22: mol_type = other RNA
gctgggtgcagacactgtctga

SEQ ID NO: 31 - RNA, 22 nt, synthetic construct
  misc_feature 1..22: note = RNA TARGET SEQUENCE
  source 1..22: mol_type = other RNA
ctgggtgcagacactgtctgag

SEQ ID NO: 32 - RNA, 22 nt, synthetic construct
  misc_feature 1..22: note = RNA TARGET SEQUENCE
  source 1..22: mol_type = other RNA
ggtgcagacactgtctgagcag

SEQ ID NO: 33 - RNA, 22 nt, synthetic construct
  misc_feature 1..22: note = RNA TARGET SEQUENCE
  source 1..22: mol_type = other RNA
gtgcagacactgtctgagcagg

SEQ ID NO: 34 - RNA, 22 nt, synthetic construct
  misc_feature 1..22: note = RNA TARGET SEQUENCE
  source 1..22: mol_type = other RNA
tgcagacactgtctgagcaggt

SEQ ID NO: 35 - RNA, 22 nt, synthetic construct
  misc_feature 1..22: note = RNA TARGET SEQUENCE
  source 1..22: mol_type = other RNA
gcagacactgtctgagcaggtg

SEQ ID NO: 36 - RNA, 22 nt, synthetic construct
  misc_feature 1..22: note = RNA TARGET SEQUENCE
  source 1..22: mol_type = other RNA
agacactgtctgagcaggtgca

SEQ ID NO: 37 - RNA, 22 nt, synthetic construct
  misc_feature 1..22: note = RNA TARGET SEQUENCE
  source 1..22: mol_type = other RNA
gacactgtctgagcaggtgcag

SEQ ID NO: 38 - RNA, 22 nt, synthetic construct
  misc_feature 1..22: note = RNA TARGET SEQUENCE
  source 1..22: mol_type = other RNA
acactgtctgagcaggtgcagg

SEQ ID NO: 39 - RNA, 22 nt, synthetic construct
  misc_feature 1..22: note = RNA TARGET SEQUENCE
  source 1..22: mol_type = other RNA
gagaccatgaaggagttgaagg

SEQ ID NO: 40 - RNA, 22 nt, synthetic construct
  misc_feature 1..22: note = RNA TARGET SEQUENCE
  source 1..22: mol_type = other RNA
accatgaaggagttgaaggcct

SEQ ID NO: 41 - RNA, 22 nt, synthetic construct
  misc_feature 1..22: note = RNA TARGET SEQUENCE
  source 1..22: mol_type = other RNA
ccatgaaggagttgaaggccta

SEQ ID NO: 42 - RNA, 22 nt, synthetic construct
  misc_feature 1..22: note = RNA TARGET SEQUENCE
  source 1..22: mol_type = other RNA
atgaaggagttgaaggcctaca

SEQ ID NO: 43 - RNA, 22 nt, synthetic construct
  misc_feature 1..22: note = RNA TARGET SEQUENCE
  source 1..22: mol_type = other RNA
tgaaggagttgaaggcctacaa

SEQ ID NO: 44 - RNA, 22 nt, synthetic construct
  misc_feature 1..22: note = RNA TARGET SEQUENCE
  source 1..22: mol_type = other RNA
gaaggagttgaaggcctacaaa

SEQ ID NO: 45 - RNA, 22 nt, synthetic construct
  misc_feature 1..22: note = RNA TARGET SEQUENCE
  source 1..22: mol_type = other RNA
aaggagttgaaggcctacaaat

SEQ ID NO: 46 - RNA, 22 nt, synthetic construct
  misc_feature 1..22: note = RNA TARGET SEQUENCE
  source 1..22: mol_type = other RNA
aggagttgaaggcctacaaatc

SEQ ID NO: 47 - RNA, 22 nt, synthetic construct
  misc_feature 1..22: note = RNA TARGET SEQUENCE
  source 1..22: mol_type = other RNA
ggagttgaaggcctacaaatcg

SEQ ID NO: 48 - RNA, 22 nt, synthetic construct
  misc_feature 1..22: note = RNA TARGET SEQUENCE
  source 1..22: mol_type = other RNA
gagttgaaggcctacaaatcgg

SEQ ID NO: 49 - RNA, 22 nt, synthetic construct
  misc_feature 1..22: note = RNA TARGET SEQUENCE
  source 1..22: mol_type = other RNA
agttgaaggcctacaaatcgga

SEQ ID NO: 50 - RNA, 22 nt, synthetic construct
  misc_feature 1..22: note = RNA TARGET SEQUENCE
  source 1..22: mol_type = other RNA
gttgaaggcctacaaatcggaa

SEQ ID NO: 51 - RNA, 22 nt, synthetic construct
  misc_feature 1..22: note = RNA TARGET SEQUENCE
  source 1..22: mol_type = other RNA
tgaaggcctacaaatcggaact

SEQ ID NO: 52 - RNA, 22 nt, synthetic construct
  misc_feature 1..22: note = RNA TARGET SEQUENCE
  source 1..22: mol_type = other RNA
gaaggcctacaaatcggaactg

SEQ ID NO: 53 - RNA, 22 nt, synthetic construct
  misc_feature 1..22: note = RNA TARGET SEQUENCE
  source 1..22: mol_type = other RNA
aaggcctacaaatcggaactgg

SEQ ID NO: 54 - RNA, 22 nt, synthetic construct
  misc_feature 1..22: note = RNA TARGET SEQUENCE
  source 1..22: mol_type = other RNA
aggcctacaaatcggaactgga

SEQ ID NO: 55 - RNA, 22 nt, synthetic construct
  misc_feature 1..22: note = RNA TARGET SEQUENCE
  source 1..22: mol_type = other RNA
ggcctacaaatcggaactggag

SEQ ID NO: 56 - RNA, 22 nt, synthetic construct
  misc_feature 1..22: note = RNA TARGET SEQUENCE
  source 1..22: mol_type = other RNA
gcctacaaatcggaactggagg

SEQ ID NO: 57 - RNA, 22 nt, synthetic construct
  misc_feature 1..22: note = RNA TARGET SEQUENCE
  source 1..22: mol_type = other RNA
cctacaaatcggaactggagga

SEQ ID NO: 58 - RNA, 22 nt, synthetic construct
  misc_feature 1..22: note = RNA TARGET SEQUENCE
  source 1..22: mol_type = other RNA
ctacaaatcggaactggaggaa

SEQ ID NO: 59 - RNA, 22 nt, synthetic construct
  misc_feature 1..22: note = RNA TARGET SEQUENCE
  source 1..22: mol_type = other RNA
acaaatcggaactggaggaaca

SEQ ID NO: 60 - RNA, 22 nt, synthetic construct
  misc_feature 1..22: note = RNA TARGET SEQUENCE
  source 1..22: mol_type = other RNA
cacggctgtccaaggagctgca

SEQ ID NO: 61 - RNA, 22 nt, synthetic construct
  misc_feature 1..22: note = RNA TARGET SEQUENCE
  source 1..22: mol_type = other RNA
acggctgtccaaggagctgcag

SEQ ID NO: 62 - RNA, 22 nt, synthetic construct
  misc_feature 1..22: note = RNA TARGET SEQUENCE
  source 1..22: mol_type = other RNA
tcgcctcccacctgcgcaagct

SEQ ID NO: 63 - RNA, 22 nt, synthetic construct
  misc_feature 1..22: note = RNA TARGET SEQUENCE
  source 1..22: mol_type = other RNA
gatgacctgcagaagcgcctgg

SEQ ID NO: 64 - RNA, 22 nt, synthetic construct
  misc_feature 1..22: note = RNA TARGET SEQUENCE
  source 1..22: mol_type = other RNA
gacctgcagaagcgcctggcag

SEQ ID NO: 65 - RNA, 22 nt, synthetic construct
  misc_feature 1..22: note = RNA TARGET SEQUENCE
  source 1..22: mol_type = other RNA
acctgcagaagcgcctggcagt

SEQ ID NO: 66 - RNA, 22 nt, synthetic construct
  misc_feature 1..22: note = RNA TARGET SEQUENCE
  source 1..22: mol_type = other RNA
ctgcagaagcgcctggcagtgt

SEQ ID NO: 67 - RNA, 22 nt, synthetic construct
  misc_feature 1..22: note = RNA TARGET SEQUENCE
  source 1..22: mol_type = other RNA
tgcagaagcgcctggcagtgta

SEQ ID NO: 68 - RNA, 22 nt, synthetic construct
  misc_feature 1..22: note = RNA TARGET SEQUENCE
  source 1..22: mol_type = other RNA
accgcctggacgaggtgaagga

SEQ ID NO: 69 - RNA, 22 nt, synthetic construct
  misc_feature 1..22: note = RNA TARGET SEQUENCE
  source 1..22: mol_type = other RNA
ctggacgaggtgaaggagcagg

SEQ ID NO: 70 - RNA, 22 nt, synthetic construct
  misc_feature 1..22: note = RNA TARGET SEQUENCE
  source 1..22: mol_type = other RNA
gacgaggtgaaggagcaggtgg

SEQ ID NO: 71 - RNA, 22 nt, synthetic construct
  misc_feature 1..22: note = RNA TARGET SEQUENCE
  source 1..22: mol_type = other RNA
cccgcctcaagagctggttcga

SEQ ID NO: 72 - RNA, 22 nt, synthetic construct
  misc_feature 1..22: note = RNA TARGET SEQUENCE
  source 1..22: mol_type = other RNA
ccgcctcaagagctggttcgag

SEQ ID NO: 73 - RNA, 22 nt, synthetic construct
  misc_feature 1..22: note = RNA TARGET SEQUENCE
  source 1..22: mol_type = other RNA
gctggtggagaaggtgcaggct

SEQ ID NO: 74 - RNA, 22 nt, synthetic construct
  misc_feature 1..22: note = RNA TARGET SEQUENCE
  source 1..22: mol_type = other RNA
tggagaaggtgcaggctgccgt

SEQ ID NO: 75 - RNA, 22 nt, synthetic construct
  misc_feature 1..22: note = RNA TARGET SEQUENCE
  source 1..22: mol_type = other RNA
cctgtgcccagcgacaatcact

SEQ ID NO: 76 - RNA, 22 nt, synthetic construct
  misc_feature 1..22: note = RNA TARGET SEQUENCE
  source 1..22: mol_type = other RNA
tgtgcccagcgacaatcactga

SEQ ID NO: 77 - RNA, 22 nt, synthetic construct
  misc_feature 1..22: note = RNA TARGET SEQUENCE
  source 1..22: mol_type = other RNA
gtgcccagcgacaatcactgaa

SEQ ID NO: 78 - RNA, 22 nt, synthetic construct
  misc_feature 1..22: note = RNA TARGET SEQUENCE
  source 1..22: mol_type = other RNA
gcccagcgacaatcactgaacg

SEQ ID NO: 79 - RNA, 22 nt, synthetic construct
  misc_feature 1..22: note = RNA TARGET SEQUENCE
  source 1..22: mol_type = other RNA
cagcctgcagcgggagaccctg

SEQ ID NO: 80 - RNA, 22 nt, synthetic construct
  misc_feature 1..22: note = RNA TARGET SEQUENCE
  source 1..22: mol_type = other RNA
agcctgcagcgggagaccctgt

SEQ ID NO: 81 - RNA, 22 nt, synthetic construct
  misc_feature 1..22: note = RNA TARGET SEQUENCE
  source 1..22: mol_type = other RNA
ggcgcggacatggaggacgtgc

SEQ ID NO: 82 - RNA, 22 nt, synthetic construct
  misc_feature 1..22: note = RNA TARGET SEQUENCE
  source 1..22: mol_type = other RNA
gcgcggacatggaggacgtgcg

SEQ ID NO: 83 - RNA, 22 nt, synthetic construct
  misc_feature 1..22: note = RNA TARGET SEQUENCE
  source 1..22: mol_type = other RNA
cgcggacatggaggacgtgcgc

SEQ ID NO: 84 - RNA, 22 nt, synthetic construct
  misc_feature 1..22: note = RNA TARGET SEQUENCE
  source 1..22: mol_type = other RNA
gcggacatggaggacgtgcgcg

SEQ ID NO: 85 - RNA, 22 nt, synthetic construct
  misc_feature 1..22: note = RNA TARGET SEQUENCE
  source 1..22: mol_type = other RNA
cggacatggaggacgtgcgcgg

SEQ ID NO: 86 - RNA, 22 nt, synthetic construct
  misc_feature 1..22: note = RNA TARGET SEQUENCE
  source 1..22: mol_type = other RNA
ggacatggaggacgtgcgcggc

SEQ ID NO: 87 - RNA, 22 nt, synthetic construct
  misc_feature 1..22: note = RNA TARGET SEQUENCE
  source 1..22: mol_type = other RNA
gacatggaggacgtgcgcggcc

SEQ ID NO: 88 - RNA, 22 nt, synthetic construct
  misc_feature 1..22: note = RNA TARGET SEQUENCE
  source 1..22: mol_type = other RNA
acatggaggacgtgcgcggccg

SEQ ID NO: 89 - RNA, 22 nt, synthetic construct
  misc_feature 1..22: note = RNA TARGET SEQUENCE
  source 1..22: mol_type = other RNA
catggaggacgtgcgcggccgc

SEQ ID NO: 90 - RNA, 22 nt, synthetic construct
  misc_feature 1..22: note = RNA TARGET SEQUENCE
  source 1..22: mol_type = other RNA
tgcgcggccgcctggtgcagta

SEQ ID NO: 91 - RNA, 22 nt, synthetic construct
  misc_feature 1..22: note = RNA TARGET SEQUENCE
  source 1..22: mol_type = other RNA
gggtggaccctagtttaataaa

SEQ ID NO: 92 - RNA, 22 nt, synthetic construct
  misc_feature 1..22: note = RNA TARGET SEQUENCE
  source 1..22: mol_type = other RNA
ggtggaccctagtttaataaag

SEQ ID NO: 93 - RNA, 22 nt, synthetic construct
  misc_feature 1..22: note = RNA TARGET SEQUENCE
  source 1..22: mol_type = other RNA
gtggaccctagtttaataaaga

SEQ ID NO: 94 - RNA, 22 nt, synthetic construct
  misc_feature 1..22: note = miAPOE_001
  source 1..22: mol_type = other RNA
ttcctgcctgtgattggccagt

SEQ ID NO: 95 - RNA, 22 nt, synthetic construct
  misc_feature 1..22: note = miAPOE_002
  source 1..22: mol_type = other RNA
cttcctgcctgtgattggccag

SEQ ID NO: 96 - RNA, 22 nt, synthetic construct
  misc_feature 1..22: note = miAPOE_003
  source 1..22: mol_type = other RNA
tcttcctgcctgtgattggcca

SEQ ID NO: 97 - RNA, 22 nt, synthetic construct
  misc_feature 1..22: note = miAPOE_004
  source 1..22: mol_type = other RNA
atcttcctgcctgtgattggcc

SEQ ID NO: 98 - RNA, 22 nt, synthetic construct
  misc_feature 1..22: note = miAPOE_005
  source 1..22: mol_type = other RNA
catcttcctgcctgtgattggc

SEQ ID NO: 99 - RNA, 22 nt, synthetic construct
  misc_feature 1..22: note = miAPOE_006
  source 1..22: mol_type = other RNA
tcatcttcctgcctgtgattgg

SEQ ID NO: 100 - RNA, 22 nt, synthetic construct
  misc_feature 1..22: note = miAPOE_007
  source 1..22: mol_type = other RNA
ttcatcttcctgcctgtgattg

SEQ ID NO: 101 - RNA, 22 nt, synthetic construct
  misc_feature 1..22: note = miAPOE_008
  source 1..22: mol_type = other RNA
cttcatcttcctgcctgtgatt

SEQ ID NO: 102 - RNA, 22 nt, synthetic construct
  misc_feature 1..22: note = miAPOE_009
  source 1..22: mol_type = other RNA
ccttcatcttcctgcctgtgat

SEQ ID NO: 103 - RNA, 22 nt, synthetic construct
  misc_feature 1..22: note = miAPOE_010
  source 1..22: mol_type = other RNA
accttcatcttcctgcctgtga

SEQ ID NO: 104 - RNA, 22 nt, synthetic construct
  misc_feature 1..22: note = miAPOE_011
  source 1..22: mol_type = other RNA
aaccttcatcttcctgcctgtg

SEQ ID NO: 105 - RNA, 22 nt, synthetic construct
  misc_feature 1..22: note = miAPOE_013
  source 1..22: mol_type = other RNA
agaaccttcatcttcctgcctg

SEQ ID NO: 106 - RNA, 22 nt, synthetic construct
  misc_feature 1..22: note = miAPOE_014
  source 1..22: mol_type = other RNA
cagaaccttcatcttcctgcct

SEQ ID NO: 107 - RNA, 22 nt, synthetic construct
  misc_feature 1..22: note = miAPOE_015
  source 1..22: mol_type = other RNA
acagaaccttcatcttcctgcc

SEQ ID NO: 108 - RNA, 22 nt, synthetic construct
  misc_feature 1..22: note = miAPOE_016
  source 1..22: mol_type = other RNA
cacagaaccttcatcttcctgc

SEQ ID NO: 109 - RNA, 22 nt, synthetic construct
  misc_feature 1..22: note = miAPOE_017
  source 1..22: mol_type = other RNA
ccacagaaccttcatcttcctg

SEQ ID NO: 110 - RNA, 22 nt, synthetic construct
  misc_feature 1..22: note = miAPOE_018
  source 1..22: mol_type = other RNA
cccacagaaccttcatcttcct

SEQ ID NO: 111 - RNA, 22 nt, synthetic construct
  misc_feature 1..22: note = miAPOE_020
  source 1..22: mol_type = other RNA
agcccacagaaccttcatcttc

SEQ ID NO: 112 - RNA, 22 nt, synthetic construct
  misc_feature 1..22: note = miAPOE_021
  source 1..22: mol_type = other RNA
cagcccacagaaccttcatctt

SEQ ID NO: 113 - RNA, 22 nt, synthetic construct
  misc_feature 1..22: note = miAPOE_024
  source 1..22: mol_type = other RNA
acgcagcccacagaaccttcat

SEQ ID NO: 114 - RNA, 22 nt, synthetic construct
  misc_feature 1..22: note = miAPOE_025
  source 1..22: mol_type = other RNA
aacgcagcccacagaaccttca

SEQ ID NO: 115 - RNA, 22 nt, synthetic construct
  misc_feature 1..22: note = miAPOE_036
  source 1..22: mol_type = other RNA
atgtgaccagcaacgcagccca

SEQ ID NO: 116 - RNA, 22 nt, synthetic construct
  misc_feature 1..22: note = miAPOE_037
  source 1..22: mol_type = other RNA
aatgtgaccagcaacgcagccc

SEQ ID NO: 117 - RNA, 22 nt, synthetic construct
  misc_feature 1..22: note = miAPOE_040
  source 1..22: mol_type = other RNA
aggaatgtgaccagcaacgcag

SEQ ID NO: 118 - RNA, 22 nt, synthetic construct
  misc_feature 1..22: note = miAPOE_042
  source 1..22: mol_type = other RNA
ccaggaatgtgaccagcaacgc

SEQ ID NO: 119 - RNA, 22 nt, synthetic construct
  misc_feature 1..22: note = miAPOE_045
  source 1..22: mol_type = other RNA
ctgccaggaatgtgaccagcaa

SEQ ID NO: 120 - RNA, 22 nt, synthetic construct
  misc_feature 1..22: note = miAPOE_053
  source 1..22: mol_type = other RNA
ctggcatcctgccaggaatgtg

SEQ ID NO: 121 - RNA, 22 nt, synthetic construct
  misc_feature 1..22: note = miAPOE_085
  source 1..22: mol_type = other RNA
acccagcgcaggtaatcccaaa

SEQ ID NO: 122 - RNA, 22 nt, synthetic construct
  misc_feature 1..22: note = miAPOE_100
  source 1..22: mol_type = other RNA
tcagacagtgtctgcacccagc

SEQ ID NO: 123 - RNA, 22 nt, synthetic construct
  misc_feature 1..22: note = miAPOE_101
  source 1..22: mol_type = other RNA
ctcagacagtgtctgcacccag

SEQ ID NO: 124 - RNA, 22 nt, synthetic construct
  misc_feature 1..22: note = miAPOE_104
  source 1..22: mol_type = other RNA
ctgctcagacagtgtctgcacc

SEQ ID NO: 125 - RNA, 22 nt, synthetic construct
  misc_feature 1..22: note = miAPOE_105
  source 1..22: mol_type = other RNA
cctgctcagacagtgtctgcac

SEQ ID NO: 126 - RNA, 22 nt, synthetic construct
  misc_feature 1..22: note = miAPOE_106
  source 1..22: mol_type = other RNA
acctgctcagacagtgtctgca

SEQ ID NO: 127 - RNA, 22 nt, synthetic construct
  misc_feature 1..22: note = miAPOE_107
  source 1..22: mol_type = other RNA
cacctgctcagacagtgtctgc

SEQ ID NO: 128 - RNA, 22 nt, synthetic construct
  misc_feature 1..22: note = miAPOE_109
  source 1..22: mol_type = other RNA
tgcacctgctcagacagtgtct

SEQ ID NO: 129 - RNA, 22 nt, synthetic construct
  misc_feature 1..22: note = miAPOE_110
  source 1..22: mol_type = other RNA
ctgcacctgctcagacagtgtc

SEQ ID NO: 130 - RNA, 22 nt, synthetic construct
  misc_feature 1..22: note = miAPOE_111
  source 1..22: mol_type = other RNA
cctgcacctgctcagacagtgt

SEQ ID NO: 131 - RNA, 22 nt, synthetic construct
  misc_feature 1..22: note = miAPOE_126
  source 1..22: mol_type = other RNA
ccttcaactccttcatggtctc

SEQ ID NO: 132 - RNA, 22 nt, synthetic construct
  misc_feature 1..22: note = miAPOE_129
  source 1..22: mol_type = other RNA
aggccttcaactccttcatggt

SEQ ID NO: 133 - RNA, 22 nt, synthetic construct
  misc_feature 1..22: note = miAPOE_130
  source 1..22: mol_type = other RNA
taggccttcaactccttcatgg

SEQ ID NO: 134 - RNA, 22 nt, synthetic construct
  misc_feature 1..22: note = miAPOE_132
  source 1..22: mol_type = other RNA
tgtaggccttcaactccttcat

SEQ ID NO: 135 - RNA, 22 nt, synthetic construct
  misc_feature 1..22: note = miAPOE_133
  source 1..22: mol_type = other RNA
ttgtaggccttcaactccttca

SEQ ID NO: 136 - RNA, 22 nt, synthetic construct
  misc_feature 1..22: note = miAPOE_134
  source 1..22: mol_type = other RNA
tttgtaggccttcaactccttc

SEQ ID NO: 137 - RNA, 22 nt, synthetic construct
  misc_feature 1..22: note = miAPOE_135
  source 1..22: mol_type = other RNA
atttgtaggccttcaactcctt

SEQ ID NO: 138 - RNA, 22 nt, synthetic construct
  misc_feature 1..22: note = miAPOE_136
  source 1..22: mol_type = other RNA
gatttgtaggccttcaactcct

SEQ ID NO: 139 - RNA, 22 nt, synthetic construct
  misc_feature 1..22: note = miAPOE_137
  source 1..22: mol_type = other RNA
cgatttgtaggccttcaactcc

SEQ ID NO: 140 - RNA, 22 nt, synthetic construct
  misc_feature 1..22: note = miAPOE_138
  source 1..22: mol_type = other RNA
ccgatttgtaggccttcaactc

SEQ ID NO: 141 - RNA, 22 nt, synthetic construct
  misc_feature 1..22: note = miAPOE_139
  source 1..22: mol_type = other RNA
tccgatttgtaggccttcaact

SEQ ID NO: 142 - RNA, 22 nt, synthetic construct
  misc_feature 1..22: note = miAPOE_140
  source 1..22: mol_type = other RNA
ttccgatttgtaggccttcaac

SEQ ID NO: 143 - RNA, 22 nt, synthetic construct
  misc_feature 1..22: note = miAPOE_142
  source 1..22: mol_type = other RNA
agttccgatttgtaggccttca

SEQ ID NO: 144 - RNA, 22 nt, synthetic construct
  misc_feature 1..22: note = miAPOE_143
  source 1..22: mol_type = other RNA
cagttccgatttgtaggccttc

SEQ ID NO: 145 - RNA, 22 nt, synthetic construct
  misc_feature 1..22: note = miAPOE_144
  source 1..22: mol_type = other RNA
ccagttccgatttgtaggcctt

SEQ ID NO: 146 - RNA, 22 nt, synthetic construct
  misc_feature 1..22: note = miAPOE_145
  source 1..22: mol_type = other RNA
tccagttccgatttgtaggcct

SEQ ID NO: 147 - RNA, 22 nt, synthetic construct
  misc_feature 1..22: note = miAPOE_146
  source 1..22: mol_type = other RNA
ctccagttccgatttgtaggcc

SEQ ID NO: 148 - RNA, 22 nt, synthetic construct
  misc_feature 1..22: note = miAPOE_147
  source 1..22: mol_type = other RNA
cctccagttccgatttgtaggc

SEQ ID NO: 149 - RNA, 22 nt, synthetic construct
  misc_feature 1..22: note = miAPOE_148
  source 1..22: mol_type = other RNA
tcctccagttccgatttgtagg

SEQ ID NO: 150 - RNA, 22 nt, synthetic construct
  misc_feature 1..22: note = miAPOE_149
  source 1..22: mol_type = other RNA
ttcctccagttccgatttgtag

SEQ ID NO: 151 - RNA, 22 nt, synthetic construct
  misc_feature 1..22: note = miAPOE_151
  source 1..22: mol_type = other RNA
tgttcctccagttccgatttgt

SEQ ID NO: 152 - RNA, 22 nt, synthetic construct
  misc_feature 1..22: note = miAPOE_175
  source 1..22: mol_type = other RNA
tgcagctccttggacagccgtg

SEQ ID NO: 153 - RNA, 22 nt, synthetic construct
  misc_feature 1..22: note = miAPOE_176
  source 1..22: mol_type = other RNA
ctgcagctccttggacagccgt

SEQ ID NO: 154 - RNA, 22 nt, synthetic construct
  misc_feature 1..22: note = miAPOE_202
  source 1..22: mol_type = other RNA
agcttgcgcaggtgggaggcga

SEQ ID NO: 155 - RNA, 22 nt, synthetic construct
  misc_feature 1..22: note = miAPOE_206
  source 1..22: mol_type = other RNA
ccaggcgcttctgcaggtcatc

SEQ ID NO: 156 - RNA, 22 nt, synthetic construct
  misc_feature 1..22: note = miAPOE_209
  source 1..22: mol_type = other RNA
ctgccaggcgcttctgcaggtc

SEQ ID NO: 157 - RNA, 22 nt, synthetic construct
  misc_feature 1..22: note = miAPOE_210
  source 1..22: mol_type = other RNA
actgccaggcgcttctgcaggt

SEQ ID NO: 158 - RNA, 22 nt, synthetic construct
  misc_feature 1..22: note = miAPOE_212
  source 1..22: mol_type = other RNA
acactgccaggcgcttctgcag

SEQ ID NO: 159 - RNA, 22 nt, synthetic construct
  misc_feature 1..22: note = miAPOE_213
  source 1..22: mol_type = other RNA
tacactgccaggcgcttctgca

SEQ ID NO: 160 - RNA, 22 nt, synthetic construct
  misc_feature 1..22: note = miAPOE_278
  source 1..22: mol_type = other RNA
tccttcacctcgtccaggcggt

SEQ ID NO: 161 - RNA, 22 nt, synthetic construct
  misc_feature 1..22: note = miAPOE_283
  source 1..22: mol_type = other RNA
cctgctccttcacctcgtccag

SEQ ID NO: 162 - RNA, 22 nt, synthetic construct
  misc_feature 1..22: note = miAPOE_286
  source 1..22: mol_type = other RNA
ccacctgctccttcacctcgtc

SEQ ID NO: 163 - RNA, 22 nt, synthetic construct
  misc_feature 1..22: note = miAPOE_336
  source 1..22: mol_type = other RNA
tcgaaccagctcttgaggcggg

SEQ ID NO: 164 - RNA, 22 nt, synthetic construct
  misc_feature 1..22: note = miAPOE_337
  source 1..22: mol_type = other RNA
ctcgaaccagctcttgaggcgg

SEQ ID NO: 165 - RNA, 22 nt, synthetic construct
  misc_feature 1..22: note = miAPOE_345
  source 1..22: mol_type = other RNA
agcctgcaccttctccaccagc

SEQ ID NO: 166 - RNA, 22 nt, synthetic construct
  misc_feature 1..22: note = miAPOE_350
  source 1..22: mol_type = other RNA
acggcagcctgcaccttctcca

SEQ ID NO: 167 - RNA, 22 nt, synthetic construct
  misc_feature 1..22: note = miAPOE_360
  source 1..22: mol_type = other RNA
agtgattgtcgctgggcacagg

SEQ ID NO: 168 - RNA, 22 nt, synthetic construct
  misc_feature 1..22: note = miAPOE_362
  source 1..22: mol_type = other RNA
tcagtgattgtcgctgggcaca

SEQ ID NO: 169 - RNA, 22 nt, synthetic construct
  misc_feature 1..22: note = miAPOE_363
  source 1..22: mol_type = other RNA
ttcagtgattgtcgctgggcac

SEQ ID NO: 170 - RNA, 22 nt, synthetic construct
  misc_feature 1..22: note = miAPOE_365
  source 1..22: mol_type = other RNA
cgttcagtgattgtcgctgggc

SEQ ID NO: 171 - RNA, 22 nt, synthetic construct
  misc_feature 1..22: note = miAPOE_368
  source 1..22: mol_type = other RNA
cagggtctcccgctgcaggctg

SEQ ID NO: 172 - RNA, 22 nt, synthetic construct
  misc_feature 1..22: note = miAPOE_369
  source 1..22: mol_type = other RNA
acagggtctcccgctgcaggct

SEQ ID NO: 173 - RNA, 22 nt, synthetic construct
  misc_feature 1..22: note = miAPOE_391
  source 1..22: mol_type = other RNA
ggcgcggacatggaggacgtgc

SEQ ID NO: 174 - RNA, 22 nt, synthetic construct
  misc_feature 1..22: note = miAPOE_392
  source 1..22: mol_type = other RNA
gcgcggacatggaggacgtgcg

SEQ ID NO: 175 - RNA, 22 nt, synthetic construct
  misc_feature 1..22: note = miAPOE_393
  source 1..22: mol_type = other RNA
cgcggacatggaggacgtgcgc

SEQ ID NO: 176 - RNA, 22 nt, synthetic construct
  misc_feature 1..22: note = miAPOE_394
  source 1..22: mol_type = other RNA
gcggacatggaggacgtgcgcg

SEQ ID NO: 177 - RNA, 22 nt, synthetic construct
  misc_feature 1..22: note = miAPOE_395
  source 1..22: mol_type = other RNA
cggacatggaggacgtgcgcgg

SEQ ID NO: 178 - RNA, 22 nt, synthetic construct
  misc_feature 1..22: note = miAPOE_396
  source 1..22: mol_type = other RNA
ggacatggaggacgtgcgcggc

SEQ ID NO: 179 - RNA, 22 nt, synthetic construct
  misc_feature 1..22: note = miAPOE_397
  source 1..22: mol_type = other RNA
ggccgcgcacgtcctccatgtc

SEQ ID NO: 180 - RNA, 22 nt, synthetic construct
  misc_feature 1..22: note = miAPOE_398
  source 1..22: mol_type = other RNA
cggccgcgcacgtcctccatgt

SEQ ID NO: 181 - RNA, 22 nt, synthetic construct
  misc_feature 1..22: note = miAPOE_399
  source 1..22: mol_type = other RNA
gcggccgcgcacgtcctccatg

SEQ ID NO: 182 - RNA, 22 nt, synthetic construct
  misc_feature 1..22: note = miAPOE_410
  source 1..22: mol_type = other RNA
tactgcaccaggcggccgcgca

SEQ ID NO: 183 - RNA, 22 nt, synthetic construct
  misc_feature 1..22: note = miAPOE_417
  source 1..22: mol_type = other RNA
tttattaaactagggtccaccc

SEQ ID NO: 184 - RNA, 22 nt, synthetic construct
  misc_feature 1..22: note = miAPOE_418
  source 1..22: mol_type = other RNA
ctttattaaactagggtccacc

SEQ ID NO: 185 - RNA, 22 nt, synthetic construct
  misc_feature 1..22: note = miAPOE_419
  source 1..22: mol_type = other RNA
tctttattaaactagggtccac

SEQ ID NO: 186 - RNA, 22 nt, synthetic construct
  misc_feature 1..22: note = miAPOE_SCR_441
  source 1..22: mol_type = other RNA
atcgcgttctcgctgttcgact

SEQ ID NO: 187 - RNA, 22 nt, synthetic construct
  misc_feature 1..22: note = miAPOE_SCR_443
  source 1..22: mol_type = other RNA
accttcgtcttacttgagttac

SEQ ID NO: 188 - RNA, 22 nt, synthetic construct
  misc_feature 1..22: note = miAPOE_SCR_455
  source 1..22: mol_type = other RNA
agtgcgcttctcctactcgcgc

SEQ ID NO: 189 - RNA, 22 nt, synthetic construct
  misc_feature 1..22: note = miAPOE_SCR_468
  source 1..22: mol_type = other RNA
agcgcactccgtcgtcgcctcc

SEQ ID NO: 190 - RNA, 66 nt, synthetic construct
  misc_feature 1..66: note = pre-miR-451
  source 1..66: mol_type = other RNA
cttgggaatggcaaggaaaccgttaccattactgagttcagtaatggtaacggttctcttgctata

SEQ ID NO: 191 - DNA, 1662 bp, synthetic construct
  misc_feature 1..1662: note = CAG promoter
  source 1..1662: mol_type = other DNA
tgtaggaaaaagaagaaggcatgaacatggttagcagaggctctagagccgccggtcacacgccagaagccgaaccccgccctgccccgtcccccccgaaggcagccgtccccccgcggacagccccgaggctggagagggagaaggggacggcggcgcggcgacgcacgaaggccctccccgcccatttccttcctgccggcgccgcaccgcttcgccccgcgcccgctagagggggtgcggcggcgcctcccagatttcggctccgcacagatttgggacaaaggaagtccctgcgccctctcgcacgattaccataaaaggcaatggctgcggcgcgccgcgcctcgacagccgccggcgctccgggggccgccgcgcccctcccccgagccctccccggcccgaggcggccccgccccgcccggcacccccacctgccgccaccccccgcccggcacggcgagccccgcgccacgccccgtacggagccccgcacccgaagccgggccgtgctcagcaactcggggaggggggtgcaggggggggttacagcccgaccgccgcgcccacaccccctgctcacccccccacgcacacaccccgcacgcagcctttgttcccctcgcagccccccccgcaccgcggggcaccgcccccggccgcgctcccctcgcgcacactgcggagcgcacaaagccccgcgccgcgcccgcagcgctcacagccgccgggcagcgcgggccgcacgcggcgctccccacgcacacacacacgcacgcaccccccgagccgctcccccccgcacaaagggccctcccggagcccctcaaggctttcacgcagccacagaaaagaaacaagccgtcattaaaccaagcgctaattacagcccggaggagaagggccgtcccgcccgctcacttgtgggagtaacgcggtcagtcagagccggggcgggcggcgcgaggcggcggcggagcggggcacggggcgaaggcagcgtcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactag

SEQ ID NO: 192 - DNA, 798 bp, synthetic construct
  misc_feature 1..798: note = P1 promoter
  source 1..798: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgg

SEQ ID NO: 193 - DNA, 877 bp, synthetic construct
  misc_feature 1..877: note = P2 promoter
  source 1..877: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgg

SEQ ID NO: 194 - DNA, 133 bp, synthetic construct
  misc_feature 1..133: note = SV40 polyA
  source 1..133: mol_type = other DNA
tgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 195 - DNA, 954 bp, synthetic construct
  misc_feature 1..954: note = APOE2 WT
  source 1..954: mol_type = other DNA
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 196 - DNA, 954 bp, synthetic construct
  misc_feature 1..954: note = APOE3 WT
  source 1..954: mol_type = other DNA
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 197 - DNA, 954 bp, synthetic construct
  misc_feature 1..954: note = APOE2 ch-wt
  source 1..954: mol_type = other DNA
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgagcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 198 - DNA, 954 bp, synthetic construct
  misc_feature 1..954: note = APOE3 ch-WT
  source 1..954: mol_type = other DNA
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgagcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 199 - DNA, 951 bp, synthetic construct
  misc_feature 1..951: note = APOE3 ch-HA-WT
  source 1..951: mol_type = other DNA
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgtggccgcctggtgcagtaccgcggcgaggtgcaggccatgctcggccagagcaccgaggagctgcgggtgagcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcac

SEQ ID NO: 200 - DNA, 954 bp, synthetic construct
  misc_feature 1..954: note = APOE2b - APOE2-Jac (V236E) -WT
  source 1..954: mol_type = other DNA
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggaggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 201 - DNA, 954 bp, synthetic construct
  misc_feature 1..954: note = APOE3b - APOE3-Jac (V236E) -WT
  source 1..954: mol_type = other DNA
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggaggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 202 - DNA, 954 bp, synthetic construct
  misc_feature 1..954: note = APOE2-V1
  source 1..954: mol_type = other DNA
atgaaggtgctgtgggccgctctgctggtcacatttctggccggctgtcaggccaaagtggaacaggccgtggaaaccgagcctgagcctgaactgagacagcagacagagtggcagtccggccagagatgggaacttgccctgggcagattctgggactacctgagatgggtgcagaccctgagcgaacaggtgcaagaggaactgctgagcagccaagtgacccaagagctgagagccctgatggacgagacaatgaaggaactgaaggcctacaagagcgagctggaagaacagctgacccctgtggccgaggaaacaagagccagactgagcaaagagctgcaggccgctcaagctagactgggcgccgatatggaagatgtgtgcggcagactggtgcagtacagaggcgaagtgcaggccatgctgggccagtctaccgaggaactgagagtgcggctggcctctcacctgagaaagctgaggaagagactgctgagggacgccgacgacctgcagaaatgtctggccgtgtatcaggcaggcgccagagaaggtgcagagagaggactgtctgccatcagagaaagactgggccctctggtcgagcagggcagagttagagctgccacagtgggaagtctggcaggccagcctctgcaagaaagagcacaggcctggggcgagagactgagggccagaatggaagagatgggcagcagaacccgggacagactggacgaagtgaaagaacaggtggccgaagtgcgcgccaagctcgaagaacaagcccagcagattcggctgcaggctgaagcctttcaggcccggctgaagagttggttcgagcccctggtggaagatatgcagagacagtgggctggactggtggaaaaggtgcaggctgccgtgggaacatctgctgctccagtgcctagcgacaaccactga

SEQ ID NO: 203 - DNA, 954 bp, synthetic construct
  misc_feature 1..954: note = APOE2-V1
  source 1..954: mol_type = other DNA
atgaaggtgctgtgggccgctctgctggtcacatttctggccggctgtcaggccaaagtggaacaggccgtggaaaccgagcctgagcctgaactgagacagcagacagagtggcagtccggccagagatgggaacttgccctgggcagattctgggactacctgagatgggtgcagaccctgagcgaacaggtgcaagaggaactgctgagcagccaagtgacccaagagctgagagccctgatggacgagacaatgaaggaactgaaggcctacaagagcgagctggaagaacagctgacccctgtggccgaggaaacaagagccagactgagcaaagagctgcaggccgctcaagctagactgggcgccgatatggaagatgtgtgcggcagactggtgcagtacagaggcgaagtgcaggccatgctgggccagtctaccgaggaactgagagtgcggctggcctctcacctgagaaagctgaggaagagactgctgagggacgccgacgacctgcagaaaagactggccgtgtatcaggcaggcgccagagaaggtgcagagagaggactgtctgccatcagagaaaggctgggccctctggttgagcagggcagagttagagctgccacagtgggaagtctggcaggccagcctctgcaagaaagagcacaggcctggggcgagagactgagggccagaatggaagagatgggcagcagaacccgggacagactggacgaagtgaaagaacaggtggccgaagtgcgcgccaagctcgaagaacaagcccagcagattcggctgcaggctgaagcctttcaggcccggctgaagagttggttcgagcccctggtggaagatatgcagagacagtgggctggactggtggaaaaggtgcaggctgccgtgggaacatctgctgctccagtgcctagcgacaaccactga

SEQ ID NO: 204 - DNA, 954 bp, synthetic construct
  misc_feature 1..954: note = APOE2 ch-V1
  source 1..954: mol_type = other DNA
atgaaggtgctgtgggccgctctgctggtcacatttctggccggctgtcaggccaaagtggaacaggccgtggaaaccgagcctgagcctgaactgagacagcagacagagtggcagtccggccagagatgggaacttgccctgggcagattctgggactacctgagatgggtgcagaccctgagcgaacaggtgcaagaggaactgctgagcagccaagtgacccaagagctgagagccctgatggacgagacaatgaaggaactgaaggcctacaagagcgagctggaagaacagctgacccctgtggccgaggaaacaagagccagactgagcaaagagctgcaggccgctcaagctagactgggcgccgatatggaagatgtgtgcggcagactggtgcagtacagaggcgaagtgcaggccatgctgggccagtctaccgaggaactgagagtgtctctggccagccacctgagaaagctgcggaagagactgctgagagatgccgacgacctgcagaaatgcctggccgtgtatcaggctggcgctagagaaggcgctgagagaggactgtctgccatccgggaaagactgggccctctggttgaacagggcagagttagagccgccacagtgggatctcttgcaggccagcctctgcaagaaagagcccaggcttggggcgagagactgagggccagaatggaagagatgggcagcagaaccagagacagactggacgaagtgaaagaacaggtggccgaagtgcgcgccaagctcgaagaacaagcccagcagattcggctgcaggctgaagcctttcaggcccggctgaagagttggttcgagcccctggtggaagatatgcagagacagtgggctggactggtggaaaaggtgcaggctgccgtgggaacatctgctgctccagtgcctagcgacaaccactga

SEQ ID NO: 205 - DNA, 954 bp, synthetic construct
  misc_feature 1..954: note = APOE3 ch-V1
  source 1..954: mol_type = other DNA
atgaaggtgctgtgggccgctctgctggtcacatttctggccggctgtcaggccaaagtggaacaggccgtggaaaccgagcctgagcctgaactgagacagcagacagagtggcagtccggccagagatgggaacttgccctgggcagattctgggactacctgagatgggtgcagaccctgagcgaacaggtgcaagaggaactgctgagcagccaagtgacccaagagctgagagccctgatggacgagacaatgaaggaactgaaggcctacaagagcgagctggaagaacagctgacccctgtggccgaggaaacaagagccagactgagcaaagagctgcaggccgctcaagctagactgggcgccgatatggaagatgtgtgcggcagactggtgcagtacagaggcgaagtgcaggccatgctgggccagtctaccgaggaactgagagtgtctctggccagccacctgagaaagctgcggaagagactgctgagagatgccgacgacctgcagaaaagactggccgtgtatcaggcaggcgccagagaaggtgcagagagaggactgtctgccatcagagaaaggctgggccctctggttgagcagggcagagttagagctgccacagtgggaagtctggcaggccagcctctgcaagaaagagcacaggcctggggcgagagactgagggccagaatggaagagatgggcagcagaacccgggacagactggacgaagtgaaagaacaggtggccgaagtgcgcgccaagctcgaagaacaagcccagcagattcggctgcaggctgaagcctttcaggcccggctgaagagttggttcgagcccctggtggaagatatgcagagacagtgggctggactggtggaaaaggtgcaggctgccgtgggaacatctgctgctccagtgcctagcgacaaccactga

SEQ ID NO: 206 - DNA, 954 bp, synthetic construct
  misc_feature 1..954: note = APOE2-V2
  source 1..954: mol_type = other DNA
atgaaagtcctctgggccgctctcctggtgacctttctggccggctgtcaggccaaagtggaacaggccgtggaaaccgagcctgagcctgaactgagacagcagacagagtggcagtccggccagagatgggaacttgccctgggcagattctgggactacctgagatgggtgcagaccctgagcgaacaggtgcaagaggaactgctgagcagccaagtgacccaagagctgagagccctgatggacgagacaatgaaagaactcaaagcttataagagcgagctcgaagagcagctgacccctgtggccgaggaaacaagagccagactgagcaaagagctgcaggccgctcaagctagactgggcgccgatatggaagatgtgtgcggcagactggtgcagtacagaggcgaagtgcaggccatgctgggccagtctaccgaggaactgagagtgcggctggccagccacctgagaaagctgcggaagagactgctgagagatgccgacgacctgcagaaatgcctggccgtgtatcaggctggcgctagagaaggcgctgagagaggactgtctgccatccgggaaagactgggccctctggttgaacagggcagagttagagccgccacagtgggatctcttgcaggccagcctctgcaagaaagagcccaggcttggggcgagagactgagggccagaatggaagagatgggcagcagaaccagagacagactggacgaagtgaaagaacaggtggccgaagtgcgcgccaagctcgaagaacaagcccagcagattcggctgcaggctgaagcctttcaggcccggctgaagagttggttcgagcccctggtggaagatatgcagagacagtgggctggactggtggaaaaggtgcaggctgccgtgggaacatctgctgctccagtgcctagcgacaaccactga

SEQ ID NO: 207 - DNA, 954 bp, synthetic construct
  misc_feature 1..954: note = APOE3-V2
  source 1..954: mol_type = other DNA
atgaaagtcctctgggccgctctcctggtgacctttctggccggctgtcaggccaaagtggaacaggccgtggaaaccgagcctgagcctgaactgagacagcagacagagtggcagtccggccagagatgggaacttgccctgggcagattctgggactacctgagatgggtgcagaccctgagcgaacaggtgcaagaggaactgctgagcagccaagtgacccaagagctgagagccctgatggacgagacaatgaaagaactcaaagcttataagagcgagctcgaagagcagctgacccctgtggccgaggaaacaagagccagactgagcaaagagctgcaggccgctcaagctagactgggcgccgatatggaagatgtgtgcggcagactggtgcagtacagaggcgaagtgcaggccatgctgggccagtctaccgaggaactgagagtgcggctggccagccacctgagaaagctgcggaagagactgctgagagatgccgacgacctgcagaaacgcctggccgtgtatcaggctggcgctagagaaggcgctgagagaggactgtctgccatccgggaaagactgggccctctggttgaacagggcagagttagagccgccacagtgggatctcttgcaggccagcctctgcaagaaagagcccaggcttggggcgagagactgagggccagaatggaagagatgggcagcagaaccagagacagactggacgaagtgaaagaacaggtggccgaagtgcgcgccaagctcgaagaacaagcccagcagattcggctgcaggctgaagcctttcaggcccggctgaagagttggttcgagcccctggtggaagatatgcagagacagtgggctggactggtggaaaaggtgcaggctgccgtgggaacatctgctgctccagtgcctagcgacaaccactga

SEQ ID NO: 208 - DNA, 954 bp, synthetic construct
  misc_feature 1..954: note = APOE2 ch-V2
  source 1..954: mol_type = other DNA
atgaaagtcctctgggccgctctcctggtgacctttctggccggctgtcaggccaaagtggaacaggccgtggaaaccgagcctgagcctgaactgagacagcagacagagtggcagtccggccagagatgggaacttgccctgggcagattctgggactacctgagatgggtgcagaccctgagcgaacaggtgcaagaggaactgctgagcagccaagtgacccaagagctgagagccctgatggacgagacaatgaaagaactcaaagcttataagagcgagctcgaagagcagctgacccctgtggccgaggaaacaagagccagactgagcaaagagctgcaggccgctcaagctagactgggcgccgatatggaagatgtgtgcggcagactggtgcagtacagaggcgaagtgcaggccatgctgggccagtctaccgaggaactgagagtgtctctggccagccacctgagaaagctgcggaagagactgctgagagatgccgacgacctgcagaaatgcctggccgtgtatcaggctggcgctagagaaggcgctgagagaggactgtctgccatccgggaaagactgggccctctggttgaacagggcagagttagagccgccacagtgggatctcttgcaggccagcctctgcaagaaagagcccaggcttggggcgagagactgagggccagaatggaagagatgggcagcagaaccagagacagactggacgaagtgaaagaacaggtggccgaagtgcgcgccaagctcgaagaacaagcccagcagattcggctgcaggctgaagcctttcaggcccggctgaagagttggttcgagcccctggtggaagatatgcagagacagtgggctggactggtggaaaaggtgcaggctgccgtgggaacatctgctgctccagtgcctagcgacaaccactga

SEQ ID NO: 209 - DNA, 954 bp, synthetic construct
  misc_feature 1..954: note = APOE3 ch-V2
  source 1..954: mol_type = other DNA
atgaaagtcctctgggccgctctcctggtgacctttctggccggctgtcaggccaaagtggaacaggccgtggaaaccgagcctgagcctgaactgagacagcagacagagtggcagtccggccagagatgggaacttgccctgggcagattctgggactacctgagatgggtgcagaccctgagcgaacaggtgcaagaggaactgctgagcagccaagtgacccaagagctgagagccctgatggacgagacaatgaaagaactcaaagcttataagagcgagctcgaagagcagctgacccctgtggccgaggaaacaagagccagactgagcaaagagctgcaggccgctcaagctagactgggcgccgatatggaagatgtgtgcggcagactggtgcagtacagaggcgaagtgcaggccatgctgggccagtctaccgaggaactgagagtgtctctggccagccacctgagaaagctgcggaagagactgctgagagatgccgacgacctgcagaaacgcctggccgtgtatcaggctggcgctagagaaggcgctgagagaggactgtctgccatccgggaaagactgggccctctggttgaacagggcagagttagagccgccacagtgggatctcttgcaggccagcctctgcaagaaagagcccaggcttggggcgagagactgagggccagaatggaagagatgggcagcagaaccagagacagactggacgaagtgaaagaacaggtggccgaagtgcgcgccaagctcgaagaacaagcccagcagattcggctgcaggctgaagcctttcaggcccggctgaagagttggttcgagcccctggtggaagatatgcagagacagtgggctggactggtggaaaaggtgcaggctgccgtgggaacatctgctgctccagtgcctagcgacaaccactga

SEQ ID NO: 210 - DNA, 954 bp, synthetic construct
  misc_feature 1..954: note = APOE2b - APOE2-Jac (V236E)-V2
  source 1..954: mol_type = other DNA
atgaaagtcctctgggccgctctcctggtgacctttctggccggctgtcaggccaaagtggaacaggccgtggaaaccgagcctgagcctgaactgagacagcagacagagtggcagtccggccagagatgggaacttgccctgggcagattctgggactacctgagatgggtgcagaccctgagcgaacaggtgcaagaggaactgctgagcagccaagtgacccaagagctgagagccctgatggacgagacaatgaaagaactcaaagcttataagagcgagctcgaagagcagctgacccctgtggccgaggaaacaagagccagactgagcaaagagctgcaggccgctcaagctagactgggcgccgatatggaagatgtgtgcggcagactggtgcagtacagaggcgaagtgcaggccatgctgggccagtctaccgaggaactgagagtgcggctggccagccacctgagaaagctgcggaagagactgctgagagatgccgacgacctgcagaaatgcctggccgtgtatcaggctggcgctagagaaggcgctgagagaggactgtctgccatccgggaaagactgggccctctggttgaacagggcagagttagagccgccacagtgggatctcttgcaggccagcctctgcaagaaagagcccaggcttggggcgagagactgagggccagaatggaagagatgggcagcagaaccagagacagactggacgaagtgaaagaacaggaggccgaagtgcgcgccaagctcgaagaacaagcccagcagattcggctgcaggctgaagcctttcaggcccggctgaagagttggttcgagcccctggtggaagatatgcagagacagtgggctggactggtggaaaaggtgcaggctgccgtgggaacatctgctgctccagtgcctagcgacaaccactga

SEQ ID NO: 211 - DNA, 954 bp, synthetic construct
  misc_feature 1..954: note = APOE3b - APOE3-Jac (V236E) -V2
  source 1..954: mol_type = other DNA
atgaaagtcctctgggccgctctcctggtgacctttctggccggctgtcaggccaaagtggaacaggccgtggaaaccgagcctgagcctgaactgagacagcagacagagtggcagtccggccagagatgggaacttgccctgggcagattctgggactacctgagatgggtgcagaccctgagcgaacaggtgcaagaggaactgctgagcagccaagtgacccaagagctgagagccctgatggacgagacaatgaaagaactcaaagcttataagagcgagctcgaagagcagctgacccctgtggccgaggaaacaagagccagactgagcaaagagctgcaggccgctcaagctagactgggcgccgatatggaagatgtgtgcggcagactggtgcagtacagaggcgaagtgcaggccatgctgggccagtctaccgaggaactgagagtgcggctggccagccacctgagaaagctgcggaagagactgctgagagatgccgacgacctgcagaaacgcctggccgtgtatcaggctggcgctagagaaggcgctgagagaggactgtctgccatccgggaaagactgggccctctggttgaacagggcagagttagagccgccacagtgggatctcttgcaggccagcctctgcaagaaagagcccaggcttggggcgagagactgagggccagaatggaagagatgggcagcagaaccagagacagactggacgaagtgaaagaacaggaggccgaagtgcgcgccaagctcgaagaacaagcccagcagattcggctgcaggctgaagcctttcaggcccggctgaagagttggttcgagcccctggtggaagatatgcagagacagtgggctggactggtggaaaaggtgcaggctgccgtgggaacatctgctgctccagtgcctagcgacaaccactga

SEQ ID NO: 212 - DNA, 954 bp, synthetic construct
  misc_feature 1..954: note = APOE2-V3
  source 1..954: mol_type = other DNA
atgaaagtcctctgggccgctctcctggtgacctttctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaagaactcaaagcttataagagcgagctcgaagagcagctgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 213 - DNA, 954 bp, synthetic construct
  misc_feature 1..954: note = APOE3-V3
  source 1..954: mol_type = other DNA
atgaaagtcctctgggccgctctcctggtgacctttctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaagaactcaaagcttataagagcgagctcgaagagcagctgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 214 - DNA, 954 bp, synthetic construct
  misc_feature 1..954: note = APOE2 ch-V3
  source 1..954: mol_type = other DNA
atgaaagtcctctgggccgctctcctggtgacctttctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaagaactcaaagcttataagagcgagctcgaagagcagctgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgagcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 215 - DNA, 954 bp, synthetic construct
  misc_feature 1..954: note = APOE3 ch-V3
  source 1..954: mol_type = other DNA
atgaaagtcctctgggccgctctcctggtgacctttctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaagaactcaaagcttataagagcgagctcgaagagcagctgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgagcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 216 - DNA, 954 bp, synthetic construct
  misc_feature 1..954: note = APOE2b - APOE2-Jac (V236E) -V3
  source 1..954: mol_type = other DNA
atgaaagtcctctgggccgctctcctggtgacctttctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaagaactcaaagcttataagagcgagctcgaagagcagctgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggaggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 217 - DNA, 954 bp, synthetic construct
  misc_feature 1..954: note = APOE3b - APOE3-Jac (V236E) -V3
  source 1..954: mol_type = other DNA
atgaaagtcctctgggccgctctcctggtgacctttctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaagaactcaaagcttataagagcgagctcgaagagcagctgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggaggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 218 - DNA, 1166 bp, synthetic construct
  misc_feature 1..1166: note = complementary APOE target region in reporter
  source 1..1166: mol_type = other DNA
ctactcagccccagcggaggtgaaggacgtccttccccaggagccgactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgcgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgca

SEQ ID NO: 219 - DNA, 22 bp, synthetic construct
  misc_feature 1..22: note = prPromotor FW
  source 1..22: mol_type = other DNA
agtaacgccaatagggactttc

SEQ ID NO: 220 - DNA, 22 bp, synthetic construct
  misc_feature 1..22: note = prPromotor RV
  source 1..22: mol_type = other DNA
ggcgtacttggcatatgataca

SEQ ID NO: 221 - DNA, 24 bp, synthetic construct
  misc_feature 1..24: note = pBbPromotor Cy5
  source 1..24: mol_type = other DNA
ttacggtaaactgcccacttggca

SEQ ID NO: 222 - DNA, 16 bp, synthetic construct
  misc_feature 1..16: note = pBbPromotor FAM
  source 1..16: mol_type = other DNA
ctagcgtgtagactct

SEQ ID NO: 223 - DNA, 24 bp, synthetic construct
  misc_feature 1..24: note = prhACTB FW
  source 1..24: mol_type = other DNA
aggtgcacagtaggtctgaacaga

SEQ ID NO: 224 - DNA, 22 bp, synthetic construct
  misc_feature 1..22: note = prhACTB RV
  source 1..22: mol_type = other DNA
tgcaaagaacacggctaagtgt

SEQ ID NO: 225 - DNA, 21 bp, synthetic construct
  misc_feature 1..21: note = prmsACTB FW
  source 1..21: mol_type = other DNA
gacactggcacsgccaacttt

SEQ ID NO: 226 - DNA, 20 bp, synthetic construct
  misc_feature 1..20: note = prmsACTB RV
  source 1..20: mol_type = other DNA
gtgtctacaccgcgggaatg

SEQ ID NO: 227 - DNA, 16 bp, synthetic construct
  misc_feature 1..16: note = pbmsACTB FAM
  source 1..16: mol_type = other DNA
ctagcgtgtagactct

SEQ ID NO: 228 - DNA, 19 bp, synthetic construct
  misc_feature 1..19: note = prAPOE FW
  source 1..19: mol_type = other DNA
ggaactgagggcgctgatg

SEQ ID NO: 229 - DNA, 18 bp, synthetic construct
  misc_feature 1..18: note = prAPOE RV
  source 1..18: mol_type = other DNA
gcctgcagctccttggac

SEQ ID NO: 230 - DNA, 24 bp, synthetic construct
  misc_feature 1..24: note = pbAPOE
  source 1..24: mol_type = other DNA
tcggaactggaggaacaactgacc

SEQ ID NO: 231 - DNA, 180 bp, synthetic construct
  misc_feature 1..180: note = Hybrid intron
  source 1..180: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggta

SEQ ID NO: 232 - DNA, 644 bp, synthetic construct
  misc_feature 1..644: note = P3 promoter
  source 1..644: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 233 - RNA, 16 nt, synthetic construct
  misc_feature 1..16: note = First flanking region
  source 1..16: mol_type = other RNA
cttgggaatggcaagg

SEQ ID NO: 234 - RNA, 16 nt, synthetic construct
  misc_feature 1..16: note = Second flanking region (M is an A or a C and W is an A or a U)
  source 1..16: mol_type = other RNA
mwcttgctatcccaga

SEQ ID NO: 235 - RNA, 86 nt, synthetic construct
  misc_feature 1..86: note = miR-144 wild type
  source 1..86: mol_type = other RNA
tggggccctggctgggatatcatcatatactgtaagtttgcgatgagacactacagtatagatgatgtactagtccgggcaccccc

SEQ ID NO: 236 - RNA, 86 nt, synthetic construct
  misc_feature 1..86: note = miR-144 mutant
  source 1..86: mol_type = other RNA
tggggccctggctgggatttcatcatatactgtaagtttgcgatgagacactacagtatagatgatgtactagtccgggcaccccc

SEQ ID NO: 237 - DNA, 92 bp, synthetic construct
  misc_feature 1..92: note = spacer
  source 1..92: mol_type = other DNA
agctctggagcctgacaaggaggacaggagagatgctgcaagcccaagaagctctctgctcagcctgtcacaacctactgactgccagggca

SEQ ID NO: 238 - DNA, 434 bp, synthetic construct
  misc_feature 1..434: note = miRNA6 in mir-451 cassette
  source 1..434: mol_type = other DNA
atcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggaaggacttgagggactcgaagacgagtccctcaagtcctctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 239 - DNA, 483 bp, synthetic construct
  misc_feature 1..483: note = miRNA6 in mir-451 cassette with mir-144WT
  source 1..483: mol_type = other DNA
atcggcgctatgcttcctgtgcccccagtggggccctggctgggatatcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggaaggacttgagggactcgaagacgagtccctcaagtcctctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 240 - DNA, 483 bp, synthetic construct
  misc_feature 1..483: note = miRNA6 in mir-451 cassette with mir-144A>T
  source 1..483: mol_type = other DNA
atcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggaaggacttgagggactcgaagacgagtccctcaagtcctctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 241 - DNA, 434 bp, synthetic construct
  misc_feature 1..434: note = miRNA1 in mir-451 cassette
  source 1..434: mol_type = other DNA
atcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 242 - DNA, 434 bp, synthetic construct
  misc_feature 1..434: note = miRNA2 in mir-451 cassette
  source 1..434: mol_type = other DNA
atcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggttgcccaactggtcctttttgaaaaggaccagttgggcactcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 243 - DNA, 434 bp, synthetic construct
  misc_feature 1..434: note = miRNA3 in mir-451 cassette
  source 1..434: mol_type = other DNA
atcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtccttggcctttgaaagtccttactttcaaaggccaaggctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 244 - DNA, 434 bp, synthetic construct
  misc_feature 1..434: note = miRNA4 in mir-451 cassette
  source 1..434: mol_type = other DNA
atcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggttggttttctcagcagcagccatgctgctgagaaaaccactcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 245 - DNA, 483 bp, synthetic construct
  misc_feature 1..483: note = Cassette A1, miRNA1 in mir-451 scaffold with mir-144A>T
  source 1..483: mol_type = other DNA
atcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 246 - DNA, 483 bp, synthetic construct
  misc_feature 1..483: note = Cassette A2, miRNA2 in mir-451 scaffold with mir-144A>T
  source 1..483: mol_type = other DNA
atcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggttgcccaactggtcctttttgaaaaggaccagttgggcactcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 247 - DNA, 483 bp, synthetic construct
  misc_feature 1..483: note = Cassette A3, miRNA3 in mir-451 scaffold with mir-144A>T
  source 1..483: mol_type = other DNA
atcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtccttggcctttgaaagtccttactttcaaaggccaaggctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 248 - DNA, 483 bp, synthetic construct
  misc_feature 1..483: note = Cassette A4, miRNA4 in mir-451 scaffold with mir-144A>T
  source 1..483: mol_type = other DNA
atcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggttggttttctcagcagcagccatgctgctgagaaaaccactcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 249 - protein, 317 aa, synthetic construct [sequence omitted]

SEQ ID NO: 250 - protein, 317 aa, synthetic construct [sequence omitted]

SEQ ID NO: 251 - protein, 317 aa, synthetic construct
  REGION 1..317: note = APOE2 ch
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVSLASHLRKLRKRLLRDADDLQKCLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 252 - protein, 317 aa, synthetic construct
  REGION 1..317: note = APOE3 ch
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVSLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 253 - protein, 317 aa, synthetic construct
  REGION 1..317: note = APOE2 Jac
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKCLAVYQAGAREGAERGLSAIRERLGPLVEQGRVRAATVGSLAGQPLQERAQAWGERLRARMEEMGSRTRDRLDEVKEQEAEVRAKLEEQAQQIRLQAEAFQARLKSWFEPLVEDMQRQWAGLVEKVQAAVGTSAAPVPSDNH

SEQ ID NO: 254 - protein, 317 aa, synthetic construct
  REGION 1..317: note = APOE3 Jac
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQEAEVRAKLEEQAQQIRLQAEAFQARLKSWFEPLVEDMQRQWAGLVEKVQAAVGTSAAPVPSDNH

SEQ ID NO: 255 - DNA, 1587 bp, synthetic construct
  misc_feature 1..1587: note = 1
  source 1..1587: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggcacagaaccttcatcttcctgcgaagatgaaggttctgtctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 256 - DNA, 1587 bp, synthetic construct
  misc_feature 1..1587: note = 2
  source 1..1587: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggcacagaaccttcatcttcctgcgaagatgaaggttctgtctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgagcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 257 - DNA, 1587 bp, synthetic construct
  misc_feature 1..1587: note = 3
  source 1..1587: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggcacagaaccttcatcttcctgcgaagatgaaggttctgtctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggaggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 258 - DNA, 1587 bp, synthetic construct
  misc_feature 1..1587: note = 4
  source 1..1587: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggcacagaaccttcatcttcctgcgaagatgaaggttctgtctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 259 - DNA, 1587 bp, synthetic construct
  misc_feature 1..1587: note = 5
  source 1..1587: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggcacagaaccttcatcttcctgcgaagatgaaggttctgtctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgagcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 260 - DNA, 1587 bp, synthetic construct
  misc_feature 1..1587: note = 6
  source 1..1587: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggcacagaaccttcatcttcctgcgaagatgaaggttctgtctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggaggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 261 - DNA, 1587 bp, synthetic construct
  misc_feature 1..1587: note = 7
  source 1..1587: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggaatgtgaccagcaacgcagccctgcgttgctggtcacatctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatgaaagtcctctgggccgctctcctggtgacctttctggccggctgtcaggccaaagtggaacaggccgtggaaaccgagcctgagcctgaactgagacagcagacagagtggcagtccggccagagatgggaacttgccctgggcagattctgggactacctgagatgggtgcagaccctgagcgaacaggtgcaagaggaactgctgagcagccaagtgacccaagagctgagagccctgatggacgagacaatgaaagaactcaaagcttataagagcgagctcgaagagcagctgacccctgtggccgaggaaacaagagccagactgagcaaagagctgcaggccgctcaagctagactgggcgccgatatggaagatgtgtgcggcagactggtgcagtacagaggcgaagtgcaggccatgctgggccagtctaccgaggaactgagagtgcggctggccagccacctgagaaagctgcggaagagactgctgagagatgccgacgacctgcagaaatgcctggccgtgtatcaggctggcgctagagaaggcgctgagagaggactgtctgccatccgggaaagactgggccctctggttgaacagggcagagttagagccgccacagtgggatctcttgcaggccagcctctgcaagaaagagcccaggcttggggcgagagactgagggccagaatggaagagatgggcagcagaaccagagacagactggacgaagtgaaagaacaggtggccgaagtgcgcgccaagctcgaagaacaagcccagcagattcggctgcaggctgaagcctttcaggcccggctgaagagttggttcgagcccctggtggaagatatgcagagacagtgggctggactggtggaaaaggtgcaggctgccgtgggaacatctgctgctccagtgcctagcgacaaccactga

SEQ ID NO: 262 - DNA, 1587 bp, synthetic construct
  misc_feature 1..1587: note = 8
  source 1..1587: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggaatgtgaccagcaacgcagccctgcgttgctggtcacatctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatgaaagtcctctgggccgctctcctggtgacctttctggccggctgtcaggccaaagtggaacaggccgtggaaaccgagcctgagcctgaactgagacagcagacagagtggcagtccggccagagatgggaacttgccctgggcagattctgggactacctgagatgggtgcagaccctgagcgaacaggtgcaagaggaactgctgagcagccaagtgacccaagagctgagagccctgatggacgagacaatgaaagaactcaaagcttataagagcgagctcgaagagcagctgacccctgtggccgaggaaacaagagccagactgagcaaagagctgcaggccgctcaagctagactgggcgccgatatggaagatgtgtgcggcagactggtgcagtacagaggcgaagtgcaggccatgctgggccagtctaccgaggaactgagagtgtctctggccagccacctgagaaagctgcggaagagactgctgagagatgccgacgacctgcagaaatgcctggccgtgtatcaggctggcgctagagaaggcgctgagagaggactgtctgccatccgggaaagactgggccctctggttgaacagggcagagttagagccgccacagtgggatctcttgcaggccagcctctgcaagaaagagcccaggcttggggcgagagactgagggccagaatggaagagatgggcagcagaaccagagacagactggacgaagtgaaagaacaggtggccgaagtgcgcgccaagctcgaagaacaagcccagcagattcggctgcaggctgaagcctttcaggcccggctgaagagttggttcgagcccctggtggaagatatgcagagacagtgggctggactggtggaaaaggtgcaggctgccgtgggaacatctgctgctccagtgcctagcgacaaccactga

SEQ ID NO: 263 - DNA, 1587 bp, synthetic construct
  misc_feature 1..1587: note = 9
  source 1..1587: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggaatgtgaccagcaacgcagccctgcgttgctggtcacatctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatgaaagtcctctgggccgctctcctggtgacctttctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaagaactcaaagcttataagagcgagctcgaagagcagctgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgagcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 264 - DNA, 1587 bp, synthetic construct
  misc_feature 1..1587: note = 10
  source 1..1587: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggaatgtgaccagcaacgcagccctgcgttgctggtcacatctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatgaaagtcctctgggccgctctcctggtgacctttctggccggctgtcaggccaaagtggaacaggccgtggaaaccgagcctgagcctgaactgagacagcagacagagtggcagtccggccagagatgggaacttgccctgggcagattctgggactacctgagatgggtgcagaccctgagcgaacaggtgcaagaggaactgctgagcagccaagtgacccaagagctgagagccctgatggacgagacaatgaaagaactcaaagcttataagagcgagctcgaagagcagctgacccctgtggccgaggaaacaagagccagactgagcaaagagctgcaggccgctcaagctagactgggcgccgatatggaagatgtgtgcggcagactggtgcagtacagaggcgaagtgcaggccatgctgggccagtctaccgaggaactgagagtgcggctggccagccacctgagaaagctgcggaagagactgctgagagatgccgacgacctgcagaaatgcctggccgtgtatcaggctggcgctagagaaggcgctgagagaggactgtctgccatccgggaaagactgggccctctggttgaacagggcagagttagagccgccacagtgggatctcttgcaggccagcctctgcaagaaagagcccaggcttggggcgagagactgagggccagaatggaagagatgggcagcagaaccagagacagactggacgaagtgaaagaacaggaggccgaagtgcgcgccaagctcgaagaacaagcccagcagattcggctgcaggctgaagcctttcaggcccggctgaagagttggttcgagcccctggtggaagatatgcagagacagtgggctggactggtggaaaaggtgcaggctgccgtgggaacatctgctgctccagtgcctagcgacaaccactga

SEQ ID NO: 265 - DNA, 1587 bp, synthetic construct
  misc_feature 1..1587: note = 11
  source 1..1587: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggaatgtgaccagcaacgcagccctgcgttgctggtcacatctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatgaaagtcctctgggccgctctcctggtgacctttctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaagaactcaaagcttataagagcgagctcgaagagcagctgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggaggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 266 - DNA, 1587 bp, synthetic construct
  misc_feature 1..1587: note = 12
  source 1..1587: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggaatgtgaccagcaacgcagccctgcgttgctggtcacatctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatgaaagtcctctgggccgctctcctggtgacctttctggccggctgtcaggccaaagtggaacaggccgtggaaaccgagcctgagcctgaactgagacagcagacagagtggcagtccggccagagatgggaacttgccctgggcagattctgggactacctgagatgggtgcagaccctgagcgaacaggtgcaagaggaactgctgagcagccaagtgacccaagagctgagagccctgatggacgagacaatgaaagaactcaaagcttataagagcgagctcgaagagcagctgacccctgtggccgaggaaacaagagccagactgagcaaagagctgcaggccgctcaagctagactgggcgccgatatggaagatgtgtgcggcagactggtgcagtacagaggcgaagtgcaggccatgctgggccagtctaccgaggaactgagagtgcggctggccagccacctgagaaagctgcggaagagactgctgagagatgccgacgacctgcagaaacgcctggccgtgtatcaggctggcgctagagaaggcgctgagagaggactgtctgccatccgggaaagactgggccctctggttgaacagggcagagttagagccgccacagtgggatctcttgcaggccagcctctgcaagaaagagcccaggcttggggcgagagactgagggccagaatggaagagatgggcagcagaaccagagacagactggacgaagtgaaagaacaggtggccgaagtgcgcgccaagctcgaagaacaagcccagcagattcggctgcaggctgaagcctttcaggcccggctgaagagttggttcgagcccctggtggaagatatgcagagacagtgggctggactggtggaaaaggtgcaggctgccgtgggaacatctgctgctccagtgcctagcgacaaccactga

SEQ ID NO: 267 - DNA, 1587 bp, synthetic construct
  misc_feature 1..1587: note = 13
  source 1..1587: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggaatgtgaccagcaacgcagccctgcgttgctggtcacatctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatgaaagtcctctgggccgctctcctggtgacctttctggccggctgtcaggccaaagtggaacaggccgtggaaaccgagcctgagcctgaactgagacagcagacagagtggcagtccggccagagatgggaacttgccctgggcagattctgggactacctgagatgggtgcagaccctgagcgaacaggtgcaagaggaactgctgagcagccaagtgacccaagagctgagagccctgatggacgagacaatgaaagaactcaaagcttataagagcgagctcgaagagcagctgacccctgtggccgaggaaacaagagccagactgagcaaagagctgcaggccgctcaagctagactgggcgccgatatggaagatgtgtgcggcagactggtgcagtacagaggcgaagtgcaggccatgctgggccagtctaccgaggaactgagagtgtctctggccagccacctgagaaagctgcggaagagactgctgagagatgccgacgacctgcagaaacgcctggccgtgtatcaggctggcgctagagaaggcgctgagagaggactgtctgccatccgggaaagactgggccctctggttgaacagggcagagttagagccgccacagtgggatctcttgcaggccagcctctgcaagaaagagcccaggcttggggcgagagactgagggccagaatggaagagatgggcagcagaaccagagacagactggacgaagtgaaagaacaggtggccgaagtgcgcgccaagctcgaagaacaagcccagcagattcggctgcaggctgaagcctttcaggcccggctgaagagttggttcgagcccctggtggaagatatgcagagacagtgggctggactggtggaaaaggtgcaggctgccgtgggaacatctgctgctccagtgcctagcgacaaccactga

SEQ ID NO: 268 - DNA, 1587 bp, synthetic construct
  misc_feature 1..1587: note = 14
  source 1..1587: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggaatgtgaccagcaacgcagccctgcgttgctggtcacatctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatgaaagtcctctgggccgctctcctggtgacctttctggccggctgtcaggccaaagtggaacaggccgtggaaaccgagcctgagcctgaactgagacagcagacagagtggcagtccggccagagatgggaacttgccctgggcagattctgggactacctgagatgggtgcagaccctgagcgaacaggtgcaagaggaactgctgagcagccaagtgacccaagagctgagagccctgatggacgagacaatgaaagaactcaaagcttataagagcgagctcgaagagcagctgacccctgtggccgaggaaacaagagccagactgagcaaagagctgcaggccgctcaagctagactgggcgccgatatggaagatgtgtgcggcagactggtgcagtacagaggcgaagtgcaggccatgctgggccagtctaccgaggaactgagagtgcggctggccagccacctgagaaagctgcggaagagactgctgagagatgccgacgacctgcagaaacgcctggccgtgtatcaggctggcgctagagaaggcgctgagagaggactgtctgccatccgggaaagactgggccctctggttgaacagggcagagttagagccgccacagtgggatctcttgcaggccagcctctgcaagaaagagcccaggcttggggcgagagactgagggccagaatggaagagatgggcagcagaaccagagacagactggacgaagtgaaagaacaggaggccgaagtgcgcgccaagctcgaagaacaagcccagcagattcggctgcaggctgaagcctttcaggcccggctgaagagttggttcgagcccctggtggaagatatgcagagacagtgggctggactggtggaaaaggtgcaggctgccgtgggaacatctgctgctccagtgcctagcgacaaccactga

SEQ ID NO: 269 - DNA, 1587 bp, synthetic construct
  misc_feature 1..1587: note = 15
  source 1..1587: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtccagttccgatttgtaggcctctacaaatcggaactggctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatgaaagtcctctgggccgctctcctggtgacctttctggccggctgtcaggccaaagtggaacaggccgtggaaaccgagcctgagcctgaactgagacagcagacagagtggcagtccggccagagatgggaacttgccctgggcagattctgggactacctgagatgggtgcagaccctgagcgaacaggtgcaagaggaactgctgagcagccaagtgacccaagagctgagagccctgatggacgagacaatgaaagaactcaaagcttataagagcgagctcgaagagcagctgacccctgtggccgaggaaacaagagccagactgagcaaagagctgcaggccgctcaagctagactgggcgccgatatggaagatgtgtgcggcagactggtgcagtacagaggcgaagtgcaggccatgctgggccagtctaccgaggaactgagagtgtctctggccagccacctgagaaagctgcggaagagactgctgagagatgccgacgacctgcagaaatgcctggccgtgtatcaggctggcgctagagaaggcgctgagagaggactgtctgccatccgggaaagactgggccctctggttgaacagggcagagttagagccgccacagtgggatctcttgcaggccagcctctgcaagaaagagcccaggcttggggcgagagactgagggccagaatggaagagatgggcagcagaaccagagacagactggacgaagtgaaagaacaggtggccgaagtgcgcgccaagctcgaagaacaagcccagcagattcggctgcaggctgaagcctttcaggcccggctgaagagttggttcgagcccctggtggaagatatgcagagacagtgggctggactggtggaaaaggtgcaggctgccgtgggaacatctgctgctccagtgcctagcgacaaccactga

SEQ ID NO: 270 - DNA, 1587 bp, synthetic construct
  misc_feature 1..1587: note = 16
  source 1..1587: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtccagttccgatttgtaggcctctacaaatcggaactggctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatgaaagtcctctgggccgctctcctggtgacctttctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaagaactcaaagcttataagagcgagctcgaagagcagctgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgagcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 271 - DNA, 1587 bp, synthetic construct
  misc_feature 1..1587: note = 17
  source 1..1587: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtccagttccgatttgtaggcctctacaaatcggaactggctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatgaaagtcctctgggccgctctcctggtgacctttctggccggctgtcaggccaaagtggaacaggccgtggaaaccgagcctgagcctgaactgagacagcagacagagtggcagtccggccagagatgggaacttgccctgggcagattctgggactacctgagatgggtgcagaccctgagcgaacaggtgcaagaggaactgctgagcagccaagtgacccaagagctgagagccctgatggacgagacaatgaaagaactcaaagcttataagagcgagctcgaagagcagctgacccctgtggccgaggaaacaagagccagactgagcaaagagctgcaggccgctcaagctagactgggcgccgatatggaagatgtgtgcggcagactggtgcagtacagaggcgaagtgcaggccatgctgggccagtctaccgaggaactgagagtgcggctggccagccacctgagaaagctgcggaagagactgctgagagatgccgacgacctgcagaaatgcctggccgtgtatcaggctggcgctagagaaggcgctgagagaggactgtctgccatccgggaaagactgggccctctggttgaacagggcagagttagagccgccacagtgggatctcttgcaggccagcctctgcaagaaagagcccaggcttggggcgagagactgagggccagaatggaagagatgggcagcagaaccagagacagactggacgaagtgaaagaacaggaggccgaagtgcgcgccaagctcgaagaacaagcccagcagattcggctgcaggctgaagcctttcaggcccggctgaagagttggttcgagcccctggtggaagatatgcagagacagtgggctggactggtggaaaaggtgcaggctgccgtgggaacatctgctgctccagtgcctagcgacaaccactga

SEQ ID NO: 272 - DNA, 1587 bp, synthetic construct
  misc_feature 1..1587: note = 18
  source 1..1587: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtccagttccgatttgtaggcctctacaaatcggaactggctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatgaaagtcctctgggccgctctcctggtgacctttctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaagaactcaaagcttataagagcgagctcgaagagcagctgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggaggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 273 - DNA, 1587 bp, synthetic construct
  misc_feature 1..1587: note = 19
  source 1..1587: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtccagttccgatttgtaggcctctacaaatcggaactggctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatgaaagtcctctgggccgctctcctggtgacctttctggccggctgtcaggccaaagtggaacaggccgtggaaaccgagcctgagcctgaactgagacagcagacagagtggcagtccggccagagatgggaacttgccctgggcagattctgggactacctgagatgggtgcagaccctgagcgaacaggtgcaagaggaactgctgagcagccaagtgacccaagagctgagagccctgatggacgagacaatgaaagaactcaaagcttataagagcgagctcgaagagcagctgacccctgtggccgaggaaacaagagccagactgagcaaagagctgcaggccgctcaagctagactgggcgccgatatggaagatgtgtgcggcagactggtgcagtacagaggcgaagtgcaggccatgctgggccagtctaccgaggaactgagagtgcggctggccagccacctgagaaagctgcggaagagactgctgagagatgccgacgacctgcagaaacgcctggccgtgtatcaggctggcgctagagaaggcgctgagagaggactgtctgccatccgggaaagactgggccctctggttgaacagggcagagttagagccgccacagtgggatctcttgcaggccagcctctgcaagaaagagcccaggcttggggcgagagactgagggccagaatggaagagatgggcagcagaaccagagacagactggacgaagtgaaagaacaggtggccgaagtgcgcgccaagctcgaagaacaagcccagcagattcggctgcaggctgaagcctttcaggcccggctgaagagttggttcgagcccctggtggaagatatgcagagacagtgggctggactggtggaaaaggtgcaggctgccgtgggaacatctgctgctccagtgcctagcgacaaccactga

SEQ ID NO: 274 - DNA, 1587 bp, synthetic construct
  misc_feature 1..1587: note = 20
  source 1..1587: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtccagttccgatttgtaggcctctacaaatcggaactggctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatgaaagtcctctgggccgctctcctggtgacctttctggccggctgtcaggccaaagtggaacaggccgtggaaaccgagcctgagcctgaactgagacagcagacagagtggcagtccggccagagatgggaacttgccctgggcagattctgggactacctgagatgggtgcagaccctgagcgaacaggtgcaagaggaactgctgagcagccaagtgacccaagagctgagagccctgatggacgagacaatgaaagaactcaaagcttataagagcgagctcgaagagcagctgacccctgtggccgaggaaacaagagccagactgagcaaagagctgcaggccgctcaagctagactgggcgccgatatggaagatgtgtgcggcagactggtgcagtacagaggcgaagtgcaggccatgctgggccagtctaccgaggaactgagagtgtctctggccagccacctgagaaagctgcggaagagactgctgagagatgccgacgacctgcagaaacgcctggccgtgtatcaggctggcgctagagaaggcgctgagagaggactgtctgccatccgggaaagactgggccctctggttgaacagggcagagttagagccgccacagtgggatctcttgcaggccagcctctgcaagaaagagcccaggcttggggcgagagactgagggccagaatggaagagatgggcagcagaaccagagacagactggacgaagtgaaagaacaggtggccgaagtgcgcgccaagctcgaagaacaagcccagcagattcggctgcaggctgaagcctttcaggcccggctgaagagttggttcgagcccctggtggaagatatgcagagacagtgggctggactggtggaaaaggtgcaggctgccgtgggaacatctgctgctccagtgcctagcgacaaccactga

SEQ ID NO: 275 - DNA, 1587 bp, synthetic construct
  misc_feature 1..1587: note = 21
  source 1..1587: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtccagttccgatttgtaggcctctacaaatcggaactggctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatgaaagtcctctgggccgctctcctggtgacctttctggccggctgtcaggccaaagtggaacaggccgtggaaaccgagcctgagcctgaactgagacagcagacagagtggcagtccggccagagatgggaacttgccctgggcagattctgggactacctgagatgggtgcagaccctgagcgaacaggtgcaagaggaactgctgagcagccaagtgacccaagagctgagagccctgatggacgagacaatgaaagaactcaaagcttataagagcgagctcgaagagcagctgacccctgtggccgaggaaacaagagccagactgagcaaagagctgcaggccgctcaagctagactgggcgccgatatggaagatgtgtgcggcagactggtgcagtacagaggcgaagtgcaggccatgctgggccagtctaccgaggaactgagagtgcggctggccagccacctgagaaagctgcggaagagactgctgagagatgccgacgacctgcagaaacgcctggccgtgtatcaggctggcgctagagaaggcgctgagagaggactgtctgccatccgggaaagactgggccctctggttgaacagggcagagttagagccgccacagtgggatctcttgcaggccagcctctgcaagaaagagcccaggcttggggcgagagactgagggccagaatggaagagatgggcagcagaaccagagacagactggacgaagtgaaagaacaggaggccgaagtgcgcgccaagctcgaagaacaagcccagcagattcggctgcaggctgaagcctttcaggcccggctgaagagttggttcgagcccctggtggaagatatgcagagacagtgggctggactggtggaaaaggtgcaggctgccgtgggaacatctgctgctccagtgcctagcgacaaccactga

SEQ ID NO: 276 - DNA, 1587 bp, synthetic construct
  misc_feature 1..1587: note = 22
  source 1..1587: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggatcgcgttctcgctgttcgactgaacagcgagaacgcgactcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgagcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 277 - DNA, 24 bp, synthetic construct
  source 1..24: mol_type = other DNA
ttacggtaaactgcccacttggca

SEQ ID NO: 278 - DNA, 26 bp, synthetic construct
  source 1..26: mol_type = other DNA
caggtccagtttttttttttttttvn

SEQ ID NO: 279 - DNA, 20 bp, synthetic construct
  source 1..20: mol_type = other DNA
agcacagaaccttcatcttc

SEQ ID NO: 280 - DNA, 25 bp, synthetic construct
  source 1..25: mol_type = other DNA
gtccagtttttttttttttttgcag

SEQ ID NO: 281 - DNA, 15 bp, synthetic construct
  source 1..15: mol_type = other DNA
gtgaccagcaacgca

SEQ ID NO: 282 - DNA, 26 bp, synthetic construct
  source 1..26: mol_type = other DNA
aggtccagtttttttttttttttagg

SEQ ID NO: 283 - DNA, 21 bp, synthetic construct
  source 1..21: mol_type = other DNA
cagtccagttccgatttgtag

SEQ ID NO: 284 - DNA, 25 bp, synthetic construct
  source 1..25: mol_type = other DNA
ggtccagtttttttttttttttgag

SEQ ID NO: 285 - DNA, 18 bp, synthetic construct
  source 1..18: mol_type = other DNA
aggcgacttgtaacgtca

SEQ ID NO: 286 - DNA, 26 bp, synthetic construct
  source 1..26: mol_type = other DNA
cagtttttttttttttttacgcctct

SEQ ID NO: 287 - DNA, 19 bp, synthetic construct
  source 1..19: mol_type = other DNA
gacccaagagctgagagcc

SEQ ID NO: 288 - DNA, 22 bp, synthetic construct
  source 1..22: mol_type = other DNA
ctgcagctctttgctcagtctg

SEQ ID NO: 289 - DNA, 19 bp, synthetic construct
  source 1..19: mol_type = other DNA
ggaactgagggcgctgatg

SEQ ID NO: 290 - DNA, 18 bp, synthetic construct
  source 1..18: mol_type = other DNA
gcctgcagctccttggac

SEQ ID NO: 291 - DNA, 23 bp, synthetic construct
  source 1..23: mol_type = other DNA
aagaactcaaagcttataagagc

SEQ ID NO: 292 - DNA, 16 bp, synthetic construct
  source 1..16: mol_type = other DNA
cgcgttctcgctgttc

SEQ ID NO: 293 - DNA, 26 bp, synthetic construct
  source 1..26: mol_type = other DNA
ggtccagtttttttttttttttagtc

SEQ ID NO: 294 - RNA, 22 nt, synthetic construct
  source 1..22: mol_type = other RNA
gcgacttgtaacgtcagaggcg

SEQ ID NO: 295 - DNA, 39 bp, synthetic construct
  source 1..39: mol_type = other DNA
cacagaaccttcatcttcctgcgaagatgaaggttctgt

SEQ ID NO: 296 - DNA, 39 bp, synthetic construct
  source 1..39: mol_type = other DNA
aatgtgaccagcaacgcagccctgcgttgctggtcacat

SEQ ID NO: 297 - DNA, 39 bp, synthetic construct
  source 1..39: mol_type = other DNA
tccagttccgatttgtaggcctctacaaatcggaactgg

SEQ ID NO: 298 - DNA, 39 bp, synthetic construct
  source 1..39: mol_type = other DNA
gcgacttgtaacgtcagaggcgtctgacgttacaagtcg

SEQ ID NO: 299 - DNA, 38 bp, synthetic construct
  source 1..38: mol_type = other DNA
atcgcgttctcgctgttcgactgaacagcgagaacgcg